(19) United States
(12) Patent Application Publication
(10) Pub. No.: US 2026/0092266 A1
(43) Pub. Date: Apr. 2, 2026

(54) CRISPR ENZYMES AND SYSTEMS

(71) Applicants: The Broad Institute, Inc., Cambridge, MA (US); Massachusetts Institute of Technology, Cambridge, MA (US)

(72) Inventors: Feng Zhang, Cambridge, MA (US); Han Altae-Tran, Cambridge, MA (US); Soumya Kannan, Cambridge, MA (US)

(21) Appl. No.: 19/388,533

(22) Filed: Nov. 13, 2025

Related U.S. Application Data
(63) Continuation of application No. PCT/US2024/029759, filed on May 16, 2024.
(60) Provisional application No. 63/502,542, filed on May 16, 2023.
(60) Provisional application No. 63/512,416, filed on Jul. 7, 2023.
(60) Provisional application No. 63/512,455, filed on Jul. 7, 2023.

(51) Int. Cl. C12N 9/22; C12N 15/11; C12N 15/82; C12N 15/86; C12N 15/90

(52) U.S. Cl. CPC C12N 9/222; C12N 15/111; C12N 15/8203; C12N 15/86; C12N 15/902; C07K 2319/09; C12N 2310/20

(57) ABSTRACT

Described herein are engineered, non-naturally occurring systems and compositions comprising multimeric CRISPR-Cas complexes comprising a β-CASP polypeptide, a plurality of Cas polypeptides, and a guide molecule, packaging and delivery systems thereof, and methods of use thereof, for modifying target polynucleotides. In addition, described herein are engineered, non-naturally occurring systems and compositions comprising a class of small Cas proteins (Type II-B, II-C, and II-D Cas proteins) and methods of modifying target sequences using the Type II-B, II-C, II-D Cas proteins and systems thereof.

### CROSS-REFERENCE TO RELATED APPLICATIONS

This application is a Continuation application of International Patent Application No. PCT/US2024/029759, filed May 16, 2024, which claims the benefit of U.S. Provisional Application Nos. 63/502,542, filed May 16, 2023, U.S. 63/512,416, filed Jul. 7, 2023, and U.S. 63/512,455, filed Jul. 7, 2023. The entire contents of the above-identified applications are hereby fully incorporated herein by reference.

### STATEMENT REGARDING FEDERALLY SPONSORED RESEARCH

This invention was made with government support under Grant No. (s) HG009761 and HL141201 awarded by the National Institutes of Health. The government has certain rights in the invention.

### REFERENCE TO AN ELECTRONIC SEQUENCE LISTING

The contents of the electronic sequence listing (“BROD-5845US_ST26.xml”; Size is 96,007,958 bytes, and created on Oct. 29, 2025) is herein incorporated by reference in its entirety.

### TECHNICAL FIELD

The subject matter disclosed herein is generally directed to novel Clustered Regularly Interspaced Short Palindromic Repeat (CRISPR) systems, methods thereof, and compositions thereof, the CRISPR systems comprising novel multimeric CRISPR-associated (Cas) enzymes used for the control of gene expression involving sequence targeting, such as perturbation of gene transcripts or nucleic acid editing, or for the rapid detection of nucleic acids, that may use particle or vector systems to deliver the CRISPR-Cas systems and components thereof.

### BACKGROUND

Recent advances in genome sequencing techniques and analysis methods have significantly accelerated the ability to catalog and map genetic factors associated with diverse functions and diseases. Precise genome targeting technologies are needed to enable systematic reverse engineering of causal genetic variations by allowing selective perturbation of individual genetic elements and advancing synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ novel strategies and molecular mechanisms and are affordable, easy to set up, scalable, and amenable to targeting multiple positions within the eukaryotic genome and transcriptome. This would provide a significant resource for new genome engineering and biotechnology applications.

The CRISPR-Cas bacterial and archaeal adaptive immunity systems show extreme protein composition and genomic loci architecture diversity. The CRISPR-Cas system loci have more than 50 gene families, and there are no strictly universal genes, indicating fast evolution and extreme diversity of loci architecture. So far, adopting a multi-pronged approach, there is comprehensive cas gene identification of about 395 profiles for 93 Cas proteins.

Citation or identification of any document in this application is not an admission that such a document is available as prior art to the present invention.

### SUMMARY OF THE INVENTION

In an embodiment, the present invention provides a non-naturally occurring, engineered composition comprising: a β-CASP polypeptide; and a plurality of Cas polypeptides, wherein (a) and (b) are capable of forming a non-naturally occurring, engineered multimeric CRISPR-Cas complex in the presence of a guide molecule, and wherein the guide molecule is capable of directing sequence-specific binding of the non-naturally occurring, engineered multimeric CRISPR-Cas complex to a target sequence in a target polynucleotide. In an embodiment, the β-CASP polypeptide comprises an N-terminal β-CASP domain and a C-terminal adapter domain. In an embodiment, the C-terminal adapter domain comprises an α-helical domain having homology to the C-terminus of a Cas10 protein. In an embodiment, the β-CASP polypeptide comprises a plurality of residues capable of coordinating with Zn²⁺ ions.

In an embodiment, the plurality of Cas polypeptides comprise a Cas5 family polypeptide, a Cas7 family polypeptide, and optionally a Cas6 family polypeptide. In an embodiment, the Cas5 family polypeptide is a Type III Csx10 polypeptide, a homolog thereof, or an ortholog thereof; wherein the Cas7 family polypeptide is a Type III Csm3 polypeptide, a homolog thereof, or an ortholog thereof; and/or wherein the Cas6 family polypeptide is a Type III Cas6 polypeptide, a homolog thereof, or an ortholog thereof. In an embodiment, one or more of the β-CASP polypeptide and/or the Cas polypeptides has catalytic activity; wherein one or more of the β-CASP polypeptide and/or the Cas polypeptides lacks catalytic activity; and/or wherein one or more of the β-CASP polypeptide and/or the Cas polypeptides is or is engineered to have nickase activity. In an embodiment, the β-CASP polypeptide has catalytic activity. In an embodiment, the Cas7 family polypeptide lacks catalytic activity. In an embodiment, the catalytic activity is RNAse activity.

In an embodiment, one or more of the β-CASP polypeptide and/or the Cas polypeptides further comprise one or more additional modifications that increase nuclease efficiency, target polynucleotide binding efficiency, or reduce off-target nuclease activity. In an embodiment, the β-CASP polypeptide and/or one or more of the Cas polypeptides is/are further linked to or otherwise capable of associating with a heterologous functional domain. In an embodiment, the heterologous functional domain is a nucleotide deaminase, a transposase, a reverse transcriptase, a recombinase, a methylase, a demethylase, an acetylase, or a deacetylase.

In an embodiment, the β-CASP polypeptide and/or one or more of the Cas polypeptides is/are derived from one or more bacteria and/or archaea. In an embodiment, the one or more bacteria each independently belong to the phylum selected from the group consisting of Bacillota; and DTHG01000077 4 candidate division White Oak River group 3 (WOR-3); the one or more archaea each independently belong to the phylum selected from the group consisting of MBU4492343 1/HEQ78297 1/Euryarchaeota; RLE40065.1 Candidatus Woesearchaeota; NHI92075 1 Candidatus Lokiarchaeota; and PKP54316 1 Candidatus Altiarchaeales archaeon; and/or the one or more archaea each independently belong to the order selected from the group consisting of: PXF52022 1/RJS85311 1/Methanophagales; and MCD4797691.1/CAG0966219 1/RLG33181 1 Methanosarcinales. In an embodiment, the one or more bacteria each independently belong to the Staphylococcus genus, and optionally one of the bacteria is 6NBT Staphylococcus epidermis; the one or more archaea each independently belong to the family MCG2727882 1 Candidatus Methanoperedenaceae, and optionally one of the archaea is WP 0972978485 1 Candidatus Methanoperedens sp BLZ2; the one or more archaea each independently belong to a genus selected from the group consisting of: WP 0972978485 1 Candidatus Methanoperedens; and/or the one or more archaea each independently belong to a species selected from the group consisting of: 4QTS (Csm3) Mathanocaldococcus jannaschii; and WP 012965105 1 Ferroglobus placidus, and optionally one of the archaea is WP 012965105 1 Ferroglobus placidus DSM 10642. In an embodiment, each of the β-CASP polypeptides and one or more Cas polypeptides are derived from the same species from one or more species. In an embodiment, the β-CASP polypeptide is derived from a first species, and the one or more Cas polypeptides are derived from a second species different from the first species.

In an embodiment, the composition further comprises one or more guide molecules, wherein the guide molecules comprise a guide sequence capable of hybridizing to a target sequence of the target molecule. The composition is optionally in the form of the non-naturally occurring, engineered multimeric CRISPR-Cas complex. In an embodiment, at least one guide molecule is a crRNA comprising a spacer sequence flanked on the 5′ and 3′ ends by direct repeat sequences.

In an embodiment, the present invention provides a nucleic acid molecule comprising a nucleotide sequence encoding one or more components of any one of the compositions of the present invention.

In an embodiment, the present invention provides a vector comprising a polynucleotide comprising one or more of any one of the nucleic acid molecules of the present invention. The vector is a viral vector.

In an embodiment, the present invention provides a delivery vehicle comprising one or more components of any one of the compositions of the present invention, any one of the non-naturally occurring, engineered multimeric CRISPR-Cas complexes of the present invention, any one of the nucleic acid molecules of the present invention, any one of the vectors of the present invention, or any combination thereof. In an embodiment, the delivery vehicle is a lipid nanoparticle, a viral capsid, an engineered retroelement vector, a polynucleotide-based nanostructure, or an extracellular contractile injection system.

In an embodiment, the present invention provides an engineered cell comprising the one or more components of any one of the compositions of the present invention, any one of the non-naturally occurring, engineered multimeric CRISPR-Cas complexes of the present invention, any one of the nucleic acid molecules of the present invention, any one of the vectors of the present invention, or any combination thereof. In an embodiment, the engineered cell is an engineered eukaryotic or engineered prokaryotic cell.

In an embodiment, the present invention provides an organism comprising any one of the engineered cells of the present invention. In an embodiment, the organism is an animal or a plant.

In an embodiment, the present invention provides a pharmaceutical composition for the treatment of a disease or disorder, comprising one or more components of any one of the compositions of the present invention, any one of the non-naturally occurring, engineered multimeric CRISPR-Cas complexes of the present invention, any one of the nucleic acid molecules the present invention, any one of the vectors of the present invention, any one of the delivery particles of the present invention, any one of the engineered cells of the present invention, or any combination thereof.

In an embodiment, the present invention provides a method of modifying a target polynucleotide, the method comprising contacting a sample comprising a target polynucleotide with one or more components of any one of the compositions of the present invention, any one of the non-naturally occurring, engineered multimeric CRISPR-Cas complexes of the present invention, any one of the nucleic acid molecules of the present invention, any one of the vectors of the present invention, any one of the delivery particles of the present invention, any one of the engineered cells of the present invention, any one of the pharmaceutical compositions of the present invention, or any combination thereof. In an embodiment, contacting results in modification of a gene product or modification of the amount or expression of a gene product. In an embodiment, the target polynucleotide is a disease-or disorder-associated target polynucleotide.

In an embodiment, the techniques described herein relate to a non-naturally occurring or engineered nucleic acid-targeting composition including a Cas polypeptide including a RuvC domain and an HNH domain, wherein the Cas polypeptide is less than 850 amino acids in size; and a nucleic acid guide molecule capable of forming a complex with the Cas polypeptide and directing sequence-specific binding of the complex to a target sequence in a target polynucleotide, wherein the Cas polypeptide is a Type II-B Cas polypeptide selected from the group consisting of (SEQ ID NO: 189-269), or wherein the Cas polypeptide is a Type II-C Cas polypeptide selected from the group consisting of (SEQ ID NO: 4583-8895).

In an embodiment, the techniques described herein relate to a composition, wherein the composition includes two or more nucleic acid guide molecules capable of hybridizing to two different target sequences or different regions of a target sequence.

In an embodiment, the techniques described herein relate to a composition, wherein the nucleic acid guide molecule is capable of hybridizing to one or more target sequences in a prokaryotic cell.

In an embodiment, the techniques described herein relate to a composition, wherein the nucleic acid guide molecule is capable of hybridizing to one or more target sequences in a eukaryotic cell.

In an embodiment, the techniques described herein relate to a composition, wherein the Cas polypeptide includes one or more nuclear localization signals.

In an embodiment, the techniques described herein relate to a composition, wherein the Cas polypeptide includes two or more nuclear localization signals.

In an embodiment, the techniques described herein relate to a composition, wherein the Cas polypeptide includes one or more nuclear export signals.

In an embodiment, the techniques described herein relate to a composition, wherein the Cas polypeptide is catalytically inactive.

In an embodiment, the techniques described herein relate to a composition, wherein the Cas polypeptide is a nickase.

In an embodiment, the techniques described herein relate to a composition, wherein the Cas polypeptide is associated with one or more functional domains.

In an embodiment, the techniques described herein relate to a composition, wherein the one or more functional domains includes one or more heterologous functional domains.

In an embodiment, the techniques described herein relate to a composition, wherein the one or more functional domains cleaves the target sequence.

In an embodiment, the techniques described herein relate to a composition, wherein the one or more functional domains modifies transcription or translation of the target sequence.

In an embodiment, the techniques described herein relate to a composition, wherein the one or more functional domains includes one or more transcriptional activation domains.

In an embodiment, the techniques described herein relate to a composition, wherein the one or more transcriptional activation domains includes VP64.

In an embodiment, the techniques described herein relate to a composition, wherein the one or more functional domains includes one or more transcriptional repression domains.

In an embodiment, the techniques described herein relate to a composition, wherein the one or more transcriptional repression domains includes a KRAB domain or a SID domain.

In an embodiment, the techniques described herein relate to a composition, wherein the one or more functional domains includes one or more nuclease domains.

In an embodiment, the techniques described herein relate to a composition, wherein the one or more nuclease domains includes Fok1.

In an embodiment, the techniques described herein relate to a composition, wherein the one or more functional domains have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity.

In an embodiment, the techniques described herein relate to a composition, further including a recombination template.

In an embodiment, the techniques described herein relate to a composition, wherein the recombination template is inserted by homology-directed repair (HDR).

In an embodiment, the techniques described herein relate to a composition, further including a tracrRNA.

In an embodiment, the techniques described herein relate to a composition, wherein the Cas polypeptide is a chimeric protein including a first fragment from a first Cas polypeptide and a second fragment from a second Cas polypeptide.

In an embodiment, the techniques described herein relate to a composition, further including a nucleotide deaminase or a catalytic domain thereof.

In an embodiment, the techniques described herein relate to a composition, wherein the nucleotide deaminase is an adenosine deaminase.

In an embodiment, the techniques described herein relate to a composition, wherein the nucleotide deaminase is a cytidine deaminase.

In an embodiment, the techniques described herein relate to a composition, wherein the nucleotide deaminase or catalytic domain thereof is covalently or non-covalently linked to the Cas polypeptide or the nucleic acid guide molecule, or is adapted to link thereof after delivered to a cell.

In an embodiment, the techniques described herein relate to a composition, wherein the nucleotide deaminase or catalytic domain thereof has been modified to increase its activity against a DNA-RNA heteroduplex.

In an embodiment, the techniques described herein relate to a composition, wherein the nucleotide deaminase or catalytic domain thereof has been modified to reduce off-target effects.

In an embodiment, the techniques described herein relate to a composition, wherein the composition is capable of modifying one or more nucleotides in the target sequence.

In an embodiment, the techniques described herein relate to a composition, wherein modification of the one or more nucleotides in the target sequence remedies a disease caused by a G→A or C→T point mutation or a pathogenic SNP.

In an embodiment, the techniques described herein relate to a composition, wherein the disease is cancer, hemophilia, beta-thalassemia, Marfan syndrome, or Wiskott-Aldrich syndrome.

In an embodiment, the techniques described herein relate to a composition, wherein modification of the one or more nucleotides in the target sequence remedies a disease caused by a T→C or A→G point mutation or a pathogenic SNP.

In an embodiment, the techniques described herein relate to a composition, wherein modification of the one or more nucleotides at the target sequence inactivates a gene.

In an embodiment, the techniques described herein relate to a composition, wherein modification of the one or more nucleotides modifies gene product encoded at the target sequence or expression of the gene product.

In an embodiment, the techniques described herein relate to a composition, further including a reverse transcriptase or functional fragment thereof.

In an embodiment, the techniques described herein relate to a non-naturally occurring or engineered nucleic acid targeting composition including one or more polynucleotide sequences encoding: a Cas polypeptide including a RuvC domain and an HNH domain, wherein the Cas polypeptide is less than 900 amino acids in size; and a nucleic acid guide molecule capable of forming a complex with the Cas polypeptide and directing sequence-specific binding of the complex to a target sequence in a target polynucleotide, wherein the one or more polynucleotide sequences encode a Type II-B Cas polypeptide and are selected from the group consisting of (SEQ ID NO: 108-188), or wherein the one or more polynucleotide sequences encode a Type II-C Cas polypeptide and are selected from the group consisting of (SEQ ID NO: 270-4582).

In an embodiment, the techniques described herein relate to a composition, wherein the one or more polynucleotide sequences are codon optimized to express in a eukaryote.

In an embodiment, the techniques described herein relate to a composition, wherein the one or more polynucleotide sequences is mRNA.

In an embodiment, the techniques described herein relate to a composition, wherein the one or more polynucleotide sequences further encode a reverse transcriptase or functional fragment thereof.

In an embodiment, the techniques described herein relate to a vector system including the one or more polynucleotide sequences described herein.

In an embodiment, the techniques described herein relate to a vector system, including: a first regulatory element operably linked to the polynucleotide sequence encoding the Cas polypeptide; and a second regulatory element operably linked to the polynucleotide sequence encoding the nucleic acid guide molecule.

In an embodiment, the techniques described herein relate to a vector system, wherein the first and/or second regulatory element is a promoter.

In an embodiment, the techniques described herein relate to a vector system, wherein the promoter is a minimal promoter.

In an embodiment, the techniques described herein relate to a vector system, wherein the minimal promoter is Mecp2 promoter, tRNA promoter, or U6 promoter.

In an embodiment, the techniques described herein relate to a vector system, which is included in a single vector.

In an embodiment, the techniques described herein relate to a vector system, wherein the one or more vectors includes viral vectors.

In an embodiment, the techniques described herein relate to a vector system, wherein the one or more vectors includes retroviral, lentiviral, adenoviral, adeno-associated, or herpes simplex viral vectors.

In an embodiment, the techniques described herein relate to a delivery system including any system described herein and a delivery vehicle.

In an embodiment, the techniques described herein relate to a delivery system, wherein the delivery vehicle includes lipids, sugars, metals, proteins, liposomes, nanoparticles, exosomes, microvesicles, nucleic acid nanoassemblies, a gene gun, an implantable device, or a vector system.

In an embodiment, the techniques described herein relate to a delivery system, wherein the delivery vehicle includes ribonucleoproteins.

In an embodiment, the techniques described herein relate to a cell including any composition described herein.

In an embodiment, the techniques described herein relate to a cell, wherein the cell is a eukaryotic cell, a human or non-human animal cell, a therapeutic T cell, antibody-producing B-cell, a stem cell, or a plant cell.

In an embodiment, the techniques described herein relate to a tissue, organ, or organism including any cell including any composition described herein.

In an embodiment, the techniques described herein relate to a cell product from any cell including any composition described herein.

In an embodiment, the techniques described herein relate to a method of modifying one or more target sequences, the method comprising contacting the one or more target sequences with a composition of any of those described herein.

In an embodiment, the techniques described herein relate to a method, wherein the composition further includes a recombination template, and wherein modifying the one or more target sequences includes insertion of the recombination template or a portion thereof.

In an embodiment, the techniques described herein relate to a method, wherein the one or more target sequences is in a prokaryotic cell.

In an embodiment, the techniques described herein relate to a method, wherein the one or more target sequences is in a eukaryotic cell.

In an embodiment, the techniques described herein relate to a method, wherein the one or more target sequences is included in a nucleic acid molecule in vitro.

In an embodiment, the techniques described herein relate to a cell obtained from any method described herein.

In an embodiment, the techniques described herein relate to a cell or progeny thereof, wherein the cell is a eukaryotic cell, a human or non-human animal cell, a therapeutic T cell, antibody-producing B-cell, a stem cell, or a plant cell.

In an embodiment, the techniques described herein relate to a non-human animal or plant including the modified cell or progeny thereof as described herein.

In an embodiment, the techniques described herein relate to a modified cell or progeny thereof as described herein for use in therapy.

In an embodiment, the techniques described herein relate to a method of treating a disease, disorder, or infection comprising administering an effective amount of the composition of any one of those described herein in a subject in need thereof.

In an embodiment, the techniques described herein relate to a method of identifying a trait of interest in an organism where the trait of interest is encoded by one or more target polynucleotides, the method comprising contacting the organism or a sample therefrom comprising polynucleotides with non-naturally occurring or engineered nucleic acid targeting composition of any one of those described herein, wherein the composition is directed to the one or more target polynucleotides by the nucleic acid guide molecule, whereby one or more target polynucleotides, and thereby one or more traits, are identified.

In an embodiment, the techniques described herein relate to a method, wherein the one or more target polynucleotides are modified by the non-naturally occurring or engineered nucleic acid targeting composition.

In an embodiment, the techniques described herein relate to a method, wherein the method is performed in vitro, in situ, ex vivo, or in vivo.

In an embodiment, the techniques described herein relate to a method, wherein the organism is a plant, non-human animal, or human.

In an embodiment, the techniques described herein relate to a method of producing a plant having a modified trait of interest encoded by a gene of interest, the method comprises contacting a plant cell with a composition of any one of those described herein, thereby either modifying or introducing the gene of interest, and regenerating a plant from the plant cell.

In an embodiment, the techniques described herein relate to an engineered nucleic acid targeting composition including: a Cas polypeptide including a RuvC domain and an HNH domain, wherein the Cas protein is about 950 amino acids or less in size; and a nucleic acid guide molecule capable of forming a complex with the Cas polypeptide and directing sequence-specific binding of the complex to a target sequence in a target polynucleotide, wherein the Cas polypeptide is selected from the group consisting of (SEQ ID NO: 8899-9520).

In an embodiment, the techniques described herein relate to an engineered nucleic acid targeting system, wherein the Cas polypeptide is less than or equal to 780 amino acids in size.

In an embodiment, the techniques described herein relate to an engineered nucleic acid targeting system, wherein the Cas polypeptide has no association with Cas1, Cas2, Cas4, or Csn2.

In an embodiment, the techniques described herein relate to an engineered nucleic acid targeting system, wherein the Cas polypeptide is capable of forming a complex with two or more nucleic guide molecules, wherein each guide molecule is capable of sequence-specific binding of a target nucleic acid sequence, wherein each target sequence is different.

In an embodiment, the techniques described herein relate to an engineered nucleic acid targeting system, wherein the target sequences are on the same or are on different target polynucleotides.

In an embodiment, the techniques described herein relate to an engineered nucleic acid targeting system, wherein the guide molecule or the two or more guide molecules are capable of sequence-specific binding a target sequence in vitro, in situ, ex vivo, or in vivo.

In an embodiment, the techniques described herein relate to an engineered nucleic acid targeting system, wherein the guide molecule or the two or more guide molecules are capable of sequence-specific binding a target sequence in a prokaryotic cell, eukaryotic cell, a virus, or a combination thereof.

In an embodiment, the techniques described herein relate to an engineered nucleic acid targeting system, wherein the Cas protein is operably coupled to one or more nuclear localization signals.

In an embodiment, the techniques described herein relate to an engineered nucleic acid targeting system, wherein the Cas protein is operably coupled to one or more nuclear export signals.

In an embodiment, the techniques described herein relate to an engineered nucleic acid targeting system, wherein the Cas protein lacks one or more catalytic activities.

In an embodiment, the techniques described herein relate to an engineered nucleic acid targeting system, wherein the Cas protein lacks nuclease activity.

In an embodiment, the techniques described herein relate to an engineered nucleic acid targeting system, wherein the Cas protein is a nickase.

In an embodiment, the techniques described herein relate to an engineered nucleic acid targeting system, wherein the Cas protein is operably coupled to or associated with one or more functional domains.

In an embodiment, the techniques described herein relate to an engineered nucleic acid targeting system, wherein the one or more functional domains is/are one or more heterologous functional domains.

In an embodiment, the techniques described herein relate to an engineered nucleic acid targeting system, wherein the one or more functional domains has one or more activities selected from deaminase activity, methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, nucleic acid binding activity, transposition activity, reverse transcription activity, or a combination thereof.

In an embodiment, the techniques described herein relate to an engineered nucleic acid targeting system, wherein the one or more functional domains is capable of cleaving the target polynucleotide.

In an embodiment, the techniques described herein relate to an engineered nucleic acid targeting system, wherein the one or more functional domains is capable of modifying transcription or translation of the target polynucleotide.

In an embodiment, the techniques described herein relate to an engineered nucleic acid targeting system, further including a recombination template.

In an embodiment, the techniques described herein relate to an engineered nucleic acid targeting system, wherein the recombination template is operably coupled to, complexed with, or is associated with the Cas protein, the nucleic acid guide molecule, or both.

In an embodiment, the techniques described herein relate to an engineered nucleic acid targeting system, wherein the recombination template is a homology-directed repair (HDR) recombination template.

In an embodiment, the techniques described herein relate to an engineered nucleic acid targeting system, wherein the nucleic acid targeting system includes a tracrRNA.

In an embodiment, the techniques described herein relate to an engineered nucleic acid targeting system, wherein the Cas protein is a chimeric protein including a first polypeptide fragment from a first Cas protein and a second polypeptide fragment from a second Cas protein.

In an embodiment, the techniques described herein relate to an engineered nucleic acid targeting system, further including a deaminase or catalytic domain thereof.

In an embodiment, the techniques described herein relate to an engineered nucleic acid targeting system, wherein the deaminase is an adenosine deaminase or a cytidine deaminase.

In an embodiment, the techniques described herein relate to an engineered nucleic acid targeting system, wherein the deaminase or catalytic domain thereof is operably coupled to, complexed with, or otherwise associated with the Cas protein, a guide molecule, or both or is capable of operably coupling to, complexing with, or otherwise associated with the Cas protein, a guide molecule, or both after delivery to a cell.

In an embodiment, the techniques described herein relate to an engineered nucleic acid targeting system, wherein the nucleotide deaminase or catalytic domain thereof has been modified to increase its activity against a DNA-RNA heteroduplex, to reduce off-target effects, or both.

In an embodiment, the techniques described herein relate to an engineered nucleic acid targeting system, further including a reverse transcriptase or functional domain thereof, wherein the reverse transcriptase or functional domain thereof is optionally operably coupled to, is capable of complexing with, or is otherwise associated with the Cas protein, the guide molecule, or both.

In an embodiment, the techniques described herein relate to an engineered nucleic acid targeting system, further including one or more nucleic acid guide molecules, wherein each of the one or more nucleic acid guide molecules is capable of capable of forming a complex or is complexed with the Cas protein, and wherein each of the one or more nucleic acid guide molecules is capable of sequence specific binding of a target sequence in a target polynucleotide.

In an embodiment, the techniques described herein relate to an engineered nucleic acid targeting system, wherein the engineered nucleic acid targeting system is capable of modifying a sequence of the target polynucleotide.

In an embodiment, the techniques described herein relate to an engineered nucleic acid targeting system, wherein the modification is (a) insertion of one or more polynucleotides; (b) deletion of one or more polynucleotides; (c) conversion of a C•G base pair to a T•A base pair; (d) conversion of an A•T base pair to a G•C base pair; or (e) a combination thereof.

In an embodiment, the techniques described herein relate to an engineered nucleic acid targeting system, wherein the modification alters a transcription product of the target polynucleotide, a translation product of the target polynucleotide, or both.

In an embodiment, the techniques described herein relate to an engineered nucleic acid targeting system, wherein the modification alters transcription, translation, or both of the target polynucleotide.

In an embodiment, the techniques described herein relate to a polynucleotide including one or more nucleic acid sequences that encode one or more components of the engineered nucleic acid system of any one of those described herein.

In an embodiment, the techniques described herein relate to a polynucleotide, wherein the polynucleotide is codon optimized for expression in a eukaryotic cell.

In an embodiment, the techniques described herein relate to a polynucleotide, wherein the eukaryotic cell is a human cell or a non-human animal cell.

In an embodiment, the techniques described herein relate to a vector system including: one or more vectors including one or more polynucleotides of any of those described herein, and optionally one or more regulatory elements operably coupled to one or more polynucleotides.

In an embodiment, the techniques described herein relate to a vector system, wherein the one or more of the one or more vectors are viral vectors.

In an embodiment, the techniques described herein relate to a vector system, wherein the viral vector(s) is/are a retroviral vector(s), lentiviral vector(s), adenoviral vector(s), adeno-associated viral vector(s), herpes simplex viral vector(s), or a combination thereof.

In an embodiment, the techniques described herein relate to a delivery composition including: (a) an engineered nucleic acid-targeting system of any one of those described herein; (b) one or more polynucleotides of any one of those described herein; (c) one or more vector systems of any one of those described herein; or (d) a combination thereof; and (e) a delivery vehicle, wherein a, b, c, d, or e, are associated with or operably coupled to the delivery vehicle.

In an embodiment, the techniques described herein relate to a cell or progeny thereof including (a) an engineered nucleic acid-targeting system of any one of those described herein; (b) one or more polynucleotides of any one of those described herein; (c) one or more vector systems of any one of those described herein; (d) a delivery formulation of any one of those described herein; (e) one or more polynucleotide modifications produced by an engineered nucleic acid-targeting system of any one of those described herein; or (f) a combination thereof.

In an embodiment, the techniques described herein relate to a cell or progeny thereof as described herein, wherein the cell or progeny thereof is a prokaryotic or eukaryotic cell.

In an embodiment, the techniques described herein relate to a tissue, organ, or organism including: a cell or progeny thereof as described herein or a population thereof.

In an embodiment, the techniques described herein relate to a pharmaceutical formulation including: (a) an engineered nucleic acid-targeting system of any one of those described herein; (b) one or more polynucleotides of any one of those described herein; (c) one or more vector systems of any one of those described herein; (d) a delivery formulation of any one of those described herein; (e) a cell or progeny thereof as described herein; (f) a tissue, an organ, or an organism of any one of those described herein; or (g) a combination thereof; and (h) a pharmaceutically acceptable carrier.

In an embodiment, the techniques described herein relate to a product produced by a cell or progeny thereof as described herein or a population thereof, a tissue, organ, or organism as described herein, or both.

In an embodiment, the techniques described herein relate to a method of modifying one or more target polynucleotides, the method comprising contacting the one or more target polynucleotides with an engineered nucleic acid targeting system of any one of those described herein, wherein the engineered nucleic acid targeting system is directed to the one or more target sequences by the guide nucleic acid guide molecule(s) of the engineered nucleic acid targeting system, whereby one or more target polynucleotides is/are modified.

In an embodiment, the techniques described herein relate to a method, wherein the modification includes: (a) insertion of one or more polynucleotides; (b) deletion of one or more polynucleotides; (c) conversion of a C•G base pair to a T•A base pair; (d) conversion of an A•T base pair to a G•C base pair; or (e) a combination thereof.

In an embodiment, the techniques described herein relate to a method, wherein contacting occurs in vitro, in situ, ex vivo, or in vivo.

In an embodiment, the techniques described herein relate to a method, wherein contacting occurs within a cell.

In an embodiment, the techniques described herein relate to a modified polynucleotide or modified cell or progeny thereof produced from a method as described herein.

In an embodiment, the techniques described herein relate to a modified cell or progeny thereof as described herein, wherein the cell is a eukaryotic cell or progeny thereof.

In an embodiment, the techniques described herein relate to a modified cell or progeny thereof as described herein, wherein the cell or progeny thereof is a human cell or progeny thereof or a non-human animal cell or progeny thereof.

In an embodiment, the techniques described herein relate to a modified cell or progeny thereof as described herein, wherein the cell or progeny thereof is a plant cell.

In an embodiment, the techniques described herein relate to a method of treating and/or preventing a disease, condition, or a symptom thereof in a subject in need thereof, the method including; (a) administering to the subject in need thereof; (b) an engineered nucleic acid targeting system of any one of those described herein; (c) one or more polynucleotides of any one of those described herein; (d) one or more vector systems of any one of those described herein; (e) a delivery formulation of any one of those described herein; (f) a cell or progeny thereof as in any one of those described herein; (g) a tissue, an organ, or an organism of any one of those described herein; (h) a pharmaceutical formulation of any one of those described herein; (i) a product of any one of those described herein; or (j) any combination thereof.

In an embodiment, the techniques described herein relate to a method of treating and/or preventing a disease, condition, or a symptom thereof in a subject or cell thereof, the method including, modifying one or more target polynucleotides in or from the subject or cell thereof by contacting the one or more target polynucleotides with an engineered nucleic acid targeting system of any one of those described herein, wherein the engineered nucleic acid targeting system is directed to the one or more target sequences in one or more target polynucleotides by the guide nucleic acid guide molecule(s) of the engineered nucleic acid targeting system, whereby one or more target polynucleotides is/are modified.

In an embodiment, the techniques described herein relate to a method, wherein contacting occurs in vitro, in situ, ex vivo, or in vivo.

In an embodiment, the techniques described herein relate to a method, wherein contacting occurs ex vivo in a cell obtained from the subject or progeny thereof and wherein the method further includes administering cell or obtained from the subject or progeny to the subject after contacting the cell or progeny thereof with the engineered targeting system.

In an embodiment, the techniques described herein relate to a method of generating a modified organism, the method including: modifying one or more target polynucleotides in a cell by a method as in any one of those described herein.

In an embodiment, the techniques described herein relate to a method, wherein the organism is a non-human animal.

In an embodiment, the techniques described herein relate to a method, wherein the organism is a plant.

In an embodiment, the techniques described herein relate to a method of identifying a trait of interest in an organism where the trait of interest is encoded by one or more target polynucleotides, the method including: contacting the organism or a sample therefrom comprising polynucleotides with an engineered nucleic acid targeting system of any one of those described herein, wherein the engineered nucleic acid targeting system is directed to the one or more target sequences by the guide nucleic acid guide molecule(s) of the engineered nucleic acid targeting system, whereby one or more target polynucleotides, and thereby the one or more traits, are identified.

In an embodiment, the techniques described herein relate to a method, wherein one or more target polynucleotides are modified by the engineered nucleic acid targeting system.

In an embodiment, the techniques described herein relate to a method, wherein the method is performed in vitro, in situ, ex vivo, or in vivo.

In an embodiment, the techniques described herein relate to a method, wherein the organism is a plant, non-human animal, or human.

In an embodiment, the techniques described herein relate to a method of identifying a polynucleotide modifier, the method including: exposing one or more polynucleotides to one or more candidate agents; and detecting one or more modified polynucleotides by contacting the one or more polynucleotides exposed to one or more candidate agents with an engineered nucleic acid targeting system of any one of those described herein, wherein the engineered nucleic acid targeting system is directed to the one or more target sequences of one or more modified target polynucleotides present in the sample by the guide nucleic acid guide molecule(s) of the engineered nucleic acid targeting system, whereby one or more modified target polynucleotides present in the sample are identified.

In an embodiment, the techniques described herein relate to a method of detecting one or more target polynucleotide present in a sample comprising polynucleotides, the method including: contacting, in vitro, one or more target polynucleotides present in the sample with an engineered nucleic acid targeting system of any one of those described herein, wherein the engineered nucleic acid targeting system is directed to the one or more target sequences of one or more target polynucleotides present in the sample by the guide nucleic acid guide molecule(s) of the engineered nucleic acid targeting system, whereby one or more target polynucleotides present in the sample are identified.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1F—Design and implementation of FLSHclust algorithm for clustering proteins. (FIG. 1A) Schematic of applications of protein clustering in biology and bioinformatic. Archetypal examples of biological systems that could be found with genome mining approaches for CRISPR are shown, including CRISPR-Associated Rossmann Fold (CARF) proteins and transposon linked genes. (FIG. 1B) (SEQ ID NO: 9521-9522) Conceptual schematic of locality-sensitive hashing. In contrast to standard hash-based bucketing, locality-sensitive hashing allows similar, non-identical objects to be bucketed together. The specific family of hash functions shown in the example is randomized positional masking (bit masking) on sequences. This family functions by dropping specific positions in each k-mer, where the positions are randomly selected per hash function. (FIG. 1C) Schematic of the steps of FLSHclust involving locality-sensitive hashing. First, all k-mers are extracted from each protein. Then for each hash function, the hash function is applied to all k-mers and k-mers with the same hash value are grouped and then processed independently to determine which sequences will be aligned in the next step. (FIG. 1D) Optimized hash functions with no false negatives as calculated using Markov Chain Monte Carlo compared to standard randomized hash functions from the same family. Probability of bucketing two k-mers together in one of the L hash tables as a function of the number of mismatches between the k-mers is shown. The parameters used for the Locality Sensitive Hashing (LSH) family functions are L=24 hash functions, k-mer length k=12, with 3 positions dropped per hash function. For the optimized hash functions, the target number of tolerated mismatches is 2, such that the family has no false negatives in identifying matches between k-mers with up to 2 mismatch positions. (FIG. 1E) Comparison of clustering algorithms for deep clustering of a sample of 1M proteins from the UniRef50 database and the full UniRef50 database (51M proteins). (FIG. 1F) Comparison of cumulative distribution of the number of proteins that are clustered with another protein as a function of the protein's percent sequence identity to its nearest neighbor. This metric was computed using a subset of 1 million proteins compared to all 51M proteins in UniRef50.

FIG. 2A-2C-Discovery of hundreds of rare novel CRISPR systems with a sensitive, scalable CRISPR association pipeline. (FIG. 2A) Schematic of CRISPR discovery pipeline using no all-to-all comparisons. (FIG. 2B) Comparison of naive and enhanced CRISPR association scores for identifying CRISPR694 associated clusters. Left: known Cas genes; right: all clusters. (FIG. 2C) Selection of CRISPR-associated clusters. Left: relative count of Cas (blue) vs non-Cas (gray) clusters as a function of enhanced CRISPR association score. An empirical threshold of 0.35 enhanced score was selected for identifying CRISPR-associated clusters. Right: relative count of all clusters with N_eff≥3. Dotted line demarcates the 0.35 enhanced score cutoff. ˜130,000 clusters with an enhanced score≥0.35 passed for further analysis.

FIG. 3A-3J—Multimeric Cas (also referred to herein by a proposed designation of “Type VII Cas”) system. (FIG. 3A) Locus diagram of the experimentally studied candidate Type VII Cas system. (FIG. 3B) UPGMA dendrogram from HHPred pairwise alignment scores of related Cas7s. (FIG. 3C) Phylogenetic tree (FastTree) of β-CASP proteins from both bacteria and archaea, including the β-CASP proteins linked to the candidate Type VII Cas system, which form a distinct clade. (FIG. 3D) Top: diagram of the domain architecture of the β-CASP protein (also referred to herein by a proposed designation of “Cas15”). Bottom: superposition of Cas15's C terminal domain with the Cas10's C-terminal from PDB: 6NUD showing the Cas10 interface with the target RNA. Both share the 4-helix bundle found in Cas10 and Cas1 1 that are known to interact with the target strand. (FIG. 3E) CDS target strand preferences of the protospacer matches for the CRISPR array of the experimentally studied Type VII locus. (FIG. 3F) Targets of the protospacer matches for the CRISPR array of the experimentally studied type VII locus. (FIG. 3G) (SEQ ID NO: 9523) Small RNA-seq of Type VII Cas7-Cas5 RNP pulldown along with the DR sequences. The apparent Cas5/Cas7 complex co-purifies with a processed crRNA as shown. (FIG. 3H) Size exclusion chromatography of the Cas7-Cas5 copurified with an expressed DR+spacer+DR or copurified with an expressed truncated DR+truncated spacer. (FIG. 3I) In vitro reconstituted Cas15 and associated effector complex RNP cleavage of Cy5-labeled RNA targets, in the presence or absence of cognate target sequences. Based on chromatogram peaks, complexes are expected in fractions 2-4. When the full crRNA is present (top gel), bands corresponding to Cas5 and Cas7 are present in fractions 2-4, suggesting they form a complex. In contrast, when only a truncated crRNA is present (bottom gel), no bands appear in fractions 2-4, suggesting complexes only form in the presence of a specific crRNA, suggesting a specific functional association of the Cas7-Cas5 proteins in this system with the crRNA component encoded nearby. (FIG. 3J) Target RNA cleavage by Cas15 and associated Cas7-Cas5 RNP at various temperatures. Cleavage is apparent in a range from 37° C. to 52° C.

FIG. 4A-4B—β-CASP system (FIG. 4A) β-CASP effector modules identified in this Type VII Cas system. All enhanced CRISPR association scores are shown below the system name as determined by the pipeline with the numerator indicating the number of CRISPR/divergent DR associated loci and the denominator indicating the effective sample size of the cluster. β-CASP (Metallo-β-lactamase) identified as novel CRISPR effector domain which was added to known CRISPR effector modules. (FIG. 4B) General evolutionary mechanism that likely gave rise to the β-CASP system-exaption of β-CASP effector domain (small pentagon) by an alternate system.

FIG. 5A-5B—Complete FLSHclust algorithm. (FIG. 5A) Complete outline of the FLSHclust algorithm. Unlike with typical LSH, which often requires materializing the entire set of L hash tables, FLSHclust only needs to materialize one hash table at a time, storing all potential matches in a reference database. As memory and disk space permits, up to T hash tables can be materialized per iteration, potentially reducing runtime. Time complexities shown (big O notation) are assuming the use of hash join implementations, however if merge join implementations are used, additional logarithmic factors are included in Step 2. (FIG. 5B) Pseudocode of the FLSHclust algorithm.

FIG. 6—Empirical scaling of time. Subsamples of UniRef50 at various dataset sizes were randomly generated and used as inputs for various clustering software running on the same 32CPU machine. Run times were plotted on a log-log scale (circles). Linear curves were fit to the linear scaling algorithms (Linclust, FLSH), while quadratic curves were fit to the quadratic scaling algorithms (MMseqs2, uclust) using least squares fit on the log-log transformed data (lines).

FIG. 7A-7F—Performance benchmarks of various CRISPR finders against synthetically generated CRISPRs. (FIG. 7A) Description of all parameter sets used for generating the 35 synthetic CRISPR array datasets. (FIG. 7B) Description of all of the CRISPR finder tools and their tested parameters for the benchmark along with their id/label (condition column). (FIG. 7C) Average runtime per ˜20 kb sequence for each of the CRISPR prediction tools. (FIG. 7D) Average recovery rates of all tools vs each of the synthetic CRISPR datasets. True Positives (real CRISPR-like arrays) vs False Positives (tandem repeats) are differentiated in the subplot titles. Error bars show 95% confidence bounds as determined by bootstrap with 2000 bootstraps. (FIG. 7E) Average fraction of correctly predicted number of DRs with error bars as in FIG. 7D. (FIG. 7F) average number of indels between predicted CRISPR DR and true CRISPR DR with error bars as in FIG. 7D.

FIG. 8A-8G—Analysis of candidate Type VII Cas proteins. (FIG. 8A) Locus diagram of a candidate Type VII Cas locus in candidate division WOR-3 bacterium isolate SpSt-780. (FIG. 8B) Top BLASTP (against NR and PDB) and HHPred results for each of the four components of the candidate type VII system. (FIG. 8C) UPGMA tree of representative Cas7 homologs across type III CRISPR and type VII systems. (FIG. 8D) (SEQ ID NO: 9524-9540) Top HHpred result of Cas7 from candidate type VII Cas systems. The catalytic aspartate (red triangles) is not conserved and is mutated to asparagine, suggesting that Cas7 is not capable of RNA cleavage as it is in many type III CRISPR systems. (FIG. 8E) (SEQ ID NO: 9541-9565) Top HHpred result for Cas5, which is most similar to the type III-D Cas5 homolog Csx10. (FIG. 8F) FastTree phylogenetic analysis of representative β-CASP domain proteins from bacteria and archaea. Cas15 proteins form a single clade, shown in expanded section. (FIG. 8G) (SEQ ID NO: 9566-9602) Top HHpred result for Cas15. The N-terminal β-CASP domain of Cas15 is similar to the yeast cleavage and polyadenylation specificity factor 100. Catalytic residues that coordinate Zn2+ ions required for catalysis are marked by triangles.

FIG. 9A-9B—Structural comparison of AlphaFold2 prediction of β-CASP domain of Cas15 and human β-CASP domain-containing proteins. Superimposition of (FIG. 9A) AlphaFold2 prediction of Cas15, with only the β-CASP domain shown, and the X-ray crystal structure of the human Artemis protein in complex with a DNA substrate (PDB: 7ABS) and (FIG. 9B) AlphaFold2 prediction of Cas15, with only the β-CASP domain shown, and the X-tray crystal structure of the human CPSF-73 protein. Insets show the catalytic centers, which coordinate two Zn2+ ions (circles). Catalytic residues responsible for Zn2+ coordination are shown as sticks with shaded heteroatoms.

FIG. 10—Spacer matches for candidate type VII system. Four examples of spacers from CRISPR arrays associated with the candidate type VII system, with matching protospacers in predicted transposon genes.

FIG. 11 shows an exemplary Type II-C Cas9.

FIG. 12 shows results of determination of PAM of the exemplary Type II-C Cas9 in FIG. 1.

FIG. 13 shows purification pull down experiments to determine small RNAs associated with the exemplary Cas9 in FIG. 11.

FIG. 14 shows DNA cleavage activity of the exemplary Cas9 in FIG. 11.

FIG. 15 shows the structure of the crRNA (SEQ ID NO: 8896) and tracrRNA (SEQ ID NO: 8897) in the form of a complex.

FIG. 16 shows exemplary Type II-B Cas9 proteins.

FIG. 17 shows an exemplary method of identifying and characterizing Cas proteins.

FIG. 18 shows exemplary Cas9-t had interference activity with NGCH PAM.

FIG. 19 shows pulldown of the Cas9-t protein bound to ncRNAs revealed processed CRISPR and tracrRNA.

FIG. 20 shows the cleavage of dsDNA by an exemplary Cas9-t in vitro using an sgRNA (SEQ ID NO: 8898).

FIG. 21—shows a sequence view of exemplary Type II-D IntCas9s (light gray bar), direct repeats (DR) (black bar), and tracrRNA (med gray bar).

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance, or substituent may or may not occur and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may be a cell lysate sample, e.g., a crude, non-isolated, and/or non-purified sample. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The terms “polynucleotide”, “nucleotide”, “nucleotide sequence”, “nucleic acid”, “nucleic acid molecule”, and “oligonucleotide” are used interchangeably. They refer to a polymeric form of nucleotides of any length, either deoxyribonucleotides or ribonucleotides, or analogs thereof. Polynucleotides may have any three-dimensional structure and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996.

A polynucleotide may comprise one or more modified nucleotides, such as methylated nucleotides and nucleotide analogs. If present, modifications to the nucleotide structure may be imparted before or after the assembly of the polymer. The sequence of nucleotides may be interrupted by non-nucleotide components. A polynucleotide may be further modified after polymerization, such as by conjugation with a labeling component. As used herein, the term “wild type” is a term of the art understood by skilled persons and means the typical form of an organism, strain, gene, or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. The percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has a functional role to play in an organism and hence is the molecular unit of heredity in living organisms. For the purpose of this invention, it may be considered that genes include regions that regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive.

As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain.

As used herein, when a protein (e.g., an enzyme) is mentioned, the term also includes a functional domain of the protein (e.g., enzyme). For example, a reverse transcriptase may refer to a reverse transcriptase protein or a reverse transcriptase domain. When a term refers to a protein, e.g., a Cas protein, a transposase, etc., the term encompasses both the full length of the protein as well as a functional fragment of the protein. The term “functional fragment” means that the sequence of the polypeptide may include less amino acid than the original sequence but still enough amino acids to confer the enzymatic activity of the original sequence of reference. It is well known in the art that a polypeptide can be modified by substitution, insertion, deletion and/or addition of one or more amino acids while retaining its enzymatic activity. For example, substitutions of one amino acid at a given position by chemically equivalent amino acids that do not affect the functional properties of a protein are common.

The terms “orthologue” (also referred to as “ortholog” herein) and “homolog” (also referred to as “homolog” herein) are well-known in the art. By means of further guidance, a “homolog” of a protein as used herein is a protein of the same species and performs the same or a similar function as the protein it is a homolog of. An “orthologue” of a protein, as used herein, is a protein or polynucleotide, respectively, of a different species that performs the same or a similar function as the protein it is an orthologue of. A homologous or orthologous protein as used herein is a protein that shares a common structure, as the protein it is a homolog or ortholog of, respectively, e.g., primary structure (i.e., a polypeptide sequence), secondary structure (i.e., a local folded structure, e.g., a-helix, b-pleated sheet), and/or tertiary structure (i.e., an overall 3-dimensional structure).

By means of further guidance, a “homolog” of a polynucleotide as used herein is a polynucleotide that shares a common nucleotide sequence as the polynucleotide it is a homolog of. Homologous genes, for example, can share a common ancestral gene. An “orthologue” of a polynucleotide as used herein is a polynucleotide of a different species which shares a common nucleotide sequence as the polynucleotide it is an orthologue of. Orthologous genes, for example, can share a common ancestral gene but occur in different species. Homologous or orthologous polynucleotides may but need not be structurally related or are only partially structurally related to the polynucleotide it is a homolog or ortholog of, respectively. A homologous or orthologous polypeptide as used herein is a polypeptide which shares a common structure as the protein it is a homolog or ortholog of, respectively, e.g., primary structure (i.e., a polynucleotide sequence), secondary structure (i.e., a local folded structure, via complementary base pairing, e.g., double helices, stem-loop structures, pseudoknots, and G-quadruplexes), and/or tertiary structure (i.e., an overall 3-dimensional structure, e.g., A-, B-, or Z-form double helices, RNA triplexes).

Homologs and orthologs may be identified by homology modelling (see, e.g., Greer, Science vol. 228 (1985)1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April;22 (4): 359-66. doi: 10.1002/pro.2225.). See also Shmakov et al. (2015) for application in the field of CRISPR-Cas loci.

As described in aspects of the invention, sequence identity is related to sequence homology. Homology comparisons may be conducted by eye, or more usually, with the aid of readily available sequence comparison programs (e.g., homology modelling, see, e.g., Greer, Science vol. 228 (1985)1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April;22 (4): 359-66. doi: 10.1002/pro.2225.)). See also Shmakov et al. (2015) for application in the field of CRISPR-Cas loci. These commercially available computer programs may calculate percent (%) homology between two or more sequences and may also calculate the sequence identity shared by two or more amino acid or nucleic acid sequences. In an embodiment, the homologue or orthologue of a protein has an amino acid sequence homology or identity, or a polynucleotide a nucleic acid sequence homology or identity, of at least 80%, more preferably at least 85%, even more preferably at least 90%, such as for instance at least 95% with the protein (e.g., a wild type protein) or the polynucleotide (e.g., a wild type gene), respectively. A protein or polynucleotide derived from a species means that the protein or nucleic acid, respectively, has a sequence identical to an endogenous protein or nucleic acid, respectively, or a portion thereof in the species. The protein or nucleic acid derived from the species may be directly obtained from an organism of the species (e.g., by isolation), or may be produced, e.g., by recombination production or chemical synthesis.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells, and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” or “an example embodiment,” means that a particular feature, structure, or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment but may. Furthermore, the particular features, structures, or characteristics may be combined in any suitable manner, as would be apparent to a person skilled in the art from this disclosure, in one or more embodiments. Furthermore, while certain example embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The present disclosure provides non-naturally occurring, engineered compositions and methods of using said compositions for nucleic acid-targeting and modification. In one aspect, a composition comprises one or more components of a non-naturally occurring, engineered multimeric CRISPR-Cas system. In an embodiment, a composition comprises a β-CASP polypeptide (also referred to herein as a metallo-β-lactamase fold like nuclease) and a plurality of Cas polypeptides. The compositions may further comprise one or more guide molecule(s). The guide molecule is capable of forming a multimeric CRISPR-Cas complex with the β-CASP polypeptide and the plurality of Cas polypeptides(s). In an embodiment, each guide molecule is capable of directing said multimeric CRISPR-Cas complex to a target sequence in a target molecule, e.g., a target polynucleotide. For ease of reference, this new CRISPR-Cas system may be referred to as a Type VII CRISPR-Cas system.

In an embodiment, the composition further comprises one or more functional domains associated with the Cas polypeptides or other components, enabling various modifications of target polynucleotides. In an embodiment, the functional domain may be a nucleotide deaminase, e.g., for modifying a single nucleotide or base pair in a target polynucleotide.

In another aspect, embodiments disclosed herein include applications of the compositions herein, including therapeutic and diagnostic applications. Methods and systems for the delivery of the compositions is also provided, including to a variety of cells and via a variety of particles, vesicles, and vectors.

In another aspect the present disclosure is directed to delivery compositions used to deliver one or more components of the Type VII CRISPR-Cas system to a cell or population of cells in vitro, ex vivo, or in vivo. In another aspect, the present disclosure is directed to methods of modifying target polynucleotides using the Type VII CRISPR-Cas systems and/or compositions thereof disclosed herein, including the use of the Type VII CRISPR-Cas systems and/or compositions for diagnostic and/or therapeutic uses.

In another aspect, embodiments disclosed herein provide non-natural or engineered compositions, systems, and methods for nucleic acid modification. In general, the compositions and systems herein comprise a subset of newly identified Class 2, Type II Cas polypeptides that are smaller in size than previously discovered Class 2, Type II Cas polypeptides. In some embodiments, the compositions and systems comprise one or more Type II Cas polypeptides that are less than 850 amino acids in size and one or more nucleic acid guide molecules. The relatively small sizes of these Cas polypeptide may allow easier engineering, multiplexing, packaging, and delivery, and use as a component in a fusion construct, e.g., fusion with a nucleotide deaminase. In some examples, the Type II Cas polypeptides are Type II-B Cas9 or Type II-C Cas9 polypeptides.

In another aspect, embodiments disclosed herein provide non-natural or engineered compositions and systems as well as their use in methods of modifying a target polynucleotide. In general, the systems include a Cas polypeptide that has a size range that is smaller than canonical Cas9 polypeptides, e.g. less than 950 amino acids in size, and in some embodiments, less than 750 amino acids in size. The large size of existing CRISPR-Cas systems can pose challenges for certain delivery methods and limit their efficacy. The smaller Cas polypeptides of the present disclosure can make them easier to deliver into target cells. The smaller size can enhance the efficiency of delivery methods, such as viral vectors or physical methods, and improve the overall delivery success rate.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Engineered Nucleic Acid Targeting Systems

Described in exemplary embodiments herein are engineered nucleic acid targeting compositions comprising a Cas polypeptide and a nucleic acid guide molecule capable of forming a complex with the Cas polypeptide and directing sequence-specific binding to a target sequence in a target polynucleotide. The Cas polypeptide comprises a split RuvC nuclease domain, and a HNH nuclease domain, and is about 950 amino acids or less in size. The Cas polypeptide may function as a nuclease. In one embodiment, the Cas functions as a DNA nuclease, although other modified functionalities are possible and as described in further detail below. The Cas polypeptide and guide molecule may be referred to as a CRISPR-Cas complex. The guide molecule generally comprises a guide sequence and a scaffold. The guide sequence determines which target sequence is bound by the complex and can be re-engineered each time a new target sequence is desired. The scaffold portion of the guide helps facilitate formation of the complex with the Cas polypeptide.

### CRISPR-Cas Systems

In general, a CRISPR-Cas system or CRISPR system as used in herein and in referenced documents, such as WO 2014/093622 (PCT/US2013/074667), refers collectively to genes, transcripts, proteins, and other elements involved in the expression or directing the activity of CRISPR-associated (“Cas”) genes or gene products, and/or the gene products themselves (e.g., Cas polypeptides). These Cas genes or gene products include, for example, sequences encoding a Cas gene, a trans-activating CRISPR sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as a Type-VII, Type II-B Cas, Type II-C Cas, Type II-D Cas, e.g. CRISPR RNA and transactivating (tracr) RNA or a single guide RNA (sgRNA) (chimeric RNA)) or other sequences and transcripts from a CRISPR locus. In general, a CRISPR-Cas system is characterized by a Cas polypeptide (used interchangeably herein with CRISPR protein, CRISPR enzyme, CRISPR-Cas protein, CRISPR-Cas enzyme, Cas protein, or Cas enzyme) and a nucleic acid guide molecule, both of which promote the formation of a CRISPR-Cas complex at the site of a target sequence (also referred to as a “protospacer” in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008 and Makarova et al., 2020. Nature Rev Microbiol. 18:67-83.

### Type VII CRISR-Cas Systems and Compositions

In one aspect, embodiments disclosed to herein are directed to non-naturally occurring, engineered CRISPR-Cas systems or compositions, referred to herein by the proposed designation of a “Type VII” CRISPR-Cas systems or compositions, comprising a β-CASP polypeptide which forms a multimeric Cas polypeptide complex in combination with one or more Cas polypeptides. As used herein a Type VII CRISPR-Cas system may also be referred to as a Type VII CRISPR system or a Type VII CRISPR effector protein system. As used herein a multimeric Cas polypeptide complex may also be referred to as a Type VII CRISPR effector protein. As used herein, the β-CASP polypeptide may also be referred to as “Cas15”. The other Cas polypeptides may include a Cas5 and a Cas7. In an embodiment, the Type VI system further comprises a Cas6. In one embodiment, the Cas15. Cas5, and Cas7 define a minimal effector complex capable of endonuclease activity.

In an embodiment, the multimeric Type VII Cas system may form a CRISPR-Cas complex with a guide molecule.

### Cas15 Polypeptides

In an embodiment, the Cas15 comprises a β-CASP domain. The β-CASP domain is a nuclease fold found in all domains of life that exhibits RNA endonuclease, 5′ to 3′ exonuclease and/or DNA nuclease activity. Dominski et al. Biochim. Biophys. Acta. 1829, 532-551 (2013). β-CASP domain containing proteins are involved in non-homologous end joining DNA repair (NHEJ), V (D) J recombination, RNA surveillance, mRNA/rRNA maturation and RNA decay. Mandel et al. Nature. 444, 953-956 (2006); Phung et al. Nucleic Acids Res. 48, 3832-3847 (2020); M. R. Lieber. Annu. Rev. Biochem. 79, 181-211 (2010); Callebaut et al. Nucleic Acids Res. 30, 3592-3601 (2002): Moshous et al. Cell. 105, 177-186 (2001).

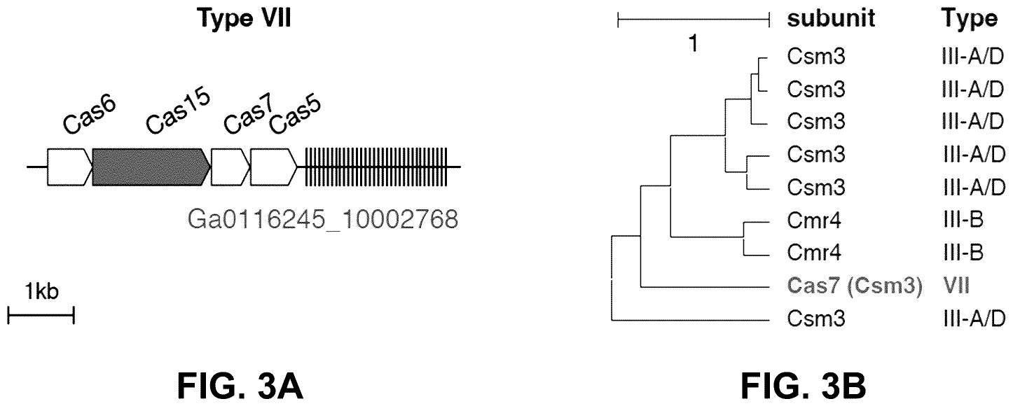

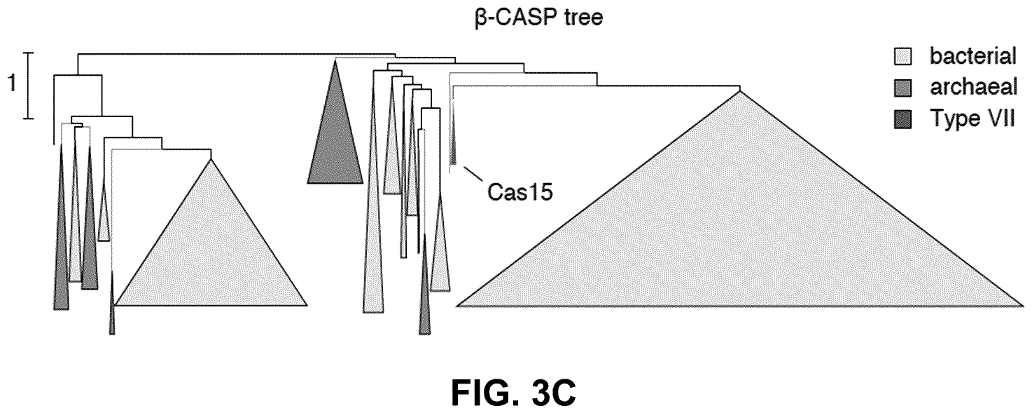

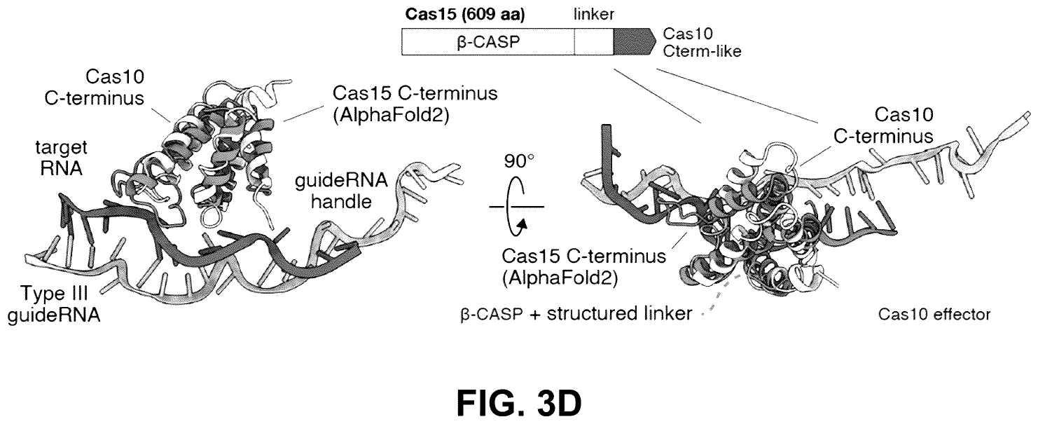

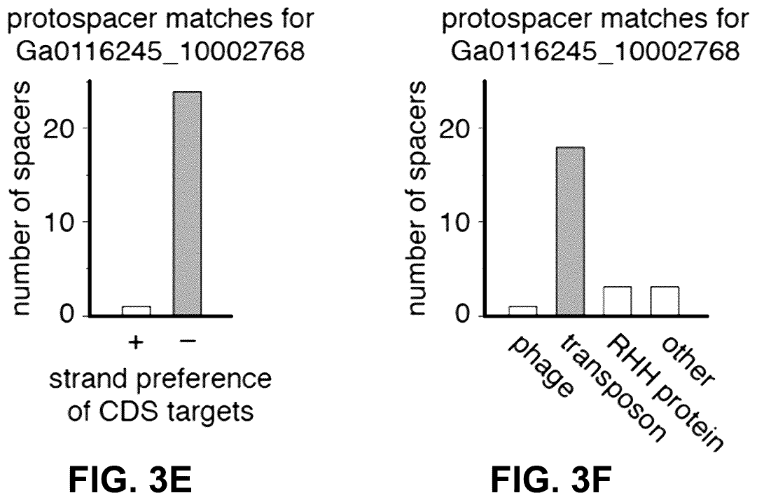

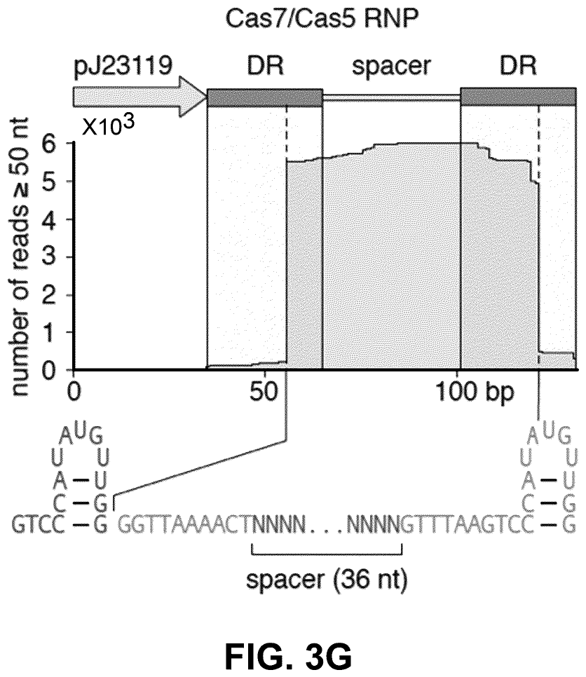

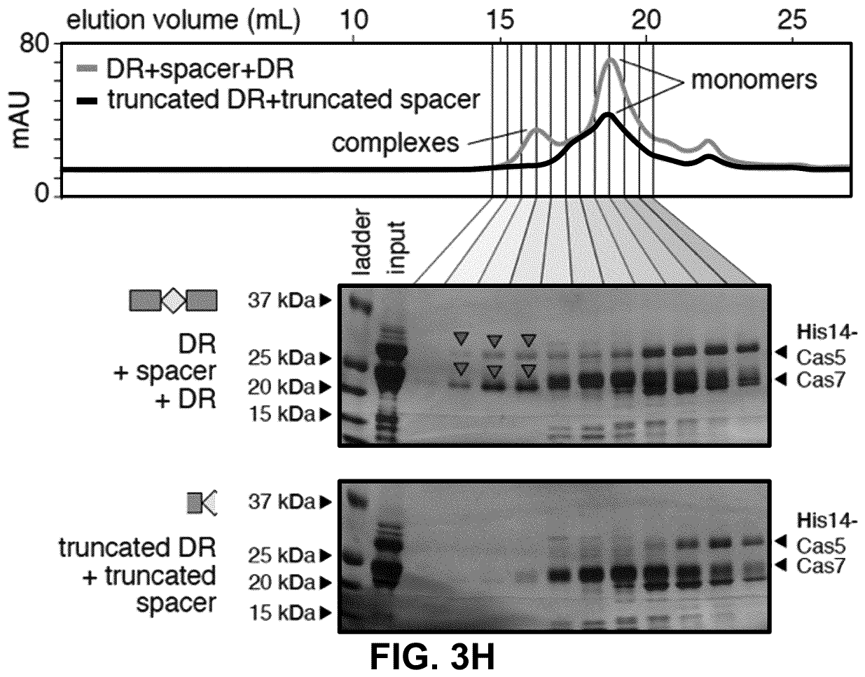

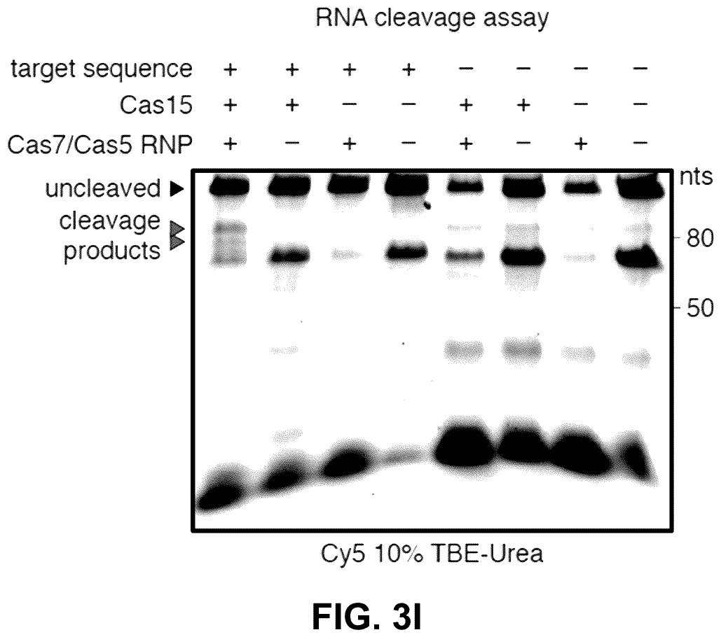

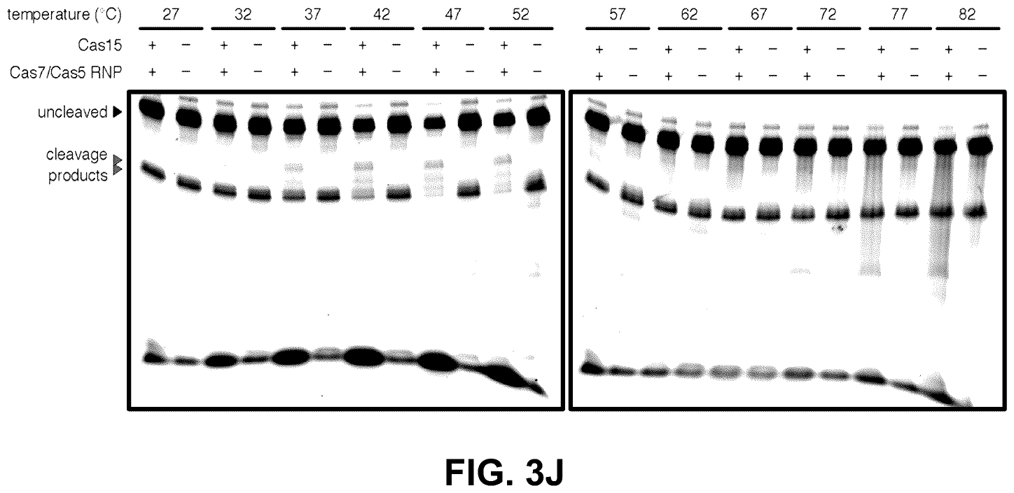

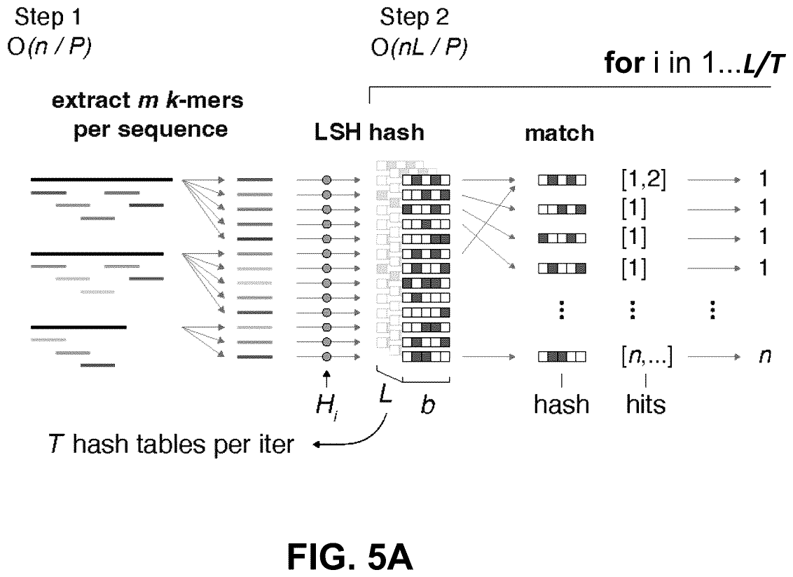

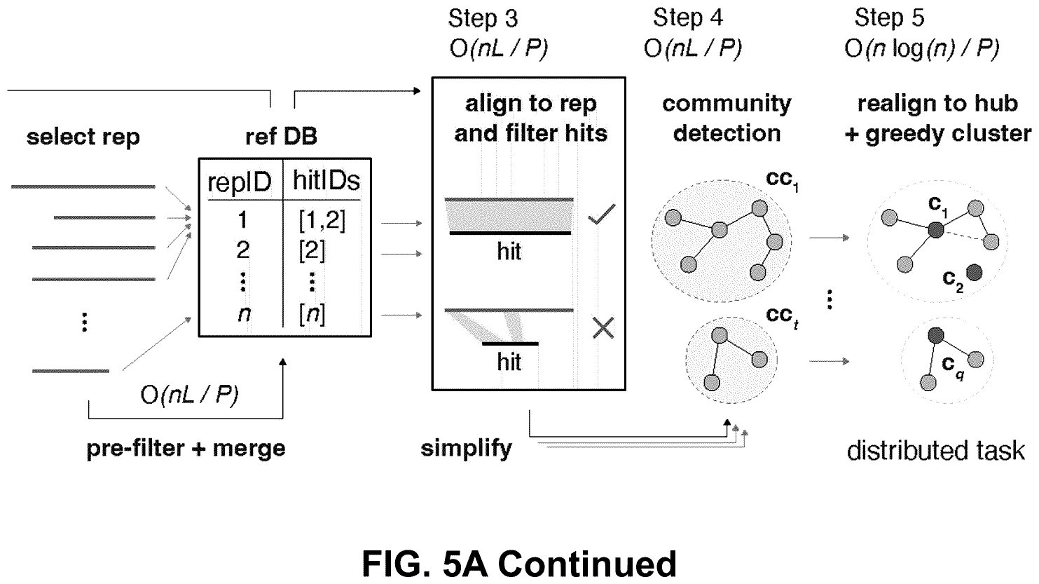

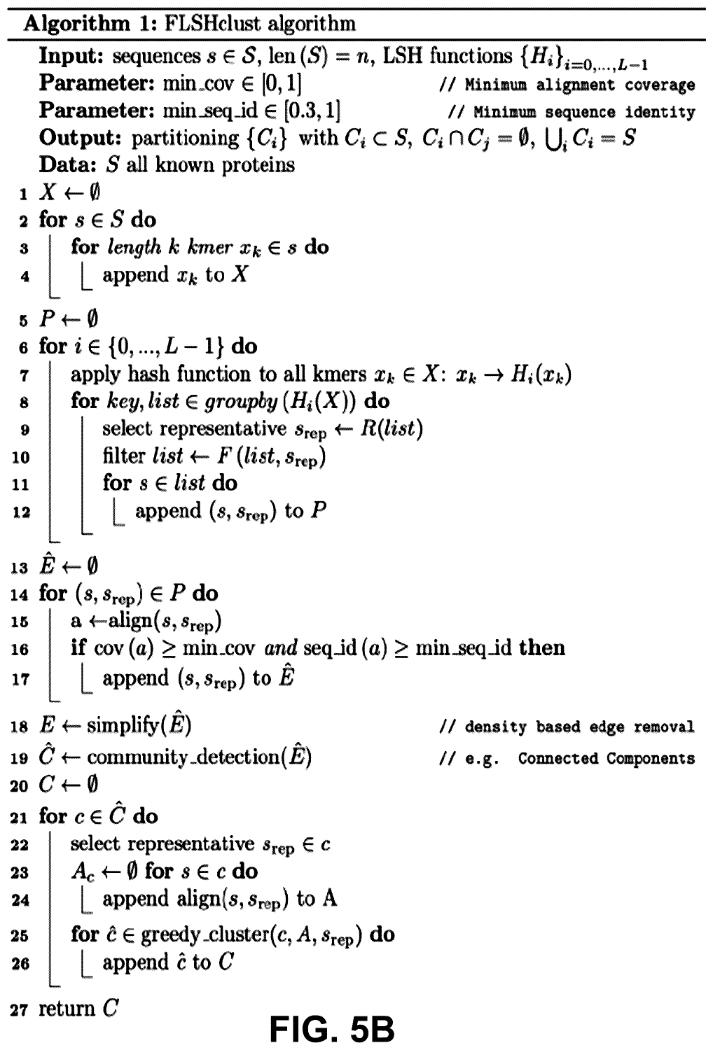

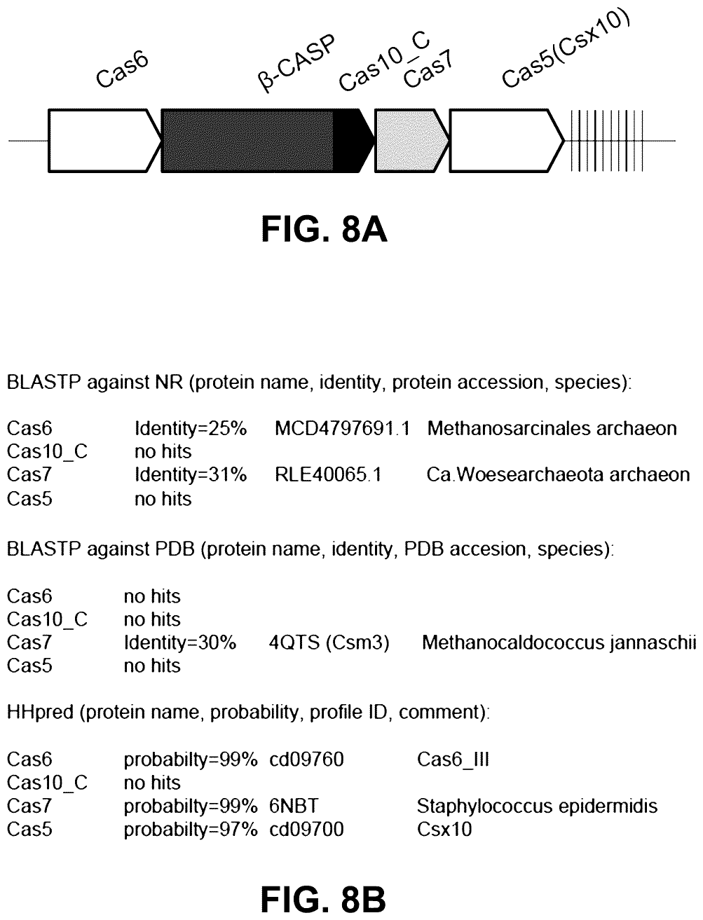

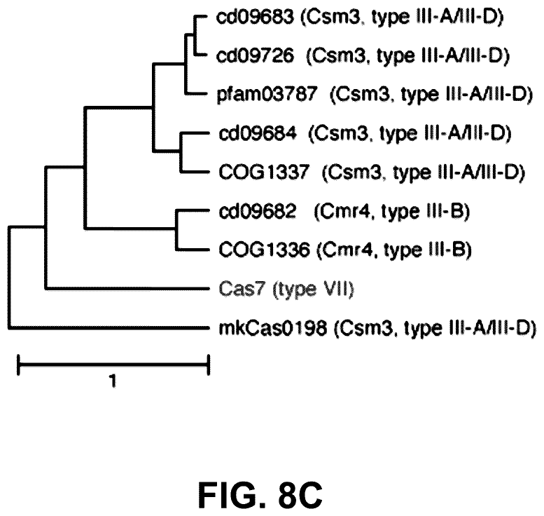

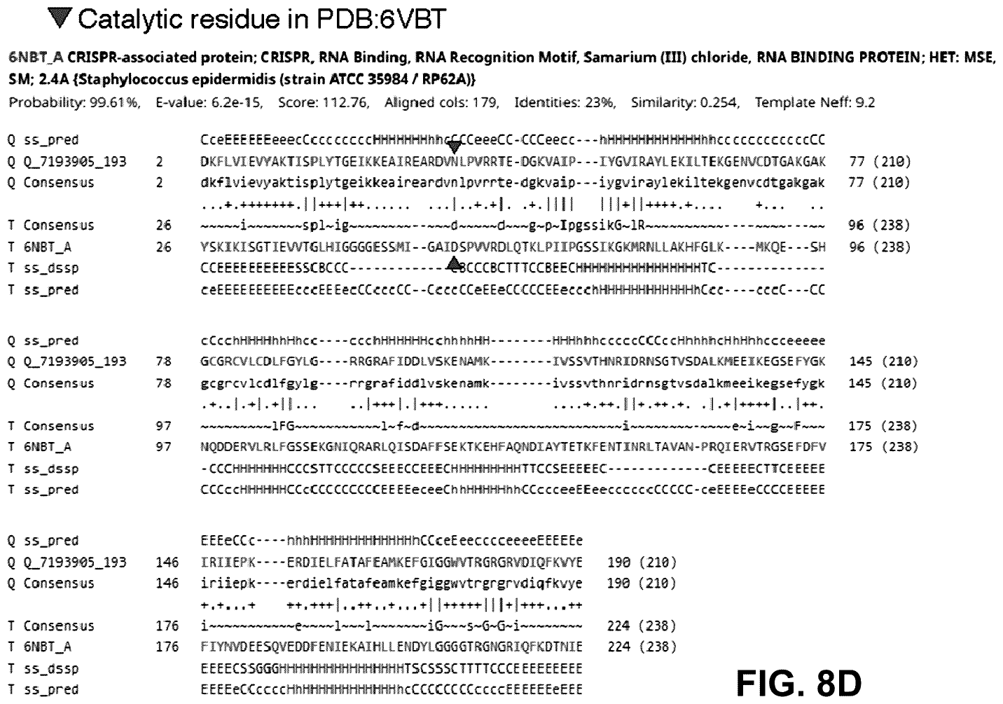

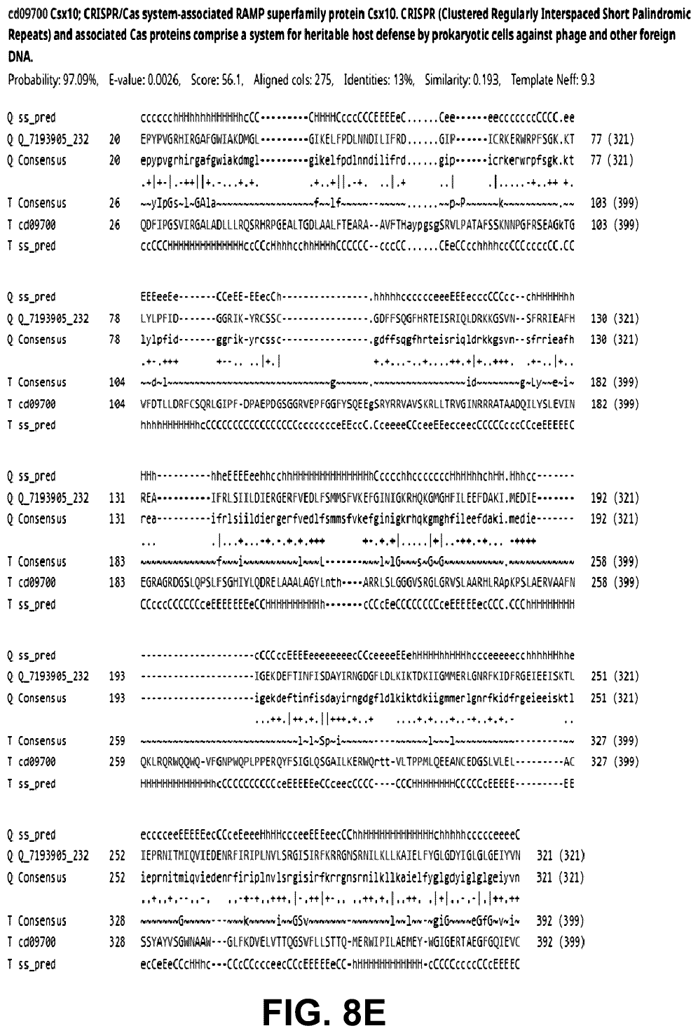

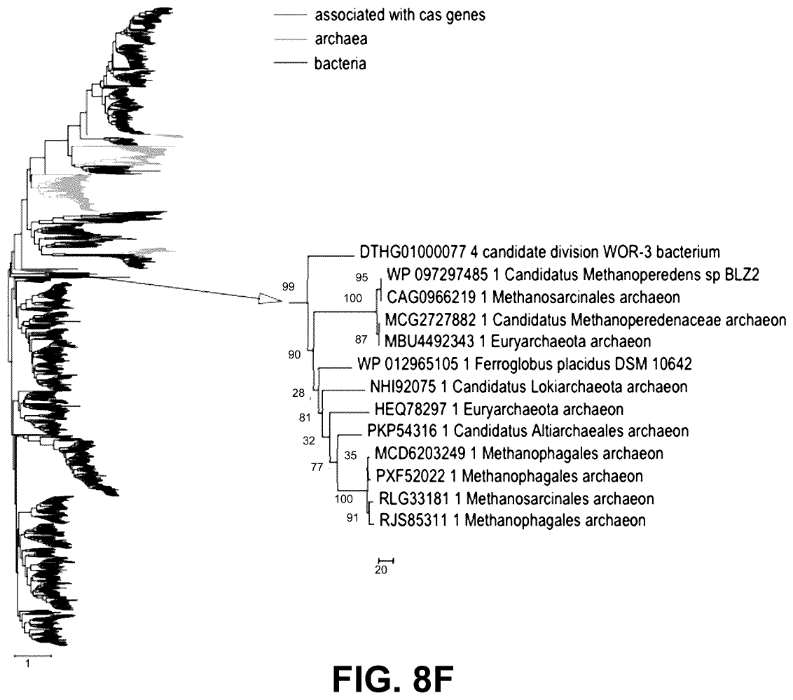

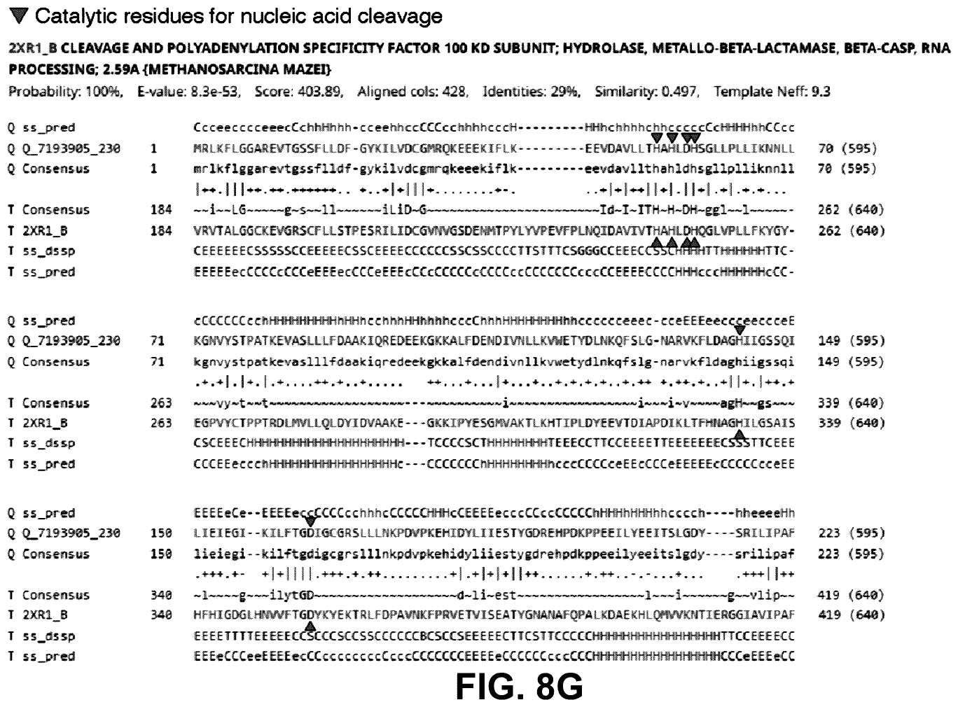

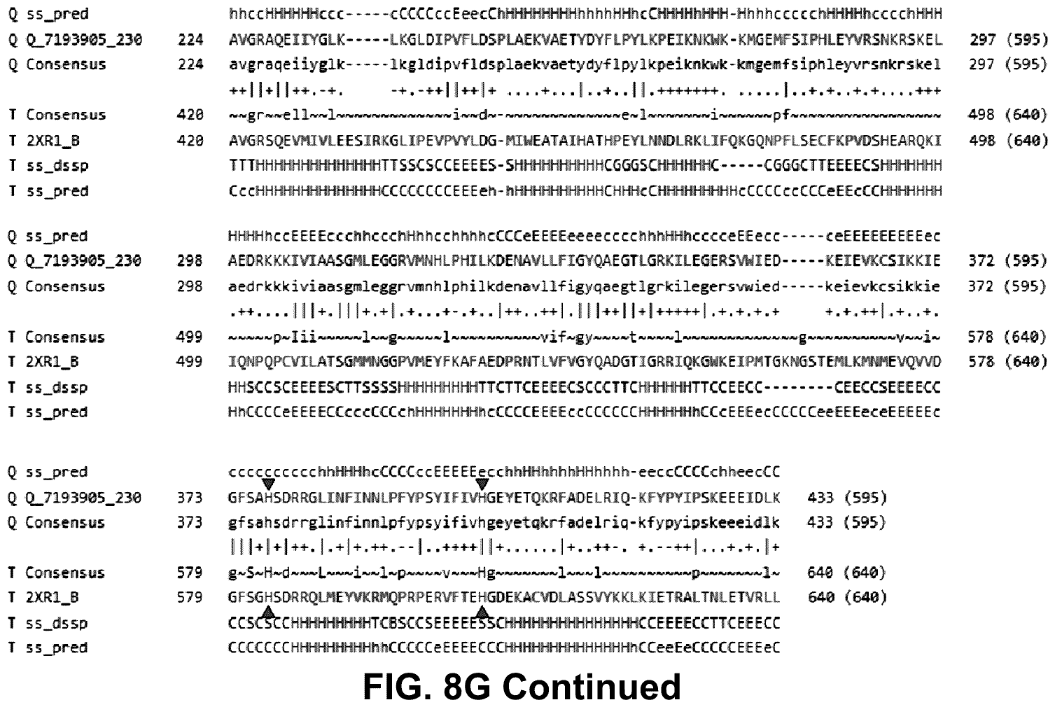

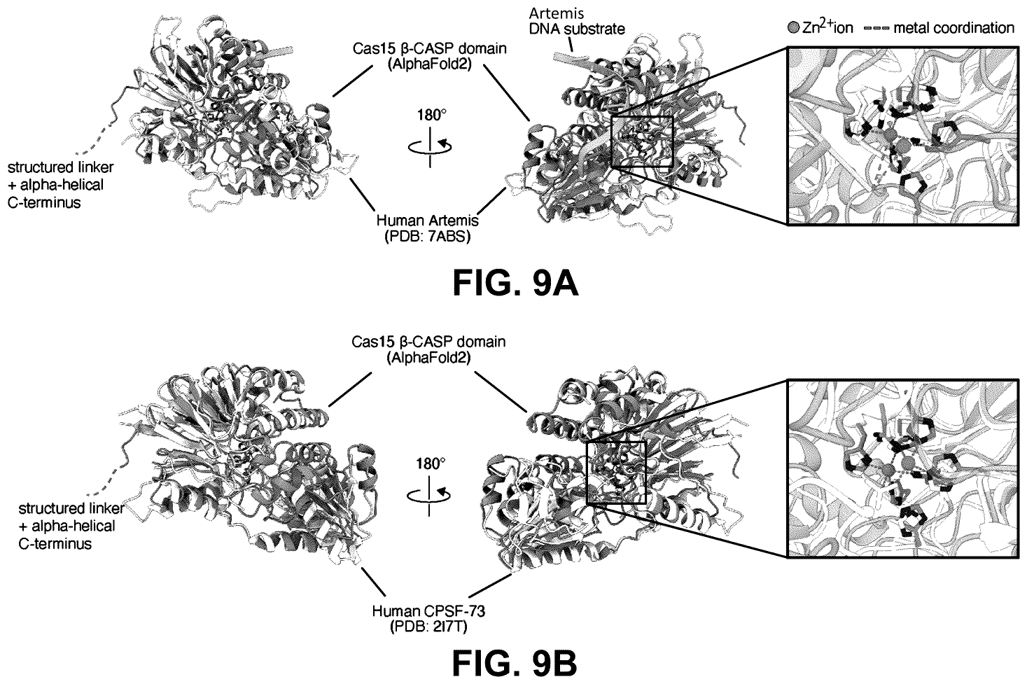

Structural modeling of Cas15 with AlphaFold2 shows two distinct domains, namely the N-terminal β-CASP domain (FIGS. 8G, 9) and a C-terminal adaptor domain with structural similarity, but not sequence similarity to, the approximately 200 amino acid C-terminal domain of Cas10 (FIG. 5D), the large subunit of type III systems that is involved in target RNA interaction. You et al. Cell. 176, 239-253.e16 (2019). In an embodiment, the Cas15 polypeptide comprises a plurality of residues capable of coordinating with Zn²⁺ ions. See FIG. 8G. The Cas15 polypeptide functions as the nuclease effector of the Type VII system. While not being bound by a theory, Cas15 is believed to function as an RNA nuclease. See FIGS. 3I, 3J.

In an embodiment, the Cas 15 polypeptide comprises one or more amino acid sequences of Table 1.

TABLE 1

| Poly- | |
|---|---|
| peptide | Amino Acid Sequence |
| Cas15 | MIWSHPQFEKGGGSGGGSGGSAWSHPQFEKSSGSMIKFIG |
| | GASKVTGSAFLLETGNAKILIDCGIEQEKGIEKDNNEIIE |
| | KKINEIGKADICILTHAHLDHSGLVPLLVKKRKVNKIIST |
| | PATKELCRLLFNDFQRIQEENNDIPLYSYDDIESSFEIWD |
| | EIDDRNTIELFDTKITFYNNSHIIGSVSVFIETHNGNYLF |
| | SGDIGSKLQQLMDYPPDMPDGNVDYLILESTYGNKSHDSS |
| | DRDRLLEIAKTTCENGGKVLIPSFAIGRLQEVLYTFSNYN |
| | FNFPVYIDSPMGSKVTNLIKEYNIYLKKKLRRLSITDDLF |
| | NNKYIAINTSNQSKELSNSKEPAVIISASGMLEGGRILNH |
| | LEQIKNDENSTLIFVGYQAQNTRGRKILDGEEKVRCRIEK |
| | LNSFSAHADQDELIDYIERLKYTPYKVFLVHGEKEQREIL |
| | AKRIISKKIRVELPENYSQGKEILIEKKVVLNINTDNMCN |
| | FASYRLMPFSGFIVEKDDRIEINDKNWFDMIWNEEYNKMR |
| | SQIVAEDFSTDQNEDSMALPDMSHDKIIENIEYLFNIKIL |
| | SKNRIKEFWEEFCKGQKAAIKYITQVHRKNPNTGRRNWNP |
| | PEGDFTDNEIEKLYETAYNTLLSLIKYDKNKVYNILINFN |
| | PKL* |
| | (SEQ ID NO: 1) |
| MLB- | MTGLTFIGATREVTGSCHLLEVNDRRILLDCGMRQGRGVS |
| like | KASEFPFKPDGINAVILSHAHIDHSGLLPLLVSQGFKGKI |
| | YSTVATRELVRILLYDSAKIQEEDYVAGGEPPLYSEDDVN |
| | ETMRLFEVFPYHEEFDLLNVGTVKFYDAGHILGSAITVIS |
| | TAKTVCYTGDLGHGMSPILRAPETPIEEVDYLIMESTYGN |
| | KLHSRDDPTLKIKEIVSEVYKNKGKLLIPVFAVGRAQEIL |
| | YSLKEMKEAGEIPHDLKVYLDSPMADKSTSLYSNMADYLK |
| | PEFYTQFLNNTSPFEFEGFEIIKGHEDSLNLATSNGTAII |
| | LAASGMLEGGRVMNHLPKILSDKNSVICFVGYQVEGTHGR |
| | TLLDGVSEIKLKEKVVEVSCKIEAISSYSAHADKKGLFNF |
| | LNSLKFNPYKVFVVHGEEDANKAVVESLKNLKLRAVAPTR |
| | GDTESFGGTVIKIIKEKEFKIDFSPEYITLKKSHLRIAPF |
| | CGALVESKNGMIRLISNSVLIGLMEAEEQAFTNEFKEKKI |
| | ELEDLQESNNIPSKTENLSENITSFISYDMYCKKLEKFYN |
| | SKDNFGDSILSKNLAHEIYCKAIREGKDSVIRLINQKKDK |
| | GKFNIDDEEIIEKFVELIKIAVINLTIREFCKGLEPYKIK |
| | KQC* |
| | (SEQ ID NO: 2) |
| | VVKIRFLGACREVTGSMHLLDTGKTKILLDCGMRQGVDSV |
| | EREKHIPFDPSEIDYIILSHAHIDHSGLIPYLYLRGFRGR |
| | VVTTTATRAIAELLLLDSAKIMKEEYEKSNIPPLFDERDV |
| | VEVMSHFDAYPYNKPVRLQDVKLSFLDAGHILGSAVTLLR |
| | INGLQICYTGDIGSGTSPLMNPPTPPKEADVLIMESTYGN |
| | RRHEDRAKAVETMKAVISDTIKAGGKVLIPVFAVGRAQEV |
| | LYVLRNLRDKIRVKVYLDTPLGSRVTDIYKSYSNMLRKEF |
| | YELFLRGKNPIEFEGLEYVTTYNRSRELAKSNEPCIILSA |
| | SGMLEGGRVLNHLPYILQDENSTVLFVGYQAEGTLGRQIV |
| | DGNKLVYVNGDEVDVRCNVVNVSAFSAHADEDGLVGFVEG |
| | MDYYPRRIYIVHGEEEAARNLLARLPKVRKSIPERGEEAD |
| | LGWGGRAGMDGEREGRAIIDFKPGFVDFMGLSIHPEPLLL |
| | VREGEMLILRRYGEVVGELMSAGEELAVEIRAAVKSAEVE |
| | KISVEAGDIARLREFMEAGILSKKLSKEIVCTLLKEGRDE |
| | VIRMLNLKRDKDRFPVKDPEVVNDFVEYMVNLLKTRKEVE |
| | IVAAFEELIPKISGMCY* |
| | (SEQ ID NO: 3) |
| | MKITFLGGVREVTGSHHLLEINGRRILLDCGLRQGKGLPK |
| | APEFPFKAGDIDAVVLSHAHLDHSGLLPFLACSGFSGKIY |
| | STSATRELARLLLLDAAKIMKEDYGKGLSDAPPLYDEEAV |
| | HRTIRMFETADYNQEIEISPEVSLKFLDAGHILGSAVSLL |
| | KIKGVGSLCYTGDIGHGRSPILNPPQVPEEDVTFLMIEST |
| | YGNRKHGRGGEKELENVITKTLSRGGKVLIPVFAVGRAQE |
| | ILFCIKRLVEEGKVNAKVYVDTPMGESATDLYSSFSNYLR |
| | EEFREHFLRGKSPFEFSGLEFVRGHKTSLDIAESEEPCII |
| | LAASGMLEGGRVLNHLPGILKDEKHSLVFVGYQAEGTLGR |
| | EILEAANSTMRRVRINDSEVELRCSVVALSSFSAHADIDG |
| | LIGFASRLKYCPYRTFVVHGEHESSLNLANELIKLKHRAR |
| | VPGIKEEFSLGFTRVVVEKTRDVELGFSPEFTCLGDAEVA |
| | VFSGGLVKTASGIKLLNKQRFLEFLDELLTKEEEMLKARV |
| | RVKSGLTDQKKEEESKKALISEREIIEKFRAFQEKGILTK |
| | RLMESIYCELLTSGDEAIRMLNEKRRKKRFNITDEKIIDE |
| | FCDFMVKLLDSVDEKVIIRCLKKFDIHPEC* |
| | (SEQ ID NO: 4) |
| | MKLKFLGATQEVTGSCILLETKEHKYLFDCGMYQGPEKRD |
| | QSNLFFNPHEIDGIFLSHAHIDHSGLLPLLWKRGYNGPIY |
| | STTATKELTRILLLDSAKIQAEEENETGIEALYNESDVKN |
| | IINNFEGIPYKRKTSLSDLEFSFCDAGHILGSAITYLNLN |
| | GITLTYTGDLGHGQSPILNPPTKIKASDYLIIESTYGNKT |
| | HSNLNIVDKFLSKEIKETMKNEGKLLIPVFAVGRSQEILY |
| | YLWKIKDQIKDIPIFFDTPMGTDVTELYSEFQDYLKPSFR |
| | EYFLKNKSIFHLENLKFVKTQKESRSIASSSNSSIILAAS |
| | GMLEGGRVMNHIESILSDTNSKILFSGYQPEGTLGRKVME |
| | KKSKLLINDKEIELKCQICKLSGLSAHADKTGLLSFFKNF |
| | GIHPQKTFIVHGEKEASLNLFNMISHEHGKAIIPSKLHRE |
| | SLSGIMVKKLEKNNIDLKFKPSFFKIGNKEIMPFVGAIIK |
| | NKEGMEVIDLNQYNQLMNKYKDQMELPLNTEIKDIIRPID |
| | QDQVLIISPEEFAKNLKSLHSDIFSKRKIKDYIKILEKEG |
| | KDQLIRTFLVDLNKNRLGKSLYVANPSIFEIEIKKFKDFF |
| | IAGLNSIERYEIIKILEEYLESK* |
| | (SEQ ID NO: 5) |
| | MRLKFLGGAREVTGSSFLLDFGYKILVDCGMRQKEEEKIF |
| | LKEEVDAVLLTHAHLDHSGLLPLLIKNNLLKGNVYSTPAT |
| | KEVASLLLFDAAKIQREDEEKGKKALFDENDIVNLLKVWE |
| | TYDLNKQFSLGNARVKFLDAGHIIGSSQILIEIEGIKILF |
| | TGDIGCGRSLLLNKPDVPKEHIDYLIIESTYGDREHPDKP |
| | PEEILYEEITSLGDYSRILIPAFAVGRAQEIIYGLKLKGL |
| | DIPVFLDSPLAEKVAETYDYFLPYLKPEIKNKWKKMGEMF |
| | SIPHLEYVRSNKRSKELAEDRKKKIVIAASGMLEGGRVMN |
| | HLPHILKDENAVLLFIGYQAEGTLGRKILEGERSVWIEDK |
| | EIEVKCSIKKIEGFSAHSDRRGLINFINNLPFYPSYIFIV |
| | HGEYETQKRFADELRIQKFYPYIPSKEEEIDLKGGAREKI |
| | IIDFEKKFQKYGMYEIMLFTGGILKDDNLIRVISKEDLFE |
| | IINEEEKRLKHKEKVEIPVDIEKEEIESITEEEFYRELKN |
| | MREEKILSKSKLQEFIENASPGIDNLFSWIRITKNKIRFY |
| | PDKEKNDMVAEILLRGINSLREIAVSIAEKVLEET* |
| | (SEQ ID NO: 6) |
| | MKICFFGATKEVTGSCFLIKTYKSSFLLDCGQRQGPDEEK |
| | LPRFQFNPKEIDFVILSHAHIDHSGLLPKMIAEGFDGEIY |
| | STVATRDVAELLLKDSAKIQKEDVEKGKILPLFTEKDVTE |
| | TMEKFTVSEWDTPIETSHDVTVTFLDAGHILGASISIIDI |
| | AGVGRVVYTGDIGHGRSPLMNAPVKLSNADYLIIESTYGA |
| | KQHPFFYPKEELRQIVQDTYNSKGKVLIPVFAVGRAQEIL |
| | YLLKLLYDENKLPNIPVYFDTPLGREATSLYRHHQNYLRT |
| | DFRSSFLKGGNPFNFGTLDFVKGHQRSLDISLSEEPCIVM |
| | AASGMLTGGRIHNHLKSTLEDTNSSLVFVGYQAEGTPGRE |
| | IIDGKRKVEIEGKEYQVKLSVKYIQGLSAHADTDGLFAFA |
| | NSAEILPKKIFVVHGEPENAEALQERIIKNLRIDTCVPEK |
| | GYIENFMEREIIKVIVKDVHLDFTPQFTRFDEQEVFPIVG |
| | ALLRKEDGIHLISKEQYTRILDEAEDKLGHLLERVTEKRD |
| | ITEVEVEGTPEEEMSEAVFKETMIKDALNGILSRSRAREL |
| | KQMIEEEGRNATIAHINKLARKDRLYPQDREKQEELRKVL |
| | VAGINSFPQLALTLLNEVIEK* |
| | (SEQ ID NO: 6) |
| | VSTCEAIPLPSLRFIGATKEVTGSCHLLEVDDRKVLLDFG |
| | MKQGEGADRAFEFREFHPGEIDAVLLSHAHIDHSGLLPYL |
| | VAQGFEGKIHSTVATKDLARLLLEDSAKIQKMDSEAGGEA |
| | PLYTAEDVVETVRRIEPAEYGKEFEIPGGSACFYDAGHIL |
| | GSAVTVISAAGKTVCYTGDLGHSRSPILNAPQVPEEEIDY |
| | LIMESTYGNRSHSDERPEEKLSELAGSAYRGRGKLLVPVF |
| | AVGRAQELVLTIKNMKEHGTIPADMKVYLDSPMAREATEL |
| | YADWANHLKPEFYSMFYRGESPFEFEGFELIKSHGSSEKL |
| | AEEEGPAIILAASGMLEGGRVLNHLPRVLKDRSSAICFVG |
| | YQAEGTLGRELVDGGSEVRVNEELVRVLCRVDSIGSYSAH |
| | AGRNGLLGFLDSLPVVPYKTFLVHGEPDAAKALAGEVHRR |
| | RLRVAVPDRGDREPFGGVVSRTVREKEFELGIKPSYTDIG |
| | GGVQVAPVVGALVTDPGGNVRLVSQAELHELMEKRKGELE |
| | GDIREMKAAPQRAAEGTAAGAGEAHERDRTASITLQGYRE |
| | KLEHFATSRKDAMGDTILTRKLAHEIYCKARREGPDSVIK |
| | FINKKLEKKKFNVDDEEIMEEFSEFIKGAVGSLRVDEFCG |
| | ELDEYRSLGGCE* |
| | (SEQ ID NO: 7) |
| | MNRIKFVGAAGEVTGSCHLLELNGKKILLDCGFKQGEGAD |
| | NSPQWPFRPQDIDAVVLSHAHIDHSGHLPTLVSQGFKGRI |
| | YATEATRELARVLLLDSAKIQEEDHENGRTAEPPLYGEED |
| | VHETVRRIEPHPYEKSFELPGGATGKFYDAGHILGSAVVV |
| | LEAGKTICYTGDLGHGESPILGAPQVPREDVDYLIMESTY |
| | GGEHRGGTSEDAENRLAELVESACVESGGRLLIPVFAVGR |
| | AQEILYAIRKLKESGRVPEDIPVYLDSPMATRVTDLHSTM |
| | ADYLRPEFYGKFVEGDSPFEFDGFEPVRSNRDSSRLAEER |
| | SPAVVLAASGMVEGGRVMNHISRVLKDDNSTICFVGFQVK |
| | GTLGRDIRDGNEEVVVGDTPVEVKCGVESLPFFSAHADTD |
| | GLMKFFDGLDPLPYKTFAVHGEPESCETVVSAVAEKKARC |
| | AAPSPDHEEAFGGVTEVVKEQGGFKMDLAPDFVRVGSRRF |
| | APVVGVMVEDEDGTLRLTNESEVIPLWEDERRSSLLRFNE |
| | SLPVGNGLRVPGDDSGGEDSADMAYEEFCDRLEELARRTD |
| | EYGDSIMTKALARDLCGEAVVGANAVIRMINSKEEKGKFN |
| | IEDPDTVAEFCETVRRAVMSLNQYEFREALSRYNERDCI* |
| | (SEQ ID NO: 8) |
| | MSSPINIQFCGATGEVTGSMHLLTIDNKKILLDAGAFQGT |
| | EAKDKNSAPLIFDPAEIDYIFLTHAHYDHTGRLPMLCQRG |
| | FKGEIITTPVTREITFRIMDDSLRIQKEEGKELLFSEDDV |
| | KKAKSLFVPLNEDYPQWQDDEKKIKIKFIPSEHILGSSSI |
| | FIEEPVSLLYSGDVGGGSSSLHSIP KPPDTCDYLII |
| | ESTYGNRNLEKSNSEILSQLKAAVESIGKNNSRLLIPIFS |
| | IDRAEEILFMLRELNIKEKIYLDTPMGIDILDIYSHNKYL |
| | LSKISDEFIKKNSKELDKIFHPDNFERLRAKKNSDELAES |
| | SESCIILASSGMLEGGRIRKYLPQFLPDEKNILLFSGFQA |
| | EGTLGRDIINGYPEVNVDGIPVKVKAQIRKIEGLSAHADK |
| | TALLNYIDCFKTLPVKIFIVHGEMVASLELSDAIKEKFRI |
| | KTVLPKINEKYDLAAGEIRERTIIKGISIGNVRLNFENIS |
| | GKKIALFAGGIIDNGNEYSLVSVREIEEMLHDLKKEMVKS |
| | LPEEIIISETHIRPETLSSATPPSPEELILGLIKIFKAGY |
| | VSKGLIRDLMDASERGISEYRKVIDKKIKNDALILDENDL |
| | RRKGIQIPDRSLISGQLEDLLKRSSLMEQLNLQLALHRMF |
| | TEIK* |
| | (SEQ ID: 9) |
| | MKIQFLGAAKEVTGSCILIETRRSKFLLDCGQRQGQGAES |
| | SPEFQFNPREIDFVILSHAHIDHSGLLPKLVAEGFDGEIY |
| | STVATRDVAELLLEDSAKIQKEAESGAGLIPMFTEEDVAL |
| | TMDKFSVYEWDVPIDASDDVTVTFLDAGHILGASISVIDI |
| | SGAGRLVYTGDIGHGHSPIMNPPAKLNNTDFLIIESTYGA |
| | KRHPAVNPKESLKQIILDTYANKGKVLIPVFAVGRAQEVL |
| | YILKQLYEESELPNIPVYFDTPLGAETTSLYQYHQNYLRA |
| | QFRSSFLKGENPFHFGTLDFIRGNNRSLDVASSEEPCVVI |
| | AASGMLTGGRILNHLKTTLEDPDSSLVFVGYQAEGTPGRE |
| | ILDGKKRVEIAGKEYEVKLKVHYIPGLSAHADADGLFSFV |
| | NSADILPKKIFVVHGEPENAEALQRRIINNLRIDTFVPEM |
| | GYTENFMDREVVKVVEKDVHLDFKPEFTKVGELEVYPFVG |
| | ALLKKQDGIHLISKEQYIGILTETEEELETMLTKMTSKEG |
| | LEAETETETAEPVEEISETEFKETLLKYADVGIFSRSRAR |
| | ELKNMLENEGRDATIAHINKLARKDRLYPLDTTKQEELRR |
| | VLVAGINSFPQKARELFNEIVER* |
| | (SEQ ID NO: 10) |

### Cas7 Polypeptides

In an embodiment, the Cas7 of Type VII systems are distantly related from a phylogenetic perspective to the Cas7 of Type III-D Cas7 proteins. However, the Cas7 of Type VII systems has an apparent inactivation of the Cas7 catalytic residues that are required for target RNA cleavage in Type III systems. See e.g., FIGS. 5B, 8B-8E. In an embodiment, one or more Cas7 polypeptides are involved in binding of the guide molecule. In an embodiment, multiple Cas7 polypeptides are involved in binding of the guide molecule.

In an example embodiment, the compositions may comprise a Cas7 ortholog or homolog. For example, the Cas7 may be a Cas7 from an ortholog or homolog of the Type VII system and heterologous to the Cas15, Cas5, and/or Cas6 polypeptides. In one embodiment, the Cas7 polypeptide may be selected from the group consisting of Cas7 (COG1857), Cas7 (COG3649), Cas7 (CT1975), Csy3, Csm3, Cmr6, Csm5, Cmr4, Cmr1, Csf2, and Csc2 polypeptides, homologs thereof, and orthologs thereof. In another embodiment, the Cas7 family polypeptide is a Csm3 polypeptide or a homolog or ortholog thereof. In one embodiment, a Cas7 is a CRISPR Type III Csm3 polypeptide or a homolog or ortholog thereof. The Cas7 polypeptide that is an ortholog or homolog of a Type VII Cas7 may also comprise one or more variations (e.g., mutations, truncations, etc.) of the wild type Cas7 family protein.

In an embodiment, the Cas7 polypeptide comprises one or more amino acid sequences of Table 2.

TABLE 2

| Poly- | |
|---|---|
| pep- | |
| tide | Amino Acid Sequence |
| Cas7 | MAKTMKKIYVTMKTLSPLYTGEVRREDKEAAQKRVNFPVRKTATN |
| | KVLIPFKGALRSALEIMLKAKGENVCDTGESRARPCGRCVTCSLF |
| | GSMGRAGRASVDFLISNDTKEQIVRESTHLRIERQTKSASDTFKG |
| | EEVIEGATFTATITISNPQEKDLSLIQSALKFIEENGIGGWLNKG |
| | YGRVSFEVKSEDVATDRFLK* |
| | (SEQ ID NO: 11) |
| Cas7/ | MSREYLYMEIEMNTESPFVSGEIKQVHQERGAAKPVRKTADGKVA |
| Csm3 | APIYGALRAYLEKTLRAKGDTVCDSGKKTCGHCVLCNLFGSLGKG |
| gr7- | GRAVIDDLISDKPASEIVKPVIHLRLNREDNTVADSLRQEEVQEA |
| like | VVFKGRIIIDNPGDRDLTLIQTGIEAINEFGLGGWRTRGRGKVNM |
| | KITKVEKRNWATFEEKGKEIADKLLTP* |
| | (SEQ ID NO: 12) |
| | LRVGGDGVNKYLVFDIKATTTLPAVTGEIKMDRKADIKLARITGD |
| | GRVAIPIYGALRGYLERILRENGENVCDTGMKDAKPCGRCVLCDL |
| | FGSLGKKGRAIIDDLVSERSYKEIVHPSVHLRISREDGVVSNTLK |
| | IEEIEEGAVFTGKIRVVDPKPRDKELIVAGLKAIEEFGIGGWVTR |
| | GRGRVKVDFSIQEREWTDFLKQARNILEKL* |
| | (SEQ ID NO: 13) |
| | MTNDEFYVLDMEMKTISPVISGEIKTSERDFKRKKDINAPCRITG |
| | DNKVAVPIYGVVRGYLERILREKGENVCDTGAKGAKGCGRCILCD |
| | LFGNLGRRGRVFFDDLKSNEDFNKVVKVSFHSRISRDDASVSDSL |
| | TIEEIQEDALFEGKIRILNPKEKDIELLSASIEAINEFGLGGWIR |
| | RGRGRVDMKIKSVSKRKWSEFYERGKEVAAKIMV* |
| | (SEQ ID NO: 14) |
| | VDEYLSIRVEATTLLPLVSGEIKSREVREGEHRIKPARITGDGKV |
| | AVPIYGALRAYLEKTLRENGEQVCDTGLPGKEGQGCGKCVLCDLF |
| | GYLGKRGRAIIDDLKSEKPYREVVARATHLKIDREKGAVNATLKM |
| | EEIVEGTKFVGYIRIIDPKPRDVELILTGLKAIEEFGLGGWLTRG |
| | RGRVKIGYAIEKKRWSDFVKKAREELKGIT* |
| | (SEQ ID NO: 15) |
| | VIKLDDITRLKIKMTTISTLISGEIKTDYINKDKSVKLIRRTSNG |
| | KVAVPIYGVLRANAEKILKEKGGNVCDAGRPGTDATCGKCKVCSL |
| | FGAMSQRGRAIIDDLISKKDAKEIVHKSFHSKIDRDTRSVVSGGT |
| | LNVEEVEENADFYGDIVILNSKEEDLNILAASMEATNMTGLGGWV |
| | TRGRGKVKMEIETIESFKWTDFIKNAGEKLLKVLK* |
| | (SEQ ID NO: 16) |
| | MDKFLVIEVYAKTISPLYTGEIKKEAIREARDVNLPVRRTEDGKV |
| | AIPIYGVIRAYLEKILTEKGENVCDTGAKGAKGCGRCVLCDLFGY |
| | LGRRGRAFIDDLVSKENAMKIVSSVTHNRIDRNSGTVSDALKMEE |
| | IKEGSEFYGKIRIIEPKERDIELFATAFEAMKEFGIGGWVTRGRG |
| | RVDIQFKVYERRWTEFINRAKETLKQIGIK* |
| | (SEQ ID NO: 17) |
| | MEKEFGKYRLEMRAVSTVISGEIKEERRRKEKSGVHLPCRLTADG |
| | KVAVPIYGALRGYAEIALRATGEEVCDTGAKGSKGCGRCSLCDLY |
| | GSLGMRGRAIIDDLRSEENFDKVVNKVMHVKLDREKGVVSDSLEA |
| | EEIQEGTIFTGTIIVLNPRERDLELINIGIQGINTFGLGGWLTRG |
| | RGRVELKIVSAERISWASLVEEARKRVKELVKTKK* |
| | (SEQ ID NO: 18) |
| | MLNEDLWILELKATAVSPLYTGENKIDSLKRRKQGNLLPTRMSGD |
| | GFASISIFGAIRGYAEKIYKDAGTCDTGKDTKGCGRCLTCDMFGN |
| | LGRKGRVSFEDLKSVRPFDKVVERTVHPHIDRETGTISSGKGASI |
| | ELEEIVEGTELTGRIIIKNPTEKDIEVLNAALAAAEDNGIGGWTR |
| | RGKGRVKFEVTAKKVKWANYKEQGAAEAKKLVSMK* |
| | (SEQ ID NO: 19) |
| | MGNKGEYFHIDVEMRTLSPLFTGEIKGTKAKGVKPVRKSSDGRVV |
| | VPIYGALRAGIERSLRASGEQVCDSGKKACGQCVVCSLFGSLSQG |
| | GRAVIDDIVSDEPASKIAHASNHVRLNRETNTVEDSLKQEEVEEG |
| | AVFRGRILVDRPDDRDIELLQTGVEAVNEFGLGGWRTRGRGKVEI |
| | SLANVEKRRWEDFKEEGSKKARELLSS* |
| | (SEQ ID NO: 20) |
| | MSEKKESERPYAVVDLEMETVSPLVTGEIKKVPNKGTKPVRKTAG |
| | GSVAVPIYGAIRAGLEKSLRDKGEKVCDSGKKTCGRCVLCGLFGS |
| | LGKGGRALIDDMVSERPASEIAHPSTHVRLNRDDGTVDDSLSQEE |
| | VEEGAKFTGRMIVDRANDRDIELIHSGIESINEFGLGGWKTRGRG |
| | RVNMRITGITWKRHRDFMDRGKEKARELLR* |
| | (SEQ ID NO: 21) |
| | MQNEYMKYELEMKTLSTVISGEIKEEERRRKERAGVHLPCRITAD |
| | GRVAVPIYGALRGYAEIALRANGEDVCDTGGKGTKGCGKCVLCDL |
| | YGSLGRRGRAMIDDLRSAENYDKVVKKVMHVKLDREKGNVSDALG |
| | AEEIQEGTAFTGNIVVLNPKERDTELINTGIEGINEFGLGGWLTR |
| | GRGRVSLKIASVEKKLWDTLVEEARKKTKELLEKK* |
| | (SEQ ID NO: 22) |
| | MEKEYMRYGLEMKTLSTVISGEIKEEERRRKEREGVHLPCRITAD |
| | NKVAVPIYGSLRGYAEIALRASGEDVCDTGGKGTKGCGKCVLCEI |
| | YGSLGRRGRALIDDLRSVENYDKVVKKVMHVKLDREEGKVSDALG |
| | AEEIQEGTVFTGNIVVLNPKERDTELLNIGIAGINEFGLGGWLTR |
| | GRGRVELKIASVEKRSWDTLVEEARKKAKELLEKKK* |
| | (SEQ ID NO: 23) |
| | MLNEDLWILELKATAVSPLYTGENKLESQKRRKQGNMLPTRMSGD |
| | GFASISIFGAVRGYAEKIYKDAGTCDTGKDTKGCGRCLTCDMFGN |
| | LGRKGRVSFEDLKSVRPFDKVVERTVHPHIDRETGAISAGKGASI |
| | ELEEIVEGTELTGKIVIKNPTEKDIEVINAALAAAEDNGIGGWTR |
| | RGKGRVKFEVTPKKVKWANYKELGAAEAKKLVNMK* |
| | (SEQ ID NO: 24) |
| | MIGVDLCNLSRITIPSSLSNNLTFIRQKYKIKRYIGYGEMDNEYM |
| | RYGLEMKTLSTVISGEIKEEERRRKERAGVHLPCRITADGRVAVP |
| | IYGALRGYAEIALRASGEDVCDTGGKGTKGCGKCVLCDLYGSLGR |
| | RGRAIIDDMRSVENYDKVVKKVTHIKLNREEGNVSDALGAEEIQE |
| | GTVFTGNIVVLNPKERDTELINTGIEGINEFGLGGWLTRGRGRVE |
| | LKIASVERRSWDTLVKEAREKAKELLGRE* |
| | (SEQ ID NO: 25) |

### Cas5 Polypeptides

The Cas5 of Type VII systems are distantly related from a phylogenetic perspective to Cas5 from Type III-D systems. See, e.g., FIGS. 5B, 8B-8E. In Type III CRISPR systems, the Cas5 appears to be implicated in binding the 5′ region of crRNA. See Kazlauskiene, et al. Spatiotemporal Control of Type III-A CRISPR-Cas Immunity: Coupling DNA Degradation with the Target RNA Recognition. Molecular Cell 2016. In an embodiment, only the Cas5 polypeptide binds the guide molecule. In an embodiment, the Cas5 polypeptide binds the guide molecule in combination with one or more Cas7 polypeptides.

In an embodiment, the composition may comprise a Cas5 ortholog or homolog that is heterologous to the Cas15, Cas7, and/or Cas6 polypeptides of the Type VII system. As used herein, when a Cas5 family polypeptide originates from a species, it may be the wild-type Cas5 family protein in the species or an ortholog or a homolog of the wild-type Cas5 family protein in the species. The Cas5 family polypeptide that is an ortholog or homolog of the wild-type Cas5 family protein in the species may comprise one or more variations (e.g., mutations, truncations, etc.) of the wild-type Cas5 family protein. In an embodiment, the Cas5 polypeptide is selected from the group consisting of Csm4, Csx10, Cmr3, Cas5, Cas5 (BH0337), Csy2, Csc1, and Csf3 polypeptides, homologs thereof, and orthologs thereof. In an embodiment, the Cas5 polypeptide is a Csx10 polypeptide, ortholog, or homolog thereof. In an embodiment, the Cas5 polypeptide is a Type III Csx 10 polypeptide, ortholog, or homolog thereof.

In an embodiment, the Cas5 polypeptide comprises one or more amino acid sequences of Table 3.

TABLE 3

| Polypeptide | Amino Acid Sequence |
|---|---|
| Cas5 | MKEIKGILESITGFSIPLDNGEYALYPAGR |
| | HLRGAIGYIAFNLDLPISSKFLDFDFDDII |
| | FRDLLPISKCGKIFYPEKNSNSLKCPSCNE |
| | IYGSSVLRNIMARGLSYKEVIEGKKYRLSI |
| | IVKDEKYLNEMEAIIRYILSYGIYLGNKVS |
| | KGYGKFKIKEYSIVDILPVKDSEVLLLSDA |
| | IIDNGEKDIVFSKKEISSSKFEIIRKRGKA |
| | KGDIIRDNNHNGFYIGKYGGLGFGEIISLK |
| | * |
| | (SEQ ID NO: 26) |

### Cas6 Polypeptides

Cas6 polypeptides are optional in the compositions disclosed. In Type III CRISPR systems, the Cas6 appears to be implicated in crRNA processing in naturally occurring systems. See, e.g., FIGS. 5A, 8A. See Pyenson & Marraffini. Type III CRISPR-Cas systems: when DNA cleavage just isn't enough. Current Opinion in Microbiology, (2016); Carte et al. Cas6 is an endoribonuclease that generates guide molecules, e.g., guide RNAs, for invader defense in prokaryotes. Genes Dev., 2008; Hatoum-Aslan, et al. Mature clustered, regularly interspaced, short palindromic repeats RNA (crRNA) length is measured by a ruler mechanism anchored at the precursor processing site. Proceedings of the National Academy of Sciences, (2011). In an embodiment, a Cas6 polypeptide may be included as part of the Type VII complex where its presence may improve stability of the complex or enhance function of the Type VII system.

In an embodiment, the Cas6 polypeptide comprises one or more amino acid sequences of Table 4.

TABLE 4

| Poly- | |
|---|---|
| peptide | Amino Acid Sequence |
| | MNEMRVEISFGTIEKPPIHSIQSFIYQCLHEEDKEYATFI |
| | HKEGIKHKWKSIKPFIFSHPYIKKDNKCYIKVSSLDSFFN |
| | YNFFSGLNKIMFKKNNAIIKPESIRIINIPKIKYDSNGNT |
| | QQNMFFISPLLVKSNSGYIKDPDDEKFLQILKKNLIDKYL |
| | AINKKECTNDTFSIFLKDKTPKLKIYKKEELPVFLSKFVL |
| | ISSKELFNIAYYLGLGCKNALGFGMIEFDREDL* |
| | (SEQ ID NO: 27). |
| | VKDIKLFPIAYYGFSFIVIDRVYFTKNICASLRGIIFSNL |
| | KGLFCEKKGKDCGECDYSSKCPVSGFFEITRDTKRYRDYP |
| | RPVVLRVDEPLNVYEASSDFEFSLGLLSKKSIDDFPYIFS |
| | AIKHIENTGIGSEEEKKRGKVKLISAYFLNPLNNEKKILY |
| | FYKENIFNERKLYIKKKEIIERAKEISKGKRIKIEFLTPL |
| | AITYEKKIMKDFSFPLFIERLYERINKISELSLKKPLDIN |
| | VPPLDSIKILYKNFTYYNSFRYSTRKGSYIPLNGIIGEAV |
| | ISGRIDKIAVPLVIGELIHVGKHTTSGFGRYKLSVL* |
| | (SEQ ID NO: 28) |
| Cas6- | MSEQSYIALIAEGTLKNITEIILGTGEKDKYNALMSKAYP |
| Cas7 | EGQNIRGAFGYTFLEADAGISQTYMENTPAVLYFRDARPR |
| Fusion | HFRDKGKLMPVMKENKFVNYRCTSCGDNIESPTYMNKIVS |
| | IKLDRKTNSVASGAFVKHEAILGRSIFDFRVALNLKRGKE |
| | YASEFIAAVEKFSNEYIRLGKRRNKGKGLFTLEDVKYSTV |
| | TLNDIKKRAEALKKKDKLIMYFFSDIVVDRKITVEMILRS |
| | IKNCAKFMHPEYESYNDPSLKIFSNTLPIKKIVFLDRKEG |
| | ISQKVIYENIIPKGGVVKLQVKDASTMFWEALALTEAFMG |
| | IGKRTSFGKGEFKIF* |
| | (SEQ ID NO: 29) |
| | VAEGVITNITEYHLGTGFKKHGLSTTHPYPQGQHIRGSVG |
| | YELLHVNAGLSKSFLDENAALIYFKDAIPLHADGGLLLPF |
| | VAEKFQNFKCSTCGQVLKHPTRKGTIIQTRVDRKTGKTSA |
| | FRLEAVTRGYSYRFKAVLNMRRGEDYAEEFVAVMELIEEN |
| | GLKLGRRSGKGKGHFRIDRLNYRMIRLEDIRRRARELERE |
| | LERKDRLTLHFISDFIGELTGETILTGVKNAGRHFHPDYE |
| | SYEDPFVGVKKECLPPRTVLSLHRKVTPNGGKNRIMKDMA |
| | IPAGAKVQVEFAEKPPEIFYECLAIAELRGIGAKTSFGKG |
| | EFVVV* |
| | (SEQ ID NO: 30) |
| | MTEKIATIVNGNFTNITETVLGAGFRNERLKVEVSHDYPL |
| | GQMLRGAFGYLFMDMNLKIADTYEADNHSVIYFRDAIPHH |
| | FKDDGIIVPFIADKRFINYKCEKCGEVLKYASYKTVINKT |
| | RINRNIGGVSQMFREEGIVRKNKFRFKNVLNLKETLNKNP |
| | EYLVDYMSAIEYVKEFGINLGRGHLKGMGKMVFDDIKYHT |
| | ITLKDIKKRSEELANKKVLTLRLMSDTIVNHNGTSVGKVD |
| | EKILIGSVKTAMKFFHPEWGITGENKLIRNEDSGFINLVN |
| | YDTILNRKMSFMDCKITKRQRRISTEDIIPRGSMFKYEIS |
| | ENMPDGFYEGLAILEMCYGIGKRIEFGKGEILIE* |
| | (SEQ ID NO: |
| | 31) |
| | MREKEVALEVRGVMENVTEVHLGKGISRNKIPLSYSYPQG |
| | QNLRGFFGYEFLRANSKVSKTFLAGNPNYLYFKDARPLHG |
| | DGGELLPVIVDGRFINYRCSSCGAVLRYPARKGIFTSVKL |
| | DRATGRVTAFIRREGIVGRNKFLFRVYLNLKRAPEFAEDL |
| | LGAVLKAREEGIRLGARKGRGKGLFTLSDFQIGVITLEDI |
| | RRRADELESQEVHTFHFLSDVVAPEGLPETIVRSIKNAGK |
| | FLHPEYVSYSDPYFKVVSKKVLPPRTVVFLDRKEDRDGYG |
| | KNRLSKVSVIPRGAEITLKFSSAPRMFYECMAVAEKIMGA |
| | GKRTGFGKGEFRVL* |
| | (SEQ ID NO: 32) |
| | MIAVELTAKFRNKDLLHFGTGYKDQRARAKNTWPYPPGQV |
| | LRGRFGYLLMNMQSDIVDSFKDGIPLLYFKDALVYHKKCG |
| | GLLYPTIEKQRFLNYKCNNCNEILRYATRISTVNKTRMSR |
| | QTYKVNQLYRLQMISNYNKFRLKIIIKLKNNEKRFEDPIA |
| | IMEFARNFGLNFGGRCHKGIGKLFIEDYQLDVISDEMINQ |
| | RAEELCIKKKFKIHLLSQYIPKQNNNTNFLTVKDFLTSIK |
| | NAGKFFLYDYTNYPDPKLKLIDSQTRKPIKINFFDTNRFI |
| | SHHALPEGSQFDFEIENAPFIFWKSLAVAEKCSGIGARTS |
| | FGKGEFIIT* |
| | (SEQ ID NO: 33) |
| | MRYLLIEGIIRNKEIFSIYEPYPVGRHIRGAFGWIAKDMG |
| | LGIKELFPDLNNDILIFRDGIPICRKERWRPFSGKKTLYL |
| | PFIDGGRIKYRCSSCGDFFSQGFHRTEISRIQLDRKKGSV |
| | NSFRRIEAFHREAIFRLSIILDIERGERFVEDLFSMMSFV |
| | KEFGINIGKRHQKGMGHFILEEFDAKIMEDIEIGEKDEFT |
| | INFISDAYIRNGDGFLDLKIKTDKIIGMMERLGNRFKIDF |
| | RGEIEEISKTLIEPRNITMIQVIEDENRFIRIPLNVLSRG |
| | ISIRFKRRGNSRNILKLLKAIELFYGLGDYIGLGLGEIYV |
| | N* |
| | (SEQ ID NO: 34) |
| | MSTDNIAVEVTGDLVNLNETILSTGELDSRTHMMTSHNYP |
| | PGQVLRGAFGHLFFVANLPIIKTYEINAEPVLYFKDALPL |
| | HWRDGGILFPTIVDGKFVNYQCQVCKEILRYASFKSMITR |
| | TRLDRHKGTVSMMWRQEGITQRTQFRFRVIVKLKTDIEDN |
| | LSALFAVLSFVEDNGLSLGKRSMKGSGKFRLDNLAYNLVT |
| | REDIDRRAKELREKEVMKIRLLSDAIVRKQGTNLTIIKGD |
| | DFLWSVKNAAKNFNYDYKPFSAEVKLIDTQTTKPYPIGFL |
| | DIKGQYEIAIPKGSTFTYRIAPGASEDFYIALALLERCHG |
| | IGNRSSSGKGEIIIE* |
| | (SEQ ID NO: 35) |
| | MGKLQRTRGSGSQEAGEHEMKPLAFKIQGELENQSELHIA |
| | TNNTYTERGSQIKESDMYPLGQNLRGAFGYLFLEMKSKIA |
| | DTLKNGHPVIYFKDALIKHHDGTFIPVVKLDKGIRQVVYE |
| | CSECLHIDKYPAIRQPMAGISLGSGGTVKNMYMTDVVSGK |
| | NRFRFEAIFNMKAKNVEEETLNEEYLAEFICALKYVEDNG |
| | LYLGRRNSKGLGKVLLKKLKILPITMDDIKKRAKIISQIV |
| | RDEDGKMWIHLFSDTISSFPLSGEDIVRD |
| | (SEQ ID NO: 36) |
| | MSSNDKVALVVEGKLQNRTEVILGTAMRGKDKVYMSKKYP |
| | EGQHLRGAFGYAFLYGGAEVIESYREDRPKSHPAIYFRDA |
| | RPVHFEDGGDLVPVVVKRRFVNYRCSRCGKVLRFPTFMQP |
| | VTSIKLDRRTNTVARGAGFVKNQAITRKSNFSFRAALNLK |
| | RGEEYAPELIAAVEKFASEGMRLGKRRSKGKGLFELKDLG |
| | YSTVDLGEIRERAGELREREELVLRLMSDTVVDGEGAITE |
| | SMLLRSIKKAAKFWHPEYEPYSDPFVKMSVDALPPQSTVF |
| | LDRKEGVGAKTLKAKVVPRGATVRLQPRDAPGMFYEALAI |
| | AEAFMGIGKRISSGKGEFAVV* |
| | (SEQ ID NO: 37) |
| | MAGEADVALVASADLVNLSEVVLGTGTKSKKGLPMSRGYP |
| | EGQHLRGAAGYTFLDAGAGILKTYEDGVPAATYWRDARPL |
| | HRDGGVLVPVNVKGRFVNYRCVKCGRVERFPTEMSPITSV |
| | KLSRTGNTVNNMFGYFGITGQHRFLFRAALPLKRGREYAH |
| | EFAGVLTKWSEEGLRLGRRKSKGKGLFSLTDLTFDTLTLD |
| | DIRDRAAHLDSVADNGGELKLSFFSDIVVGEDGALTEKMI |
| | LRSIKNAAKFQHPMYEPYEDPTVSTRIHALPARREVYLDT |
| | KGKKRRVDKPLVVPRGAEVRMRVRGAPDMFWEALALAESC |
| | MGIGKRTSSGKGEFHCLV* |
| | (SEQ ID NO: 38) |
| | MSGESTAVEVMGELVTQNEAILSTGEFDRRAHAMRSYGYP |
| | PGQAIRGAFGYLFLAAGLPIIKTYEVNAEPVLYFKDALPY |
| | HYRDGGILYPVITPEKYVNYQCQECKEVLRYPSFKSVITK |
| | TRLDRHKGTVSMMWRQEGITKRARFRFKIIVKLKRDAEEN |
| | LAALMTVLKFVEENGLALGKRSMKGSGKLRLENLSYKLVT |
| | AGDIEDRAAELREKDIIRVRLLSEAIAREKGANLTLIRGN |
| | NFLWSVKNAAKNFWYDYKNFSAEVKLINYETTRPYPVGFL |
| | DLKLAGGRPEIAIPKGSTFTYKIARDAPAEFYTALALLER |
| | CHGIGNRASSGKGEIIVE* |
| | (SEQ ID NO: 39) |
| | MSSDNIAVEVTGELANQNEATLSTGEFDRRAHAARSYDYP |
| | PGQAIRGAFGYLFLAAGLPIIKTYEVNAEPVLYFRDALPY |
| | HYRDGGILYPVITPEKYVNYQCQKCKEVLRYPTFKSVVTK |
| | TRLDRYKGVVSMMWRQEGITRRENFRFLVIVKLKRDAEDN |
| | LSALIAALRFVEENGLAIGKRSMKGSGKLLLKNLSYELIT |
| | RRDIEGRAAELREKEVIKVRLLSEAIVREKGTNLTTIPGK |
| | NFLWSVKNAAKNFNYNYQNFAVDVKLINYETTRPSSVGFL |
| | DLKLSGGGLEIAIPKGSTFTYKIERGAPSEFYTALALLER |
| | CHGVGNRVSSGKGEIIIE* |
| | (SEQ ID NO: 40) |
| | MKPLAFKIQGELENQSELHIATNNTYNERGSRIKASDIYP |
| | LGQNLRGAFGYLFLEMKSKIADTLENGHPVIYFRDALIKH |
| | HDGIFIPVVKLDKGIRQVVYECCECLHIDKYPALRQPMAG |
| | ISLGIGGTVRNMYTTDVISSKNRFRFEAIFNMKAKSAQEE |
| | KLNEEYLAEFISALKYVEDNGLYIGKRNSKGLGKVILKKL |
| | TIVPITMEDIKKRAKIISQIVRDEDGKMWIHLFSDTLSSF |
| | PLSGEDIVRDTKNAAKFFDPDFTQYKDPKITHIEKPVELL |
| | VNLSFLDLKVKTGKPRFEAPKSVISRGTRFRYQVTDAVPE |
| | FFNAFAMAELLRGLGDRTSFGKGEFVVS* |
| | (SEQ ID NO: 41) |
| | MSNENIAVEVTGKLVTQNEVTLSTGEFDRRAHAMRSYGYP |
| | PGQTIRGAFGYLFLAAGLPIIKTYEVNAEPVLYFKDALPY |
| | HYRDGGILYPVITPEKYVNYQCQECKEVLRYPSFKSVITK |
| | TRLDRHKGTVSMMWRHEGITKRAQFRFKVIVKLKRDGEEN |
| | LAALMAVLKFVEENGLAIGKRSMKGSGRLRLENLSYKPIT |
| | RDDIEDRAAELRDKEVIKVRLLSEAIVREKGTNLTIIRGK |
| | NFLWSVKNAAKNFYYDYKNFSAEVKLIDYVTTRPYPVGFL |
| | DLELAGGRPEIAIPKGSTFTYRVERGAPAEFYTALALLER |
| | CHGIGNRASSGKGEIIVE* |
| | (SEQ ID NO: 42) |

### Other Type VII Cas Polypeptides

Additional Cas polypeptides are optional in the compositions disclosed. However, in an embodiment, a CARF/Csa3 polypeptide may be included as part of the Type VII system.

In an embodiment, the CARF/Csa3 polypeptide comprises one or more amino acid sequences of Table 5.

TABLE 5

| Polypeptide | Amino Acid Sequence |
|---|---|
| CARF/Csa3 | MAYILKPDEKTAKLFSDKTRLKILELLSEKELTNSQLAG |
| | MLNLSKPTISHHLKLLLDGGIVKISRIEHEEHGIAMKFY |
| | AVNPNILSVEALKDNKLTEQISKEIQEAMDARSGLSGGH |
| | ANSAFLRMLKSTILNTGIDMDKPFYDAGYKIGVNVISKQ |
| | VKANTLKDVLKELAVLWEKLKLGTVELVSENKIKVADCY |
| | QCGNMPNMGKTLCPSDAGIIAGVLNTVCQKRYSVKETKC |
| | WGTGYDFCEFEIKEL*(SEQ ID NO: 43) |

### Type II CRISPR-Cas Systems and Compositions

Described in example embodiments herein are small Cas polypeptides that have at least one RuvC domain and at least one HNH domain. In one embodiment, the small Cas polypeptide may be a Type II Cas polypeptide. See e.g., Makarova et al., Evolution and classification of the CRISPR-Cas systems, Nature Reviews Microbiology, 2011, Vol. 9, No. 6, 467-477, doi: 10.1038/nrmicro2577; Chylinski et al., Classification and evolution of type II CRISPR-Cas systems, Nucleic Acids Research, 2014, Vol. 42, No. 10, 6091-6105, doi: 10.1093/nar/gku241; Shmakov et al., Discovery and Functional Characterization of Diverse Class 2 CRISPR-Cas Systems, Molecular Cell, 2015, Vol. 60, No. 3, 385-397, doi: 10.1016/j.molcel.2015.10.008. The Cas polypeptides disclosed herein are newly identified small Type II-B and Type II-C Cas polypeptides.

In some embodiments, the Cas polypeptide is a Type II-B Cas polypeptide. A Type II-B Cas polypeptide may be a Cas polypeptide of a CRISPR-Cas system that comprises Cas9, Cas1, Cas2, and Cas4. In some embodiments, the Type II-B Cas polypeptide is selected from the group consisting of SEQ ID NOs: 189-269. In some embodiments, the Type II-B Cas polypeptide is encoded by a polynucleotide selected from the group consisting of SEQ ID NOs: 108-188.

In some embodiments, the Cas polypeptide is Type II-C Cas polypeptide. A Type II-C Cas polypeptide may be a Cas polypeptide of a CRISPR-Cas system that comprises Cas9, Cas1, Cas2, but not Csn2 or Cas4. In some embodiments, the Type II-C Cas polypeptide is selected from the group consisting of SEQ ID NOs: 4583-8895. In some embodiments, the Type II-C Cas polypeptide may be encoded by a polynucleotide selected from the group consisting of SEQ ID NOs: 270-4582.

In some embodiments, the Cas polypeptide is less than 1000 amino acids in size. For example, the Cas polypeptide may be less than 950, less than 900, less than 890, less than 880, less than 870, less than 860, less than 850, less than 840, less than 830, less than 820, less than 810, less than 800, less than 790, less than 780, less than 770, less than 760, less than 750, less than 700, less than 650, or less than 600 amino acids in size. In some examples, the Cas polypeptide is less than 850 amino acids in size. As used herein, small Cas9 polypeptides are also referred to as Cas9-t. In some examples, Cas9-t include Cas9 that have less than 850 amino acids in size.

The Cas polypeptides disclosed herein are distinct from existing Type II sub-types e.g. Type II-A, B, and C, and may be considered a new sub-type referred to as Type II-D. In one embodiment, the Cas polypeptide is selected from the group consisting of SEQ ID NO: 8899-9520.

In some embodiments, the Cas polypeptide is less than 1000 amino acids in size. For example, the Cas polypeptide may be less than about 950, less than 900, less than 890, less than 880, less than 870, less than 860, less than 850, less than 840, less than 830, less than 820, less than 810, less than 800, less than 790, less than 780, less than 770, less than 760, less than 750, less than 700, less than 650, or less than 600 amino acids in size. In some examples, the Cas polypeptide is less than 780 amino acids in size.

In some embodiments, the Cas polypeptide is about 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 amino acids in size or less.

In one embodiment, the Cas polypeptide is at least 100, at least 200, at least 300, at least 400, at least 500, at least 600, at least 700, at least 800, or at least 900, amino acids but are less than 1000 amino acids in size.

### Modified Cas Polypeptides

In an embodiment, one or more or all of the Cas polypeptides are mutant, modified, or variant forms of the Cas polypeptides relative to their natural forms. The types of mutations in the Cas proteins can be conservative mutations or non-conservative mutations. In an embodiment, the amino acid which is mutated is mutated into alanine (A). In an embodiment, if the amino acid to be mutated is an aromatic amino acid, it is mutated into alanine or another aromatic amino acid (e.g., H, Y, W, or F). In an embodiment, if the amino acid to be mutated is a charged amino acid, it is mutated into alanine or another charged amino acid (e.g., H, K, R, D, or E). In an embodiment, if the amino acid to be mutated is a charged amino acid, it is mutated into alanine, or another charged amino acid having the same charge. In an embodiment, if the amino acid to be mutated is a charged amino acid, it is mutated into alanine, or another charged amino acid having the opposite charge.

The invention also provides for methods and compositions wherein one or more amino acid residues of the effector protein may be modified e.g., an engineered or non-naturally-occurring effector protein or Cas. In an embodiment, the modification may comprise mutation of one or more amino acid residues of one or more of the Cas polypeptides. The one or more mutations may be in one or more catalytically active domains of the effector protein, or a domain interacting with the crRNA (such as the guide sequence or direct repeat sequence). The effector protein may have reduced or abolished nuclease activity or alternatively increased nuclease activity compared with an effector protein lacking said one or more mutations. The effector protein may not direct cleavage of the RNA strand at the target locus of interest. In a preferred embodiment, the one or more mutations may comprise two mutations.

The Cas polypeptides herein may comprise one or more amino acids mutated. In an embodiment, the amino acid is mutated to A, P, or V, preferably A. In an embodiment, the amino acid is mutated to a hydrophobic amino acid. In an embodiment, the amino acid is mutated to an aromatic amino acid. In an embodiment, the amino acid is mutated to a charged amino acid. In an embodiment, the amino acid is mutated to a positively charged amino acid. In an embodiment, the amino acid is mutated to a negatively charged amino acid. In an embodiment, the amino acid is mutated to a polar amino acid. In an embodiment, the amino acid is mutated to an aliphatic amino acid.

One or more characteristics of the engineered Cas protein may be different from a corresponding wild type Cas protein. Examples of such characteristics include catalytic activity, gRNA binding, specificity of the Cas protein (e.g., specificity of editing a defined target), stability of the Cas protein, off-target binding, target binding, protease activity, nickase activity, PFS recognition. In an embodiment, an engineered Cas protein may comprise one or more mutations of the corresponding wild type Cas protein. In an embodiment, the catalytic activity of the engineered Cas protein is increased as compared to a corresponding wild type Cas protein. In an embodiment, the catalytic activity of the engineered Cas protein is decreased as compared to a corresponding wild type Cas protein.

### Modifications for Guide Molecule Binding

The guide molecule binding of the mutated Cas polypeptide may be increased or decreased as compared to a corresponding wild type Cas polypeptide. In an embodiment, the guide molecule binding of the mutated Cas protein is increased as compared to a corresponding wild type Cas polypeptide. Guide molecule binding can be determined by means known in the art. By means of example, and without limitation, guide molecule binding can be determined by calculating binding strength or affinity (such as based on equilibrium constants, Ka, Kd, etc.). In an embodiment, guide molecule binding is increased. In an embodiment, guide molecule binding is increased by at least 5%, preferably at least 10%, more preferably at least 20%, such as at least 30%, at least 40%, at least 50%, at least 60%, at least 70%, at least 80%, at least 90%, or at least 100%. In an embodiment, guide molecule binding is decreased. In an embodiment, guide molecule binding is decreased by at least 5%, preferably at least 10%, more preferably at least 20%, such as at least 30%, at least 40%, at least 50%, at least 60%, at least 70%, at least 80%, at least 90%, or (substantially)100%.

### Modifications for Specificity

In an embodiment, the specificity of the mutated Cas polypeptide is increased as compared to a corresponding wild type Cas polypeptide. In an embodiment, the specificity of the mutated Cas polypeptide is decreased as compared to a corresponding wild type Cas polypeptide. In one example embodiment, the stability of the mutated Cas polypeptide is increased as compared to a corresponding wild type Cas polypeptide. In one example embodiment, the stability of the mutated Cas polypeptide is decreased as compared to a corresponding wild type Cas polypeptide. In one example embodiments, the mutated Cas protein, e.g., the Cas15 protein, further comprises one or more mutations which inactivate catalytic activity. In an embodiment, the off-target binding of the mutated Cas polypeptide is increased as compared to a corresponding wild type Cas polypeptide. In an embodiment, the off-target binding of the mutated Cas polypeptide is decreased as compared to a corresponding wild type Cas polypeptide. In an embodiment, the target binding of the mutated Cas polypeptide is increased as compared to a corresponding wild type Cas polypeptide. In an embodiment, the target binding of the mutated Cas polypeptide is decreased as compared to a corresponding wild type Cas polypeptide. In an embodiment, the mutated Cas polypeptide has a higher nuclease activity or polynucleotide-binding capability compared with a corresponding wild type Cas polypeptide.

### Modifications for Stability

The stability of the Cas polypeptide of the invention is altered or modified. It is to be understood that mutated Cas has an altered or modified stability if the stability is different than the stability of the corresponding wild type Cas (i.e., unmutated Cas). Stability can be determined by means known in the art. By means of example, and without limitation, stability can be determined by determining the half-life of the Cas protein. In an embodiment, stability is increased. In an embodiment, stability is increased by at least 5%, preferably at least 10%, more preferably at least 20%, such as at least 30%, at least 40%, at least 50%, at least 60%, at least 70%, at least 80%, at least 90%, or at least 100%. In an embodiment, stability is decreased. In an embodiment, stability is decreased by at least 5%, preferably at least 10%, more preferably at least 20%, such as at least 30%, at least 40%, at least 50%, at least 60%, at least 70%, at least 80%, at least 90%, or (substantially)100%.

### Modifications for Target Polynucleotide Binding

In an embodiment, the target binding of the Cas polypeptide of the invention is altered or modified. It is to be understood that mutated Cas has an altered or modified target binding if the target binding is different than the target binding of the corresponding wild type Cas (i.e., unmutated Cas). target binding can be determined by means known in the art. By means of example, and without limitation, target binding can be determined by calculating binding strength or affinity (such as based on equilibrium constants, Ka, Kd, etc.). In an embodiment, target bindings increased. In an embodiment, target binding is increased by at least 5%, preferably at least 10%, more preferably at least 20%, such as at least 30%, at least 40%, at least 50%, at least 60%, at least 70%, at least 80%, at least 90%, or at least 100%. In an embodiment, target binding is decreased. In an embodiment, target binding is decreased by at least 5%, preferably at least 10%, more preferably at least 20%, such as at least 30%, at least 40%, at least 50%, at least 60%, at least 70%, at least 80%, at least 90%, or (substantially)100%.

### Modifications for Off-Target Binding

In an embodiment, the off-target binding of the Cas polypeptide and/or the complexes of the invention is altered or modified. It is to be understood that mutated Cas has an altered or modified off-target binding if the off-target binding is different than the off-target binding of the corresponding wild type Cas (i.e., unmutated Cas). Off-target binding can be determined by means known in the art. By means of example, and without limitation, off-target binding can be determined by calculating binding strength or affinity (such as based on equilibrium constants, Ka, Kd, etc.). In an embodiment, off-target bindings increased. In an embodiment, off-target binding is increased by at least 5%, preferably at least 10%, more preferably at least 20%, such as at least 30%, at least 40%, at least 50%, at least 60%, at least 70%, at least 80%, at least 90%, or at least 100%. In an embodiment, off-target binding is decreased. In an embodiment, off-target binding is decreased by at least 5%, preferably at least 10%, more preferably at least 20%, such as at least 30%, at least 40%, at least 50%, at least 60%, at least 70%, at least 80%, at least 90%, or (substantially)100%.

### Modifications for Cellular Localization and Trafficking

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 44) or PKKKRKVEAS (SEQ ID NO: 45); the NLS from nucleoplasmin (e.g., the nucleoplasmin bipartite NLS with the sequence KRPAATKKAGQAKKKK (SEQ ID NO:46)); the c-myc NLS having the amino acid sequence PAAKRVKLD (SEQ ID NO: 47) or RQRRNELKRSP (SEQ ID NO: 48); the hRNPAI M9 NLS having the sequence NQSSNFGPMKGGNFGGRSSGPYGGGGQYFAKPRNQGGY (SEQ ID NO: 49); the sequence RMRIZFKNKGKDTAELRRRRVEVSVELRKAKKDEQILKRRNV (SEQ ID NO: 50) of the IBB domain from importin-alpha; the sequences VSRKRPRP (SEQ ID NO: 51) and PPKKARED (SEQ ID NO: 52) of the myoma T protein; the sequence PQPKKKPL (SEQ ID NO: 53) of human p53; the sequence SALIKKKKKMAP (SEQ ID NO: 54) of mouse c-abl IV; the sequences DRLRR (SEQ ID NO: 55) and PKQKKRK (SEQ ID NO: 56) of the influenza virus NS1; the sequence RKLKKKIKKL (SEQ ID NO: 57) of the Hepatitis virus delta antigen; the sequence REKKKFLKRR (SEQ ID NO: 58) of the mouse Mx1 protein; the sequence KRKGDEVDGVDEVAKKKSKK (SEQ ID NO: 59) of the human poly(ADP-ribose) polymerase; and the sequence RKCLQAGMNLEARKTKK (SEQ ID NO: 60) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The Cas polypeptides may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

Catalytically Inactive (dead Cas) and Nickase Variants

In an embodiment, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Methods for generating catalytically dead Cas9 or a nickase Cas9 (WO 2014/204725, Ran et al. Cell. 2013 Sep. 12; 154 (6): 1380-1389), Cas12 (Liu et al. Nature Communications, 8, 2095 (2017), and Cas13 (International Patent Publication Nos. WO 2019/005884 and WO2019/060746) are known in the art and incorporated herein by reference. Such methods can be adapted for modifying the small Cas polypeptides described herein.

Association with Functional Domains

In an embodiment, one or more or all of the Cas proteins of the CRISPR-Cas system is associated with one or more functional domains. For the purposes of the following discussion, reference to a functional domain could be a functional domain associated with one or more of the Cas proteins, e.g., the Cas15, of the CRISPR-Cas system of the present invention, or a functional domain associated with the adaptor protein. The functional domain may be associated with a dead Cas or nickase Cas variant.

In an embodiment, the functional domain may be selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In an embodiment, the functional domain is a transcription repression domain, preferably KRAB. In an embodiment, the transcription repression domain is SID, or concatemers of SID (e.g., SID4X). In an embodiment, the functional domain is an epigenetic modifying domain, such that an epigenetic modifying enzyme is provided. In an embodiment, the functional domain is an activation domain, which may be the P65 activation domain.

In an embodiment, one or more of the Cas polypeptides are associated with a ligase or functional fragment thereof. The ligase may ligate a single-strand break (a nick) generated by the nuclease active Cas protein (e.g., the Cas15 polypeptide). In certain cases, the ligase may ligate a double-strand break generated by the nuclease active Cas protein (e.g., the Cas15 polypeptide). In certain examples, one or more or all of the Cas proteins are associated with a reverse transcriptase or functional fragment thereof.

In an embodiment, the one or more functional domains is a transcriptional activation domain comprises VP64, p65, MyoD1, HSF1, RTA, SET7/9 and a histone acetyltransferase. Other references herein to activation (or activator) domains in respect of those associated with the CRISPR enzyme include any known transcriptional activation domain and specifically VP64, p65, MyoD1, HSF1, RTA, SET7/9 or a histone acetyltransferase.

In an embodiment, the one or more functional domains is a transcriptional repressor domain. In an embodiment, the transcriptional repressor domain is a KRAB domain. In an embodiment, the transcriptional repressor domain is a NuE domain, NcoR domain, SID domain or a SID4X domain.

In an embodiment, the one or more functional domains have one or more activities comprising methylase activity, demethylase activity, transcription activation activity, transcription repression activity, transcription release factor activity, histone modification activity, RNA cleavage activity, DNA cleavage activity, DNA integration activity or nucleic acid binding activity.

Histone modifying domains are also preferred in an embodiment. Exemplary histone modifying domains are discussed below. Transposase domains, HR (Homologous Recombination) machinery domains, recombinase domains, and/or integrase domains are also preferred as the present functional domains. In an embodiment, DNA integration activity includes HR machinery domains, integrase domains, recombinase domains and/or transposase domains. Histone acetyltransferases are preferred in an embodiment.

In an embodiment, cleavage activity is due to a nuclease of the CRISPR-Cas system, e.g., the Cas15 polypeptide. In an embodiment, the nuclease comprises a Fok1 nuclease. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32 (6): 569-77 (2014), relates to dimeric RNA-guided FokI Nucleases that recognize extended sequences and can edit endogenous genes with high efficiencies in human cells.

In an embodiment, one or more functional domains are attached to one or more of the Cas proteins of the CRISPR-Cas system so that upon binding to the guide molecule (e.g., a crRNA) and target, the functional domain is in a spatial orientation allowing it to function in its attributed function.

In an embodiment, the one or more functional domains are attached to the adaptor protein so that upon binding of one or more of the Cas effector proteins of the CRISPR-Cas system to the guide molecule and target, the functional domain is in a spatial orientation allowing it to function in its attributed function.

In an embodiment, the invention provides a composition as herein discussed wherein the one or more functional domains are attached to one or more or all of the Cas effector proteins of the CRISPR-Cas system or adaptor protein via a linker, optionally a GlySer linker, as discussed herein.

### Linkers

The functional domain may be linked to the Cas polypeptide by a linker. The term “associated with” is used here in relation to the association of the functional domain to the Cas effector protein or the adaptor protein. It is used in respect of how one molecule ‘associates’ with respect to another, for example between an adaptor protein and a functional domain, or between the Cas effector protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e., between the enzyme and the functional domain or between the adaptor protein and the functional domain). Thus, in an embodiment, the Cas effector protein or adaptor protein is associated with a functional domain by binding thereto. In other embodiments, the Cas effector protein or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, In an embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In an embodiment, the linker is used to separate the Cas protein and the nucleotide deaminase by a distance sufficient to ensure that each protein retains its required functional property. Preferred peptide linker sequences adopt a flexible extended conformation and do not exhibit a propensity for developing an ordered secondary structure. In an embodiment, the linker can be a chemical moiety which can be monomeric, dimeric, multimeric or polymeric. Preferably, the linker comprises amino acids. Typical amino acids in flexible linkers include Gly, Asn and Ser. Accordingly, in an embodiment, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers GGS, GGGS (SEQ ID NO: 61) or GSG can be used. GGS, GSG, GGGS (SEQ ID NO: 61) or GGGGS (SEQ ID NO: 62) linkers can be used in repeats of 3 (such as (GGS)₃ (SEQ ID NO: 63), (GGGGS)₃ (SEQ ID NO: 64)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 62), (GGGGS)₂ (SEQ ID NO: 65), (GGGGS)₃ (SEQ ID NO: 64), (GGGGS)₄ (SEQ ID NO: 66), (GGGGS)₅ (SEQ ID NO: 67), (GGGGS)₆ (SEQ ID NO: 68), (GGGGS)₇ (SEQ ID NO: 69), (GGGGS)₈ (SEQ ID NO: 70), (GGGGS)₉ (SEQ ID NO: 71), (GGGGS)₁₀ (SEQ ID NO: 72), or (GGGGS)₁₁ (SEQ ID NO: 73).

In an embodiment, linkers such as (GGGGS)₃ (SEQ ID NO: 64) are preferably used herein. (GGGGS)₆ (SEQ ID NO: 68), (GGGGS)₉ (SEQ ID NO: 71) or (GGGGS)₁₂ (SEQ ID NO: 74) may preferably be used as alternatives. Other preferred alternatives are (GGGGS)₁ (SEQ ID NO: 62), (GGGGS)₂ (SEQ ID NO: 65), (GGGGS)₄ (SEQ ID NO: 66), (GGGGS)₅; (SEQ ID NO: 67), (GGGGS)₇ (SEQ ID NO: 69), (GGGGS)₈ (SEQ ID NO: 70), (GGGGS)₁₀ (SEQ ID NO: 72), or (GGGGS)₁₁ (SEQ ID NO: 73). In yet a further embodiment, LEPGEKPYKCPECGKSFSQSGALTRHQRTHTR (SEQ ID NO: 75) is used as a linker. In yet an additional embodiment, the linker is an XTEN linker. In an embodiment, the Cas protein is linked to the deaminase protein or its catalytic domain by means of an LEPGEKPYKCPECGKSFSQSGALTRHQRTHTR (SEQ ID NO: 75) linker. In further particular embodiments, the Cas protein is linked C-terminally to the N-terminus of a deaminase protein or its catalytic domain by means of an LEPGEKPYKCPECGKSFSQSGALTRHQRTHTR (SEQ ID NO: 75) linker. In addition, N-and C-terminal NLSs can also function as linker (e.g., PKKKRKVEASSPKKRKVEAS (SEQ ID NO 76)). Examples of linkers are shown in Table 6 below.

TABLE 6

| | |
|---|---|
| GGS | GGTGGTAGT |
| GGSx3(9) | GGTGGTAGTGGAGGGAGCGGCGGTTCA |
| | (SEQ ID NO: 77) |
| GGSx7(21) | ggtggaggaggctctggtggaggcggt |
| (SEQ ID | agcggaggcggagggtcgGGTGGTAGT |
| NO: 78) | GGAGGGAGCGGCGGTTCA |
| | (SEQ ID NO: 79) |
| XTEN | TCGGGATCTGAGACGCCTGGGACCTCG |
| | GAATCGGCTACGCCCGAAAGT |
| | (SEQ ID NO: 80) |
| Z-EGFR_ | Gtggataacaaatttaacaaagaaatg |
| Short | tgggcggcgtgggaagaaattcgtaac |
| | ctgccgaacctgaacggctggcagatg |
| | accgcgtttattgcgagcctggtggat |
| | gatccgagccagagcgcgaacctgctg |
| | gcggaagcgaaaaaactgaacgatgcg |
| | caggcgccgaaaaccggcggtggttct |
| | ggt |
| | (SEQ ID NO: 81) |
| GSAT | Ggtggttctgccggtggctccggttct |
| | ggctccagcggtggcagctctggtgcg |
| | tccggcacgggtactgcgggtggcact |
| | ggcagcggttccggtactggctctggc |
| | (SEQ ID NO: 82) |

### Guide Molecules

In an embodiment, a CRISPR-Cas system comprises one or more guide molecules (also referred to herein as guides). Guide molecules comprise a guide sequence and a scaffold. The guide sequence is an engineered sequence designed to change the target sequence recognized by the complex to a target sequence other than a sequence defined by the protospacer of a naturally occurring crRNA. In some embodiments, one or more of the crRNAs independently comprise the sequence of FIG. 3G.

In some embodiments, the system includes two guide molecules that can each be splint or bridge molecules. In some embodiments, the first and second guide molecules comprise a region capable of hybridizing to a cleaved strand of the target polynucleotide and a region capable of hybridizing to the donor sequence. In some embodiments, the composition comprises a splint oligonucleotide that has a region capable of hybridizing to a cleaved strand of the target polynucleotide and a region capable of hybridizing to the donor molecule.

The ability of a guide molecule to direct sequence-specific binding of the CRISPR-Cas complex to a target nucleic acid sequence may be assessed by any suitable assay. For example, the components of a CRISPR-complex may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36 (4)702-707). Similarly, cleavage of a target nucleic acid sequence may be evaluated in a test tube by providing the target nucleic acid sequence, components of the CRISPR-Cas complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) are included in the CRISPR-Cas has sufficient complementarity with a target nucleic acid sequence to hybridize with the target nucleic acid sequence and direct sequence-specific binding of a nucleic acid-targeting complex to the target nucleic acid sequence. The degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence may be selected to target any target nucleic acid sequence in a target polynucleotide. In one embodiment, the target polynucleotide may be DNA. In one embodiment, the target polynucleotide is an RNA polynucleotide. The target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In one embodiment, the target sequence may be a sequence within an RNA molecule selected from the group consisting of mRNA, pre-mRNA, and rRNA. In one embodiment, the target sequence may be a sequence within an RNA molecule selected from the group consisting of ncRNA, and lncRNA. In one example embodiment, the target sequence may be a sequence within an mRNA molecule or a pre-mRNA molecule.

In one embodiment, the scaffold is located 5′ of the guide sequence. In one embodiment, the scaffold is located 3′ of the guide sequence. The direct repeat may comprise one or more modifications. The modifications may remove unnecessary secondary structure or otherwise minimize the overall size of the scaffold component of the guide molecule. The direct repeat may have one or more modifications that increase the stability of the guide molecule, enhance complex formation with the Cas polypeptides described herein, for example by modulating nuclease activity (either by increasing or decreasing), and/or reducing off-target effects.

In an embodiment, the guide sequence length of the guide molecule is from 15 to 35 nt. In an embodiment, the guide sequence length of the guide molecule is at least 15 nucleotides. In an embodiment, the guide sequence length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

In some embodiments, the degree of complementarity between a guide sequence and its corresponding target sequence can be about or more than about 50%, 60%, 75%, 80%, 85%, 90%, 95%, 97.5%, 99%, or 100%; a guide sequence can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide sequence can be less than about 75, 50, 45, 40, 35, 30, 25, 20, 15, 12, or fewer nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the target sequence and the guide sequence, with it being advantageous that off target is 100% or 99.9% or 99.5% or 99% or 99% or 98.5% or 98% or 97.5% or 97% or 96.5% or 96% or 95.5% or 95% or 94.5% complementarity between the target sequence and the guide sequence.

### Guide Molecule Modifications

Many modifications to guide molecules are known in the art and are further contemplated within the context of this invention. Various modifications may be used to increase the specificity of binding to the target sequence and/or increase the activity of the Cas protein and/or reduce off-target effects. Example guide molecule modifications are described in International Patent Application No. PCT US2019/045582, specifically paragraphs [0178]-[0333], which is incorporated herein by reference as if expressed in its entirety herein. Additional guide molecule modifications are described in detail below.

The guide molecule may be designed to reduce the degree secondary structure within the guide molecule. In some embodiments, about or less than about 75%, 50%, 40%, 30%, 25%, 20%, 15%, 10%, 5%, 1%, or fewer of the nucleotides of the guide molecule participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A.R. Gruber et al., 2008, Cell 106 (1): 23-24; and PA Carr and GM Church, 2009, Nature Biotechnology 27 (12): 1151-62).

The guide molecule is configured to minimize or reduce off-target effects. Guide sequences and strategies to minimize toxicity and off-target effects can be as in WO 2014/093622 (PCT/US2013/074667); or, via mutation as described herein.

### Non-Naturally Occurring Nucleic Acids

In an embodiment, guide molecules of the invention comprise non-naturally occurring nucleic acids and/or non-naturally occurring nucleotides and/or nucleotide analogs, and/or chemical modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog, such as a nucleotide with phosphorothioate linkage, boranophosphate linkage, locked nucleic acid (LNA) nucleotide comprising a methylene bridge between the 2′ and 4′ carbons of the ribose ring or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (Ψ), N1-methylpseudouridine (me¹Ψ), 5-methoxyuridine (5moU), inosine, 7-methylguanosine. Examples of guide RNA chemical modifications include, without limitation, incorporation of 2′-O-methyl (M), 2′-O-methyl-3′-phosphorothioate (MS), phosphorothioate (PS), S-constrained ethyl (cEt), or 2′-O-methyl-3′-thioPACE (MSP) at one or more terminal nucleotides. Such chemically modified guides can comprise increased stability and increased activity as compared to unmodified guide molecules, though on-target vs. off-target specificity is not predictable. (See, Hendel, 2015, Nat Biotechnol. 33 (9): 985-9, doi: 10.1038/nbt.3290, published online 29 Jun. 2015; Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015)33(9): 985-989; Li et al., Nature Biomedical Engineering, 2017, 1, 0066 DOI: 10.1038/s41551-017-0066).

### 5′ and 3′ Modifications

In some embodiments, the 5′ and/or 3′ end of a guide molecule is modified by a variety of functional moieties including fluorescent dyes, polyethylene glycol, cholesterol, proteins, or detection tags. (See Kelly et al., 2016, J. Biotech. 233:74-83). In an embodiment of the invention, deoxyribonucleotides and/or nucleotide analogs are incorporated in engineered guide structures, such as, without limitation, 5′ and/or 3′ end, stem-loop regions, and the seed region. In an embodiment, the modification is not in the 5′-handle of the stem-loop regions. Chemical modification in the 5′-handle of the stem-loop region of a guide may abolish its function (see Li, et al., Nature Biomedical Engineering, 2017, 1:0066). In an embodiment, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide are chemically modified. In some embodiments, only minor modifications are introduced in the seed region, such as 2′-F modifications. In some embodiments, 2′-F modification is introduced at the 3′ end of a guide. In an embodiment, three to five nucleotides at the 5′ and/or the 3′ end of the guide are chemically modified with 2′-O-methyl (M), 2′-O-methyl-3′-phosphorothioate (MS), S-constrained ethyl (cEt), or 2′-O-methyl-3′-thioPACE (MSP). Such modification can enhance genome editing efficiency (see Hendel et al., Nat. Biotechnol. (2015)33 (9): 985-989). In an embodiment, all of the phosphodiester bonds of a guide are substituted with phosphorothioates (PS) for enhancing levels of gene disruption. In an embodiment, more than five nucleotides at the 5′ and/or the 3′ end of the guide are chemically modified with 2′-O-Me, 2′-F or S-constrained ethyl (cEt). Such chemically modified guides can mediate enhanced levels of gene disruption (see Rahdar et al., 2015, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In an embodiment, the chemical moiety is conjugated to the guide by a linker, such as an alkyl chain. In an embodiment, the chemical moiety of the modified guide can be used to attach the guide to another molecule, such as DNA, RNA, protein, or nanoparticles. Such chemically modified guides can be used to identify or enrich cells genetically edited by a CRISPR system (see Lee et al., eLife, 2017, 6: e25312, DOI: 10.7554).

In some embodiments, the loop of the 5′-handle of the guide molecule is modified. In some embodiments, the loop of the 5′-handle of the guide molecule is modified to have a deletion, an insertion, a split, or chemical modifications. In an embodiment, the loop comprises 3, 4, or 5 nucleotides. In an embodiment, the loop comprises the sequence of UCUU, UUUU, UAUU, or UGUU.

### Mixed RNA-DNA Guide Molecules

In one embodiment, the guide sequence comprises a mixture of RNA and DNA. The partial replacement of RNA nucleotides with DNA nucleotides has been shown to enhance CRISPR-Cas specificity by reducing off-target effects. See Rueda et al., Nat Commun 8, 1610 (2017), DOI: 10.1038/s41467-017-01732-9; Kartje et al., Biochemistry 2018, 57, 21, 3027-3031, DOI: 10.1021/acs.biochem.8b00107; and Yin et al., Nat Chem Biol. 2018 March; 14 (3): 311-316, DOI: 10.1038/nchembio.2559.

### Truncated Guide Molecules

In an embodiment, use is made of a truncated guide (tru-guide), i.e. a guide molecule which comprises a guide sequence which is truncated in length with respect to the canonical guide sequence length. As described by Nowak et al. (Nucleic Acids Res (2016)44 (20): 9555-9564), such guides may allow catalytically active CRISPR-Cas enzyme to bind its target without cleaving the target RNA. In an embodiment, a truncated guide is used which allows the binding of the target but retains only nickase activity of the CRISPR-Cas enzyme.

### Functionalized Guide Molecules

In an embodiment, guide portions can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable linkers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In an embodiment, the linker has a length equivalent to about 0-16 nucleotides. In an embodiment, the linker has a length equivalent to about 0-8 nucleotides. In an embodiment, the linker has a length equivalent to about 0-4 nucleotides. In an embodiment, the linker has a length equivalent to about 2 nucleotides. Example linker design is also described in WO2011/008730.

In an embodiment, the guide molecule comprises portions that are chemically linked or conjugated via a non-phosphodiester bond. In one aspect, the guide molecule comprises, in non-limiting examples, direct repeat sequence portion and a targeting sequence portion that are chemically linked or conjugated via a non-nucleotide loop. In an embodiment, the portions are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C-C bond forming groups such as Diels-Alder cyclo-addition pairs or ring-closing metathesis pairs, and Michael reaction pairs.

In an embodiment, portions of the guide molecule are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In an embodiment, the non-targeting guide portions can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once a non-targeting portion of a guide is functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C-C bond forming groups such as Diels-Alder cyclo-addition pairs or ring-closing metathesis pairs, and Michael reaction pairs.

In an embodiment, one or more portions of a guide molecule can be chemically synthesized. In an embodiment, the chemical synthesis uses automated, solid-phase oligonucleotide synthesis machines with 2′-acetoxyethyl orthoester (2′-ACE) (Scaringe et al., J. Am. Chem. Soc. (1998)120:11820-11821; Scaringe, Methods Enzymol. (2000)317:3-18) or 2′-thionocarbamate (2′-TC) chemistry (Dellinger et al., J. Am. Chem. Soc. (2011)133:11540-11546; Hendel et al., Nat. Biotechnol. (2015)33:985-989).

In an embodiment, portions of the guide molecule may be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009)48:6974-6998; Manoharan, M. Curr. Opin. Chem. Biol. (2004)8:570-9; Behlke et al., Oligonucleotides (2008)18:305-19; Watts, et al., Drug. Discov. Today (2008)13:842-55; Shukla, et al., ChemMedChem (2010)5:328-49.

In one embodiment, portions of the guide molecule may be covalently linked using click chemistry. In one embodiment, portions of the guide molecule may be covalently linked using a triazole linker. In one embodiment, portion of the guide molecule may be covalently linked using Huisgen 1,3-dipolar cycloaddition reaction involving an alkyne and azide to yield a highly stable triazole linker (He et al., ChemBioChem (2015)17:1809-1812; WO 2016/186745). In one embodiment, portions of the guide molecule may be covalently linked by ligating a 5′-hexyne portion and a 3′-azide portion. In one embodiment, either or both of the 5′-hexyne and the 3′-azide portion of the guide molecule may be protected with 2′-acetoxyethl orthoester (2′-ACE) group, which can be subsequently removed using Dharmacon protocol (Scaringe et al., J. Am. Chem. Soc. (1998)120:11820-11821; Scaringe, Methods Enzymol. (2000)317:3-18).

In one embodiment, the guide molecule is designed or selected to modulate intermolecular interactions among guide molecules, such as among stem-loop regions of different guide molecules. It will be appreciated that nucleotides within a guide molecule that base-pair to form a stem-loop are also capable of base-pairing to form an intermolecular duplex with a second guide molecule and that such an intermolecular duplex would not have a secondary structure compatible with CRISPR complex formation. Accordingly, is useful to select or design scaffold sequences in order to modulate stem-loop formation and CRISPR complex formation. In an embodiment, about or less than about 75%, 50%, 40%, 30%, 25%, 20%, 15%, 10%, 5%, 1%, or fewer of guide molecule are in intermolecular duplexes. It will be appreciated that stem-loop variation will often be within limits imposed by scaffold-Cas effector interactions. One way to modulate stem-loop formation or change the equilibrium between stem-loop and intermolecular duplex is to vary nucleotide pairs in the stem of the stem-loop of a scaffold. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a scaffold. Without being bound by theory, the loop can be viewed as an intervening sequence flanked by two sequences that are complementary to each other. When that intervening sequence is not self-complementary, its effect will be to destabilize intermolecular duplex formation. The same principle applies when guide molecules are multiplexed: while the targeting sequences may differ, it may be advantageous to modify the stem-loop region in the scaffold of the different guide molecules. Moreover, when guide molecules are multiplexed, the relative activities of the different guide molecules may be modulated by balancing the activity of each individual guide molecule. In an embodiment, the equilibrium between intermolecular stem-loops vs. intermolecular duplexes is determined. The determination may be made by physical or biochemical means and can be in the presence or absence of a CRISPR effector.

### Dead Guide Sequences

In one aspect, the invention provides guide molecules which are modified in a manner which allows for formation of the CRISPR Cas complex and successful binding to the target, while at the same time, not either allowing for or not allowing for successful nuclease activity (i.e., without nuclease activity/without indel activity). Such modified guide molecules are referred to as “dead guides” or “dead guide molecules”. These dead guide molecules can be thought of as catalytically inactive or conformationally inactive with regard to nuclease activity. Indeed, dead guide molecules may not sufficiently engage in productive base pairing with respect to the ability to promote catalytic activity or to distinguish on-target and off-target binding activity. Briefly, the assay involves synthesizing a CRISPR target RNA and guide molecules comprising mismatches with the target RNA, combining these with the enzyme and analyzing cleavage based on gels based on the presence of bands generated by cleavage products, and quantifying cleavage based upon relative band intensities.

Hence, in a related aspect, the invention provides a non-naturally occurring or engineered CRISPR-Cas system comprising a functional multimeric Cas enzyme as described herein, and guide molecule, e.g., a guide molecule wherein the guide molecules comprises a dead guide sequence whereby the guide molecule is capable of hybridizing to a target sequence such that the CRISPR-Cas system is directed to a genomic locus of interest in a cell without detectable cleavage activity of a non-mutant enzyme of the system. It is to be understood that any of the guide molecules according to the invention as described herein elsewhere may be used as dead guide molecules comprising a dead guide sequence.

The ability of a dead guide molecules to direct sequence-specific binding of a CRISPR complex to a target sequence may be assessed by any suitable assay. For example, the components of a CRISPR-Cas system sufficient to form a CRISPR-Cas complex, including the dead guide molecule to be tested, may be provided to a host cell having the corresponding target sequence, such as by transfection with vectors encoding the components of the system, followed by an assessment of preferential cleavage within the target sequence.

As explained further herein, several structural parameters allow for a proper framework to arrive at such dead guide molecules. Dead guide molecule sequences can be typically shorter than respective “active” guide molecules which result in active cleavage. In an embodiment, dead guide molecules are 5%, 10%, 20%, 30%, 40%, 50%, shorter than respective active guide molecules directed to the same target sequence.

As explained below and known in the art, one aspect of guide molecules specificity is the scaffold sequence, which is to be appropriately linked to guide sequences. In particular, this implies that the scaffold sequences are designed dependent on the origin of the enzyme. Structural data available for validated dead guide molecules may be used for designing CRISPR-Cas specific equivalents. Structural similarity between, e.g., the orthologous nuclease domains of two or more CRISPR-Cas proteins may be used to transfer design equivalent dead guide molecules. Thus, the dead guide molecule herein may be appropriately modified in length and sequence to reflect such CRISPR-Cas specific equivalents, allowing for formation of the CRISPR-Cas complex and successful binding to the target sequence, while at the same time, not allowing for successful nuclease activity.

Dead guide molecules allow one to use guide molecules as a means for gene targeting, without the consequence of nuclease activity, while at the same time providing directed means for activation or repression. Dead guide molecule may be modified to further include elements in a manner which allow for activation or repression of gene activity, in particular protein adaptors (e.g., aptamers) as described herein elsewhere allowing for functional placement of gene effectors (e.g., activators or repressors of gene activity). One example is the incorporation of aptamers, as explained herein and in the state of the art. By engineering the dead guide molecule to incorporate protein-interacting aptamers (Konermann et al., “Genome-scale transcription activation by an engineered CRISPR-Cas9 complex,” doi: 10.1038/nature 14136, incorporated herein by reference), one may assemble multiple distinct effector domains. Such may be modeled after natural processes.

In the practice of the invention, loops of the guide molecule may be extended, without colliding with the Cas protein by the insertion of distinct RNA loop(s) or distinct sequence(s) that may recruit adaptor proteins that can bind to the distinct RNA loop(s) or distinct sequence(s). The adaptor proteins may include but are not limited to orthogonal RNA-binding protein/aptamer combinations that exist within the diversity of bacteriophage coat proteins. A list of such coat proteins includes, but is not limited to: QB, F2, GA, fr, JP501, M12, R17, BZ13, JP34, JP500, KUI, M11, MX1, TW18, VK, SP, FI, ID2, NL95, TW19, AP205, 4Cb5, ¢Cb8r, ¢Cb12r, ¢Cb23r, 7s and PRR1. These adaptor proteins or orthogonal RNA binding proteins can further recruit effector proteins or fusions which comprise one or more functional domains.

The functional domain may be selected from the group consisting of: transposase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. The functional domain is a transcriptional activation domain, such as, without limitation, VP64, p65, MyoD1, HSF1, RTA, SET7/9 or a histone acetyltransferase. The functional domain may be a transcription repression domain, preferably KRAB. In one embodiment, the transcription repression domain is SID, or concatemers of SID (e.g., SID4X). In one embodiment, the functional domain is an epigenetic modifying domain, such that an epigenetic modifying enzyme is provided. In an embodiment, the functional domain is an activation domain, which may be the P65 activation domain.

### Mismatches

In one embodiment, the guide molecule may comprise a mismatch. The mismatch may be up-or downstream of a single nucleotide variation on the one or more guide sequences. Modulations of cleavage efficiency can be exploited by introduction of mismatches, e.g., 1 or more mismatches, such as 1 or 2 mismatches between the guide sequence and target sequence. The more central (i.e., not 3′ or 5′) for instance a double mismatch is, the more cleavage efficiency is affected. Accordingly, by choosing mismatch position along the guide sequence, cleavage efficiency can be modulated. By means of example, if less than 100% cleavage of targets is desired (e.g., in a cell population), 1 or more, such as preferably 2 mismatches between the guide sequence and target sequence may be introduced in the guide sequences. The more central along the guide sequence of the mismatch position, the lower the cleavage percentage. In an embodiment, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide molecule may be designed with a nucleotide sequence that is complementary to one of the targets i.e., the on-target SNP. The guide molecule is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e., adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as, but not limited to, disease associated SNPs, such as without limitation cancer associated SNPs.

In an embodiment, the guide molecule is designed such that the mismatch (e.g., the synthetic mismatch, e.g., an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In one embodiment, the guide molecule is designed such that the mismatch is located on position 1, 2, 3, 4, 5, 6, 7, 8, or 9 of the spacer sequence (starting at the 5′ end). In one embodiment, the guide molecule is designed such that the mismatch is located on position 4, 5, 6, or 7 of the spacer sequence (starting at the 5′ end. In an embodiment, the guide molecule is designed such that the mismatch is located on position 5 of the guide sequence (starting at the 5′ end).

In an embodiment, the guide molecule is designed such that the mismatch is located 2 nucleotides upstream of the SNP (i.e., one intervening nucleotide). In one embodiment, the guide molecule is designed such that the mismatch is located 2 nucleotides downstream of the SNP (i.e., one intervening nucleotide). In one embodiment, the guide molecule is designed such that the mismatch is located on position 5 of the guide sequence (starting at the 5′ end) and the SNP is located on position 3 of the guide sequence (starting at the 5′ end).

### Determination of PAM

A protospacer adjacent motif (PAM) or PAM-like motif directs binding of the CRISPR-Cas complex as disclosed herein to the target locus of interest. In an embodiment, the PAM may be a 5′ PAM (i.e., located upstream of the 5′ end of the protospacer). In other embodiments, the PAM may be a 3′ PAM (i.e., located downstream of the 5′ end of the protospacer). In other embodiments, both a 5′ PAM and a 3′ PAM are required. In one example, the PAM comprises or is AAG. In certain examples, the PAM is or comprises CTT.

In an embodiment, a PAM or PAM-like motif may not be required for directing binding of the CRISPR-Cas complex. In an embodiment, a 5′ PAM is D (e.g., A, G, or U). In an embodiment of the invention, cleavage at repeat sequences may generate crRNAs (e.g., short or long crRNAs) containing a full spacer sequence flanked by a short nucleotide (e.g., 5, 6, 7, 8, 9, or 10 nt or longer if it is a dual repeat) repeat sequence at the 5′ end (this may be referred to as a crRNA “tag”) and the rest of the repeat at the 3′end. In an embodiment, targeting by the effector proteins described herein may require the lack of homology between the crRNA tag and the target 5′ flanking sequence. This requirement may be similar to that described further in Samai et al. “Co-transcriptional DNA and RNA Cleavage during Type VI CRISPR-Cas Immunity” Cell 161, 1164-1174 May 21, 2015, where the requirement is thought to distinguish between bona fide targets on invading nucleic acids from the CRISPR array itself, and where the presence of repeat sequences will lead to full homology with the crRNA tag and prevent autoimmunity.

In an embodiment, determination of PAM can be performed as follows. This experiment closely parallels similar work in E. coli for the heterologous expression of StCas9 (Sapranauskas, R. et al. Nucleic Acids Res 39, 9275-9282 (2011)). Applicants introduce a plasmid containing both a PAM and a resistance gene into the heterologous E. coli, and then plate on the corresponding antibiotic. If there is DNA cleavage of the plasmid, Applicants observe no viable colonies.

In further detail, the assay is as follows for a DNA target. Two E. coli strains are used in this assay. One carries a plasmid that encodes the endogenous effector protein locus from the bacterial strain. The other strain carries an empty plasmid (e.g., pACYC184, control strain). All possible 7 or 8 bp PAM sequences are presented on an antibiotic resistance plasmid (pUC19 with ampicillin resistance gene). The PAM is located next to the sequence of proto-spacer 1 (the DNA target to the first spacer in the endogenous effector protein locus). Two PAM libraries were cloned. One has an 8 random bp 5′ of the proto-spacer (e.g., total of 65536 different PAM sequences=complexity). The other library has 7 random bp 3′ of the proto-spacer (e.g., total complexity is 16384 different PAMs). Both libraries were cloned to have in average 500 plasmids per possible PAM. Test strain and control strain were transformed with 5′PAM and 3′PAM library in separate transformations and transformed cells were plated separately on ampicillin plates. Recognition and subsequent cutting/interference with the plasmid renders a cell vulnerable to ampicillin and prevents growth. Approximately 12h after transformation, all colonies formed by the test and control strains where harvested and plasmid DNA was isolated. Plasmid DNA was used as template for PCR amplification and subsequent deep sequencing. Representation of all PAMs in the untransformed libraries showed the expected representation of PAMs in transformed cells. Representation of all PAMs found in control strains showed the actual representation. Representation of all PAMs in test strain showed which PAMs are not recognized by the enzyme and comparison to the control strain allows extracting the sequence of the depleted PAM.

### Multiplex Targeting Approach

The CRISPR-Cas systems or complexes herein can employ more than one guide molecule, e.g., guide RNA, without losing activity. This may enable the use of the CRISPR-Cas systems or complexes as defined herein for targeting multiple targets (e.g., RNA targets, DNA targets), genes or gene loci, with a single system or complex as defined herein. The guide molecules, e.g., guide RNAs, may be tandemly arranged, optionally separated by a nucleotide sequence such as a direct repeat as defined herein. The position of the different guide molecules, e.g., guide RNAs, is the tandem does not influence the activity.

In any of the described methods the multimeric CRISPR-Cas effector protein, or Cas polypeptides thereof, may be delivered with multiple guides for multiplexed use. In any of the described methods more than one multimeric CRISPR-Cas effector protein, or Cas polypeptides thereof, may be used. In an embodiment, one CRISPR-Cas effector protein, or Cas polypeptides thereof, may be delivered with multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides. In an embodiment, a system or complex herein may comprise a multimeric CRISPR-Cas effector protein, or Cas polypeptides thereof, and multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides.

The multimeric CRISPR-Cas effector protein may form part of a multiplexed CRISPR-Cas system or complex, which further comprises tandemly arranged guide molecules, e.g., guide RNAs (gRNAs), comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In an embodiment, the functional CRISPR-Cas system or complex binds to the multiple target sequences. In an embodiment, the functional CRISPR-Cas system or complex may edit the multiple target sequences, e.g., the target sequences may comprise a genomic locus, and in an embodiment, there may be an alteration of gene expression. In an embodiment, the functional CRISPR-Cas system or complex may comprise further functional domains. In an embodiment, the invention provides a method for altering or modifying expression of multiple gene products. The method may comprise introducing into a cell containing said target nucleic acids, e.g., RNA molecules, or DNA molecules, or containing and expressing target nucleic acid, e.g., RNA molecules, or DNA molecules; for instance, the target nucleic acids may encode gene products or provide for expression of gene products (e.g., regulatory sequences).

In some general embodiments, one or more of the Cas enzymes used for multiplex targeting is associated with one or more functional domains.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

### Multiplex Targeting Approach

The CRISPR-Cas systems or complexes herein can employ more than one RNA guide without losing activity. This may enable the use of the CRISPR-Cas systems or complexes as defined herein for targeting multiple targets (e.g., RNA targets, DNA targets), genes or gene loci, with a single system or complex as defined herein. The guide molecules, e.g., guide RNAs, may be tandemly arranged, optionally separated by a nucleotide sequence such as a direct repeat as defined herein. The position of the different guide molecules, e.g., guide RNA is the tandem does not influence the activity.

In any of the described methods the multimeric CRISPR-Cas effector protein, or Cas polypeptides thereof, may be delivered with multiple guides for multiplexed use. In any of the described methods more than one multimeric CRISPR-Cas effector protein, or Cas polypeptides thereof, may be used. In an embodiment, one CRISPR-Cas effector protein, or Cas polypeptides thereof, may be delivered with multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides. In an embodiment, a system or complex herein may comprise a multimeric CRISPR-Cas effector protein, or Cas polypeptides thereof, and multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides.

The multimeric CRISPR-Cas effector protein may form part of a multiplexed CRISPR-Cas system or complex, which further comprises tandemly arranged guide molecules, e.g., guide RNAs (gRNAs), comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In an embodiment, the functional CRISPR-Cas system or complex binds to the multiple target sequences. In an embodiment, the functional CRISPR-Cas system or complex may edit the multiple target sequences, e.g., the target sequences may comprise a genomic locus, and in an embodiment there may be an alteration of gene expression. In an embodiment, the functional CRISPR-Cas system or complex may comprise further functional domains. In an embodiment, the invention provides a method for altering or modifying expression of multiple gene products. The method may comprise introducing into a cell containing said target nucleic acids, e.g., RNA molecules, or DNA molecules, or containing and expressing target nucleic acid, e.g., RNA molecules, or DNA molecules; for instance, the target nucleic acids may encode gene products or provide for expression of gene products (e.g., regulatory sequences).

In some general embodiments, one or more of the Cas enzymes used for multiplex targeting is associated with one or more functional domains. In some more specific embodiments, the CRISPR enzyme used for multiplex targeting is a dead Cas (dCas) as defined herein elsewhere. In an embodiment, each of the guide sequence is at least 16, 17, 18, 19, 20, 25 nucleotides, or between 16-30, or between 16-25, or between 16-20 nucleotides in length. Examples of multiplex genome engineering using CRISPR effector proteins are provided in Cong et al. (Science February 15; 339 (6121): 819-23 (2013) and other publications cited herein.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

In the practice of the invention, loops of the guide molecule may be extended, without colliding with the Cas protein by the insertion of distinct RNA loop(s) or distinct sequence(s) that may recruit adaptor proteins that can bind to the distinct RNA loop(s) or distinct sequence(s). The adaptor proteins may include, but are not limited to, orthogonal RNA-binding protein/aptamer combinations that exist within the diversity of bacteriophage coat proteins. A list of such coat proteins includes, but is not limited to: QB, F2, GA, fr, JP501, M12, R17, BZ13, JP34, JP500, KUI, M11, MX1, TW18, VK, SP, FI, ID2, NL95, TW19, AP205, φCb5, φCb8r, φCb12r, φCb23r, 7s and PRR1. These adaptor proteins or orthogonal RNA binding proteins can further recruit effector proteins or fusions which comprise one or more functional domains.

### RNA Base Editing

The present disclosure also provides for a base editing system. Such base editing systems can be adapted for use with the CRISPR-Cas system described herein or a variant thereof. In general, such a system may comprise a deaminase (e.g., an adenosine deaminase or cytidine deaminase) fused with a Cas15, a Cas5, a Cas7, or a Cas 6 protein or a variant thereof. The Cas15 protein may be a dead Cas15 protein or a Cas15 nickase protein. In certain examples, the system comprises a mutated form of an adenosine deaminase fused with a dead Cas15 or Cas15 nickase. The mutated form of the adenosine deaminase may have both adenosine deaminase and cytidine deaminase activities.

In one aspect, the present disclosure provides an engineered adenosine deaminase. The engineered adenosine deaminase may comprise one or more mutations herein. In an embodiment, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In an embodiment, compositions herein comprise nucleotide sequence comprising encoding sequences for one or more components of a base editing system.

Examples of base editing systems include those described in WO2019071048, WO2019084063, WO2019126716, WO2019126709, WO2019126762, WO2019126774, Cox DBT, et al., RNA editing with CRISPR-Cas13, Science. 2017 Nov. 24; 358 (6366): 1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A•T to G•C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19; 533 (7603): 420-4.

### Polynucleotides and Vectors

The systems and compositions herein may comprise one or more polynucleotides (aka nucleic acid molecules). In an embodiment, one or more polynucleotide(s) comprise one or more nucleotide sequences encoding one or more components of a CRISPR-Cas system or a composition thereof as disclosed herein. In an embodiment, a polynucleotide comprises one or more nucleic acid sequences encoding one or more of the β-CAPS polypeptide(s), the one or more Cas protein(s), one or more guide sequences, or any combination thereof. In an embodiment, the present disclosure further provides vectors or vector systems comprising one or more polynucleotides as disclosed herein. The vectors or vector systems include those described in the delivery sections as disclosed herein. In an embodiment, a vector is a viral vector.

In an embodiment, the polynucleotide sequence is recombinant DNA. In further embodiments, the polynucleotide sequence further comprises additional sequences as described elsewhere herein. In an embodiment, the nucleic acid sequence is synthesized in vitro.

Aspects of the invention relate to polynucleotide molecules that encode one or more components of the CRISPR-Cas system or composition thereof as referred to in any embodiment herein. In an embodiment, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In an embodiment, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In an embodiment, the 5′ and/or 3′ end of the isolated polynucleotide sequence may be modified to improve the stability of the sequence of actively avoid degradation. In an embodiment, the isolated polynucleotide sequence may be comprised in a bacteriophage. In other embodiments, the isolated polynucleotide sequence may be contained in agrobacterium species. In an embodiment, the isolated polynucleotide sequence is lyophilized.

### Codon Optimization

Aspects of the invention relate to polynucleotide molecules that encode one or more components of one or more CRISPR-Cas systems as described in any of the embodiments herein, wherein at least one or more regions of the polynucleotide molecule may be codon optimized for expression in a eukaryotic cell. In an embodiment, the polynucleotide molecules that encode one or more components of one or more CRISPR-Cas systems as described in any of the embodiments herein are optimized for expression in a mammalian cell or a plant cell.

An example of a codon optimized sequence, is in this instance a sequence optimized for expression in a eukaryote, e.g., humans (i.e., being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In an embodiment, an enzyme coding sequence encoding a DNA/RNA-targeting Cas protein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In an embodiment, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. e Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In an embodiment, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Delivery

In an embodiment, the present disclosure also provides delivery systems for introducing one or more components of the CRISPR-Cas systems, compositions, polynucleotides, vectors, or any combination thereof, disclosed herein, to cells, tissues, organs, or organisms. A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties. In an embodiment, a delivery vehicle is a lipid nanoparticle, a viral capsid, an engineered retroelement vector, a polynucleotide-based nano-structure, or an extracellular contractile injection system.

In an embodiment, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February;9 (1): 11-9; Klein R M, et al., Biotechnology. 1992; 24:384-6; Casas A M et al., Proc Natl Acad Sci USA. 1993 Dec. 1; 90 (23): 11212-11216; and U.S. Pat. No. 5,563,055, Davey M R et al., Plant Mol Biol. 1989 September; 13 (3): 273-85, which are incorporated by reference herein in their entireties.

The example delivery compositions, systems, and methods described herein related to composition or nucleic acid-guided nuclease also apply to functional domains and other components (e.g., other proteins and polynucleotides related to the nucleic acid-guided nuclease, such as reverse transcriptase, nucleotide deaminase, retrotransposon, donor polynucleotide, etc.).

### Cargos

The delivery systems may comprise one or more cargos. The cargos may comprise one or more components of the systems and compositions herein. A cargo may comprise one or more of the following: i) a plasmid encoding one or more proteins components in the compositions and systems such as the nucleic acid-guided nuclease and/or functional domains; ii) a plasmid encoding one or more guide molecules, iii) mRNA of one or more one or more proteins components in the compositions and systems such as the nucleic acid-guided nuclease and/or functional domains; iv) one or more guide molecules, e.g., guide RNAs; v) one or more proteins components in the compositions and systems such as the nucleic acid-guided nuclease and/or functional domains; vi) any combination thereof. The one or more protein components may include the nuclei acid-guided nuclease (e.g., Cas), reverse transcriptase, nucleotide deaminase, retrotransposon protein, other functional domain, or any combination thereof.

In an embodiment, a cargo may comprise a plasmid encoding one or more proteins components in the compositions and systems such as the nucleic acid-guided nuclease and/or functional domains and one or more (e.g., a plurality of) guide molecules, e.g., guide RNAs. In some cases, the plasmid may also encode a recombination template (e.g., for HDR). In an embodiment, a cargo may comprise mRNA encoding one or more protein components and one or more guide molecules, e.g., guide RNAs.

In an embodiment, a cargo may comprise one or more protein components and one or more guide molecules, e.g., guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November;33 (11): 1162-4.

### Physical Delivery

In an embodiment, the cargos may be introduced to cells by physical delivery methods. Examples of physical methods include microinjection, electroporation, and hydrodynamic delivery. Both nucleic acid and proteins may be delivered using such methods. For example, one or more protein components may be prepared in vitro, isolated, (refolded, purified if needed), and introduced to cells.

### Microinjection

Microinjection of the cargo directly to cells can achieve high efficiency, e.g., above 90% or about 100%. In an embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for one or more protein components and/or guide molecules, e.g., guide RNAs, and/or mRNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and mRNA to the cytoplasm, e.g., facilitating translation and shuttling of one or more protein components to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up-or down-regulate a specific gene within the genome of a cell, e.g., using CRISPRa and CRISPRi.

### Electroporation

In an embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In an embodiment, hydrodynamic delivery may be performed by rapidly pushing a large volume (8-10% body weight) solution containing the gene editing cargo into the bloodstream of a subject (e.g., an animal or human), e.g., for mice via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may have a greatest dimension (e.g., diameter) of less than 100 microns (μm). In an embodiment, the delivery vehicles have a greatest dimension of less than 10 μm. In an embodiment, the delivery vehicles may have a greatest dimension of less than 2000 nanometers (nm). In an embodiment, the delivery vehicles may have a greatest dimension of less than 1000 nanometers (nm). In an embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In an embodiment, the delivery vehicles may have a greatest dimension ranging between 25 nm and 200 nm.

In an embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008042156, US Publication Application No. U.S. Pat. No. 20,130,185823, and International Patent Publication No WO 2015/089419.

### Vectors

The systems, compositions, and/or delivery systems may comprise one or more vectors. The present disclosure also includes vector systems. A vector system may comprise one or more vectors. In an embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) one or more protein components encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the complex. When provided by a separate vector, the RNA that targets nucleic acid-guided nuclease expression can be administered sequentially or simultaneously. When administered sequentially, the RNA that targets nucleic acid-guided nuclease expression is to be delivered after the RNA that is intended for gene editing or gene engineering. This period may be a period of minutes (e.g., 5 minutes, 10 minutes, 20 minutes, 30 minutes, 45 minutes, 60 minutes). This period may be a period of hours (e.g., 2 hours, 4, hours, 6 hours, 8 hours, 12 hours, 24 hours). This period may be a period of days (e.g., 2 days, 3 days, 4 days, 7 days). This period may be a period of weeks (e.g., 2 weeks, 3 weeks, 4 weeks). This period may be a period of months (e.g., 2 months, 4 months, 6 months, 12 months). This period may be a period of years (2 years, 3 years, 4 years). In this fashion, the nucleic acid-guided nuclease associates with a first gRNA capable of hybridizing to a first target, such as a genomic locus or loci of interest and undertakes the function(s) desired of the system (e.g., gene engineering); and subsequently the nucleic acid-guided nuclease may then associate with the second gRNA capable of hybridizing to the sequence comprising at least part of the nucleic acid-guided nuclease. Where the guide RNA targets the sequences encoding expression of the nucleic acid-guided nuclease, the enzyme becomes impeded, and the system becomes self-inactivating. In the same manner, RNA that targets nucleic acid-guided nuclease expression applied via, for example liposome, lipofection, particles, microvesicles as explained herein, may be administered sequentially or simultaneously. Similarly, self-inactivation may be used for inactivation of one or more guide RNA used to target one or more targets.

### Regulatory Elements

A vector may comprise one or more regulatory elements. The regulatory element(s) may be operably linked to coding sequences of nucleic acid-guided nuclease, accessary proteins, guide molecules, e.g., guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In certain examples, a vector may comprise: a first regulatory element operably linked to a nucleotide sequence encoding a nucleic acid-guided nuclease, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In an embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In an embodiment, AAV may provide a persistent source of the provided DNA, as AAV delivered genomic material can exist indefinitely in cells, e.g., either as exogenous DNA or, with some modification, be directly integrated into the host DNA. In an embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008)) and shown as follows in Table 7.

TABLE 7

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

The AAV particles may be created in HEK 293 T cells. Once particles with specific tropism have been created, they are used to infect the target cell line much in the same way that native viral particles do. This may allow for persistent presence of the components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In an embodiment, coding sequences of nucleic acid-guided nuclease and gRNA may be packaged directly onto one DNA plasmid vector and delivered via one AAV particle. In an embodiment, AAVs may be used to deliver gRNAs into cells that have been previously engineered to express nucleic acid-guided nuclease. In an embodiment, coding sequences of nucleic acid-guided nuclease and gRNA may be made into two separate AAV particles, which are used for co-transfection of target cells. In an embodiment, markers, tags, and other sequences may be packaged in the same AAV particles as coding sequences of nucleic acid-guided nuclease and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2: 36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second-and third-generation lentiviral systems may split essential genes across three plasmids, which may reduce the likelihood of accidental reconstitution of viable viral particles within cells.

In an embodiment, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In an embodiment, adenoviruses do not integrate into the genome of host cells, which may be used for limiting off-target effects of systems in gene editing applications.

### Viral Vehicles for Delivery to Plants

The systems and compositions may be delivered to plant cells using viral vehicles. In an embodiment, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In an embodiment, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In an embodiment. LNPs may be used for delivering DNA molecules (e.g., those comprising coding sequences of nucleic acid-guided nuclease and/or gRNA) and/or RNA molecules (e.g., mRNA of nucleic acid-guided nuclease, gRNAs). In certain cases, LNPs may be use for delivering RNP complexes of nucleic acid-guided nuclease/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N, N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 December 2011).

### Liposomes

In an embodiment, a lipid particle may be liposome. Liposomes are spherical vesicle structures composed of a uni-or multilamellar lipid bilayer surrounding internal aqueous compartments and a relatively impermeable outer lipophilic phospholipid bilayer. In an embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In an embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In an embodiment, SNALPs may comprise synthetic cholesterol, dipalmitoylphosphatidylcholine, 3-N-[(w-methoxy polyethylene glycol)2000) carbamoyl]-1,2-dimyrestyloxypropylamine, and cationic 1,2-dilinoleyloxy-3-N,Ndimethylaminopropane. In an embodiment, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In an embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca2+ microcomplexes), polyethenimine (PEI) (e.g., branched PEI), and poly(L-lysine) (PLL).

### Cell Penetrating Peptides

In an embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In an embodiment, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In an embodiment, CPPs may be covalently attached to the nucleic acid-guided nuclease directly, which is then complexed with the gRNA and delivered to cells. In an embodiment, separate delivery of CPP-Cas and CPP-gRNA to multiple cells may be performed. CPP may also be used to delivery RNPs.

CPPs may be used to deliver the compositions and systems to plants. In an embodiment, CPPs may be used to deliver the components to plant protoplasts, which are then regenerated to plant cells and further to plants.

### DNA Nanoclews

In an embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136 (42): 14722-5; and Sun W et al, Angew Chem Int Ed Engl. 2015 Oct. 5; 54 (41): 12029-33. DNA nanoclew may have a palindromic sequences to be partially complementary to the gRNA within the nucleic acid-guided nuclease: gRNA ribonucleoprotein complex. A DNA nanoclew may be coated, e.g., coated with PEI to induce endosomal escape.

### Gold Nanoparticles

In an embodiment, the delivery vehicles comprise gold nanoparticles (also referred to AuNPs or colloidal gold). Gold nanoparticles may form complex with cargos, e.g., nucleic acid-guided nuclease: gRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In an embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D′Astolfo D S, Pagliero R J, Pras A, et al. (2015). Cell 161:674-690.

### Polymer-Based Particles

In an embodiment, the delivery vehicles may comprise polymer-based particles (e.g., nanoparticles). In an embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In an embodiment, the polymer-based particles may comprise alkylated and carboxyalkylated branched polyethylenimine. In an embodiment, the polymer-based particles are VIROMER, e.g., VIROMER RNAi, VIROMER RED, VIROMER mRNA, VIROMER CRISPR. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection-Factbook 2018: technology, product overview, users' data., doi: 10.13140/RG.2.2.23912.16642.

### Streptolysin ((SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6: e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In an embodiment, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In an embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267 (1): 9-21; El-Andaloussi S, et al., Nat Protoc. 2012 December;7 (12): 2112-26; Uno Y, et al., Hum Gene Ther. 2011 June;22 (6): 711-9; Zou W, et al., Hum Gene Ther. 2011 April;22 (4): 465-75.

In an embodiment, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater. Sci. 2020 Apr. 28. doi: 10.1039/d0bm00427h.

### Genetically Modified Cells, Tissues, and Organisms

The present disclosure further provides cells comprising one or more components of the CRISPR-Cas systems, compositions, polynucleotides, vectors, delivery systems, or any combination thereof, as described herein. Also provided include cells modified by the CRISPR-Cas systems (i.e., “engineered cells”) and methods disclosed herein, and cell cultures, tissues, organs, organism comprising such engineered cells or progeny thereof.

In an embodiment, the present disclosure provides a method of modifying a cell or organism. a cell, a tissue, or an organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish, or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree, or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

In an embodiment, one or more polynucleotide molecules, vectors, or vector systems driving expression of one or more elements of the compositions, systems, or delivery systems comprising one or more elements of the nucleic acid-targeting system are introduced into a host cell such that expression of the elements of the nucleic acid-targeting system direct formation of a nucleic acid-targeting complex at one or more target sites. In an embodiment of the invention the host cell may be a eukaryotic cell, a prokaryotic cell, or a plant cell.

In an embodiment, the host cell is a cell of a cell line. Cell lines are available from a variety of sources known to those with skill in the art (see, e.g., the American Type Culture Collection (ATCC) (Manassas, Va.)). In an embodiment, a cell transfected with one or more vectors described herein is used to establish a new cell line comprising one or more vector-derived sequences. In an embodiment, a cell transiently transfected with the components of a system as described herein (such as by transient transfection of one or more vectors, or transfection with RNA), and modified through the activity of a complex, is used to establish a new cell line comprising cells containing the modification but lacking any other exogenous sequence. In an embodiment, cells transiently or non-transiently transfected with one or more vectors described herein, or cell lines derived from such cells are used in assessing one or more test compounds.

Further intended are isolated human cells or tissues, or organisms comprising the engineered cells disclosed herein (e.g., plants or animals (e.g., non-human animals)) comprising one or more of the components of the CRISPR-Cas systems, compositions, polynucleotide molecules, vectors, vector systems, or cells described in any of the embodiments herein. In an embodiment, host cells and cell lines modified by or comprising the compositions, systems or modified enzymes of present invention are provided, including (isolated) stem cells, and progeny thereof.

In an embodiment, the plants or non-human animals comprise at least one of the system components, polynucleotide molecules, vectors, vector systems, or cells described in any of the embodiments herein at least one tissue type of the plant or non-human animal. In an embodiment, non-human animals comprise at least one of the system components, polynucleotide molecules, vectors, vector systems, or cells described in any of the embodiments herein in at least one tissue type. In an embodiment, the presence of the system components is transient, in that they are degraded over time. In an embodiment, expression of the components of the systems and compositions described in any of the embodiments comprised in polynucleotide molecules, vectors, vector systems, or cells is limited to certain tissue types or regions in the plant or non-human animal. In an embodiment, the expression of the components of the systems and compositions described in any of the embodiments comprised in polynucleotide molecules, vectors, vector systems, or cells is dependent of a physiological cue. In an embodiment, expression of the components of the systems and compositions described in any of the embodiments comprised in polynucleotide molecules, vectors, vector systems, or cells may be triggered by an exogenous molecule. In an embodiment, expression of the components of the systems and compositions described in any of the embodiments comprised in polynucleotide molecules, vectors, vector systems, or cells is dependent on the expression of a non-cas molecule in the plant or non-human animal.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fugus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In an embodiment, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi: 10.1186/1746-4811-9-39); Brooks, “Efficient gene editing in tomato in the first generation using the CRISPR-Cas9 system,” Plant Physiology September 2014 pp 114247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013)23:1229-1232. doi: 10.1038/cr.2013.114; published online 20 Aug. 2013; Xie, “RNA-guided genome editing in plants using a CRISPR-Cas system,” Mol Plant. 2013 November;6 (6): 1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum)1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061—Agrobacterium-Mediated Plant Transformation Method; U.S. Pat. No. 7,868,149-Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29; 13 (2): 85-96, all the contents and disclosure of each of which are herein incorporated by reference in their entirety. Aspects of utilizing the compositions, systems, and methods may be analogous to the use of the CRISPR-Cas system in plants, and mention is made of the University of Arizona website “CRISPR-PLANT” (www.genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate, regenerate, and grow into a whole plant under proper growing conditions.

The compositions, systems, and methods may be used for screening genes (e.g., endogenous, mutations) of interest. In an embodiment, genes of interest include those encoding enzymes involved in the production of a component of added nutritional value or generally genes affecting agronomic traits of interest, across species, phyla, and plant kingdom. By selectively targeting e.g., genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September;172 (1): 62-77.

The components (e.g., Cas proteins) in the compositions and systems may further comprise one or more functional domains described herein. In an embodiment, the functional domains may be an exonuclease. Such exonuclease may increase the efficiency of the Cas proteins' function, e.g., mutagenesis efficiency. An example of the functional domain is Trex2, as described in Weiss T et al., www.biorxiv.org/content/10.1101/2020.04.11.037572v1, doi: doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular, or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, nocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In an embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In an embodiment, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In an embodiment, the plant promoter is a regulated promoter, which directs gene expression not constitutively, but in a temporally- and/or spatially-regulated manner, and includes tissue-specific, tissue-preferred, and inducible promoters. Different promoters may direct the expression of a gene in different tissues or cell types, or at different stages of development, or in response to different environmental conditions. In an embodiment, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In an embodiment, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, some of the components of a light inducible system include a Cas protein, a light-responsive cytochrome heterodimer (e.g., from Arabidopsis thaliana), and a transcriptional activation/repression domain.

In an embodiment, the promoter may be a chemical-regulated promotor (where the application of an exogenous chemical induces gene expression) or a chemical-repressible promoter (where application of the chemical represses gene expression). Examples of chemical-inducible promoters include maize In2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In an embodiment, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the Cas gene are expressed. In some cases, the polynucleotides may be integrated into an organelle of a plant, such as a plastid, mitochondrion, or a chloroplast. The elements of the expression system may be on one or more expression constructs which are either circular such as a plasmid or transformation vector, or non-circular such as linear double stranded DNA.

In an embodiment, the method of integration generally comprises the steps of selecting a suitable host cell or host tissue, introducing the construct(s) into the host cell or host tissue, and regenerating plant cells or plants therefrom. In an embodiment, the expression system for stable integration into the genome of a plant cell may contain one or more of the following elements: a promoter element that can be used to express the RNA and/or Cas enzyme in a plant cell; a 5′ untranslated region to enhance expression; an intron element to further enhance expression in certain cells, such as monocot cells; a multiple-cloning site to provide convenient restriction sites for inserting the guide molecule, e.g., guide RNA, and/or the Cas gene sequences and other desired elements; and a 3′ untranslated region to provide for efficient termination of the expressed transcript.

### Transient Expression in Plants

In an embodiment, the components of the compositions and systems may be transiently expressed in the plant cell. In an embodiment, the compositions and systems may modify a target nucleic acid only when both the guide molecule, e.g., guide RNA, and the Cas protein are present in a cell, such that genomic modification can further be controlled. As the expression of the Cas protein is transient, plants regenerated from such plant cells typically contain no foreign DNA. In certain examples, the Cas protein is stably expressed, and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a PEAQ vector (may be tailored for Agrobacterium-mediated transient expression) and Cabbage Leaf Curl virus (CaLCuV), and vectors described in Sainsbury F. et al., Plant Biotechnol J. 2009 September;7 (7): 682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In an embodiment, it is envisaged that the compositions and systems are used to specifically modify chloroplast genes or to ensure expression in the chloroplast. The compositions and systems (e.g., Cas proteins, guide molecules, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In an embodiment, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61:157-180, and US20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, guide molecules targeting one or more locations in a genome may be provided and introduced into plant cells together with the Cas effector protein. For example, a collection of genome-scale point mutations and gene knock-outs can be generated. In an embodiment, the compositions, systems, and methods may be used to generate a plant part or plant from the cells so obtained and screening the cells for a trait of interest. The target genes may include both coding and non-coding regions. In some cases, the trait is stress tolerant and the method is a method for the generation of stress-tolerant crop varieties.

In an embodiment, the compositions, systems, and methods are used to modify endogenous genes or to modify their expression. The expression of the components may induce targeted modification of the genome, either by direct activity of the Cas nuclease and optionally introduction of recombination template DNA, or by modification of genes targeted. The different strategies described herein above allow Cas-mediated targeted genome editing without requiring the introduction of the components into the plant genome.

In some cases, the modification may be performed without the permanent introduction into the genome of the plant of any foreign gene, including those encoding CRISPR components, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In an embodiment, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In an embodiment, genes that confer resistance to pests or diseases may be introduced to plants. In cases there are endogenous genes that confer such resistance in plants, their expression and function may be enhanced (e.g., by introducing extra copies, modifications that enhance expression and/or activity).

Examples of genes that confer resistance include plant disease resistance genes (e.g., Cf-9, Pto, RSP2, SIDMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In an embodiment, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In an embodiment, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvate dioxygenases (HPPD) inhibitors, e.g., naturally occurring HPPD resistant enzymes, and genes encoding a mutated or chimeric HPPD enzyme.

In an embodiment, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In an embodiment, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In an embodiment, the compositions, systems, and methods may be used to produce nutritionally improved plants. In an embodiment, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g., by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In an embodiment, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., ω-3 fatty acids, Conjugated linoleic acid, GLA,), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g., as many as six, such as in wheat). In some cases, the compositions, systems, and methods can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-Al, TaMLO-Bl and TaMLO-Dl nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In an embodiment, the compositions, systems, and methods are used to reduce ethylene production. In an embodiment, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In an embodiment, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products, and elevated levels of acrylamide, which is a potential carcinogen. In an embodiment, the methods provided herein are used to reduce or inhibit expression of the vacuolar invertase gene (VInv), which encodes a protein that breaks down sucrose to glucose and fructose.

### Reducing Allergens in Plants

In an embodiment, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11 (3): 222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12 (3): 321-342; and Kim Y J, et al., Trends Plant Sci. 2018 January;23 (1): 53-65.

### Increasing the Fertility Stage in Plants

In an embodiment, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In an embodiment, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January;49 (1): 162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In an embodiment, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In an embodiment, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ‘tesA, tesB, fatB, fatB2, fatB3, fatAl, or fatA), acyl-CoA synthases (e.g., fadD, JadK, BH3103, pfl-4354, EAV15023, fadDI, fadD2, RPC_4074,fadDD35, fadDD22, faa39), ester synthases (e.g., synthase/acyl-CoA: diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In an embodiment, plants may be modified to produce organic acids such as lactic acid. The plants may produce organic acids using sugars, pentose or hexose sugars. To this end, one or more genes may be introduced (e.g., and overexpressed) in the plants. An example of such genes includes LDH gene.

In an embodiment, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (1-1dh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In an embodiment, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In an embodiment, lignin biosynthesis may be downregulated in the plant so as to increase fermentable carbohydrates.

In an embodiment, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In an embodiment, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In an embodiment, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In an embodiment, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos, or parts thereof.

### Detecting Modifications in the Plant Genome-Selectable Markers

When the compositions, systems, and methods are used to modify a plant, suitable methods may be used to confirm and detect the modification made in the plant. In an embodiment, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or CI genes).

### Applications in Fungi

The compositions, systems, and methods described herein can be used to perform efficient and cost-effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In an embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In an embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In an embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In an embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In an embodiment, the abundance of guide molecule, e.g., guide RNA, may more often be a rate-limiting component in genome engineering of polyploid cells than in haploid cells, and thus the methods using the CRISPR system described herein may take advantage of using certain fungal cell types.

In an embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In an embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-December; 1 (6): 395-403.

In an embodiment, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2u plasmids, yeast integrative plasmids, yeast replicative plasmids, shuttle vectors, and episomal plasmids.

### Biofuel and Materials Production by Fungi

In an embodiment, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced into fungi. In an embodiment, the genes may encode enzymes involved in the conversion of pyruvate to ethanol or another product of interest, degrade cellulose (e.g., cellulase), endogenous metabolic pathways which compete with the biofuel production pathway.

In an embodiment, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S.J., et al. (2011) Proc. Natl. Acad. Sci. USA 108 (2): 504-9 and Galazka, J. M., et al. (2010) Science 330 (6000): 84-6; Jakočiūnas T et al., Metab Eng. 2015 March;28:213-222; Stovicek V, et al., FEMS Yeast Res. 2017 Aug. 1; 17 (5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi or may result from sexual reproduction by crossing with other individuals of the same species to introgress further desirable traits into their offspring. The cell may be in vivo or ex vivo in the cases of multicellular organisms, particularly plants.

### Further Applications of the CRISPR-Cas System in Plants

Further applications of the compositions, systems, and methods on plants and fungi include visualization of genetic element dynamics (e.g., as described in Chen B, et al., Cell. 2013 Dec. 19; 155 (7): 1479-91), targeted gene disruption positive-selection in vitro and in vivo (as described in Malina A et al., Genes Dev. 2013 Dec. 1; 27 (23): 2602-14), epigenetic modification such as using fusion of Cas and histone-modifying enzymes (e.g., as described in Rusk N, Nat Methods. 2014 January; 11 (1): 28), identifying transcription regulators (e.g., as described in Waldrip Z J, Epigenetics. 2014 September;9 (9): 1207-11), anti-virus treatment for both RNA and DNA viruses (e.g., as described in Price A A, et al., Proc Natl Acad Sci USA. 2015 May 12; 112 (19): 6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112 (36): 11211-6; Anton T, et al., Nucleus. 2014 March-April;5 (2): 163-72), self-cleavage of the CRISPR system for controlled inactivation/activation (e.g., as described Sugano S S et al., Plant Cell Physiol. 2014 March;55 (3): 475-81), multiplexed gene editing (as described in Kabadi A M et al., Nucleic Acids Res. 2014 Oct. 29; 42 (19): e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August;8 (8): 1274-84), regulation of non-coding genes and sequences (e.g., as described in Lowder L G, et al., Plant Physiol. 2015 October; 169 (2): 971-85), editing genes in trees (e.g., as described in Belhaj K et al., Plant Methods. 2013 Oct. 11; 9 (1): 39; Harrison M M, et al., Genes Dev. 2014 Sep. 1; 28 (17): 1859-72; Zhou X et al., New Phytol. 2015 October;208 (2): 298-301), introduction of mutations for resistance to host-specific pathogens and pests.

Additional examples of modifications of plants and fungi that may be performed using the compositions, systems, and methods include those described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In an embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0—genome editing for fitter, healthier, and more productive farmed animals. Genome Biol. 2018 Nov. 26; 19 (1): 204; Lillico S, Agricultural applications of genome editing in farmed animals. Transgenic Res. 2019 August;28 (Suppl 2): 57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats, horses, wolves, rabbits, ferrets, gerbils, hamsters, chinchillas, fancy rats, guinea pigs, canaries, parakeets, and parrots.

In an embodiment, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g., in pigs (such as described in Li M, et al., Genetics. 2018; 8:1747-54). Phytase (e.g., from E coli) xylanase (e.g., from Aspergillus niger), beta-glucanase (e.g., from Bacillus lichenformis) may be introduced to reduce the environmental impact through phosphorous and nitrogen release reduction, e.g., pigs (such as described in Golovan S P, et al., Nat Biotechnol. 2001; 19:741-5; Zhang X et al., elife. 2018). shRNA decoy may be introduced to induce avian influenza resilience e.g., in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLOS One. 2017; 12: e0169317). CD163 may be modified (e.g., inactivated or removed) to introduce PRRSV resilience in pigs (such as described in Prather R S et al . . . , Sci Rep. 2017 Oct. 17; 7 (1): 13371). Similar approaches may be used to inhibit or remove viruses and bacteria (e.g., Swine Influenza Virus (SIV) strains which include influenza C and the subtypes of influenza A known as HIN1, H1N2, H2N1, H3N1, H3N2, and H2N3, as well as pneumonia, meningitis, and oedema) that may be transmitted from animals to humans.

In an embodiment, one or more genes may be modified or edited for disease resistance and production traits. Myostatin (e.g., GDF8) may be modified to increase muscle growth, e.g., in cow, sheep, goat, catfish, and pig (such as described in Crispo M et al., PLOS One. 2015; 10: e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISSIR may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2). RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMPI may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December;7 (6): 580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111 (20): 7260-5; and Schomberg et al. FASEB Journal, April 2016; 30 (1): Suppl 571.1.

SNPs in the animals may be modified. Examples of methods and approaches include those described Tan W. et al., Proc Natl Acad Sci USA. 2013 Oct. 8; 110 (41): 16526-31; Mali P, et al., Science. 2013 Feb. 15; 339 (6121): 823-6.

Stem cells (e.g., induced pluripotent stem cells) may be modified and differentiated into desired progeny cells, e.g., as described in Heo Y T et al., Stem Cells Dev. 2015 Feb. 1; 24 (3): 393-402.

Profile analysis (such as Igenity) may be performed on animals to screen and identify genetic variations related to economic traits. The genetic variations may be modified to introduce or improve the traits, such as carcass composition, carcass quality, maternal and reproductive traits, and average daily gain.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In an embodiment, the pharmaceutical formulation can include, such as an active ingredient, a CRISPR-Cas system or component thereof or composition thereof described in greater detail elsewhere herein. In an embodiment, the pharmaceutical formulation can include, such as an active ingredient, a polynucleotide comprising one or more nucleic acid sequences encoding a CRISPR-Cas system or component thereof, or a vector comprising one or more of said polynucleotides, described in greater detail elsewhere herein. In an embodiment, the pharmaceutical formulation can include, such as an active ingredient, a delivery particle comprising one or more components, systems, compositions, polynucleotides, or vectors described in greater detail elsewhere herein. In an embodiment, the pharmaceutical formulation can include, such as an active ingredient one or more modified cells, such as one or more modified cells described in greater detail elsewhere herein.

In an embodiment, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In an embodiment, the subject in need thereof has or is suspected of having a hematopoietic disease, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In an embodiment, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In an embodiment, the amount of the primary active agent and/or optional secondary agent can be an effective amount, least effective amount, and/or therapeutically effective amount. As used herein, “effective amount”, “effective concentration”, and/or the like refers to the amount, concentration, etc. of the primary and/or optional secondary agent included in the pharmaceutical formulation that achieve one or more therapeutic effects or desired effect. As used herein, “least effective”, “least effective concentration”, and/or the like amount refers to the lowest amount, concentration, etc. of the primary and/or optional secondary agent that achieves the one or more therapeutic or other desired effects. As used herein, “therapeutically effective amount”, “therapeutically effective concentration” and/or the like refers to the amount, concentration, etc. of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value or subrange within any of these ranges.

In an embodiment, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In an embodiment, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In an embodiment where a cell or cell population is present in the pharmaceutical formulation (e.g., as a primary and/or or secondary active agent), the effective amount of cells can be any amount ranging from about 1 or 2 cells to 1x101 cells/mL, 1x1020 cells/mL or more, such as about 1x101 cells/mL, 1x102 cells/mL, 1x103 cells/mL, 1x104 cells/mL, 1x10⁵ cells/mL, 1x106 cells/mL, 1x107 cells/mL, 1x108 cells/mL, 1x109 cells/mL, 1x1010 cells/mL, 1x1011 cells/mL, 1x1012 cells/mL, 1x1013 cells/mL, 1x1014 cells/mL, 1x1015 cells/mL, 1x1016 cells/mL, 1x1017 cells/mL, 1x1018 cells/mL, 1x1019 cells/mL, to/or about 1x1020/cells/mL or any numerical value or subrange within any of these ranges.

In an embodiment, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In an embodiment, the effective amount can be about 1X101 particles per pL, nL, μL, mL, or L to 1X1020/particles per pL, nL, μL, mL, or L or more, such as about 1×101, 1x102, 1x103, 1x104, 1x105, 1x106, 1x107, 1x108, 1x109, 1x1010, 1x1011, 1x1012, 1x1013, 1x1014, 1x1015, 1x1016, 1x1017, 1x1018, 1x1019, to/or about 1×1020 particles per pL, nL, μL, mL, or L. In an embodiment, the effective titer can be about 1X101 transforming units per pL, nL, μL, mL, or L to 1X1020/transforming units per pL, nL, uL, mL, or L or more, such as about 1×101, 1x102, 1x103, 1x104, 1x105, 1x106, 1x107, 1x108, 1x109, 1x1010, 1x1011, 1x1012, 1x013, 1x1014, 1x1015, 1x1016, 1x1017, 1x1018, 1x1019, to/or about 1x1020 transforming units per pL, nL, uL, mL, or L or any numerical value or subrange within these ranges. In an embodiment, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In an embodiment, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In an embodiment, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In an embodiment, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In an embodiment, the given site is proximal to the administration site. In an embodiment, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In an embodiment, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In an embodiment, compounds, molecules, compositions, vectors, vector systems, cells, or a combination thereof described herein can be the ingredient whose release is delayed. In an embodiment the primary active agent is the ingredient whose release is delayed. In an embodiment, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20^{th} ed., Lippincott Williams & Wilkins, Baltimore, MD, 2000, and “Pharmaceutical dosage forms and drug delivery systems”, 6^{th} Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In an embodiment for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In an embodiment, a primary active ingredient, optional secondary active ingredient, and/or pharmaceutically acceptable salt thereof where appropriate can be in a dosage form adapted for inhalation is in a particle-size-reduced form that is obtained or obtainable by micronization. In an embodiment, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In an embodiment, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulation. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In an embodiment, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared In an embodiment, from sterile powders, granules, and tablets. The parenteral formulations can be administered to a subject in need thereof.

For certain example embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In an embodiment, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In an embodiment, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In an embodiment, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In an embodiment, the first one or a few initial amount(s) administered can be a higher dose than subsequent doses. This is typically referred to in the art as a loading dose or doses and a maintenance dose, respectively. In an embodiment, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Methods of Modifying a Target Polynucleotide and Therapeutic Uses Thereof

In an embodiment, the present disclosure provides a method of modifying a target polynucleotide. In an embodiment, a target polynucleotide can comprise any target nucleic acid sequence disclosed herein, e.g., a target nucleic sequence corresponding to a CRISPR-Cas guide molecule and susceptible to nuclease activity of a multimeric CRISPR-Cas complex. In an embodiment, the method comprises modifying a target polynucleotide in a cell, tissue, or organism. In an embodiment, the method of modifying the target polynucleotide is in vivo, ex vivo, or in vitro. In an embodiment, the cell or tissue is a host cell or tissue isolated from a subject.

In an embodiment, the method of modifying the target polynucleotide comprises contacting a sample comprising a target polynucleotide with one or more components of a CRISPR-Cas system (e.g., the CRISPR-Cas polypeptides and guide molecules), one or more or composition thereof (e.g., the multimeric CRISPR-Cas complex), one or more polynucleotides, vectors, delivery particles, engineered cells, or pharmaceutical compositions disclosed herein, or any combination thereof.

### Therapeutic Methods of Use

In an embodiment, contacting results in modification of a gene product or modification of the amount or expression of a gene product. In an embodiment, the target polynucleotide is a disease-or disorder-associated target polynucleotide.

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a target polynucleotide in a subject or cell thereof using a composition, system, or component thereof described herein and/or include detecting a diseased or healthy polynucleotide in a subject or cell thereof using a composition, system, or component thereof described herein. In an embodiment, the method of treatment or prevention can include using a composition, system, or component thereof to modify a polynucleotide of an infectious organism (e.g., bacterial or virus) within a subject or cell thereof. In an embodiment, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In an embodiment, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

In an embodiment, CRISPR-Cas is provided or expressed in an in vitro system or in a cell, transiently or stably, and targeted or triggered to non-specifically cleave cellular nucleic acids. In one embodiment, CRISPR-Cas is engineered to knock down ssDNA, for example viral ssDNA. In another embodiment, CRISPR-Cas is engineered to knock down RNA. The system can be devised such that the knockdown is dependent on a target DNA or RNA present in the cell or in vitro system or triggered by the addition of a target nucleic acid (e.g., DNA or RNA) to the system or cell.

In an embodiment, the CRISPR-Cas system is engineered to non-specifically cleave RNA (or DNA) in a subset of cells distinguishable by the presence of an aberrant DNA sequence, for instance where cleavage of the aberrant DNA might be incomplete or ineffectual. In one non-limiting example, a DNA translocation that is present in a cancer cell and drives cell transformation is targeted. Whereas a subpopulation of cells that undergoes chromosomal DNA and repair may survive, non-specific collateral ribonuclease (or deoxyribonuclease) activity advantageously leads to cell death of potential survivors.

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple Cas effectors. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the Cas effector(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the Cas effector(s) advantageously encoding and expressing in vivo the remaining portions of the composition, system, (e.g., RNA, guides); advantageously In an embodiment the CRISPR enzyme is a catalytically inactive Cas effector and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (lentiviral vector, adenoviral vector, AAV vector). As explained herein, use of different selection markers (e.g., lentiviral gRNA selection) and concentration of gRNA (e.g., dependent upon whether multiple gRNAs are used) may be advantageous for eliciting an improved effect.

Thus, also described herein are methods of inducing one or more polynucleotide modifications in a eukaryotic or prokaryotic cell or component thereof (a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In an embodiment, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiments or the cell of any one of the above embodiments.

Also provided herein is the use of the particle delivery system or the delivery system or the virus particle of any one of the above embodiments or the cell of any one of the above embodiments in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In an embodiment, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In an embodiment, the modifications can include the introduction, deletion, or substitution of nucleotides at each target sequence of said cell(s) via nucleic acid components (e.g., guide(s) RNA(s) or sgRNA(s)), such as those mediated by a composition, system, or a component thereof described elsewhere herein. In an embodiment, the modifications can include the introduction, deletion, or substitution of nucleotides at a target or random sequence of said cell(s) via a composition, system, or technique.

In an embodiment, the composition, system, or component thereof can promote Non-Homologous End-Joining (NHEJ). In an embodiment, modification of a polynucleotide by a composition, system, or a component thereof, such as a diseased polynucleotide, can include NHEJ. In an embodiment, promotion of this repair pathway by the composition, system, or a component thereof can be used to target gene or polynucleotide specific knock-outs and/or knock-ins. In an embodiment, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In an embodiment the indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58,f 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In an embodiment, composition, system, mediated NHEJ can be used in the method to delete small sequence motifs. In an embodiment, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide molecule, e.g., guide RNA, and Cas effector generate a double strand break for the purpose of inducing NHEJ-mediated indels, a guide molecule, e.g., guide RNA, may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide molecules, e.g., guide RNAs, complexing with one or more Cas nickases induce two single strand breaks for the purpose of inducing NHEJ-mediated indels, two guide molecules, e.g., guide RNAs, may be configured to position two single-strand breaks to provide for NHEJ repair a nucleotide of the target position.

For minimization of toxicity and off-target effect, it may be important to control the concentration of Cas mRNA and guide molecule, e.g., guide RNA, delivered. Optimal concentrations of Cas mRNA and guide molecule, e.g., guide RNA, can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide molecules, e.g., guide RNAs, targeting a site of interest. Guide sequences and strategies to minimize toxicity and off-target effects can be as in International Patent Publication No. WO 2014/093622 (PCT/US2013/074667); or, via mutation. Others are as described elsewhere herein. Typically, in the context of an endogenous CRISPR or system, formation of a CRISPR complex (comprising a guide sequence hybridized to a target sequence and complexed with one or more Cas proteins) results in cleavage, nicking, and/or another modification of one or both strands in or near (e.g., within 1, 2, 3, 4, 5, 6, 7, 8, 9, 10, 20, 50, or more base pairs from) the target sequence. In an embodiment, the tracr sequence, which may comprise or consist of all or a portion of a wild type tracr sequence (e.g., about or more than about 20, 26, 32, 45, 48, 54, 63, 67, 85, or more nucleotides of a wild type tracr sequence), can also form part of a CRISPR complex, such as by hybridization along at least a portion of the tracr sequence to all or a portion of a tracr mate sequence that is operably linked to the guide sequence.

In an embodiment, a method of modifying a target polynucleotide in a cell to treat or prevent a disease can include allowing a composition, system, or component thereof to bind to the target polynucleotide, e.g., to effect cleavage, nicking, or other modification as the composition, system is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some of these embodiments, the composition, system, or component thereof can be or include a CRISPR-Cas effector complexed with a guide sequence. In an embodiment, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In an embodiment, modification of transcription can include decreasing transcription of a target polynucleotide. In an embodiment, modification can include increasing transcription of a target polynucleotide. In an embodiment, the method includes repairing said cleaved target polynucleotide by homologous recombination with a recombination template polynucleotide, wherein said repair results in a modification such as, but not limited to, an insertion, deletion, or substitution of one or more nucleotides of said target polynucleotide. In an embodiment, said modification results in one or more amino acid changes in a protein expressed from a gene comprising the target sequence. In an embodiment, the modification imparted by the composition, system, or component thereof provides a transcript and/or protein that can correct a disease or a symptom thereof, including but not limited to, any of those described in greater detail elsewhere herein.

In an embodiment, the method of treating or preventing a disease can include delivering one or more vectors or vector systems to a cell, such as a eukaryotic or prokaryotic cell, wherein one or more vectors or vector systems include the composition, system, or component thereof. In an embodiment, the vector(s) or vector system(s) can be a viral vector or vector system, such as an AAV or lentiviral vector system, which are described in greater detail elsewhere herein. In an embodiment, the method of treating or preventing a disease can include delivering one or more viral particles, such as an AAV or lentiviral particle, containing the composition, system, or component thereof. In an embodiment, the viral particle has a tissue specific tropism. In an embodiment, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the invention as described herein, such as the composition and system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In an embodiment, the composition, system, and/or component thereof described herein can be used to treat and/or prevent a circulatory system disease. Exemplary disease is provided, for example. In an embodiment the plasma exosomes of Wahlgren et al. (Nucleic Acids Research, 2012, Vol. 40, No. 17 e130) can be used to deliver the composition, system, and/or component thereof described herein to the blood. In an embodiment, the circulatory system disease can be treated by using a lentivirus to deliver the composition, system, described herein to modify hematopoietic stem cells (HSCs) in vivo or ex vivo (See, e.g., Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi: 10.4061/2011/987980, which can be adapted for use with the composition, system, herein in view of the description herein). In an embodiment, the circulatory system disorder can be treated by correcting HSCs as to the disease using a composition, system, herein or a component thereof, wherein the composition, system, optionally includes a suitable HDR repair template (See, e.g., Cavazzana, “Outcomes of Gene Therapy for β-Thalassemia Major via Transplantation of Autologous Hematopoietic Stem Cells Transduced Ex Vivo with a Lentiviral βA-T87Q-Globin Vector.”; Cavazzana-Calvo, “Transfusion independence and HMGA2 activation after gene therapy of human β-thalassaemia”, Nature 7, 318-322 (16 Sep. 2010) doi: 10.1038/nature09328; Nienhuis, “Development of Gene Therapy for Thalassemia, Cold Spring Perspectives in Medicine, doi: 10.1101/cshperspect.a011833 (2012), LentiGlobin BB305, a lentiviral vector containing an engineered β-globin gene (βA-T87Q); and Xie et al., “Seamless gene correction of β-thalassaemia mutations in patient-specific iPSCs using CRISPR/Cas9 and piggyback” Genome Research gr.173427.114 (2014) www.genome.org/cgi/doi/10.1101/gr.173427.114 (Cold Spring Harbor Laboratory Press; Watts, “Hematopoietic Stem Cell Expansion and Gene Therapy” Cytotherapy 13 (10): 1164-1171. doi: 10.3109/14653249.2011.620748 (2011), which can be adapted for use with the composition, system, herein in view of the description herein). In an embodiment, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24 (9): 1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,-the receptor for stem cell factor. Hematopoietic stem cells are negative for the markers that are used for detection of lineage commitment, and are, thus, called Lin-; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. Mouse HSC markers: CD34lo/—, SCA-1+, Thy 1.1+/lo, CD38+, C-kit+, lin−, and Human HSC markers: CD34+, CD59+, Thyl/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In an embodiment, the treatment or prevention for treating a circulatory system or blood disease can include modifying a human cord blood cell with any modification described herein. In an embodiment, the treatment or prevention for treating a circulatory system or blood disease can include modifying a granulocyte colony-stimulating factor-mobilized peripheral blood cell (mPB) with any modification described herein. In an embodiment, the human cord blood cell or mPB can be CD34+. In an embodiment, the cord blood cell(s) or mPB cell(s) modified can be autologous. In an embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010)857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The CRISPR-Cas (system may be engineered to target genetic locus or loci in HSCs. In an embodiment, the Cas effector(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the Cas effector protein and the gRNA being admixed. The gRNA and Cas effector protein mixture can be, for example, admixed with a mixture comprising or consisting essentially of or consisting of surfactant, phospholipid, biodegradable polymer, lipoprotein and alcohol, whereby particles containing the gRNA and Cas effector protein may be formed. The invention comprehends so making particles and particles from such a method as well as uses thereof. Particles suitable delivery of the CRISRP-Cas systems in the context of blood or circulatory system or HSC delivery to the blood or circulatory system are described in greater detail elsewhere herein.

In an embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121 (20): 4082-9. doi: 10.1182/blood-2012-09-455204. Epub 2013 Mar. 21.

In an embodiment, the HSCs or iPSCs modified can be autologous. In an embodiment, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010)857-862, which can be adapted for use with the composition, system, herein.

### Treating Neurological Diseases

In an embodiment, the compositions, systems, described herein can be used to treat diseases of the brain and CNS. Delivery options for the brain include encapsulation of CRISPR enzyme and guide molecule, e.g., guide RNA, in the form of either DNA or RNA, into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing CRISPR enzyme and guide molecule, e.g., guide RNA. For instance, Xia CF and Boado R J, Pardridge W M (“Antibody-mediated targeting of siRNA via the human insulin receptor using avidin-biotin technology.” Mol Pharm. 2009 May-June;6 (3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g., Zhang et al. (Mol Ther. 2003 January;7 (1): 11-8.)), the teachings of which can be adapted for use with the compositions, systems, herein.

### Treating Hearing Diseases

In an embodiment the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In an embodiment, the composition, system, or modified cells can be delivered to one or both ears for treating or preventing hearing disease or loss by any suitable method or technique. Suitable methods and techniques include, but are not limited to, those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g., McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g., Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In an embodiment, a catheter or pump can be positioned, e.g., in the ear (e.g., the outer, middle, and/or inner ear) of a patient during a surgical procedure. In an embodiment, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318 (5858): 1917-1920 (2007); Nakagawa et al., Nat. Biotechnol. 26:101-106 (2008); and Zaehres and Scholer, Cell 131 (5): 834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-lgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In an embodiment the pharmaceutical composition is applied to the ear canal. Delivery to the ear may also be referred to as aural or otic delivery.

In an embodiment, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June;228 (1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In an embodiment, the system set forth in Mukherjea et al. (Antioxidants & Redox Signaling, Volume 13, Number 5, 2010) can be adapted for transtympanic administration of the composition, system, or component thereof to the ear. In an embodiment, a dosage of about 2 mg to about 4 mg of CRISPR Cas for administration to a human.

In an embodiment, the system set forth in [Jung et al. (Molecular Therapy, vol. 21 no. 4, 834-841, April 2013) can be adapted for vestibular epithelial delivery of the composition, system, or component thereof to the ear. In an embodiment, a dosage of about 1 to about 30 mg of CRISPR Cas for administration to a human.

### Treating Diseases in Non-Dividing Cells

In an embodiment, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the Cas compositions, systems, described herein.

Thus, reactivation of HR in cells, especially non-dividing, fully differentiated cell types is preferred, in an embodiment. In an embodiment, promotion of the BRCA1-PALB2 interaction is preferred in an embodiment. In an embodiment, the target ell is a non-dividing cell. In an embodiment, the target cell is a neuron or muscle cell. In an embodiment, the target cell is targeted in vivo. In an embodiment, the cell is in G1 and HR is suppressed. In an embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in an embodiment, especially where the target cells are non-dividing, or where removal and return (ex vivo gene targeting) is problematic, for example neuron or muscle cells. KEAP1 siRNA is available from ThermoFischer. In an embodiment, a BRCA1-PALB2 complex may be delivered to the G1 cell. In an embodiment, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In an embodiment, the disease to be treated is a disease that affects the eyes. Thus, In an embodiment, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In an embodiment, the condition to be treated or targeted is an eye disorder. In an embodiment, the eye disorder may include glaucoma. In an embodiment, the eye disorder includes a retinal degenerative disease. In an embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In an embodiment, the retinal degenerative disease is Leber Congenital Amaurosis (LCA) or Retinitis Pigmentosa. Other exemplary eye diseases are described in greater detail elsewhere herein.

In an embodiment, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×1010 or 1.0-1.4×109 transducing units (TU)/ml.

In an embodiment, for administration to the eye, lentiviral vectors. In an embodiment, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In an embodiment, the dosage can be 1.1×105 transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In an embodiment, the dose can range from about 106 to 109.5 particle units. In the context of the Millington-Ward AAV vectors, a dose of about 2×1011 to about 6×1013 virus particles can be administered. In the context of Dalkara vectors, a dose of about 1×1015 to about 1×1016 vg/ml administered to a human.

In an embodiment, the sd-rxRNA® system of R×i Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNAR system may be applied to the nucleic acid-targeting system of the present invention, contemplating a dose of about 3 to 20 mg of CRISPR administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc: 193933, prdmla, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In an embodiment, the composition, system can be used to treat and/or prevent a muscle disease and associated circulatory or cardiovascular disease or disorder. The present invention also contemplates delivering the composition, system, described herein, e.g., Cas effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×1014 vector genomes is contemplated for systemic administration. See also, e.g., Eulalio et al. (2012) Nature 492:376 and Somasuntharam et al. (2013) Biomaterials 34:7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease, or the protein encoded by any chromosomal sequence involved in cardiovascular disease, may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present invention also contemplates delivering the composition, system, described herein, effector protein systems, to muscle(s).

In an embodiment, the muscle disease to be treated is a muscle dystrophy such as DMD. In an embodiment, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In an embodiment, exon skipping may include the masking of key sequences involved in the splicing of targeted exons by using a composition, system, described herein capable of RNA modification. In an embodiment, exon skipping can be achieved in dystrophin mRNA. In an embodiment, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In an embodiment, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In an embodiment, for treatment of a muscle disease, the method of Bortolanza et al. Molecular Therapy vol. 19 no. 11, 2055-264 November 2011) may be applied to an AAV expressing CRISPR Cas and injected into humans at a dosage of about 2×1015 or 2× 1016 vg of vector. The teachings of Bortolanza et al., can be adapted for and/or applied to the compositions, systems, described herein.

In an embodiment, the method of Dumonceaux et al. (Molecular Therapy vol. 18 no. 5, 881-887 May 2010) may be applied to an AAV expressing CRISPR Cas and injected into humans, for example, at a dosage of about 1014 to about 1015 vg of vector. The teachings of Dumonceaux described herein can be adapted for and/or applied to the compositions, systems, described herein.

In an embodiment, the method of Kinouchi et al. (Gene Therapy (2008)15, 1126-1130) may be applied to CRISPR Cas systems described herein and injected into a human, for example, at a dosage of about 500 to 1000 ml of a 40 uM solution into the muscle.

In an embodiment, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In an embodiment, the method comprises treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The Cas protein is inserted and directed by an RNA guide to the mutated point and then it cuts the DNA at that point. Simultaneously, a healthy version of the sequence is inserted. This sequence is used by the cell's own repair system to fix the induced cut. In this way, the CRISPR-Cas allows the correction of the mutation in the previously obtained stem cells. The methods and systems may be used to correct HSCs as to sickle cell anemia using a systems that targets and corrects the mutation (e.g., with a suitable HDR template that delivers a coding sequence for β-globin, advantageously non-sickling β-globin); specifically, the guide molecule, e.g., guide RNA, can target mutation that give rise to sickle cell anemia, and the HDR can provide coding for proper expression of β-globin. A guide molecule, e.g., guide RNA, that targets the mutation-and-Cas protein containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., βA-T87Q), or β-globin.

### Treating Diseases of the Liver and Kidney

In an embodiment, the composition, system, or component thereof described herein can be used to treat a disease of the kidney or liver. Thus, in an embodiment, delivery of the CRISRP-Cas system or component thereof described herein is to the liver or kidney.

Delivery strategies to induce cellular uptake of the therapeutic nucleic acid include physical force or vector systems such as viral-, lipid-or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the CRISPR Cas system of the present invention contemplating a 1-2 g subcutaneous injection of CRISPR Cas conjugated with cholesterol to a human for delivery to the kidneys. In an embodiment, the method of Molitoris et al. (J Am Soc Nephrol 20:1754-1764, 2009) can be adapted to the CRISRP-Cas system of the present invention and a cumulative dose of 12-20 mg/kg to a human can be used for delivery to the proximal tubule cells of the kidneys. In an embodiment, the methods of Thompson et al. (Nucleic Acid Therapeutics, Volume 22, Number 4, 2012) can be adapted to the CRISRP-Cas system of the present invention and a dose of up to 25 mg/kg can be delivered via i.v. administration. In an embodiment, the method of Shimizu et al. (J Am Soc Nephrol 21:622-633, 2010) can be adapted to the CRISRP-Cas system of the present invention and a dose of about of 10-20 μmol CRISPR Cas complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g., Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In an embodiment, delivery is to liver cells. In an embodiment, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology-abstract available online at ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In an embodiment, the disease treated or prevented by the composition and system described herein can be a lung or epithelial disease. The compositions and systems described herein can be used for treating epithelial and/or lung diseases. The present invention also contemplates delivering the composition, system, described herein, to one or both lungs.

In an embodiment, as viral vector can be used to deliver the composition, system, or component thereof to the lungs. In an embodiment, the AAVis an AAV-1, AAV-2, AAV-5, AAV-6, and/or AAV-9 for delivery to the lungs (see, e.g., Li et al., Molecular Therapy, vol. 17 no. 12, 2067-277 December 2009). In an embodiment, the MOI can vary from 1×103 to 4×105 vector genomes/cell. In an embodiment, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized CRISPR Cas, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1α promoter for Cas, U6 or H1 promoter for guide RNA): A preferred arrangement is to use a CFTRdelta508 targeting guide, a repair template for deltaF508 mutation and a codon optimized Cas enzyme, with optionally one or more nuclear localization signal or sequence(s) (NLS(s)), e.g., two (2) NLSs.

### Treating Diseases of the Skin

The compositions and systems described herein can be used for the treatment of skin diseases. The present invention also contemplates delivering the composition and system, described herein, to the skin.

In an embodiment, delivery to the skin (intradermal delivery) of the composition, system, or component thereof can be via one or more microneedles or microneedle containing device. For example, In an embodiment the device and methods of Hickerson et al. (Molecular Therapy-Nucleic Acids (2013)2, e129) can be used and/or adapted to deliver the composition, system, described herein, for example, at a dosage of up to 300 μl of 0.1 mg/ml CRISPR-Cas system to the skin.

In an embodiment, the methods and techniques of Leachman et al. (Molecular Therapy, vol. 18 no. 2, 442-446 February 2010) can be used and/or adapted for delivery of a CIRPSR-Cas system described herein to the skin.

In an embodiment, the methods and techniques of Zheng et al. (PNAS, Jul. 24, 2012, vol. 109, no. 30, 11975-11980) can be used and/or adapted for nanoparticle delivery of a CIRPSR-Cas system described herein to the skin. In an embodiment, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 8 and 9. In an embodiment, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an embodiment of the invention, methods and compositions which involve editing a target nucleic acid sequence, or modulating expression of a target nucleic acid sequence, and applications thereof in connection with cancer immunotherapy are comprehended by adapting the composition, system, of the present invention. In an embodiment, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In an embodiment, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for-thalassemia, Nat Commun. 2017 Sept 4; 8 (1): 424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June;24 (6): 724-730; Besser et al., (2010) Clin. Cancer Res 16 (9)2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314 (5796)126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In an embodiment, allogenic cells immune cells are transferred (see, e.g., Ren et al., (2017) Clin Cancer Res 23 (9)2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32:189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12 (4): 269-281; and Jenson and Riddell, 2014, Design and implementation of adoptive therapy with chimeric antigen receptor-modified T cells. Immunol Rev. 257 (1): 127-144; and Rajasagi et al., 2014, Systematic identification of personal tumor-specific neoantigens in chronic lymphocytic leukemia. Blood. 2014 July 17; 124 (3): 453-62).

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTAI (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac (2-8) aNeu5Ac (2-3) bDGalp (1-4) bDGlcp (1-1) Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac (2-3) bDGalp (1-4) bDGlcp (1-1) Cer); TGS5; high molecular weight-melanoma-associated antigen (HMWMAA); 0-acetyl-GD2 ganglioside (OAcGD2); Folate receptor alpha; Folate receptor beta; tumor endothelial marker 1 (TEMI/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member IA (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SARTI, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1,-2,-3 or 4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAPI (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2,-3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); LICAM (LI cell adhesion molecule); MC1R (melanocortin 1 receptor); Myosin/m (myosin mutated); MUM-1,-2,-3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AMLI (translocation Ets-family leukemia/acute myeloid leukemia 1); TPI/m (triosephosphate isomerase mutated); CD70; and any combination thereof.

In an embodiment, an antigen to be targeted in adoptive cell therapy (such as particularly CAR or TCR T-cell therapy) of a disease (such as particularly of tumor or cancer) is a tumor-specific antigen (TSA).

In an embodiment, an antigen to be targeted in adoptive cell therapy (such as particularly CAR or TCR T-cell therapy) of a disease (such as particularly of tumor or cancer) is a neoantigen.

In an embodiment, an antigen to be targeted in adoptive cell therapy (such as particularly CAR or TCR T-cell therapy) of a disease (such as particularly of tumor or cancer) is a tumor-associated antigen (TAA).

In an embodiment, an antigen to be targeted in adoptive cell therapy (such as particularly CAR or TCR T-cell therapy) of a disease (such as particularly of tumor or cancer) is a universal tumor antigen. In an embodiment, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (Dl), and any combinations thereof.

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WTI, CD22, CD171, ROR1, MUC16, and SSX2. In an embodiment, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLLI may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WTI may be targeted in acute myeloid leukemia (AML), myelodysplastic syndromes (MDS), chronic myeloid leukemia (CML), non-small cell lung cancer, breast, pancreatic, ovarian, or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753, 162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in an embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD33-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In an embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In an embodiment, the primary signaling domain comprises a functional signaling domain of CD3ζ or FcRγ. In an embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGLI, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In an embodiment, the one or more costimulatory signaling domains comprise a functional signaling domain of a protein selected, each independently, from the group consisting of: 4-1BB, CD27, and CD28. In an embodiment, a chimeric antigen receptor may have the design as described in U.S. Pat. No. 7,446,190, comprising an intracellular domain of CD3% chain (such as amino acid residues 52-163 of the human CD3 zeta chain, as shown in SEQ ID NO: 14 of U.S. Pat. No. 7,446,190), a signaling region from CD28 and an antigen-binding element (or portion or domain, such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD35 chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 83) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an Xhol site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with Xhol and NotI. To form the MSGV-FMC63-28Z retroviral vector, the XhoI and NotI-digested fragment encoding the FMC63 scFv was ligated into a second Xhol and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-ζ (molecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in an embodiment, cells intended for adoptive cell therapies, more particularly immunoresponsive cells such as T cells, may express the FMC63-28Z CAR as described by Kochenderfer et al. (supra). Hence, In an embodiment, cells intended for adoptive cell therapies, more particularly immunoresponsive cells such as T cells, may comprise a CAR comprising an extracellular antigen-binding element (or portion or domain, such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM 006139 (sequence version 1, 2 or 3) starting with the amino acid sequence IEVMYPPPY (SEQ ID NO: 83) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3; 4-1BB-CD3ζ; CD27-CD3ζ; CD28-CD27-CD3ζ, 4-1BB-CD27-CD3ζ; CD27-4-1BB-CD3ζ; CD28-CD27-FceRI gamma chain; or CD28-FceRI gamma chain) were disclosed. Hence, In an embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of No. WO 2015/187528. Preferably, the antigen is CD19, more preferably the antigen-binding element is an anti-CD19 scFv, even more preferably the mouse or human anti-CD19 scFv as described in Example 1 of. WO 2015/187528. In an embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, a chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March;78:145-150; and Jin et al., CD70, a novel target of CAR T-cell therapy for gliomas, Neuro Oncol. 2018 January 10; 20 (1): 55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In an embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In an embodiment, the chimeric inhibitory receptor comprises an extracellular antigen-binding element (or portion or domain) configured to specifically bind to a target antigen, a transmembrane domain, and an intracellular immunosuppressive or repressive signaling domain. In an embodiment, the second target antigen is an antigen that is not expressed on the surface of a cancer cell or infected cell or the expression of which is downregulated on a cancer cell or an infected cell. In an embodiment, the second target antigen is an MHC-class I molecule. In an embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in an embodiment, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids, or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may, for example, include vectors based on HIV, SV40, EBV, HSV or BPV.

Cells that are targeted for transformation may, for example, include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may, for example, be selected through co-culture with γ-irradiated activating and propagating cells (AaPC), which co-express the cancer antigen and co-stimulatory molecules. The engineered CAR T-cells may be expanded, for example, by co-culture on AaPC in presence of soluble factors, such as IL-2 and IL-21. This expansion may for example be carried out so as to provide memory CAR+ T cells (which may, for example, be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CAR T cells of this kind may, for example, be used in animal models, for example, to treat tumor xenografts.

In an embodiment, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6 (10): e160).

In an embodiment, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112 (2): 362-73; and Martin-Orozco N, et al., T helper 17 cells promote cytotoxic T cell activation in tumor immunity. Immunity. 2009 Nov. 20; 31 (5): 787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In an embodiment, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem. 2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In an embodiment, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257 (1): 56-71. doi: 10.1111/imr.12132).

Approaches such as the foregoing may be adapted to provide methods of treating and/or increasing survival of a subject having a disease, such as a neoplasia, for example, by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In an embodiment, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3 (10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In an embodiment, the immunosuppressive treatment provides for the selection and expansion of the immunoresponsive T cells within the patient.

In an embodiment, the treatment can be administered before primary treatment (e.g., surgery or radiation therapy) to shrink a tumor before the primary treatment. In another embodiment, the treatment can be administered after primary treatment to remove any remaining cancer cells.

In an embodiment, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation, or transplantation. The cells or population of cells may be administered to a patient subcutaneously, intradermally, intratumorally, intranodally, intramedullary, intramuscularly, intrathecally, by intravenous or intralymphatic injection, or intraperitoneally. In an embodiment, the disclosed CARs may be delivered or administered into a cavity formed by the resection of tumor tissue (i.e., intracavity delivery) or directly into a tumor prior to resection (i.e., tumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells is administrated as a single dose. In another embodiment, the effective amount of cells is administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28 (6): 1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23 (9): 2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 January 25; 9 (374); Legut, et al., 2018, CRISPR-mediated TCR replacement generates superior anticancer transgenic T cells. Blood, 131 (3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g., TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In an embodiment, editing may result in inactivation of a gene. By inactivating a gene it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In an embodiment, homology directed repair (HDR) is used to concurrently inactivate a gene (e.g., TRAC) and insert an endogenous TCR or CAR into the inactivated locus.

Hence, in an embodiment, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and B, which assemble to form a heterodimer and associates with the CD3-transducing subunits to form the T cell receptor complex present on the cell surface. Each a and B chain of the TCR consists of an immunoglobulin-like N-terminal variable (V) and constant (C) region, a hydrophobic transmembrane domain, and a short cytoplasmic region. As for immunoglobulin molecules, the variable region of the a and B chains are generated by V (D) J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in an embodiment, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112 (12): 4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid, or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In an embodiment, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In an embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 April 15; 44 (2): 356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MTI and/or MT2 inhibitors to increase proliferation and/or activity of exhausted CD8+ T-cells and to decrease CD8+ T-cell exhaustion (e.g., decrease functionally exhausted or unresponsive CD8+ immune cells). In an embodiment, metallothioneins are targeted by gene editing in adoptively transferred T cells.

In an embodiment, targets of gene editing may be at least one targeted locus involved in the expression of an immune checkpoint protein. Such targets may include, but are not limited to CTLA4, PPP2CA, PPP2CB, PTPN6, PTPN22, PDCDI, ICOS (CD278), PDL1, KIR, LAG3, HAVCR2, BTLA, CD160, TIGIT, CD96, CRTAM, LAIRI, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, ILIORA, ILIORB, HMOX2, IL6R, IL6ST, EIF2AK4, CSK, PAGI, SITI, FOXP3, PRDMI, BATF, VISTA, GUCY1A2, GUCY1A3, GUCY1B2, GUCY1B3, MTI, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT. By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIRI, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PDI simultaneously, to generate gene-disrupted allogeneic CAR T cells deficient of TCR, HLA class I molecule and PD1.

In an embodiment, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In an embodiment, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In an embodiment, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUCI, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In an embodiment, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRa, PD1 and TCRB, CTLA-4 and TCRa, CTLA-4 and TCRB, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In an embodiment, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In an embodiment, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In an embodiment of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In an embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In an embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead: cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In an embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×105/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In an embodiment, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. U.S. Pat. No. 20,040,224,402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274 (5284): 94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled β2-microglobulin (β2m) into MHC class I/β2m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000-fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In an embodiment, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In an embodiment, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In an embodiment, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In an embodiment, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m2/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In an embodiment, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In an embodiment, modifying one or more copies of one or more genes associated with a genetic disease or a disease with a genetic and/or epigenetic aspect in the subject can eliminate a genetic disease or a symptom thereof in the subject. In an embodiment, modifying one or more copies of one or more genes associated with a genetic disease or a disease with a genetic and/or epigenetic aspect in the subject can decrease the severity of a genetic disease or a symptom thereof in the subject. In an embodiment, the compositions, systems, or components thereof can modify one or more genes or polynucleotides associated with one or more diseases, including genetic diseases and/or those having a genetic aspect and/or epigenetic aspect, including but not limited to, any one or more set forth in Table 8. It will be appreciated that those diseases and associated genes listed herein are non-exhaustive and non-limiting. Further some genes play roles in the development of multiple diseases.

TABLE 8
Exemplary Genetic and Other Diseases and Associated Genes

| | Primary | | |
|---|---|---|---|
| | Tissues or | Additional | |
| | System | Tissues/Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g., |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; ALS7; |
| | | | STEX; FUS; TARDBP; VEGF |
| | | | (VEGF-a, |
| | | | VEGF-b, VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, |
| | | | :GA:S1, CD44, TP53, TLR3, GRIA1, |
| | | | GAPDH, AMPA, GRIK1, DES, |
| | | | CHAT, FLT4, CHMP2B, BAG1, |
| | | | CRNA COMPONENT4, GSS, BAK1, |
| | | | KDR, GSTP1, OGG1, IL6 |
| Alzheimer's Disease | Brain | | E1; CHIP; UCH; UBB; Tau; LRP; |
| | | | PICALM; CLU; PS1; SORL1; CR1; |
| | | | VLDLR; UBA1; UBA3; CHIP28; |
| | | | AQP1; UCHL1; UCHL3; APP, AAA, |
| | | | CVAP, AD1, APOE, AD2, DCP1, |
| | | | ACE1, MPO, PACIP1, PAXIP1L, |
| | | | PTIP, A2M, BLMH, BMH, PSEN1, |
| | | | AD3, ALAS2, ABCA1, BIN1, BDNF, |
| | | | BTNL8, C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | IRA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| | | | FMR1, FMR2; FXR1; FXR2; |
| Pervasive Developmental | | | MGLUR5, ATP10C, CDH10, GRM6, |
| Disorders (PDDs) | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, See also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tuberous sclerosis complex | | | |
| disease) | | | |
| Autosomal Recessive Polycystic | kidney | liver | PKDH1 |
| Kidney Disease (ARPKD) | | | |
| Ataxia-Telangiectasia (a/k/a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammaglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAMI, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CRNA COMPONENT4, CACNA1C, |
| | | | PRKAG2, CHAT, PTGDS, NR1H2, |
| | | | TEK, VEGFB, MEF2C, MAPKAPK2, |
| | | | TNFRSF11A, HSPA9, CYSLTR1, |
| | | | MAT1A, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chediak-Higashi Syndrome | Immune system | Skin, hair, eyes, | LYST |
| | | neurons | |
| Choroideremia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: MSH2, |
| adenomatous polyposis (FAP) | | | MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g., CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubral-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; GRIA2; |
| | | | Grm5; Grin1; Htr1b; Grin2a; Drd3; |
| | | | Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, See also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholesterolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Disease Types | Metabolism | | SLC2A2, GLUT2, G6PC, G6PT, |
| I-VI -See also Cori's Disease, | Diseases | | G6PT1, GAA, LAMP2, LAMPB, |
| Pompe's Disease, McArdle's | | | AGL, GDE, GBE1, GYS2, PYGL, |
| disease, Hers Disease, and Von | | | PFKM, See also Cori's Disease, |
| Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup disease | Malabsorption | Various- brain, | SLC6A19 |
| | disease | gastrointestinal, | |
| | | skin. | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g., galactosemia), Amino |
| organic acidemias), fatty acid | | | acid Metabolism disorders (e.g., |
| oxidation defects, | | | phenylketonuria), Fatty acid |
| aminoacidopathies, carbohydrate | | | metabolism (e.g., MCAD deficiency), |
| disorders, mitochondrial | | | Urea Cycle disorders (e.g., |
| disorders | | | Citrullinemia), Organic acidemias |
| | | | (e.g., Maple Syrup Urine disease), |
| | | | Mitochondrial disorders (e.g., |
| | | | MIELAS), peroxisomal disorders (e.g., |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eve | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulonephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCAIB, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinese | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDCIC, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; Notch1; Notch2; |
| | | | Notch3; Notch4; AKT; AKT2; AKT3; |
| | | | HIF; HIF1a; HIF3a; Met; HRG; Bcl2; |
| | | | PPAR alpha; PPAR gamma; WT1 |
| | | | (Wilms Tumor); FGF Receptor Family |
| | | | members (5 members: 1, 2, 3, 4, 5); |
| | | | CDKN2a; APC; RB (retinoblastoma); |
| | | | MEN1; VHL; BRCA1; BRCA2; AR |
| | | | (Androgen Receptor); TSG101; IGF; |
| | | | IGF Receptor; Igf1 (4 variants); Igf2 (3 |
| | | | variants); Igf 1 Receptor; Igf 2 |
| | | | Receptor; Bax; Bc12; caspases family |
| | | | (9 members: 1, 2, 3, 4, 6, 7, 8, 9, 12); |
| | | | Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCAIB, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROMI, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, See also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); Complexin1 (Cplx1); |
| | | | Tph1 Tryptophan hydroxylase; Tph2 |
| | | | Tryptophan hydroxylase 2; Neurexin |
| | | | 1; GSK3; GSK3a; GSK3b; 5-HTT |
| | | | (Slc6a4); COMT; DRD (Drd1a); |
| | | | SLC6A3; DAOA; DTNBP1; Dao |
| | | | (Dao1); TCF4; COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APHIB, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 |
| | | | and −130 of the HBG Transcription |
| | | | Start Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| Trimethylaminuria | Metabolism | | FMO3 |
| | disease | | |
| Trinucleotide Repeat Disorders | Various | | HTT; SBMA/SMAX1/AR; FXN/X25 |
| (generally) | | | ATX3; ATXN1; ATXN2; DMPK; |
| | | | Atrophin-1 and Atn1 (DRPLA Dx); |
| | | | CBP (Creb-BP - global instability); |
| | | | VLDLR; Atxn7; Atxn10; FEN1, |
| | | | TNRC6A, PABPN1, JPH3, MED15, |
| | | | ATXN1, ATXN3, TBP, CACNA1A, |
| | | | ATXN80S, PPP2R2B, ATXN7, |
| | | | TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, See also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler Cardiofacial | | | |
| Syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In an embodiment, the compositions, systems, or components thereof can be used treat or prevent a disease in a subject by modifying one or more genes associated with one or more cellular functions, such as any one or more of those in Table 9. In an embodiment, the disease is a genetic disease or disorder. In some of embodiments, the composition, system, or component thereof can modify one or more genes or polynucleotides associated with one or more genetic diseases such as any set forth in Table 9.

TABLE 9
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; |
| | MAPK9; CDK2; PPP2CA; PIM1; ITGB7; YWHAZ; ILK; TP53; RAF1; |
| | IKBKG; RELB; DYRK1A; CDKN1A; ITGB1; MAP2K2; JAK1; AKT1; JAK2; |
| | PIK3R1; CHUK; PDPK1; PPP2R5C; CTNNB1; MAP2K1; NFKB1; PAK3; |
| | ITGB3; CCND1; GSK3A; FRAP1; SFN; ITGA2; TTK; CSNK1A1; BRAF; |
| | GSK3B; AKT3; FOXO1; SGK; HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Signaling | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Signaling | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| CAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkarla; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

In an embodiment, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiments or the cell of any one of the above embodiments, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In an embodiment, the composition, system(s) or component(s) thereof can be used to diagnose, prognose, treat, and/or prevent an infectious disease caused by a microorganism, such as bacteria, virus, fungi, parasites, or combinations thereof.

In an embodiment, the system(s) or component(s) thereof can be capable of targeting specific microorganism within a mixed population. Exemplary methods of such techniques are described, e.g., in Gomaa A A, Klumpe H E, Luo M L, Selle K, Barrangou R, Beisel C L. 2014. Programmable removal of bacterial strains by use of genome-targeting composition, systems, mBio 5: e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In an embodiment, the composition, system(s), and/or components thereof can be capable of targeting pathogenic and/or drug-resistant microorganisms, such as bacteria, virus, parasites, and fungi. In an embodiment, the composition, system(s), and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In an embodiment, the pathogenic bacteria that can be targeted and/or modified by the composition, system, (s) and/or component(s) thereof disclosed herein include, but are not limited to, those of the genus Actinomyces (e.g., A. israelii), Bacillus (e.g., B. anthracis, B. cereus), Bactereoides (e.g., B. fragilis), Bartonella (B. henselae, B. quintana), Bordetella (B. pertussis), Borrelia (e.g., B. burgdorferi, B. garinii, B. afzelii, and B. recurreentis), Brucella (e.g., B. abortus, B. canis, B. melitensis, and B. suis), Campylobacter (e.g., C. jejuni), Chlamydia (e.g., C. pneumoniae and C. trachomatis), Chlamydophila (e.g., C. psittaci), Clostridium (e.g., C. botulinum, C. difficile, C. perfringens. C. tetani), Corynebacterium (e.g., C. diptheriae), Enterococcus (e.g., E. Faecalis, E. faecium), Ehrlichia (E. canis and E. chaffeensis) Escherichia (e.g., E. coli), Francisella (e.g., F. tularensis), Haemophilus (e.g., H. influenzae), Helicobacter (H. pylori), Klebsiella (e.g., K. pneumoniae), Legionella (e.g., L. pneumophila), Leptospira (e.g., L. interrogans, L. santarosai, L. weilii, L. noguchii), Listeria (e.g., L. monocytogeenes), Mycobacterium (e.g., M. leprae, M. tuberculosis, M. ulcerans), Mycoplasma (M. pneumoniae), Neisseria (N. gonorrhoeae and N. meningitidis), Nocardia (e.g., N. asteroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streptococcus (S. agalactiaee, S. pneumoniae, S. pyogenes), Treponema (T. pallidum), Ureaplasma (e.g., U. urealyticum), Vibrio (e.g., V. cholerae), Yersinia (e.g., Y. pestis, Y. enterocolitica, and Y. pseudotuberculosis).

In one embodiment, the pathogenic virus that can be targeted and/or modified by the composition, system(s) and/or component(s) thereof disclosed herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In one embodiment, the pathogenic virus can be from the family Adenoviridae (e.g., Adenovirus), Herpesviridae (e.g., Herpes simplex, type 1, Herpes simplex, type 2, Varicella-zoster virus, Epstein-Barr virus, Human cytomegalovirus, Human herpesvirus, type 8), Papillomaviridae (e.g., Human papillomavirus), Polyomaviridae (e.g., BK virus, JC virus), Poxviridae (e.g., smallpox), Hepadnaviridae (e.g., Hepatitis B), Parvoviridae (e.g., Parvovirus B19), Astroviridae (e.g., Human astrovirus), Caliciviridae (e.g., Norwalk virus), Picornaviridae (e.g., coxsackievirus, hepatitis A virus, poliovirus, rhinovirus), Coronaviridae (e.g., Severe acute respiratory syndrome-related coronavirus, strains: Severe acute respiratory syndrome virus, Severe acute respiratory syndrome coronavirus 2 (COVID-19 and variants)), Flaviviridae (e.g., Hepatitis C virus, yellow fever virus, dengue virus, West Nile virus, TBE virus), Togaviridae (e.g., Rubella virus), Hepeviridae (e.g., Hepatitis E virus), Retroviridae (Human immunodeficiency virus (HIV)), Orthomyxoviridae (e.g., Influenza virus), Arenaviridae (e.g., Lassa virus), Bunyaviridae (e.g., Crimean-Congo hemorrhagic fever virus, Hantaan virus), Filoviridae (e.g., Ebola virus and Marburg virus), Paramyxoviridae (e.g., Measles virus, Mumps virus, Parainfluenza virus, Respiratory syncytial virus), Rhabdoviridae (Rabies virus), Hepatitis D virus, Reoviridae (e.g., Rotavirus, Orbivirus, Coltivirus, Banna virus). In one embodiment, the pathogenic fungi that can be targeted and/or modified by the composition, system(s), and/or component(s) thereof disclosed herein include, but are not limited to, those of the genus Candida (e.g, C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum).

In one embodiment, the pathogenic parasites that can be targeted and/or modified by the composition, system(s) and/or component(s) thereof disclosed herein include, but are not limited to, protozoa, helminths, and ectoparasites. In one embodiment, the pathogenic protozoa that can be targeted and/or modified by the composition, system(s) and/or component(s) thereof disclosed herein include, but are not limited to, those from the groups Sarcodina (e.g., amoeba such as Entamoeba), Mastigophora (e.g., flagellates such as Giardia and Leishmania), Cilophora (e.g., ciliates such as Balantidum), and sporozoa (e.g., plasmodium and cryptosporidium). In one embodiment, the pathogenic helminths that can be targeted and/or modified by the composition, system(s) and/or component(s) thereof disclosed herein include, but are not limited to, flatworms (platyhelminths), thorny-headed worms (acanthocephalans), and roundworms (nematodes). In one embodiment, the pathogenic ectoparasites that can be targeted and/or modified by the composition, system(s) and/or component(s) thereof disclosed herein include, but are not limited to, ticks, fleas, lice, and mites. In one embodiment, the pathogenic parasite that can be targeted and/or modified by the composition, system(s), and/or component(s) thereof disclosed herein include, but are not limited to, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium Seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g., Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g., Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g., Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g. Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus). In an embodiment the gene targets can be any of those as set forth in Table 1 of Strich and Chertow. 2019. J. Clin. Microbio. 57:4 e01307-18, which is incorporated herein as if expressed in its entirety herein.

In an embodiment, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In an embodiment, delivery of the composition, system, occurs in vivo (i.e., in the subject being treated). In an embodiment occurs by an intermediary, such as microorganism or phage that is non-pathogenic to the subject but is capable of transferring polynucleotides and/or infecting the pathogenic microorganism. In an embodiment, the intermediary microorganism can be an engineered bacteria, virus, or phage that contains the composition, system(s) and/or component(s) thereof and/or CRISPR-Cas vectors and/or vector systems. The method can include administering an intermediary microorganism containing the composition, system(s) and/or component(s) thereof and/or CRISPR-Cas vectors and/or vector systems to the subject to be treated. The intermediary microorganism can then produce the CRISPR-system and/or component thereof or transfer a composition, system, polynucleotide to the pathogenic organism. In embodiments, where the CRISPR-system and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In an embodiment, where the pathogenic microorganism inserts its genetic material into the host cell's genome (e.g., a virus), the composition, system, can be designed such that it modifies the host cell's genome such that the viral DNA or cDNA cannot be replicated by the host cell's machinery into a functional virus. In an embodiment, where the pathogenic microorganism inserts its genetic material into the host cell's genome (e.g., a virus), the composition, system can be designed such that it modifies the host cell's genome such that the viral DNA or cDNA is deleted from the host cell's genome. It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In an embodiment, mtDNA mutations can be modified using a composition, system, described herein. In an embodiment, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In an embodiment, the mtDNA of a subject can be modified in vivo or ex vivo. In an embodiment, where the mtDNA is modified ex vivo, after modification the cells containing the modified mitochondria can be administered back to the subject. In an embodiment, the composition, system, or component thereof can be capable of correcting an mtDNA mutation, or a combination thereof.

In an embodiment, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-(SEQ ID NO: 84) tandem repeats at positions 305-314 and/or 956-965, deletion at positions from 8,469-13,447, 4,308-14,874, and/or 4,398-14,822, 96lins/delC, the mitochondrial common deletion (e.g., mtDNA 4,977 bp deletion), and combinations thereof.

In an embodiment, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In an embodiment, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In an embodiment, the modification can alter a function of the mitochondria such that the mitochondria functions normally or at least is/are less dysfunctional as compared to unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals; thus, detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In an embodiment, the microbe population of a microbiome in a subject can be modified using a composition, system, and/or component thereof described herein. In an embodiment, the composition, system, and/or component thereof can be used to identify and select one or more cell types in the microbiome and remove them from the microbiome population. Exemplary methods of selecting cells using a composition, system, and/or component thereof are described elsewhere herein. In this way, the make-up or microorganism profile of the microbiome can be altered. In an embodiment, the alteration causes a change from a diseased microbiome composition to a healthy microbiome composition. In this way, the ratio of one type or species of microorganism to another can be modified, such as going from a diseased ratio to a healthy ratio. In an embodiment, the cells selected are pathogenic microorganisms.

In an embodiment, the compositions and systems described herein can be used to modify a polynucleotide in a microorganism of a microbiome in a subject. In an embodiment, the microorganism is a pathogenic microorganism. In an embodiment, the microorganism is a commensal and non-pathogenic microorganism. Methods of modifying polynucleotides in a cell in the subject are described elsewhere herein and can be applied to these embodiments.

### Models of Diseases and Conditions

In an embodiment, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiments.

In one aspect, the invention provides a method of generating a model eukaryotic cell that can include one or more a mutated disease genes and/or infectious microorganisms. In an embodiment, a disease gene is any gene associated an increase in the risk of having or developing a disease. In an embodiment, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a guide sequence optionally linked to a tracr mate sequence, a tracr sequence, one or more Cas effectors, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more CRISPR-Cas effectors complexed with (1) one or more guide sequences that is/are hybridized to the target sequence(s) within the target polynucleotide(s), and optionally (2) the tracr mate sequence(s) that is/are hybridized to the tracr sequence(s), thereby generating a model eukaryotic cell comprising one or more mutated disease gene(s). Thus, in an embodiment the composition and system, contains nucleic acid molecules for and drives expression of one or more of: a Cas effector, a guide sequence linked to a tracr mate sequence, and a tracr sequence and/or a Homologous Recombination template and/or a stabilizing ligand if the Cas effector has a destabilization domain. In an embodiment, said cleavage comprises cleaving one or two strands at the location of the target sequence by the Cas effector(s). In an embodiment, nicking comprises nicking one or two strands at the location of the target sequence by the Cas effector(s). In an embodiment, said cleavage or nicking results in modified transcription of a target polynucleotide. In an embodiment, modification results in decreased transcription of the target polynucleotide. In an embodiment, the method further comprises repairing said cleaved or nicked target polynucleotide by homologous recombination with a recombination template polynucleotide, wherein said repair results in a mutation comprising an insertion, deletion, or substitution of one or more nucleotides of said target polynucleotide. In an embodiment, said mutation results in one or more amino acid changes in a protein expression from a gene comprising the target sequence.

The disease modeled can be any disease with a genetic or epigenetic component. In an embodiment, the disease modeled can be any as discussed elsewhere herein, including but not limited to any as set forth in Tables 8 and 9 herein.

### In Situ Disease Detection

The compositions, systems, and/or components thereof can be used for diagnostic methods of detection such as in CASFISH (see Deng et al. 2015. PNAS USA 112 (38): 11870-11875), CRISPR-Live FISH (see e.g., Wang et al. 2020. Science; 365 (6459): 1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g., Deng et al. 2015. PNAS USA 112 (38): 11870-11875; Knight et al., Science, 350 (2015), pp. 823-826), RNA-aptamer based methods (e.g. Ma et al., ll Biol, 214 (2016), pp. 529-537), molecular beacon-based methods (e.g. Zhao et al. Bioials, 100 (2016), pp. 172-183; Wu et al. Nucleic Acids Res (2018)), Quantum Dot-based systems (e.g. Ma et al. Anal Chem, 2017), pp. 12896-12901), multiplexed methods (e.g. Ma et al., Proc Natl AcadU S A, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), ç, and other in situ CRISPR-hybridization based methods (e.g. Chen et al. Cell, 155 (2013), 479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In an embodiment, the composition, system, or component thereof can be used in a detection method, such as an in situ detection method described herein. In an embodiment, the composition, system, or component thereof can include a catalytically inactivate Cas effector described herein and use this system in detection methods such as fluorescence in situ hybridization (FISH) or any other described herein. In an embodiment, the inactivated Cas effector, which lacks the ability to produce DNA double-strand breaks may be fused with a marker, such as fluorescent protein, such as the enhanced green fluorescent protein (eEGFP) and co-expressed with small guide molecules, e.g., guide RNAs, to target pericentric, centric and telomeric repeats in vivo. The dCas effector or system thereof can be used to visualize both repetitive sequences and individual genes in the human genome. Such new applications of labelled dCas effector and compositions, systems, thereof can be important in imaging cells and studying the functional nuclear architecture, especially in cases with a small nucleus volume or complex 3-D structures.

### Cell Selection

In an embodiment, the compositions, systems, and/or components thereof described herein can be used in a method to screen and/or select cells. In an embodiment, composition, system-based screening/selection method can be used to identify diseased cells in a cell population. In an embodiment, selection of the cells results in a modification in the cells such that the selected cells die. In this way, diseased cells can be identified, and removed from the healthy cell population. In an embodiment, the diseased cells can be a cancer cell, pre-cancerous cell, a virus or other pathogenic organism infected cells, or otherwise abnormal cell. In an embodiment, the modification can impart another detectable change in the cells to be selected (e.g., a functional change and/or genomic barcode) that facilitates selection of the desired cells. In an embodiment a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In an embodiment, a method of selecting one or more cell(s) containing a polynucleotide modification can include: introducing one or more composition, system(s), and/or components thereof, and/or vectors or vector systems into the cell(s), wherein the composition, system(s), and/or components thereof, and/or vectors or vector systems contains and/or is capable of expressing one or more of: a Cas effector, a guide sequence optionally linked to a tracr mate sequence, a tracr sequence, and an recombination template; wherein, for example that which is being expressed is within and expressed in vivo by the composition, system, vector or vector system and/or the recombination template comprises the one or more mutations that abolish Cas effector cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the Cas effector complexed with (1) the guide sequence that is hybridized to the target sequence within the target polynucleotide, and (2) the tracr mate sequence that is hybridized to the tracr sequence, wherein binding of the complex to the target polynucleotide induces cell death or imparts some other detectable change to the cell, thereby allowing one or more cell(s) in which one or more mutations have been introduced to be selected. In an embodiment, the cell to be selected may be a eukaryotic cell. In an embodiment, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop CRISPR-Cas-based and non-CRISPR-Cas-based biologically active agents, such as small molecule therapeutics. Thus, described herein are methods for developing a biologically active agent that modulates a cell function and/or signaling event associated with a disease and/or disease gene. In an embodiment, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In an embodiment, the diseased cell is a model cell described elsewhere herein. In an embodiment, the diseased cell is a diseased cell isolated from a subject in need of treatment. In an embodiment, the test compound is a small molecule agent. In an embodiment, test compound is a small molecule agent. In an embodiment, the test compound is a biologic molecule agent.

In an embodiment, the method involves developing a therapeutic based on the composition, system, described herein. In an embodiment, the therapeutic comprises a Cas effector and/or a guide molecule, e.g., guide RNA, capable of hybridizing to a target sequence of interest. In an embodiment, the therapeutic is a vector or vector system that can contain a) a first regulatory element operably linked to a nucleotide sequence encoding the Cas effector protein(s); and b) a second regulatory element operably linked to one or more nucleotide sequences encoding one or more nucleic acid molecules comprising a guide molecule, e.g., guide RNA, comprising a guide sequence, a direct repeat sequence; wherein components (a) and (b) are located on same or different vectors. In an embodiment, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In an embodiment, the complex can include the Cas effector protein(s) as described herein, guide molecule, e.g., guide RNA, comprising the guide sequence, and a direct repeat sequence. In any such compositions, the delivery system can be a yeast system, a lipofection system, a microinjection system, a biolistic system, virosomes, liposomes, immunoliposomes, polycations, lipid: nucleic acid conjugates or artificial virions, or any other system as described herein. In an embodiment, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, and optionally validating one or more of the (sub) selected target sites for an individual subject, optionally designing one or more gRNA recognizing one or more of said (sub) selected target sites.

In an embodiment, the method for developing or designing a gRNA for use in a composition, system, optionally a composition, system, based therapy or therapeutic, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more gRNA recognizing one or more of said (sub) selected target sites.

In an embodiment, the method for developing or designing a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more gRNA recognizing one or more of said (sub) selected target sites.

In an embodiment the method for developing or designing a gRNA for use in a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more gRNA recognizing one or more of said (sub) selected target sites.

In an embodiment, the method for developing or designing a composition, system, such as a composition, system, based therapy or therapeutic, optionally in a population; or for developing or designing a gRNA for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e., platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of CRISP-Cas systems prepared is based (at least in part) on the size of a target population.

In an embodiment, off-target candidates/off-targets, PFS, PAM restrictiveness, target cleavage efficiency, or effector protein specificity is identified or determined using a sequencing-based double-strand break (DSB) detection assay, such as described herein elsewhere. In an embodiment, off-target candidates/off-targets are identified or determined using a sequencing-based double-strand break (DSB) detection assay, such as described herein elsewhere. In an embodiment, off-targets, or off target candidates have at least 1, preferably 1-3, mismatches or (distal) PFS or PAM mismatches, such as 1 or more, such as 1, 2, 3, or more (distal) PFS or PAM mismatches. In an embodiment, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the guide sequence of the gRNA is 100% complementary to the target site, i.e., does not comprise any mismatch with the target site. It will be further understood that “recognition” of an (off-) target site by a gRNA presupposes composition, system, functionality, means the (off-) target site is only recognized by a gRNA if binding of the gRNA to the (off-) target site leads to composition, system, activity (such as induction of single or double strand DNA cleavage, transcriptional modulation, etc.).

In an embodiment, the target sites having minimal sequence variation across a population are characterized by absence of sequence variation in at least 99%, preferably at least 99.9%, more preferably at least 99.99% of the population. In an embodiment, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In an embodiment, said population comprises at least 1000 individuals, such as at least 5000 individuals, such as at least 10000 individuals, such as at least 50000 individuals.

In an embodiment, the off-target sites are characterized by at least one mismatch between the off-target site and the gRNA. In an embodiment, the off-target sites are characterized by at most five, preferably at most four, more preferably at most three mismatches between the off-target site and the gRNA. In an embodiment, the off-target sites are characterized by at least one mismatch between the off-target site and the gRNA and by at most five, preferably at most four, more preferably at most three mismatches between the off-target site and the gRNA.

In an embodiment, said minimal number of off-target sites across said population is determined for high-frequency haplotypes in said population. In an embodiment, said minimal number of off-target sites across said population is determined for high-frequency haplotypes of the off-target site locus in said population. In an embodiment, said minimal number of off-target sites across said population is determined for high-frequency haplotypes of the target site locus in said population. In an embodiment, the high-frequency haplotypes are characterized by occurrence in at least 0.1% of the population.

In an embodiment, the number of (sub) selected target sites needed to treat a population is estimated based on based low frequency sequence variation, such as low frequency sequence variation captured in large scale sequencing datasets. In an embodiment, the number of (sub) selected target sites needed to treat a population of a given size is estimated.

In an embodiment, the method further comprises obtaining genome sequencing data of a subject to be treated; and treating the subject with a composition, system, selected from the set of compositions, systems, wherein the composition, system, selected is based (at least in part) on the genome sequencing data of the individual. In an embodiment, the ((sub) selected) target is validated by genome sequencing, preferably whole genome sequencing.

In an embodiment, target sequences or loci as described herein are (further) selected based on optimization of one or more parameters, such as PFS or PAM type (natural or modified), PFS or PAM nucleotide content, PFS or PAM length, target sequence length, PFS or PAM restrictiveness, target cleavage efficiency, and target sequence position within a gene, a locus or other genomic region. Methods of optimization are discussed in greater detail elsewhere herein.

In an embodiment, target sequences or loci as described herein are (further) selected based on optimization of one or more of target loci location, target length, target specificity, and PFS or PAM characteristics. As used herein, PFS or PAM characteristics may comprise for instance PFS or PAM sequence, PFS or PAM length, and/or PFS or PAM GC contents. In an embodiment, optimizing PFS or PAM characteristics comprises optimizing nucleotide content of a PFS or PAM. In an embodiment, optimizing nucleotide content of PFS or PAM is selecting a PFS or PAM with a motif that maximizes abundance in the one or more target loci, minimizes mutation frequency, or both. Minimizing mutation frequency can for instance be achieved by selecting PFS or PAM sequences devoid of or having low or minimal CpG.

In an embodiment, the effector protein for each composition and system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

The activity of the composition and/or system, such as CRISPR-Cas system-based therapy or therapeutics may involve target disruption, such as target mutation, such as leading to gene knockout. The activity of the composition and/or system, such as CRISPR-Cas system-based therapy or therapeutics may involve replacement of particular target sites, such as leading to target correction. CRISPR-Cas system-based therapy or therapeutics may involve removal of particular target sites, such as leading to target deletion. The activity of the composition and/or system, such as CRISPR-Cas system-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve CRISPR effector mutation (such as for instance generation of a catalytically inactive CRISPR effector) and/or functionalization (such as for instance fusion of the CRISPR effector with a heterologous functional domain, such as a transcriptional activator or repressor), as described herein elsewhere.

Accordingly, in an embodiment, the invention relates to a method as described herein, comprising selection of one or more (therapeutic) target, selecting one or more functionality of the composition and/or system, and optimization of selected parameters or variables associated with the CRISPR-Cas system and/or its functionality. In a related aspect, the invention relates to a method as described herein, comprising (a) selecting one or more (therapeutic) target loci, (b) selecting one or more CRISPR-Cas system functionalities, (c) optionally selecting one or more modes of delivery, and preparing, developing, or designing a CRISPR-Cas system selected based on steps (a)-(c).

In an embodiment, the functionality of the composition and/or system comprises genomic mutation. In an embodiment, the functionality of the composition and/or system comprises single genomic mutation. In an embodiment, the functionality of the composition and/or system functionality comprises multiple genomic mutation. In an embodiment, the functionality of the composition and/or system comprises gene knockout. In an embodiment, the functionality of the composition and/or system comprises single gene knockout. In an embodiment, the functionality of the composition and/or system comprises multiple gene knockout. In an embodiment, the functionality of the composition and/or system comprises gene correction. In an embodiment, the functionality of the composition and/or system comprises single gene correction. In an embodiment, the functionality of the composition and/or system comprises multiple gene correction. In an embodiment, the functionality of the composition and/or system comprises genomic region correction. In an embodiment, the functionality of the composition and/or system comprises single genomic region correction. In an embodiment, the functionality of the composition and/or system comprises multiple genomic region correction. In an embodiment, the functionality of the composition and/or system comprises gene deletion. In an embodiment, the functionality of the composition and/or system comprises single gene deletion. In an embodiment, the functionality of the composition and/or system comprises multiple gene deletion. In an embodiment, the functionality of the composition and/or system comprises genomic region deletion. In an embodiment, the functionality of the composition and/or system comprises single genomic region deletion. In an embodiment, the functionality of the composition and/or system comprises multiple genomic region deletion. In an embodiment, the functionality of the composition and/or system comprises modulation of gene or genomic region functionality. In an embodiment, the functionality of the composition and/or system comprises modulation of single gene or genomic region functionality. In an embodiment, the functionality of the composition and/or system comprises modulation of multiple gene or genomic region functionality. In an embodiment, the functionality of the composition and/or system comprises gene or genomic region functionality, such as gene or genomic region activity. In an embodiment, the functionality of the composition and/or system comprises single gene or genomic region functionality, such as gene or genomic region activity. In an embodiment, the functionality of the composition and/or system comprises multiple gene or genomic region functionality, such as gene or genomic region activity. In an embodiment, the functionality of the composition and/or system comprises modulation gene activity or accessibility optionally leading to transcriptional and/or epigenetic gene or genomic region activation or gene or genomic region silencing. In an embodiment, the functionality of the composition and/or system comprises modulation single gene activity or accessibility optionally leading to transcriptional and/or epigenetic gene or genomic region activation or gene or genomic region silencing. In an embodiment, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as CRISPR-Cas system-based therapy or therapeutic, specificity, efficacy, and/or safety. In an embodiment, one or more of the following parameters or variables are taken into account, are selected, or are optimized in the methods of the invention as described herein: Cas protein allosteric interactions, Cas protein functional domains and functional domain interactions, CRISPR effector specificity, gRNA specificity, CRISPR-Cas complex specificity, PFS or PAM restrictiveness, PFS or PAM type (natural or modified), PFS or PAM nucleotide content, PFS or PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR effector spatiotemporal expression, gRNA spatiotemporal expression, CRISPR-Cas comples spatiotemporal expression.

By means of example, and without limitation, parameter or variable optimization may be achieved as follows. CRISPR effector specificity may be optimized by selecting the most specific CRISPR effector. This may be achieved for instance by selecting the most specific CRISPR effector ortholog or by specific CRISPR effector mutations which increase specificity. gRNA specificity may be optimized by selecting the most specific gRNA. This can be achieved for instance by selecting gRNA having low homology, i.e., at least one or preferably more, such as at least 2, or preferably at least 3, mismatches to off-target sites. CRISPR-Cas complex specificity may be optimized by increasing CRISPR effector specificity and/or gRNA specificity as above. PFS or PAM restrictiveness may be optimized by selecting a CRISPR effector having to most restrictive PFS or PAM recognition. This can be achieved for instance by selecting a CRISPR effector ortholog having more restrictive PFS or PAM recognition or by specific CRISPR effector mutations which increase or alter PFS or PAM restrictiveness. PFS or PAM type may be optimized for instance by selecting the appropriate CRISPR effector, such as the appropriate CRISPR effector recognizing a desired PFS or PAM type. The CRISPR effector or PFS or PAM type may be naturally occurring or may for instance be optimized based on CRISPR effector mutants having an altered PFS or PAM recognition, or PFS or PAM recognition repertoire. PFS or PAM nucleotide content may for instance be optimized by selecting the appropriate CRISPR effector, such as the appropriate CRISPR effector recognizing a desired PFS or PAM nucleotide content. The CRISPR effector or PFS or PAM type may be naturally occurring or may for instance be optimized based on CRISPR effector mutants having an altered PFS or PAM recognition, or PAM recognition repertoire. PFS or PAM length may for instance be optimized by selecting the appropriate CRISPR effector, such as the appropriate CRISPR effector recognizing a desired PFS or PAM nucleotide length. The CRISPR effector or PFS or PAM type may be naturally occurring or may for instance be optimized based on CRISPR effector mutants having an altered PFS or PAM recognition, or PFS or PAM recognition repertoire.

Target length or target sequence length may be optimized, for instance, by selecting the appropriate CRISPR effector, such as the appropriate CRISPR effector recognizing a desired target or target sequence nucleotide length. Alternatively, or in addition, the target (sequence) length may be optimized by providing a target having a length deviating from the target (sequence) length typically associated with the CRISPR effector, such as the naturally occurring CRISPR effector. The CRISPR effector or target (sequence) length may be naturally occurring or may for instance be optimized based on CRISPR effector mutants having an altered target (sequence) length recognition, or target (sequence) length recognition repertoire. For instance, increasing or decreasing target (sequence) length may influence target recognition and/or off-target recognition. CRISPR effector activity may be optimized by selecting the most active CRISPR effector. This may be achieved for instance by selecting the most active CRISPR effector ortholog or by specific CRISPR effector mutations which increase activity. The ability of the CRISPR effector protein to access regions of high chromatin accessibility, may be optimized by selecting the appropriate CRISPR effector or mutant thereof, and can consider the size of the CRISPR effector, charge, or other dimensional variables etc. The degree of uniform CRISPR effector activity may be optimized by selecting the appropriate CRISPR effector or mutant thereof, and can consider CRISPR effector specificity and/or activity, PFS or PAM specificity, target length, mismatch tolerance, epigenetic tolerance, CRISPR effector and/or gRNA stability and/or half-life, CRISPR effector and/or gRNA immunogenicity and/or toxicity, etc. gRNA activity may be optimized by selecting the most active gRNA. In an embodiment, this can be achieved by increasing gRNA stability through RNA modification. CRISPR-Cas complex activity may be optimized by increasing CRISPR effector activity and/or gRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e., having low variability, such as polymorphisms, within a population).

In an embodiment, optimizing target (sequence) length comprises selecting a target sequence within one or more target loci between 5 and 25 nucleotides. In an embodiment, a target sequence is 20 nucleotides.

In an embodiment, optimizing target specificity comprises selecting targets loci that minimize off-target candidates.

In an embodiment, the target site may be selected by minimization of off-target effects (e.g., off-targets qualified as having 1-5, 1-4, or preferably 1-3 mismatches compared to target and/or having one or more PFS or PAM mismatches, such as distal PFS or PAM mismatches), preferably also considering variability within a population. CRISPR effector stability may be optimized by selecting CRISPR effector having appropriate half-life, such as preferably a short half-life while still capable of maintaining sufficient activity. In an embodiment, this can be achieved by selecting an appropriate CRISPR effector ortholog having a specific half-life or by specific CRISPR effector mutations or modifications which affect half-life or stability, such as inclusion (e.g. fusion) of stabilizing or destabilizing domains or sequences. CRISPR effector mRNA stability may be optimized by increasing or decreasing CRISPR effector mRNA stability. In an embodiment, this can be achieved by increasing or decreasing CRISPR effector mRNA stability through mRNA modification. gRNA stability may be optimized by increasing or decreasing gRNA stability. In an embodiment, this can be achieved by increasing or decreasing gRNA stability through RNA modification. CRISPR-Cas complex stability may be optimized by increasing or decreasing CRISPR effector stability and/or gRNA stability as above. CRISPR effector protein or mRNA immunogenicity or toxicity may be optimized by decreasing CRISPR effector protein or mRNA immunogenicity or toxicity. In an embodiment, this can be achieved by mRNA or protein modifications. Similarly, in case of DNA based expression systems, DNA immunogenicity or toxicity may be decreased. gRNA immunogenicity or toxicity may be optimized by decreasing gRNA immunogenicity or toxicity. In an embodiment, this can be achieved by gRNA modifications. Similarly, in case of DNA based expression systems, DNA immunogenicity or toxicity may be decreased. CRISPR-Cas complex immunogenicity or toxicity may be optimized by decreasing CRISPR effector immunogenicity or toxicity and/or gRNA immunogenicity or toxicity as above, or by selecting the least immunogenic or toxic CRISPR effector/gRNA combination. Similarly, in the case of DNA based expression systems, DNA immunogenicity or toxicity may be decreased. CRISPR effector protein or mRNA dose or titer may be optimized by selecting dosage or titer to minimize toxicity and/or maximize specificity and/or efficacy. gRNA dose or titer may be optimized by selecting dosage or titer to minimize toxicity and/or maximize specificity and/or efficacy. CRISPR-Cas complex dose or titer may be optimized by selecting dosage or titer to minimize toxicity and/or maximize specificity and/or efficacy. CRISPR effector protein size may be optimized by selecting minimal protein size to increase efficiency of delivery, in particular for virus mediated delivery. CRISPR effector, gRNA, or CRISPR-Cas complex expression level may be optimized by limiting (or extending) the duration of expression and/or limiting (or increasing) expression level. This may be achieved for instance by using self-inactivating compositions, systems, such as including a self-targeting (e.g. CRISPR effector targeting) gRNA, by using viral vectors having limited expression duration, by using appropriate promoters for low (or high) expression levels, by combining different delivery methods for individual CRISP-Cas system components, such as virus mediated delivery of CRISPR-effector encoding nucleic acid combined with non-virus mediated delivery of gRNA, or virus mediated delivery of gRNA combined with non-virus mediated delivery of CRISPR effector protein or mRNA. CRISPR effector, gRNA, or CRISPR-Cas complex spatiotemporal expression may be optimized by appropriate choice of conditional and/or inducible expression systems, including controllable CRISPR effector activity optionally a destabilized CRISPR effector and/or a split CRISPR effector, and/or cell- or tissue-specific expression systems.

In an embodiment, the invention relates to a method as described herein, comprising selection of one or more (therapeutic) target, selecting the functionality of the composition and/or system, selecting CRISPR-Cas system mode of delivery, selecting CRISPR-Cas system delivery vehicle or expression system, and optimization of selected parameters or variables associated with the CRISPR-Cas system and/or its functionality, optionally wherein the parameters or variables are one or more selected from CRISPR effector specificity, gRNA specificity, CRISPR-Cas complex specificity, PFS or PAM restrictiveness, PFS or PAM type (natural or modified), PFS or PAM nucleotide content, PFS or PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an embodiment, the invention relates to a method as described herein, comprising selecting one or more (therapeutic) target, selecting one or more the functionality of the composition and/or system, selecting one or more CRISPR-Cas system mode of delivery, selecting one or more delivery vehicle or expression system, and optimization of selected parameters or variables associated with the CRISPR-Cas system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an embodiment, the invention relates to a method as described herein, comprising optionally selecting one or more (therapeutic) target, optionally selecting one or more the functionality of the composition and/or system, optionally selecting one or more mode of delivery, optionally selecting one or more delivery vehicle or expression system, and optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an embodiment, the invention relates to a method as described herein, comprising optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

It will be understood that the parameters or variables to be optimized as well as the nature of optimization may depend on the (therapeutic) target, the functionality of the composition and/or system, the system mode of delivery, and/or the CRISPR-Cas system delivery vehicle or expression system.

In an embodiment, the invention relates to a method as described herein, comprising optimization of gRNA specificity at the population level. Preferably, said optimization of gRNA specificity comprises minimizing gRNA target site sequence variation across a population and/or minimizing gRNA off-target incidence across a population.

In an embodiment, optimization can result in selection of a CRISPR-Cas effector that is naturally occurring or is modified. In an embodiment, optimization can result in selection of a CRISPR-Cas effector that has nuclease, nickase, deaminase, transposase, and/or has one or more effector functionalities deactivated or eliminated. In an embodiment, optimizing a PFS or PAM specificity can include selecting a CRISPR-Cas effector with a modified PFS or PAM specificity. In an embodiment, optimizing can include selecting a CRISPR-Cas effector having a minimal size. In an embodiment, optimizing effector protein stability comprises selecting an effector protein having a short half-life while maintaining sufficient activity, such as by selecting an appropriate CRISPR effector ortholog having a specific half-life or stability. In an embodiment, optimizing immunogenicity or toxicity comprises minimizing effector protein immunogenicity or toxicity by protein modifications. In an embodiment, optimizing functional specific comprises selecting a protein effector with reduced tolerance of mismatches and/or bulges between the guide molecule, e.g., guide RNA, and one or more target loci.

In an embodiment, optimizing efficacy comprises optimizing overall efficiency, epigenetic tolerance, or both. In an embodiment, maximizing overall efficiency comprises selecting an effector protein with uniform enzyme activity across target loci with varying chromatin complexity, selecting an effector protein with enzyme activity limited to areas of open chromatin accessibility. In an embodiment, chromatin accessibility is measured using one or more of ATAC-seq, or a DNA-proximity ligation assay. In an embodiment, optimizing epigenetic tolerance comprises optimizing methylation tolerance, epigenetic mark competition, or both. In an embodiment, optimizing methylation tolerance comprises selecting an effector protein that modify methylated DNA. In an embodiment, optimizing epigenetic tolerance comprises selecting an effector protein unable to modify silenced regions of a chromosome, selecting an effector protein able to modify silenced regions of a chromosome, or selecting target loci not enriched for epigenetic markers.

In an embodiment, selecting an optimized guide molecule, e.g., guide RNA, comprises optimizing gRNA stability, gRNA immunogenicity, or both, or other gRNA associated parameters or variables as described herein elsewhere.

In an embodiment, optimizing gRNA stability and/or gRNA immunogenicity comprises RNA modification, or other gRNA associated parameters or variables as described herein elsewhere. In an embodiment, the modification comprises removing 1-3 nucleotides form the 3′ end of a target complementarity region of the gRNA. In an embodiment, modification comprises an extended gRNA and/or trans RNA/DNA element that create stable structures in the gRNA that compete with gRNA base pairing at a target of off-target loci or extended complimentary nucleotides between the gRNA and target sequence, or both.

In an embodiment, the mode of delivery comprises delivering gRNA and/or CRISPR effector protein, delivering gRNA and/or CRISPR effector mRNA, or delivery gRNA and/or CRISPR effector as a DNA based expression system. In an embodiment, the mode of delivery further comprises selecting a delivery vehicle and/or expression systems from the group consisting of liposomes, lipid particles, nanoparticles, biolistics, or viral-based expression/delivery systems. In an embodiment, expression is spatiotemporal expression is optimized by choice of conditional and/or inducible expression systems, including controllable CRISPR effector activity optionally a destabilized CRISPR effector and/or a split CRISPR effector, and/or cell- or tissue-specific expression system.

The methods as described herein may further involve selection of the mode of delivery. In an embodiment, gRNA (and tracr, if and where needed, optionally provided as a sgRNA) and/or CRISPR effector protein are or are to be delivered. In an embodiment, gRNA (and tracr, if and where needed, optionally provided as a sgRNA) and/or CRISPR effector mRNA are or are to be delivered. In an embodiment, gRNA (and tracr, if and where needed, optionally provided as a sgRNA) and/or CRISPR effector provided in a DNA-based expression system are or are to be delivered. In an embodiment, delivery of the individual system components comprises a combination of the above modes of delivery. In an embodiment, delivery comprises delivering gRNA and/or CRISPR effector protein, delivering gRNA and/or CRISPR effector mRNA, or delivering gRNA and/or CRISPR effector as a DNA based expression system.

The methods as described herein may further involve selection of the CRISPR-Cas system delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g., adenoviral, AAV, lentiviral), etc. the skilled person will understand that selection of the mode of delivery, as well as delivery vehicle or expression system may depend on for instance the cell or tissues to be targeted. In an embodiment, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a Cas nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels is needed to gain a clinically relevant response. In an embodiment, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a CRISPR-Cas based therapeutic as described in greater detail elsewhere herein.

CRISPR-Cas-based polynucleotide modification application can include combinations of proteins, small RNA molecules, and/or repair templates, and can make, in an embodiment, delivery of these multiple parts substantially more challenging than, for example, traditional small molecule therapeutics. Two main strategies for delivery of compositions, systems, and components thereof have been developed: ex vivo and in vivo. In an embodiment of ex vivo treatments, diseased cells are removed from a subject, edited and then transplanted back into the patient. In other embodiments, cells from a healthy allogeneic donor are collected, modified using a CRISPR-Cas system or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In an embodiment, such as those where viral vector systems are used to generate viral particles to deliver the CRISPR-Cas system and/or component thereof to a cell, the total cargo size of the CRISPR-Cas system and/or component thereof should be considered as vector systems can have limits on the size of a polynucleotide that can be expressed therefrom and/or packaged into cargo inside of a viral particle. In an embodiment, the tropism of a vector system, such as a viral vector system, should be considered as it can impact the cell type to which the CRISPR-Cas system or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In an embodiment, it can be important to consider the immunogenicity of the system or component thereof. In embodiments where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the CRISPR enzyme in the host species (human or other species).

### Xenotransplantation

The present invention also contemplates use of the CRISPR-Cas system described herein, e.g., Cas effector protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e., xenoantigen genes. Candidate porcine genes for disruption may for example include a (1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA•DNA hybrids. Mclvor EI, Polak U, Napierala M. RNA Biol. 2010 September-October; 7 (5): 551-8). The present effector protein systems may be harnessed to correct these defects of genomic instability.

Several further aspects of the invention relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Immune Orthogonal Orthologs

In an embodiment, when the Cas need to be expressed or administered in a subject, immunogenicity of the Cas may be reduced by sequentially expressing or administering immune orthogonal orthologs of the Cas to the subject. As used herein, the term “immune orthogonal orthologs” refer to orthologous proteins that have similar or substantially the same function or activity, but have no or low cross-reactivity with the immune response generated by one another. In an embodiment, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host's immune system (e.g., by activated CTLs). In an embodiment, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively or additionally, immune overlap among candidates may be assessed by determining B-cell epitopes for the candidate orthologs. In one example, immune orthogonal orthologs may be identified using the method described in Moreno A M et al., BioRxiv, published online Jan. 10, 2018, doi: doi.org/10.1101/245985.

### Patient-Specific Screening Methods

A nucleic acid-targeting system that targets RNA or single stranded DNA can be used to screen patients or patient samples for the presence of particular RNA or single stranded DNA. Methods may comprise detection of one or more viruses in a sample from the patient. Advantageously, rapid detection using one or more CRISPR Cas systems can identify those patients with particular viral infections.

### Detection Systems and Methods of Use Thereof for Detecting a Target Polynucleotide or Target Polypeptide

In one aspect, the embodiments disclosed herein are directed to a nucleic acid detection system comprising a multimeric Cas polypeptide complex (e.g., CRISPR effector protein) as described herein which is expected to be an RNA-guided nuclease having collateral cleavage activity. Certain example embodiments disclosed herein are expected to utilize Cas effector proteins as described herein which are RNA targeting effectors to provide a robust CRISPR-based diagnostic. In an embodiment, CRISPR-based diagnostics comprising CRISPR effector proteins as described herein are expected to have attomolar sensitivity. It is expected that embodiments disclosed herein can detect broth DNA and RNA with comparable levels of sensitivity and can differentiate targets from non-targets based on single base pair differences. Moreover, it is expected that the embodiments disclosed herein can be prepared in freeze-dried format for convenient distribution and point-of-care (POC) applications. Such embodiments are expected to be useful in multiple scenarios in human health including, for example, viral detection, bacterial strain typing, sensitive genotyping, and detection of disease-associated cell free DNA. For ease of reference, the embodiments disclosed herein may also be referred to as a SHERLOCK (Specific High-sensitivity Enzymatic Reporter unLOCKing) system.

In an embodiment, the nucleic acid detection system further comprises one or more guide molecules, e.g., guide RNAs as, described herein designed to bind to corresponding target nucleic acids (e.g., target RNAs or target DNAs) and to form a CRISPR-Cas complex with the CRISPR effector proteins as described herein, a masking construct (e.g., an RNA or DNA masking construct), and optional amplification reagents to amplify target nucleic acid molecules (e.g., RNA or DNA) in a sample. In an embodiment, the nucleic acid detection system comprises amplification reagents. In an embodiment, the nucleic acid detection system does not comprise amplification reagents.

In another aspect, the embodiments disclosed herein are directed to a polypeptide detection system comprising a CRISPR effector protein as described herein which is an RNA-guided nuclease, e.g., an RNase, having collateral cleavage activity, one or more guide molecules, e.g., guide RNAs, as described herein, designed to bind to corresponding trigger nucleic acids (e.g., trigger RNAs or trigger DNAs), a masking construct, and one or more detection aptamers. In an embodiment, the polypeptide detection system further comprises amplification reagents to amplify the aptamer sequences and/or the trigger nucleic acids. In an embodiment, the polypeptide detection system does not further comprise amplification reagents. In an embodiment, the RNA-guided nuclease is an RNase, and the one or more detection aptamers comprise an RNA polymerase promoter binding site or a primer binding site. The one or more detection aptamers specifically bind one or more target polypeptides and are configured such that the RNA polymerase promoter binding site or primer binding site is exposed only upon binding of the detection aptamer to a target polypeptide. Exposure of the RNA polymerase promoter binding site facilitates generation of a trigger RNA using the aptamer sequence as a template. Accordingly, in such embodiments the one or more guide RNAs are configured to bind to corresponding trigger RNAs.

In another aspect, the embodiments disclosed herein are expected to be directed to a diagnostic device comprising a plurality of individual discrete volumes. Each individual discrete volume comprises a CRISPR effector protein as described herein, one or more guide molecules, e.g., guide RNAs, as described herein, designed to bind to a corresponding target molecule and to form a CRISPR-Cas complex with the CRISPR effector protein, and a masking construct. In an embodiment, reagents to amplify the target molecules (e.g., RNA or DNA amplification reagents) may be pre-loaded into the individual discrete volumes or be added to the individual discrete volumes concurrently with or subsequent to addition of a sample to each individual discrete volume. The device may be a microfluidic based device, a wearable device, or device comprising a flexible material substrate on which the individual discrete volumes are defined.

In another aspect, the embodiments disclosed herein are expected to be directed to a method for detecting target nucleic acids in a sample comprising distributing a sample or set of samples into a set of individual discrete volumes, each individual discrete volume comprising a CRISPR effector protein as described herein, one or more guide molecules, e.g., guide RNAs, as described herein, designed to bind a corresponding target molecule and to form a CRISPR-Cas complex with the CRISPR effector protein, and a masking construct. The set of samples are then maintained under conditions sufficient to allow binding of the one or more guide RNAs to one or more target molecules. Binding of the one or more guide RNAs to a target nucleic acid in turn activates the CRISPR effector protein. The expected collateral activity of the activated CRISPR effector protein then deactivates the masking construct, for example, by cleaving the masking construct such that a detectable positive signal is unmasked, released, or generated. Detection of the positive detectable signal in an individual discrete volume indicates the presence of the target molecules.

In yet another aspect, the embodiments disclosed herein are expected to be directed to a method for detecting polypeptides. The method for detecting polypeptides is similar to the method for detecting target nucleic acids described above. However, one or more peptide detection aptamers are also included. The peptide detection aptamers function as described above and facilitate generation of a trigger molecule (e.g., trigger oligonucleotide) upon binding to a target polypeptide. The guide RNAs are designed to recognize the trigger oligonucleotides thereby activating the CRISPR effector protein. Deactivation of the masking construct by the expected collateral activity of the activated multimeric Cas polypeptide complex leads to unmasking, release, or generation of a detectable positive signal.

The CRISPR effector proteins and systems of the invention are expected to be useful for specific detection of target polynucleotides, e.g., target RNAs or DNAs, or polypeptides, in a cell or other sample. In the presence of an RNA target of interest, guide-dependent CRISPR effector protein nuclease activity may be accompanied by non-specific nuclease, e.g., RNAse or DNAse, activity against collateral targets. To take advantage of the collateral RNase (or DNAse) activity, all that is needed is a reporter substrate that can be detectably cleaved.

In one exemplary method, a reporter molecule can comprise RNA (or DNA), tagged with a fluorescent reporter molecule (fluor) on one end and a quencher on the other. In the absence of CRISPR effector protein RNase (or DNAse) activity, the physical proximity of the quencher dampens fluorescence from the fluor to low levels. When CRISPR effector protein target-specific cleavage is activated by the presence of an RNA (or DNA) target-of-interest and suitable guide molecule, e.g., guide RNA, the RNA (or DNA)-containing reporter molecule is non-specifically cleaved and the fluor and quencher are spatially separated. This causes the fluor to emit a detectable signal when excited by light of the appropriate wavelength. In one exemplary assay method, CRISPR-Cas effector, target-of-interest-specific guide molecule, e.g., guide RNA, and reporter molecule are added to a cellular sample. An increase in fluorescence indicates the presence of the RNA (or DNA) target-of-interest. In another exemplary method, a detection array is provided. Each location of the array is provided with CRISPR-Cas effector, reporter molecule, and a target-of-interest-specific guide molecule, e.g., guide RNA. Depending on the assay to be performed, the target-of-interest-specific guide molecules, e.g., guide RNAs, at each location of the array can be the same, different, or a combination thereof. Different target-of-interest-specific guide molecules, e.g., guide RNAs, might be provided, for example when it is desired to test for one or more targets in a single source sample. The same target-of-interest-specific guide molecule, e.g., guide RNA, might be provided at each location, for example when it is desired to test multiple samples for the same target.

### RNA-Based Masking Constructs

As used herein, a “masking construct” refers to a molecule that can be cleaved or otherwise deactivated by an activated CRISPR-Cas system effector protein as described herein (e.g., a multimeric Cas polypeptide complex). The term “masking construct” may also be referred to in the alternative as a “detection construct.” In an embodiment, the masking construct is an RNA-based masking construct. The RNA-based masking construct comprises an RNA element that is cleavable by a CRISPR effector protein. Cleavage of the RNA element releases agents or produces conformational changes that allow a detectable signal to be produced. Example constructs demonstrating how the RNA element may be used to prevent or mask generation of detectable signal are described below and embodiments of the invention comprise variants of the same. Prior to cleavage, or when the masking construct is in an ‘active’ state, the masking construct blocks the generation or detection of a positive detectable signal. It will be understood that in an embodiment a minimal background signal may be produced in the presence of an active RNA masking construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the masking construct. For example, in an embodiment a first signal may be detected when the masking agent is present (i.e., a negative detectable signal), which then converts to a second signal (e.g., the positive detectable signal) upon detection of the target molecules and cleavage or deactivation of the masking agent by the activated CRISPR effector protein.

In an embodiment, the masking construct may suppress generation of a gene product. The gene product may be encoded by a reporter construct that is added to the sample. The masking construct may be an interfering RNA involved in an RNA interference pathway, such as a short hairpin RNA (shRNA) or small interfering RNA (siRNA). The masking construct may also comprise microRNA (miRNA). While present, the masking construct suppresses expression of the gene product. The gene product may be a fluorescent protein or other RNA transcript or proteins that would otherwise be detectable by a labeled probe, aptamer, or antibody but for the presence of the masking construct. Upon activation of the effector protein the masking construct is cleaved or otherwise silenced allowing for expression and detection of the gene product as the positive detectable signal.

In an embodiment, the masking construct may sequester one or more reagents needed to generate a detectable positive signal such that release of the one or more reagents from the masking construct results in generation of the detectable positive signal. The one or more reagents may combine to produce a colorimetric signal, a chemiluminescent signal, a fluorescent signal, or any other detectable signal and may comprise any reagents known to be suitable for such purposes. In an embodiment, the one or more reagents are sequestered by RNA aptamers that bind the one or more reagents. The one or more reagents are released when the effector protein is activated upon detection of a target molecule and the RNA aptamers are degraded.

In an embodiment, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In an embodiment, the immobilized masking agent is an RNA-based aptamer that can be cleaved by the activated effector protein upon detection of a target molecule.

In certain other example embodiments, the masking construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the masking construct is cleaved to a degree sufficient to interfere with the ability of the masking construct to bind the reagent thereby allowing the labeled binding partner to bind to the immobilized reagent. Thus, the labeled binding partner remains after the wash step indicating the presence of the target molecule in the sample. In certain aspects, the masking construct that binds the immobilized reagent is an RNA aptamer. The immobilized reagent may be a protein and the labeled minding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In an embodiment, the masking construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In an embodiment, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more RNA aptamers to the protein. Upon activation of the effector proteins disclosed herein, the RNA aptamers are cleaved or degraded to an extent that they no longer inhibit the protein's ability to generate the detectable signal. In an embodiment, the aptamer is a thrombin inhibitor aptamer. In an embodiment the thrombin inhibitor aptamer has a sequence of GGGAACAAAGCUGAAGUACUUACCC (SEQ ID NO: 85). When this aptamer is cleaved, thrombin will become active and will cleave a peptide colorimetric or fluorescent substrate. In an embodiment, the colorimetric substrate is para-nitroanilide (pNA) covalently linked to the peptide substrate for thrombin. Upon cleavage by thrombin, pNA is released and becomes yellow in color and easily visible to the eye. In an embodiment, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In an embodiment, RNAse activity is detected colorimetrically via cleavage of enzyme-inhibiting aptamers. One potential mode of converting RNAse activity into a colorimetric signal is to couple the cleavage of an RNA aptamer with the re-activation of an enzyme that is capable of producing a colorimetric output. In the absence of RNA cleavage, the intact aptamer will bind to the enzyme target and inhibit its activity. The advantage of this readout system is that the enzyme provides an additional amplification step: once liberated from an aptamer via collateral activity (e.g., expected multimeric Cas polypeptide complex collateral activity), the colorimetric enzyme will continue to produce colorimetric product, leading to a multiplication of signal.

In an embodiment, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In an embodiment, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In an embodiment, RNAse activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to RNAse activity, colorimetric enzyme and inhibitor pairs can be engineered into RNAse sensors. The colorimetric RNAse sensor based upon small-molecule inhibitors involves three components: the colorimetric enzyme, the inhibitor, and a bridging RNA that is covalently linked to both the inhibitor and enzyme, tethering the inhibitor to the enzyme. In the uncleaved configuration, the enzyme is inhibited by the increased local concentration of the small molecule; when the RNA is cleaved (e.g., by Cas13a collateral cleavage), the inhibitor will be released, and the colorimetric enzyme will be activated.

In an embodiment, RNAse activity is detected colorimetrically via formation and/or activation of G-quadruplexes. G quadraplexes in DNA can complex with heme (iron (III)-protoporphyrin IX) to form a DNAzyme with peroxidase activity. When supplied with a peroxidase substrate (e.g., ABTS: (2,2′-Azinobis [3-ethylbenzothiazoline-6-sulfonic acid]-diammonium salt)), the G-quadraplex-heme complex in the presence of hydrogen peroxide causes oxidation of the substrate, which then forms a green color in solution. An example G-quadraplex forming DNA sequence is: GGGTAGGGCGGGTTGGGA (SEQ ID NO: 86). By hybridizing an RNA sequence to this DNA aptamer, formation of the G-quadraplex structure will be limited. Upon RNAse collateral activation (e.g., C2c2-complex collateral activation), the RNA staple will be cleaved allowing the G quadraplex to form and heme to bind. This strategy is particularly appealing because color formation is enzymatic, meaning there is additional amplification beyond RNAse activation.

In an embodiment, the masking construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the masking construct are able to generate a detectable signal, for example by aggregation or simple increase in solution concentration. In an embodiment, the immobilized masking agent is an RNA-based aptamer that can be cleaved by the activated effector protein upon detection of a target molecule.

In one example embodiment, the masking construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in an embodiment, such detection agents may be held in aggregate by one or more bridge molecules. At least a portion of the bridge molecule comprises RNA. Upon activation of the effector proteins disclosed herein, the RNA portion of the bridge molecule is cleaved allowing the detection agent to disperse and resulting in the corresponding change in color. In an embodiment, the bridge molecule is an RNA molecule. In an embodiment, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel, and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA bridge is cut by the activated CRISPR effector protein, the beforementioned color shift is observed. In an embodiment the particles are colloidal metals. In certain other example embodiments, the colloidal metal is a colloidal gold. In an embodiment, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In an embodiment the nanoparticles are modified to include DNA linkers extending from the surface of the nanoparticle. Individual particles are linked together by single-stranded RNA (ssRNA) bridges that hybridize on each end of the RNA to at least a portion of the DNA linkers. Thus, the nanoparticles will form a web of linked particles and aggregate, appearing as a dark precipitate. Upon activation of the CRISPR effector proteins disclosed herein, the ssRNA bridge will be cleaved, releasing the AU NPS from the linked mesh, and producing a visible red color. Example DNA linkers and RNA bridge sequences are listed below. Thiol linkers on the end of the DNA linkers may be used for surface conjugation to the AuNPS. Other forms of conjugation may be used. In an embodiment, two populations of AuNPs may be generated, one for each DNA linker. This will help facilitate proper binding of the ssRNA bridge with proper orientation. In an embodiment, a first DNA linker is conjugated by the 3′ end while a second DNA linker is conjugated by the 5′ end.

TABLE 10

| | |
|---|---|
| C2c2 | TTATAACTATTCCTAAAAAAAAAAA/ |
| colorimetric | 3ThioMC3-D/ |
| DNA1 | (SEQ ID NO: 87) |
| C2c2 | /5ThioMC6-D/AAAAAAAAAACTC |
| colorimetric | CCCTAATAACAAT |
| DNA2 | (SEQ ID NO: 88) |
| C2c2 | GGGUAGGAAUAGUUAUAAUUUCCCU |
| colorimetric | UUCCCAUUGUUAUUA |
| bridge | GGGAG(SEQ ID NO: 89) |

In certain other example embodiments, the masking construct may comprise an RNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the masking construct may comprise one or more RNA oligonucleotides to which are attached one or more metal nanoparticles, such as gold nanoparticles. In some embodiments, the masking construct comprises a plurality of metal nanoparticles crosslinked by a plurality of RNA oligonucleotides forming a closed loop. In one embodiment, the masking construct comprises three gold nanoparticles crosslinked by three RNA oligonucleotides forming a closed loop. In some embodiments, the cleavage of the RNA oligonucleotides by the CRISPR effector protein leads to a detectable signal produced by the metal nanoparticles.

In certain other example embodiments, the masking construct may comprise one or more RNA oligonucleotides to which are attached one or more quantum dots. In some embodiments, the cleavage of the RNA oligonucleotides by the CRISPR effector protein leads to a detectable signal produced by the quantum dots.

In one example embodiment, the masking construct may comprise a quantum dot. The quantum dot may have multiple linker molecules attached to the surface. At least a portion of the linker molecule comprises RNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art Upon activation of the effector proteins disclosed herein, the RNA portion of the linker molecule is cleaved thereby eliminating the proximity between the quantum dot and one or more quenchers needed to maintain the quenching effect. In an embodiment, the quantum dot is streptavidin conjugated. In an embodiment, the RNAs are attached via biotin linkers and recruit quenching molecules with the following sequences:/5Biosg/UCUCGUACGUUC/31AbRQSp/(SEQ ID NO: 90) or/5Biosg/UCUCGUACGUUCUCUCGUACGUUC/31AbRQSp/(SEQ ID NO: 91), where/5Biosg/is a biotin tag and/31AbRQSp/is an Iowa black quencher. Upon cleavage, by the activated effectors disclosed herein the quantum dot will fluoresce visibly.

In a similar fashion, fluorescence energy transfer (FRET) may be used to generate a detectable positive signal. FRET is a non-radiative process by which a photon from an energetically excited fluorophore (i.e., “donor fluorophore”) raises the energy state of an electron in another molecule (i.e., “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the masking construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In an embodiment, the masking construct comprises the use of intercalating dyes which change their absorbance in response to cleavage of long RNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in an embodiment the masking construct comprises an RNA and intercalating dye complex that changes absorbance upon the cleavage of RNA by the effector proteins disclosed herein.

In an embodiment, the masking construct may comprise an initiator for an HCR reaction. See, e.g., Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one species. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9 (1): 167-175, Wang et al. “An enzyme-free colorimetric assay using hybridization chain reaction amplification and split aptamers” Analyst 2015, 150, 7657-7662, and Song et al. “Non-Covalent fluorescent labeling of hairpin DNA probe coupled with hybridization chain reaction for sensitive DNA detection.” Applied Spectroscopy, 70 (4): 686-694 (2016).

In an embodiment, the masking construct may comprise an HCR initiator sequence and a cleavable structural element, such as a loop or hairpin, that prevents the initiator from initiating the HCR reaction. Upon cleavage of the structure element by an activated CRISPR effector protein, the initiator is then released to trigger the HCR reaction, detection thereof indicating the presence of one or more targets in the sample. In an embodiment, the masking construct comprises a hairpin with an RNA loop. When an activated CRISPR effector protein cuts the RNA loop, the initiator can be released to trigger the HCR reaction.

### Amplification of Target

In an embodiment, target nucleic acids (or aptamer sequences or trigger nucleic acids), e.g., RNAs and/or DNAs, may be amplified prior to activating the CRISPR effector protein. In an embodiment, the CRISPR effector system is an RNA targeting system and the target nucleic acids/aptamer sequences/trigger nucleic acids are RNAs, transcribed DNAs, amplicons thereof, or any combination thereof. In an embodiment, the CRISPR effector system is a DNA targeting system and the target nucleic acids/aptamer sequences/trigger nucleic acids are DNAs, reverse transcribed RNAs, amplicons thereof, or any combination thereof. As used herein, amplification techniques as described herein which are suitable for target nucleic acids of nucleic acid detection systems are also suitable for aptamer sequences and/or trigger nucleic acids of polypeptide detection systems.

Any suitable RNA or DNA amplification technique may be used. In an embodiment, the RNA or DNA amplification is an isothermal amplification. In an embodiment, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In an embodiment, non-isothermal amplification methods may be used which include, but are not limited to, PCR, multiple displacement amplification (MDA), rolling circle amplification (RCA), ligase chain reaction (LCR), or ramification amplification method (RAM).

In an embodiment, the RNA or DNA amplification is NASBA, which is initiated with reverse transcription of target RNA by a sequence-specific reverse primer to create an RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In an embodiment, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in an embodiment the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgC12), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4) 2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In an embodiment, detection of DNA with the methods or systems of the invention requires transcription of the (amplified) DNA into RNA prior to detection.

### Target RNA/DNA Enrichment

In an embodiment, target nucleic acids, aptamer sequences, and/or trigger nucleic acids (e.g., RNA or DNA) may first be enriched prior to detection or amplification of the target nucleic acids, aptamer sequences, and/or trigger nucleic acids (e.g., RNA or DNA). In an embodiment, this enrichment may be achieved by binding of the target nucleic acids, aptamer sequences, and/or trigger nucleic acids (e.g., RNA or DNA) by a CRISPR effector system. As used herein, target RNA/DNA enrichment techniques as described herein which are suitable for target nucleic acids of nucleic acid detection systems are also suitable for aptamer sequences and/or trigger nucleic acids of polypeptide detection systems.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In an embodiment enrichment may take place at temperatures as low as 20-37° C. In an embodiment, a set of guide RNAs to different target nucleic acids are used in a single assay, allowing for detection of multiple targets and/or multiple variants of a single target.

In an embodiment, a dead CRISPR effector protein, e.g., a dead CRISPR effector protein, may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid support comprises a patterned surface suitable for immobilization of molecules in an ordered pattern. In an embodiment a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein refers to a chamber comprising a solid surface across which one or more fluid reagents can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprises microspheres or beads. “Microspheres,” “bead,” “particles,” are intended to mean within the context of a solid substrate to mean small discrete particles made of various material including, but not limited to, plastics, ceramics, glass, and polystyrene. In an embodiment, the microspheres are magnetic microspheres or beads. Alternatively or additionally, the beads may be porous. The bead sizes range from nanometers, e.g., 100 nm, to millimeters, e.g., 1 mm.

A sample containing, or suspected of containing, the target nucleic acids may then be exposed to the substrate to allow binding of the target nucleic acids to the bound dead CRISPR effector protein. Non-target molecules may then be washed away. In an embodiment, the target nucleic acids may then be released from the dead CRISPR effector protein/guide RNA complex for further detection using the methods disclosed herein. In an embodiment, the target nucleic acids may first be amplified as described herein.

In an embodiment, the dead CRISPR effector may be labeled with a binding tag. In an embodiment the dead CRISPR effector may be chemically tagged. For example, the dead CRISPR effector may be chemically biotinylated. In another example embodiment, a fusion may be created by adding additional sequence encoding a fusion to the dead CRISPR effector. One example of such a fusion is an AviTag™, which employs a highly targeted enzymatic conjugation of a single biotin on a unique 15 amino acid peptide tag. In an embodiment, the dead CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the dead CRISPR effector system once it has bound a target nucleic acid or to fix the dead CRISPR effector system on the solid substrate.

In an embodiment, the guide RNA may be labeled with a binding tag. In an embodiment, the entire guide RNA may be labeled using in vitro transcription (IVT) incorporating one or more biotinylated nucleotides, such as, biotinylated uracil. In some embodiments, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

Amplification and/or Enhancement of Detectable Positive Signal

In an embodiment, further modification may be introduced that further amplify the detectable positive signal. For example, activated CRISPR effector protein collateral activation may be used to generate a secondary target (e.g., target nucleic acid, aptamer sequence, or trigger nucleic acid) or additional guide sequence, or both. In one example embodiment, the reaction solution would contain a secondary target that is spiked in at high concentration. The secondary target may be distinct from the primary target (i.e., the target for which the assay is designed to detect) and in certain instances may be common across all reaction volumes. A secondary guide sequence for the secondary target may be protected, e.g., by a secondary structural feature such as a hairpin with a RNA loop, and unable to bind the second target or the CRISPR effector protein. Cleavage of the protecting group by an activated CRISPR effector protein (i.e., after activation by formation of complex with the primary target(s) in solution) and formation of a complex with free CRISPR effector protein in solution and activation from the spiked in secondary target. In certain other example embodiments, a similar concept is used with a second guide sequence to a secondary target sequence. The secondary target sequence may be protected a structural feature or protecting group on the secondary target. Cleavage of a protecting group off the secondary target then allows additional CRISPR effector protein/second guide sequence/secondary target complex to form. In yet another example embodiment, activation of CRISPR effector protein by the primary target(s) may be used to cleave a protected or circularized primer, which is then released to perform an isothermal amplification reaction, such as those disclosed herein, on a template that encodes a secondary guide sequence, secondary target sequence, or both. Subsequent transcription of this amplified template would produce more secondary guide sequence and/or secondary target sequence, followed by additional CRISPR effector protein collateral activation.

### Detection of Proteins

The systems, devices, and methods disclosed herein may also be adapted for detection of polypeptides (or other molecules) in addition to detection of nucleic acids, via incorporation of a specifically configured polypeptide detection aptamer. The polypeptide detection aptamers are distinct from the masking construct aptamers discussed above. First, the aptamers are designed to specifically bind to one or more target molecules. In one example embodiment, the target molecule is a target polypeptide. In another example embodiment, the target molecule is a target chemical compound, such as a target therapeutic molecule. Methods for designing and selecting aptamers with specificity for a given target, such as SELEX, are known in the art. In addition to specificity to a given target the aptamers are further designed to incorporate an RNA polymerase promoter binding site. In an embodiment, the RNA polymerase promoter is a T7 promoter. Prior to binding the aptamer binding to a target, the RNA polymerase promoter binding site is not accessible or otherwise recognizable to an RNA polymerase. However, the aptamer is configured so that upon binding of a target the structure of the aptamer undergoes a conformational change such that the RNA polymerase promoter binding site is then exposed. An aptamer sequence downstream of the RNA polymerase promoter binding site acts as a template for generation of a trigger RNA oligonucleotide by an RNA polymerase. Thus, the template portion of the aptamer may further incorporate a barcode or other identifying sequence that identifies a given aptamer and its target. Guide RNAs as described above may then be designed to recognize these specific trigger oligonucleotide sequences. Binding of the guide RNAs to the trigger oligonucleotides activates the CRISPR effector proteins which proceeds to deactivate the masking constructs and generate a positive detectable signal as described previously.

Accordingly, in an embodiment, the methods disclosed herein comprise the additional step of distributing a sample or set of sample into a set of individual discrete volumes, each individual discrete volume comprising peptide detection aptamers, a CRISPR effector protein, one or more guide RNAs, a masking construct, and incubating the sample or set of samples under conditions sufficient to allow binding of the detection aptamers to the one or more target molecules (e.g., target polypeptides), wherein binding of the aptamer to a corresponding target molecule (e.g., target polypeptide) results in exposure of the RNA polymerase promoter binding site such that synthesis of a trigger RNA is initiated by the binding of a RNA polymerase to the RNA polymerase promoter binding site.

In another example embodiment, binding of the aptamer may expose a primer binding site upon binding of the aptamer to a target polypeptide. For example, the aptamer may expose a RPA primer binding site. Thus, the addition or inclusion of the primer will then feed into an amplification reaction, such as the RPA reaction outlined above.

In an embodiment, the aptamer may be a conformation-switching aptamer, which upon binding to the target of interest may change secondary structure and expose new regions of single-stranded DNA. In an embodiment, these new-regions of single-stranded DNA may be used as substrates for ligation, extending the aptamers and creating longer ssDNA molecules which can be specifically detected using the embodiments disclosed herein. The aptamer design could be further combined with ternary complexes for detection of low-epitope targets, such as glucose (Yang et al. 2015: pubs.acs.org/doi/abs/10.1021/acs.analchem.5b01634http://pubs.acs.org/doi/abs/10.1021/acs.anal chem.5b01634). Example conformation shifting aptamers and corresponding guide RNAs (crRNAs) are shown below.

TABLE 11

| | |
|---|---|
| Thrombin aptamer | tgtggttggtgtggttggttcatggtcata |
| | ttggtttttttttttttttccaaccacagt |
| | (SEQ ID NO: 92) |
| Thrombin ligation | ggttggtagtctcgaattgctctctttcac |
| probe | tggcc (SEQ ID NO: 93) |
| Thrombin RPA | gaaattaatacgactcactatagggggttg |
| forward 1 primer | gttcatggtcatattggt |
| | (SEQ ID NO: 94) |
| Thrombin RPA | gaaattaatacgactcactatagggggttg |
| forward 2 primer | gtgtggttggttcatggtcatattggt |
| | (SEQ ID NO: 95) |
| Thrombin RPA | ggccagtgaaagagagcaattegagactac |
| reverse 1 primer | c (SEQ ID NO: 96) |
| Thrombin crRNA 1 | gauuuagacuaccccaaaaacgaaggggac |
| | uaaaacccagugaaagagagcaauucgaga |
| | cuac |
| | (SEQ ID NO: 97) |
| Thrombin crRNA 2 | gauuuagacuaccccaaaaacgaagggga |
| | cuaaaacaaagagagcaauucgagacuac |
| | caacca |
| | (SEQ ID NO: 98) |
| Thrombin crRNA 3 | gauuuagacuaccccaaaaacgaagggga |
| | cuaaaacagacuaccaaccacagagacug |
| | ugguug |
| | (SEQ ID NO: 99) |
| PTK7 full length | gttagatcgcaagcatatcattgcgcttg |
| amplicon control | cgatctaactgctgcgccgccgggaaaat |
| | actgtacggttagatcgcatagtctcgaa |
| | ttgctctctttcactggcc |
| | (SEQ ID NO: 100) |
| PTK7 aptamer | gttagatcgcaagcatatcattgcgcttg |
| | cgatctaactgctgcgccgccgggaaaat |
| | actgtacggttag |
| | (SEQ ID NO: 101) |
| PTK7 ligation | atcgcatagtctcgaattgctctctttca |
| probe | ctggcc (SEQ ID NO: 102) |
| PTK7 RPA | gaaattaatacgactcactatagggatcg |
| forward 1 primer | caagcatatcattgcgcttgc |
| | (SEQ ID NO: 103) |
| PTK7 RPA | ggccagtgaaagagagcaattcgagacta |
| reverse 1 primer | tg (SEQ ID NO: 104) |
| PTK7 crRNA 1 | gauuuagacuaccccaaaaacgaagggga |
| | cuaaaacccagugaaagagagcaauucga |
| | gacuau |
| | (SEQ ID NO: 105) |
| PTK7 crRNA 2 | gauuuagacuaccccaaaaacgaagggga |
| | cuaaaacagagcaauucgagacuaugcga |
| | ucuaac |
| | (SEQ ID NO: 106) |
| PTK7 crRNA 3 | gauuuagacuaccccaaaaacgaagggga |
| | cuaaaacacuaugcgaucuaaccguacag |
| | uauuuu |
| | (SEQ ID NO: 107) |

### Devices

The systems described herein can be embodied on diagnostic devices. A number of substrates and configurations may be used. The devices may be capable of defining multiple individual discrete volumes within the device. As used herein an “individual discrete volume” refers to a discrete space, such as a container, receptacle, or other defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a sample within a defined space. Individual discrete volumes may be identified by molecular tags, such as nucleic acid barcodes. By “diffusion rate limited” (for example diffusion defined volumes) is meant spaces that are only accessible to certain molecules or reactions because diffusion constraints effectively defining a space or volume as would be the case for two parallel laminar streams where diffusion will limit the migrationCRn of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example polyethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In an embodiment, the compartment is an aqueous droplet in a water-in-oil emulsion. In specific embodiments, any of the applications, methods, or systems described herein requiring exact or uniform volumes may employ the use of an acoustic liquid dispenser.

In an embodiment, the device comprises a flexible material substrate on which a number of spots may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g., multiple clinical samples from different individuals) for the presence of the same target, or a limited number of targets, or aliquots of a single sample (or multiple samples from the same source) for the presence of multiple different targets in the sample. In an embodiment, the elements of the systems described herein are freeze dried onto the paper or cloth substrate. Example flexible material-based substrates that may be used in certain example devices are disclosed in Pardee et al. Cell. 2016, 165 (5): 1255-66 and Pardee et al. Cell. 2014, 159 (4): 950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34 (11): 909-21 (2016). Further flexible based materials may include nitrocellulose, polycarbonate, methylethyl cellulose, polyvinylidene fluoride (PVDF), polystyrene, or glass (see e.g., US20120238008). In an embodiment, discrete volumes are separated by a hydrophobic surface, such as but not limited to wax, photoresist, or solid ink.

In some embodiments, a dosimeter or badge may be provided that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous agents as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

Samples sources that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

In other example embodiments, the elements of the systems described herein may be place on a single use substrate, such as swab or cloth that is used to swab a surface or sample fluid. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient-such as a saliva sample from the mouth—or a swab of the skin. In other embodiments, a sample or swab may be taken of a meat product on order to detect the presence of absence of contaminants on or within the meat product.

Near-real-time microbial diagnostics are needed for food, clinical, industrial, and other environmental settings (see e.g., Lu T K, Bowers J, and Koeris M S., Trends Biotechnol. 2013 June; 31 (6): 325-7). In an embodiment, the present invention is used for rapid detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulnificus, Yersinia enterocolitica and Yersinia pseudotuberculosis, Brucella spp., Corynebacterium ulcerans, Coxiella burnetii, or Plesiomonas shigelloides).

In an embodiment, the device is or comprises a flow strip. For instance, a lateral flow strip allows for CRISPR effector protein (e.g., CRISPR nuclease, e.g., CRISPR RNAse) detection by color. The nucleic acid reporter (e.g., RNA reporter) is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g., anti-FITC) antibodies hybridized at the first line and anti-second molecule (e.g., anti-biotin) antibodies at the second downstream line. As the reaction flows down the strip, uncleaved reporter will bind to anti-first molecule antibodies at the first capture line, while cleaved reporters will liberate the second molecule and allow second molecule binding at the second capture line. Second molecule sandwich antibodies, for instance conjugated to nanoparticles, such as gold nanoparticles, will bind any second molecule at the first or second line and result in a strong readout/signal (e.g., color). As more reporter is cleaved, more signal will accumulate at the second capture line and less signal will appear at the first line. In certain aspects, the invention relates to the use of a follow strip as described herein for detecting nucleic acids or polypeptides. In certain aspects, the invention relates to a method for detecting nucleic acids or polypeptides with a flow strip as defined herein, e.g. (lateral) flow tests or (lateral) flow immunochromatographic assays.

In an embodiment, the device is a microfluidic device that generates and/or merges different droplets (i.e., individual discrete volumes). For example, a first set of droplets may be formed containing samples to be screened and a second set of droplets formed containing the elements of the systems described herein. The first and second set of droplets are then merged, and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methyl methacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-maltoside (DDM), pluronic, Tween-20, other similar surfactants, polyethylene glycol (PEG), albumin, collagen, and other similar proteins and peptides.

In an embodiment, the system and/or device may be adapted for conversion to a flow-cytometry readout in or allow to all of sensitive and quantitative measurements of millions of cells in a single experiment and improve upon existing flow-based methods, such as the PrimeFlow assay. In an embodiment, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiator) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hour et al. “Direct Detection and drug-resistance profiling of bacteremias using inertial microfluidics” Lap Chip. 15 (10): 2297-2307 (2016).

In systems described herein, may further be incorporated into wearable medical devices that assess biological samples, such as biological fluids, of a subject outside the clinic setting and report the outcome of the assay remotely to a central server accessible by a medical care professional. The device may include the ability to self-sample blood, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresis” to Andrew Conrad.

In an embodiment, the device may comprise individual wells, such as microplate wells. The size of the microplate wells may be the size of standard 6, 24, 96, 384, 1536, 3456, or 9600 sized wells. In an embodiment, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In an embodiment, the devices are connected to controllers with programmable valves that work together to move fluids through the device. In an embodiment, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

In an embodiment, the CRISPR effector protein is bound to each discrete volume in the device. Each discrete volume may comprise a different guide RNA specific for a different target molecule. In an embodiment, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

The present invention may be used with a wireless lab-on-chip (LOC) diagnostic sensor system (see e.g., U.S. Pat. No. 9,470,699 “Diagnostic radio frequency identification sensors and applications thereof”). In an embodiment, the present invention is performed in a LOC controlled by a wireless device (e.g., a cell phone, a personal digital assistant (PDA), a tablet) and results are reported to said device.

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather, a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

In preferred embodiments, the LOC may be a microfluidic device. The LOC may be a passive chip, wherein the chip is powered and controlled through a wireless device. In an embodiment, the LOC includes a microfluidic channel for holding reagents and a channel for introducing a sample. In an embodiment, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In an embodiment, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In an embodiment, if DNA or RNA is used then the conductive molecules can be attached directly to the matching DNA or RNA strands. The release of the conductive molecules may be detected across a sensor. The assay may be a one step process.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In an embodiment, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In an embodiment, an optical sensor detects unmasking of a fluorescent masking agent.

In an embodiment, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4 (3), 104-128; mReader from Mobile Assay; and Holomic Rapid Diagnostic Test Reader).

As noted herein, certain embodiments allow detection via colorimetric change which has certain attendant benefits when embodiments are utilized in POC situations and/or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in an embodiment utilizing quantum dot-based masking constructs, use of a hand-held UV light, or other suitable device, may be successfully used to detect a signal owing to the near complete quantum yield provided by quantum dots.

### Kits

In one aspect, the invention provides kits containing any one or more of the elements disclosed in the above methods and compositions. In an embodiment, the kit comprises a vector system as taught herein or one or more of the components of the CRISPR/Cas system or complex as taught herein, such as crRNAs and/or CRISPR-Cas effector protein or CRISPR-Cas effector protein encoding mRNA, and instructions for using the kit. Elements may be provided individually or in combinations, and may be provided in any suitable container, such as a vial, a bottle, or a tube. In an embodiment, the kit includes instructions in one or more languages, for example in more than one language. The instructions may be specific to the applications and methods described herein. In an embodiment, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In an embodiment, the buffer is alkaline. In an embodiment, the buffer has a pH from about 7 to about 10. In an embodiment, the kit comprises one or more oligonucleotides corresponding to a guide sequence for insertion into a vector so as to operably link the guide or crRNA sequence and a regulatory element. In an embodiment, the kit comprises a homologous recombination template polynucleotide. In an embodiment, the kit comprises one or more of the vectors and/or one or more of the polynucleotides described herein. The kit may advantageously allow to provide all elements of the systems of the invention.

The invention further relates to the following aspects, which are described hereinbelow as numbered statements:
1. A non-naturally occurring, engineered composition comprising: a B-CASP polypeptide; and a plurality of Cas polypeptides, wherein (a) and (b) are capable of forming a non-naturally occurring, engineered multimeric CRISPR-Cas complex in the presence of a guide molecule, and wherein the guide molecule is capable of directing sequence-specific binding of the non-naturally occurring, engineered multimeric CRISPR-Cas complex to a target sequence in a target polynucleotide.
2. The composition according to Statement 1, wherein the β-CASP polypeptide comprises an N-terminal β-CASP domain and a C-terminal adapter domain.
3. The composition according to Statement 2, wherein the C-terminal adapter domain comprises an α-helical domain having homology to the C-terminus of a Cas10 protein.
4. The composition according to any one of the preceding Statements, wherein the β-CASP polypeptide comprises a plurality of residues capable of coordinating with Zn2+ ions.
5 The composition according to any one of the preceding Statements, wherein the plurality of Cas polypeptides comprise a Cas5 family polypeptide, a Cas7 family polypeptide, and optionally a Cas6 family polypeptide.
6. The composition according to any one of the preceding Statements, wherein the Cas5 family polypeptide is a Type III Csx10 polypeptide, a homolog thereof, or an ortholog thereof; wherein the Cas7 family polypeptide is a Type III Csm3 polypeptide, a homolog thereof, or an ortholog thereof; and/or wherein the Cas6 family polypeptide is a Type III Cas6 polypeptide, a homolog thereof, or an ortholog thereof.
7. The composition according to any one of the preceding Statements, wherein one or more of the β-CASP polypeptide and/or the Cas polypeptides has catalytic activity; wherein one or more of the β-CASP polypeptide and/or the Cas polypeptides lacks catalytic activity; and/or wherein one or more of the β-CASP polypeptide and/or the Cas polypeptides is or is engineered to have nickase activity.
8. The composition according to Statement 7, wherein the β-CASP polypeptide has catalytic activity.
9. The composition according to Statement 7 or Statement 8, wherein the Cas7 family polypeptide lacks catalytic activity.
10. The composition according to any one of Statements 7 to 9, wherein the catalytic activity is RNAse activity.
11. The composition according to any one of the preceding Statements, wherein one or more of the β-CASP polypeptide and/or the Cas polypeptides further comprise one or more additional modifications that increase nuclease efficiency, target polynucleotide binding efficiency, or reduce off-target nuclease activity.
12. The composition according to any one of the preceding Statements, wherein the β-CASP polypeptide and/or one or more of the Cas polypeptides is/are further linked to or otherwise capable of associating with a heterologous functional domain.
13. The composition according to Statement 12, wherein the heterologous functional domain is a nucleotide deaminase, a transposase, a reverse transcriptase, a recombinase, a methylase, a demethylase, an acetylase, or a deacetylase.
14. The composition according to any one of the preceding Statements, wherein the β-CASP polypeptide and/or one or more of the Cas polypeptides is/are derived from one or more bacteria and/or one or more archaea.
15. The composition according to Statements 14, wherein:
the one or more bacteria each independently belong to the phylum selected from the group consisting of: Bacillota; and DTHG01000077 4 candidate division White Oak River group 3 (WOR-3); the one or more archaea each independently belong to the phylum selected from the group consisting of MBU4492343 1/HEQ78297 1/Euryarchaeota; RLE40065.1 Candidatus Woesearchaeota; NHI92075 1 Candidatus Lokiarchaeota; and PKP54316 1 Candidatus Altiarchaeales archaeon; and/or the one or more archaea each independently belong to the order selected from the group consisting of: PXF52022 1/RJS85311 1/Methanophagales; and MCD4797691.1/CAG0966219 1/RLG33181 1 Methanosarcinales.
16. The composition according to Statements 14 or Statements 15, wherein: the one or more bacteria each independently belong to the Staphylococcus genus, and optionally one of the bacteria is 6NBT Staphylococcus epidermis; the one or more archaea each independently belong to the family MCG2727882 1 Candidatus Methanoperedenaceae, and optionally one of the archaea is WP 0972978485 1 Candidatus Methanoperedens sp BLZ2; the one or more archaea each independently belong to a genus selected from the group consisting of: WP 0972978485 1 Candidatus Methanoperedens; and/or the one or more archaea each independently belong to a species selected from the group consisting of: 4QTS (Csm3) Mathanocaldococcus jannaschii; and WP 012965105 1 Ferroglobus placidus, and optionally one of the archaea is WP 012965105 1 Ferroglobus placidus DSM 10642.
17. The composition according to any one of Statements 14-16, wherein each of the β-CASP polypeptide and the one or more Cas polypeptides are derived from a same species or from one or more different species.
18. The composition according to any one of Statements 14-17, wherein the β-CASP polypeptide is derived from a first species, and the one or more Cas polypeptides are derived from a second species different from the first species.
19. The composition according to any one of Statements 1-18, further comprising one or more guide molecules, wherein the guide molecules comprise a guide sequence capable of hybridizing to a target sequence of the target molecule, and wherein the composition is optionally in the form of the non-naturally occurring, engineered multimeric CRISPR-Cas complex.
20. The composition according to Statement 19, wherein the at least one guide molecule is a crRNA comprising a spacer sequence flanked on the 5′ and 3′ ends by direct repeat sequences.
21. A nucleic acid molecule comprising a nucleotide sequence encoding one or more components of the composition of any one of Statements 1-20.
22. A vector comprising a polynucleotide comprising one or more nucleic acid molecules of Statement 21.
23. The vector of Statement 22, wherein the vector is a viral vector.
24 A delivery vehicle comprising one or more components of the composition of any one of Statements 1-20, the non-naturally occurring, engineered multimeric CRISPR-Cas complex of Statement 19 or Statement 20, the nucleic acid molecule of Statement 21, the vector of Statement 22 or Statement 23, or any combination thereof.
25. The delivery vehicle of Statement 24, wherein the delivery vehicle is a lipid nanoparticle, a viral capsid, an engineered retroelement vector, a polynucleotide-based nano-structure, or an extracellular contractile injection system.
26. An engineered cell comprising the one or more components of the composition of any one of Statements 1-20, the non-naturally occurring, engineered multimeric CRISPR-Cas complex of Statement 19 or Statement 20, the nucleic acid molecule of Statement 21, the vector of Statement 22 or Statement 23, or any combination thereof.
27. The engineered cell according to Statement 26, wherein the engineered cell is an engineered eukaryotic cell or an engineered prokaryotic cell.
28. An organism comprising the cell according to Statement 26 or Statement 27.
29. The organism of Statement 28, wherein the organism is an animal or a plant.
30. A pharmaceutical composition for treatment of a disease or disorder, comprising one or more components of the composition of any one of Statements 1-20, the non-naturally occurring, engineered multimeric CRISPR-Cas complex of Statement 19 or Statements20, the nucleic acid molecule of Statement 21, the vector of Statement 22 or Statement 23, the delivery particle of Statement 24 or Statement 25, the engineered cell of Statement 26 or Statements 27, or any combination thereof.
31. A method of modifying a target polynucleotide, the method comprising contacting a sample comprising a target polynucleotide with one or more components of the composition of any one of Statements 1-20, the non-naturally occurring, engineered multimeric CRISPR-Cas complex of Statement 19 or Statement 20, the nucleic acid molecule of Statement 21, the vector of Statement 22 or Statement 23, the delivery particle of Statement 24 or Statement 25, the engineered cell of Statement 26 or Statement 27, the pharmaceutical composition of Statement 30, or any combination thereof.
32. The method of Statement 31, wherein contacting results in modification of a gene product or modification of the amount or expression of a gene product.
33. The method of Statement 31 or Statement 32, wherein the target polynucleotide is a disease- or disorder-associated target polynucleotide.

### EXAMPLES

Further embodiments are illustrated in the following Examples which are given for illustrative purposes only and are not intended to limit the scope of the invention.

### Example 1

Uncovering the functional diversity of CRISPR-Cas systems with deep terascale clustering.

Microbial systems have formed the foundation of widely applicable biotechnologies such as CRISPR, but the exponential growth of sequence databases has made exploring their diversity increasingly challenging. To address this limitation, Applicant developed the Fast Locality-Sensitive Hashing-based clustering algorithm (FLSHclust) that can perform deep protein clustering on massive datasets in linear (linearithmically O (N logN), with N protein sequences) time. Applicant incorporated FLSHclust into a sensitive, scalable CRISPR discovery pipeline and identified previously unreported CRISPR-linked genes, including new CRISPR effector modules, transposon-linked systems, CRISPR adaptation-linked genes, and diverse auxiliary genes, indicating that many more biochemical functions are coupled to adaptive immunity in prokaryotes than previously thought. Applicant identified and characterized a candidate Type VII CRISPR system from archaea and demonstrated that it acts on RNA. This work opens new avenues for harnessing CRISPR diversity for biotechnology and medicine and for broader exploration of the vast functional diversity of microbial proteins.

### INTRODUCTION

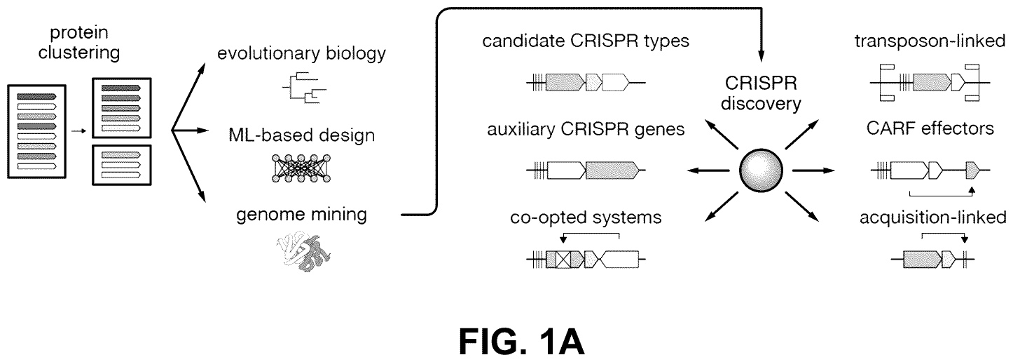

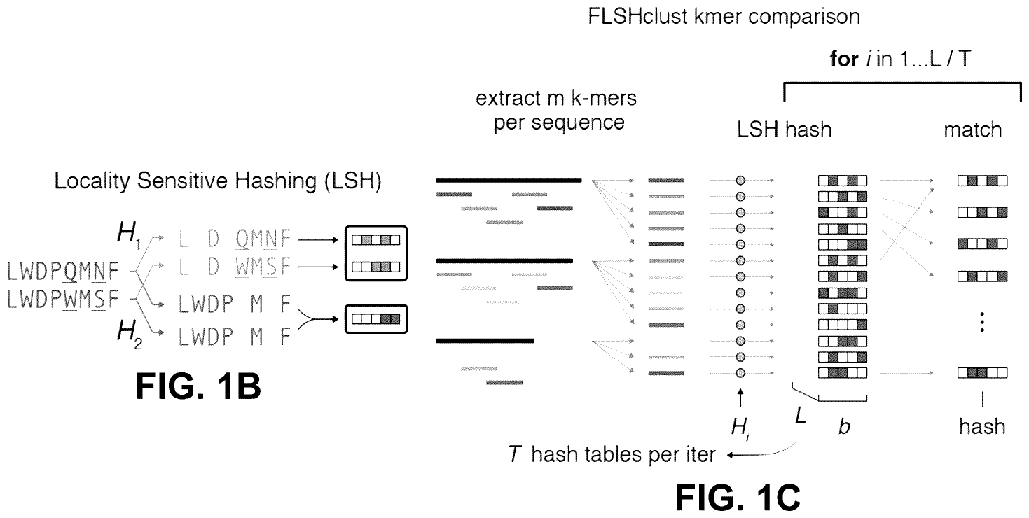

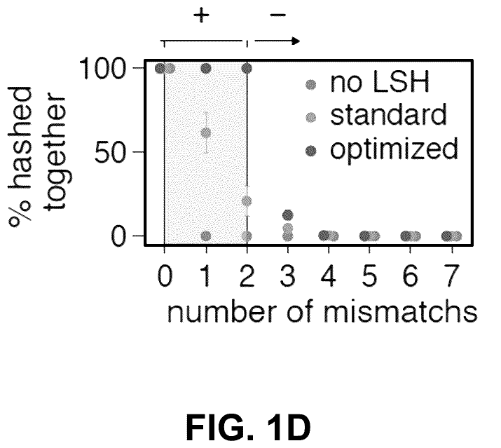

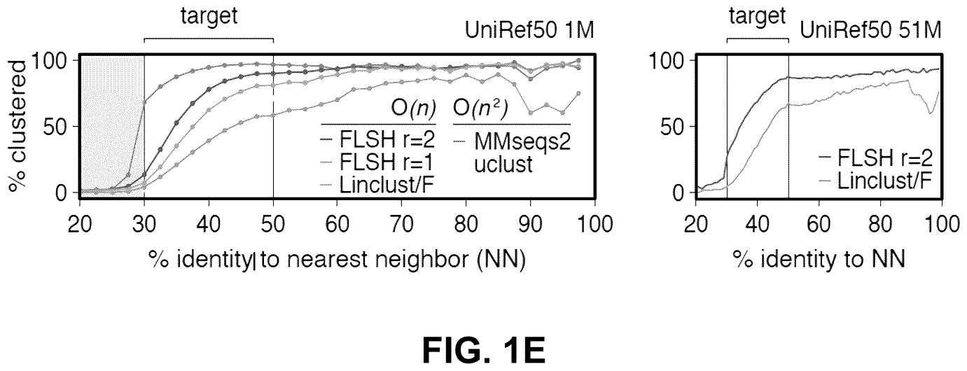

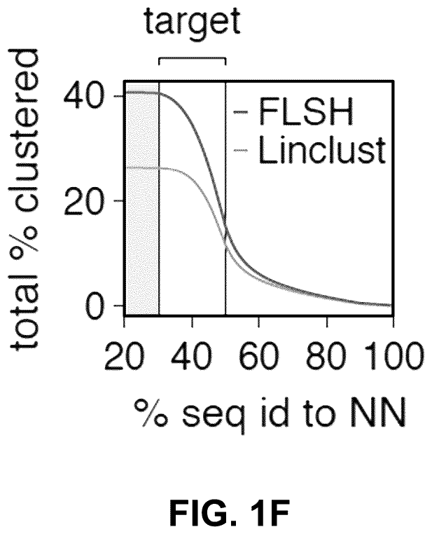

Discovery of enzymes and natural biochemical systems advances molecular evolution studies, shines new light on biological processes, and provides a starting point for the development of molecular technologies. Over the past few decades, an enormous variety of previously unknown protein families and entire functional systems has been discovered through the systematic mining of the rapidly growing nucleic acid and protein sequence databases. Many of these efforts employ protein clustering to group similar protein sequences in large datasets (FIG. 1A). The output of these algorithms can then be used to inform efforts aimed at deep learning on protein sequences, 3D protein structure prediction, and genome mining. One prime example of the latter is the discovery of novel CRISPR systems, which has led to the development of transformative biotechnologies and therapeutic approaches (1-4).

CRISPR systems, which are widely found in bacteria and archaea, are RNA-guided adaptive immune systems (5). They are composed of a CRISPR array, which encodes the CRISPR (cr) RNAs that gives rise to the guides, an adaptation module, which integrates new spacers into the CRISPR array, and an interference module that consists of effector components guided by the crRNAs to matching targets, which are then cleaved. CRISPR effectors can be either complexes of Cas proteins (e.g., Cascade) in Class 1 CRISPR systems or single, multidomain proteins (e.g., Cas9, Cas12, Cas13) in Class 2 CRISPR systems (6). This inherent modularity and programmability of CRISPR systems has been capitalized on to develop a suite of RNA-guided molecular technologies, starting with Cas9-mediated genome editing (1).

This toolbox was considerably expanded through computational searches that uncovered many new CRISPR systems (3, 7-9), but existing computational methods have relied on algorithms that have quadratic runtime, such as all-against-all comparisons and protein clustering (9). These methods quickly become impractical for mining exponentially growing datasets containing billions of proteins (10). Linear scaling clustering methods like LinClust (11) can address some of these issues but produce small clusters of highly similar sequences that limit the ability to study deep evolutionary relationships. Protein domain profiles, such as PFAM, can be used to identify broad abundant associations (12), but group remote homologs, leading to spurious associations while missing rare ones (13).

To address these limitations and take advantage of the explosive increase of the known structural and functional diversity of proteins resulting from the exponential growth of sequence databases, Applicant developed FLSHclust (pronounced “flash clust”), a parallelized, deep clustering algorithm that scales linearithmically, O(N logN). FLSHclust can handle billions of proteins, enabling efficient analysis of the vast, rapidly growing sequence databases. Applicant applied FLSHclust to identify previously uncharacterized CRISPR systems, including a candidate type VII CRISPR system, generating a catalog of RNA-guided proteins that expand the understanding of the biology and evolution of these systems and provide the starting point for the development of new biotechnologies.

### Fast Locality-Sensitive Hashing Allows for Deep Clustering of all Known Proteins at Terabyte Scale.

To circumvent the need for all-to-all comparisons, Applicant used locality sensitive hashing (LSH), which efficiently groups together similar objects in linear time (14), to develop FLSHclust (Fast LSH clustering) (FIG. 1B). FLSHclust first converts all amino acids in each protein to a reduced alphabet and extracts all kmers of size k from each protein (FIG. 1C). An optimal LSH family with no false negatives is generated using Markov Chain Monte Carlo, and for each hash function, all hashed kmers are mapped to buckets (FIG. 1D) (14). Two representative sequences are then selected per bucket, and for all sequences in the bucket, a graph edge is formed if an alignment between the sequence and each of the representatives satisfies the clustering criteria. The resulting graph is simplified using a density-based transformation and community detection is applied to form groups of sequences, which are then clustered using greedy clustering to produce a final set of clusters (FIG. 5A for schematic depiction of full algorithm and FIG. 5B for pseudocode, see herein Extended Discussion of FLSHclust Algorithm).

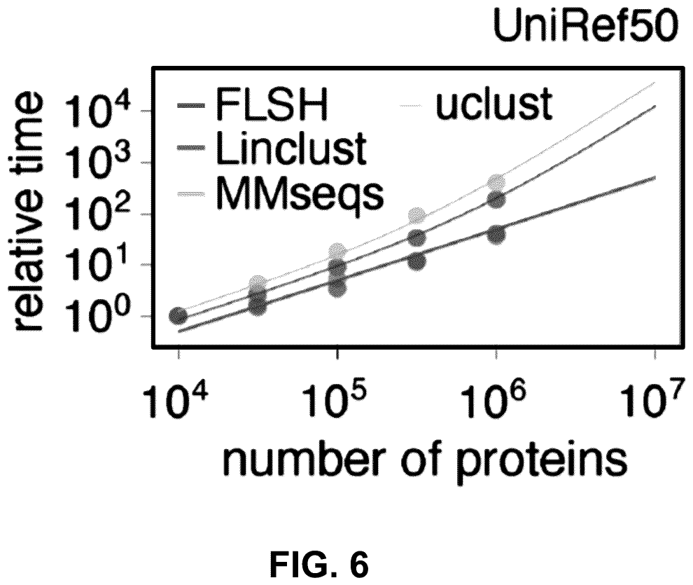

The clustering performance and scalability of FLSHclust were compared to those of other commonly used algorithms (MMSeqs2, uclust, and LinClust) using a random sample of 1 million UniRef50 proteins clustered at 30% sequence identity (FIG. 1E) (11, 15-17). The clustering quality was assessed by the percentage of proteins in clusters of size≥2 as a function of sequence identity to the nearest neighbor protein. FLSHclust clustered a substantially higher fraction of sequences than LinClust when using 2 tolerated mismatches in the LSH family with a kmer length of 13 (FIG. 1E). When attempting to cluster the entire UniRef50 database (51M sequences) down to 30% sequence identity, MMSeqs2 and uclust did not finish within 2 weeks on 32 CPUs. Furthermore, FLSHclust was substantially more complete in clustering the full database than LinClust (which does not cluster effectively below 50% sequence identity, as previously reported (11)), with a 58% increase in the total number of clustered sequences (FIG. 1F). Although FLSHclust's run times are dependent on the number of tolerated mismatches, r, it scales pseudo-linearly with the number of sequences and exhibits linear scaling behavior empirically, in contrast to MMSeq2 and uclust, which scale quadratically (FIG. 6). The ability of FLSHclust to deeply cluster sequences down to 30-35% while scaling linearly with the number of sequences makes it suitable for a wide range of big data applications in biology such as genome mining.

### Discovery of Previously Unreported, Rare CRISPR Systems

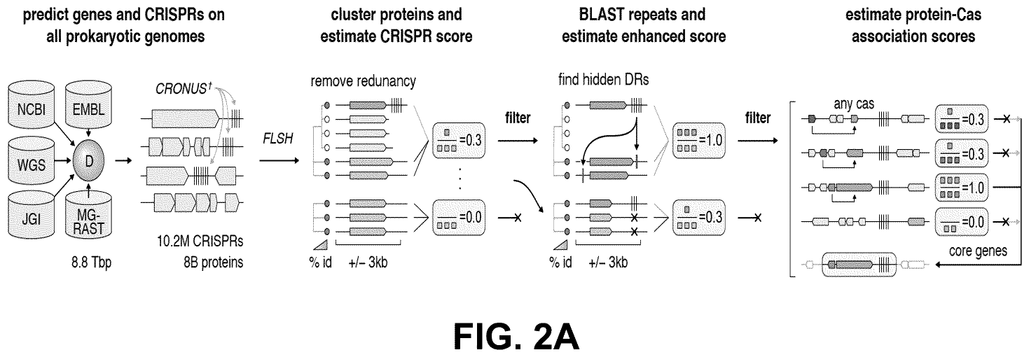

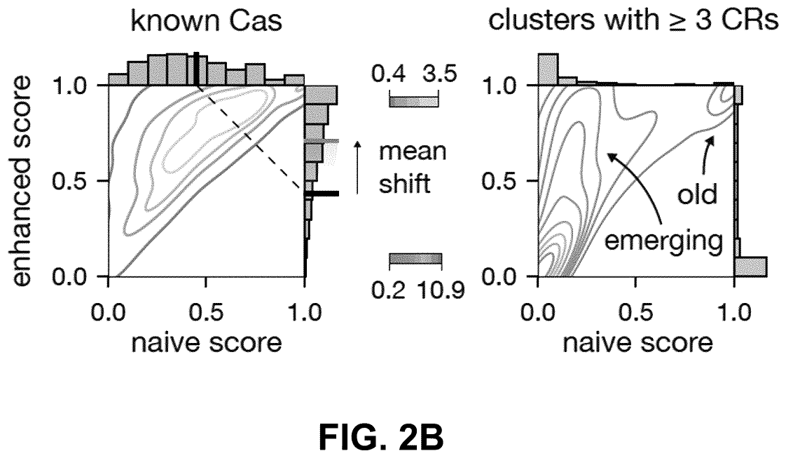

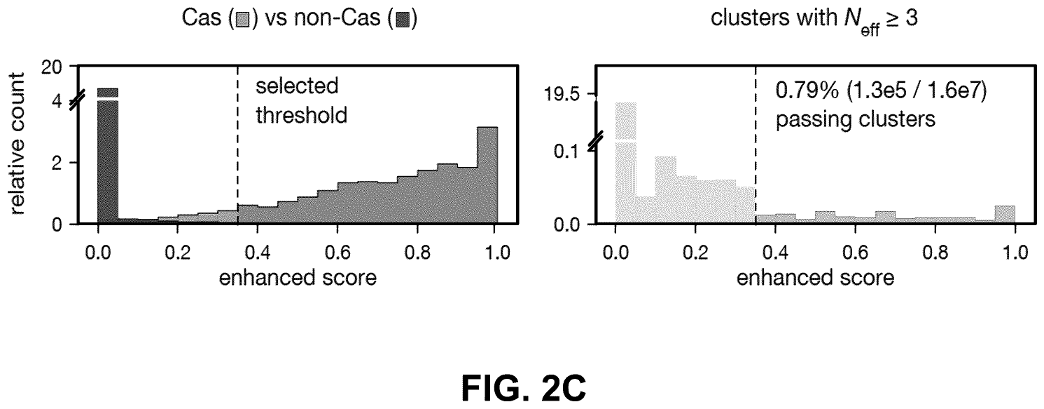

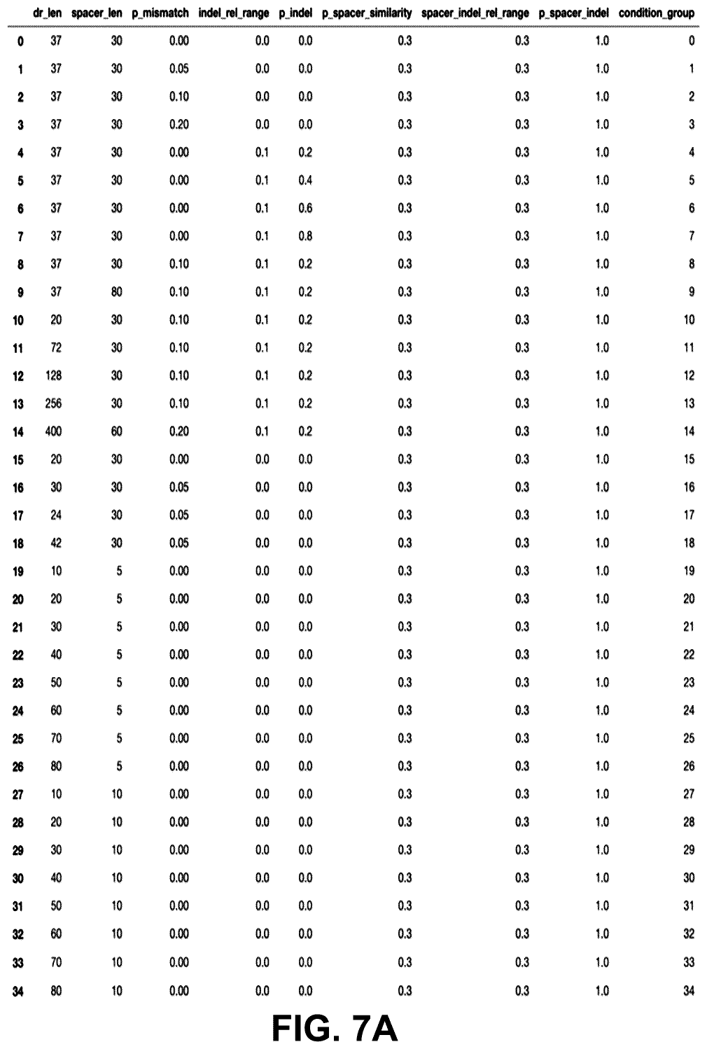

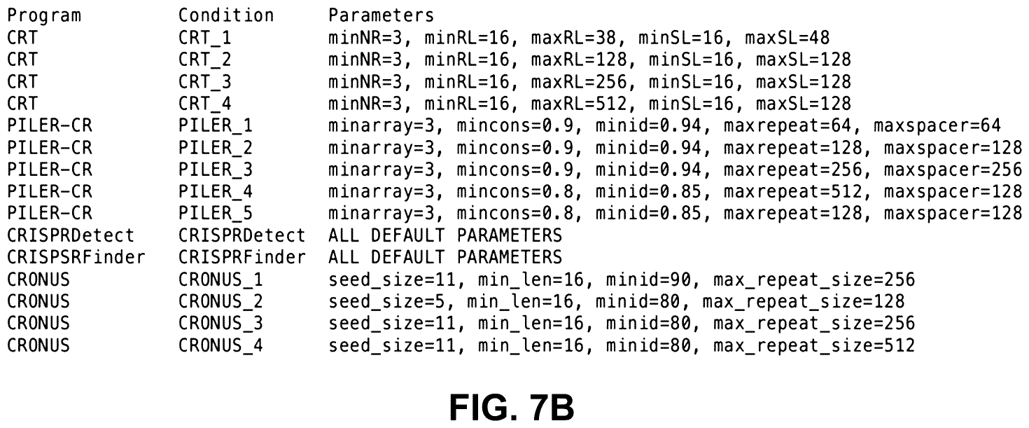

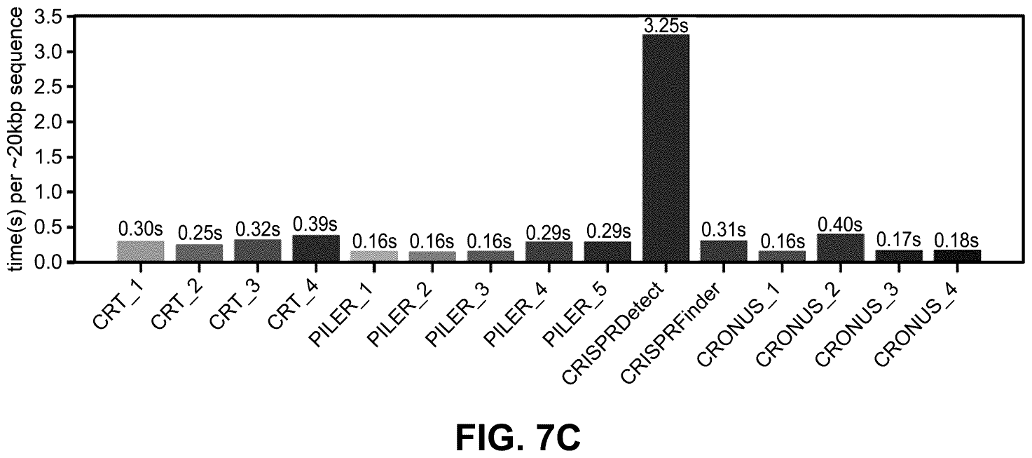

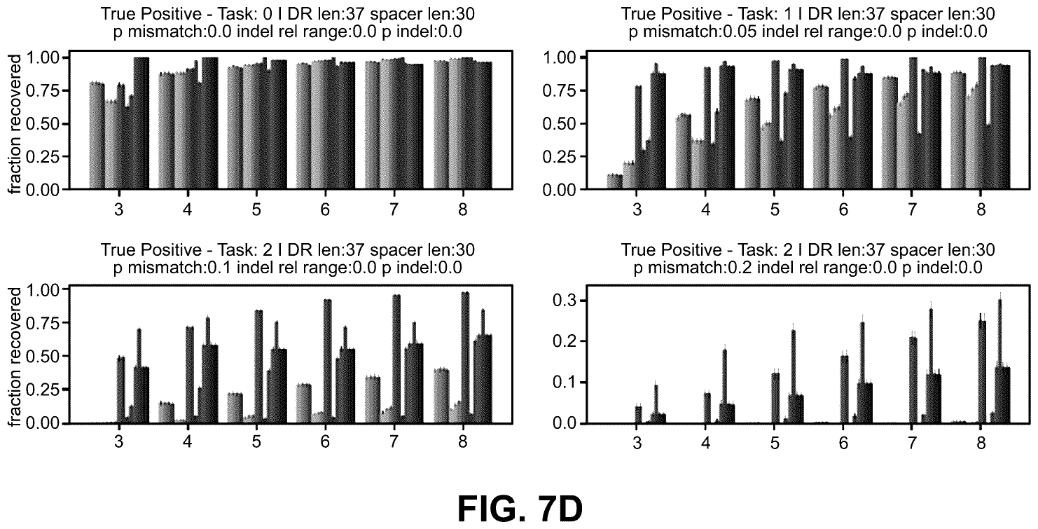

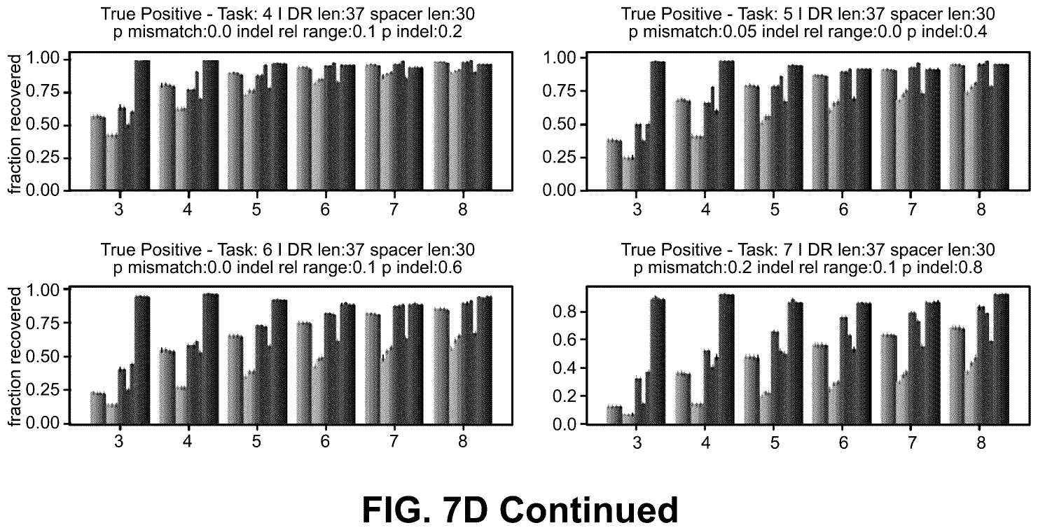

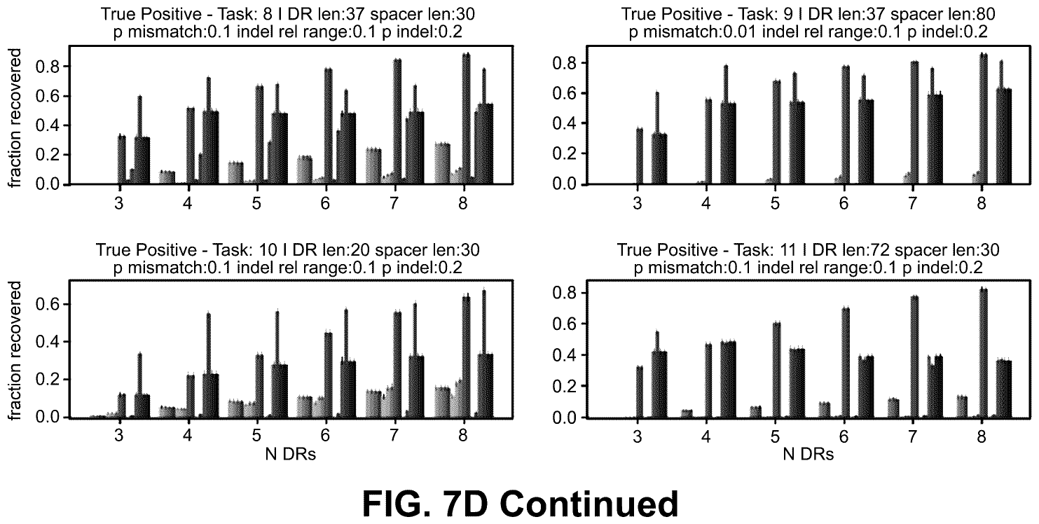

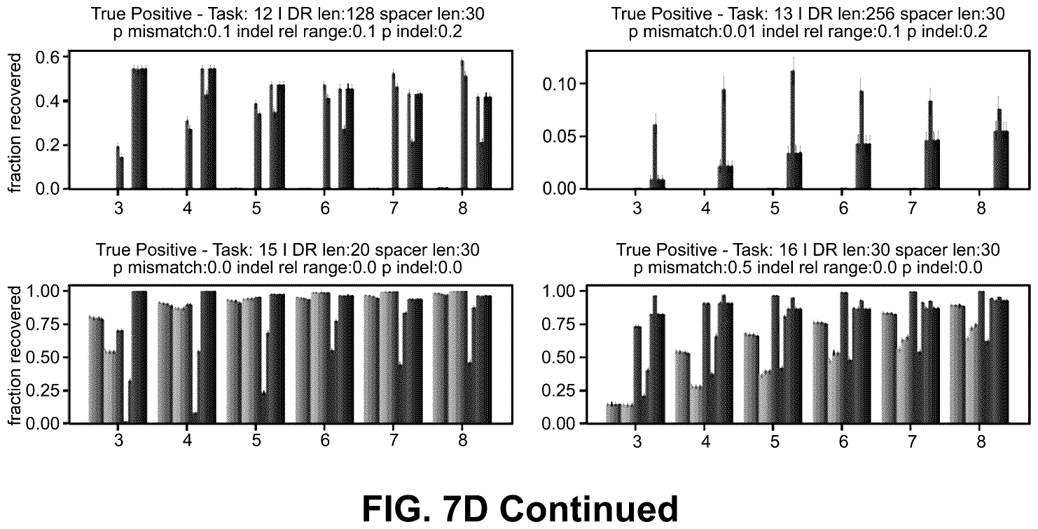

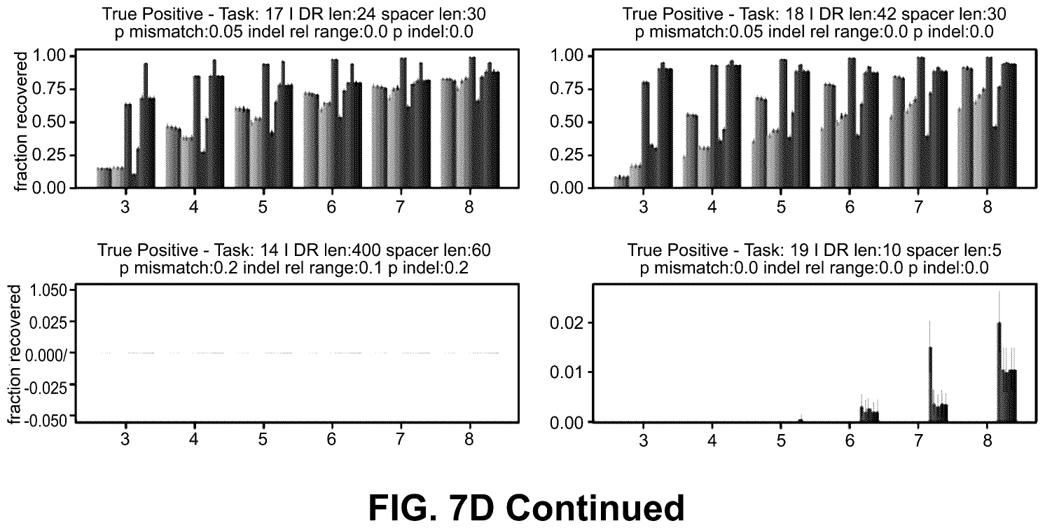

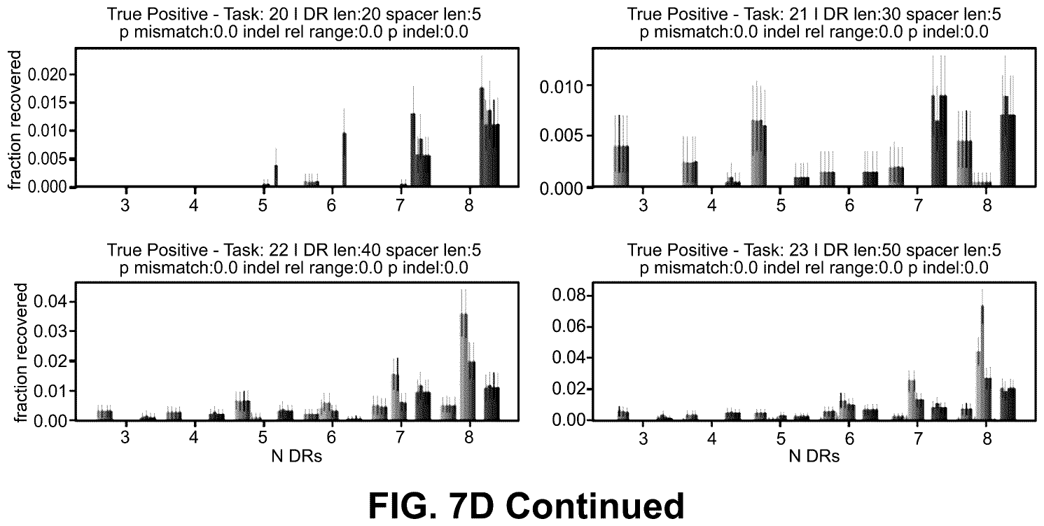

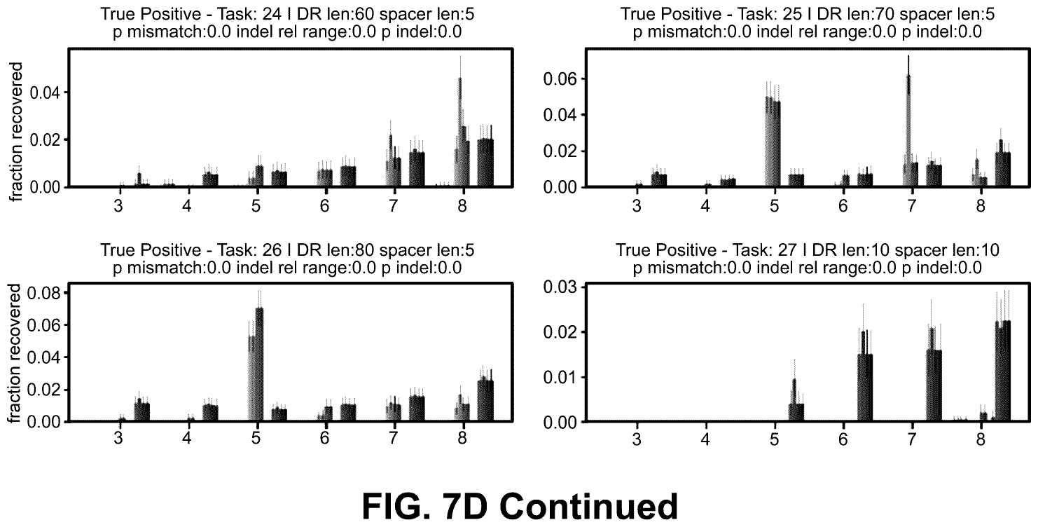

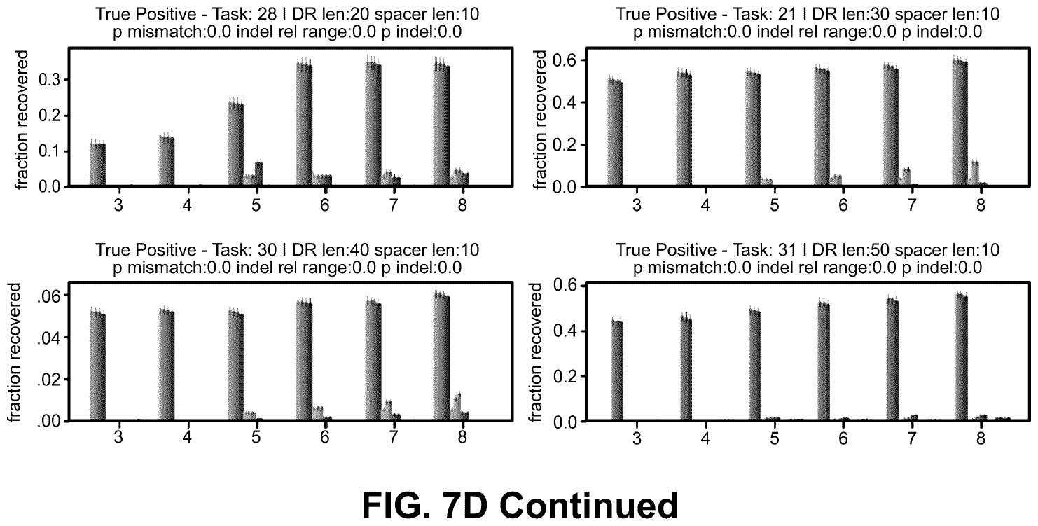

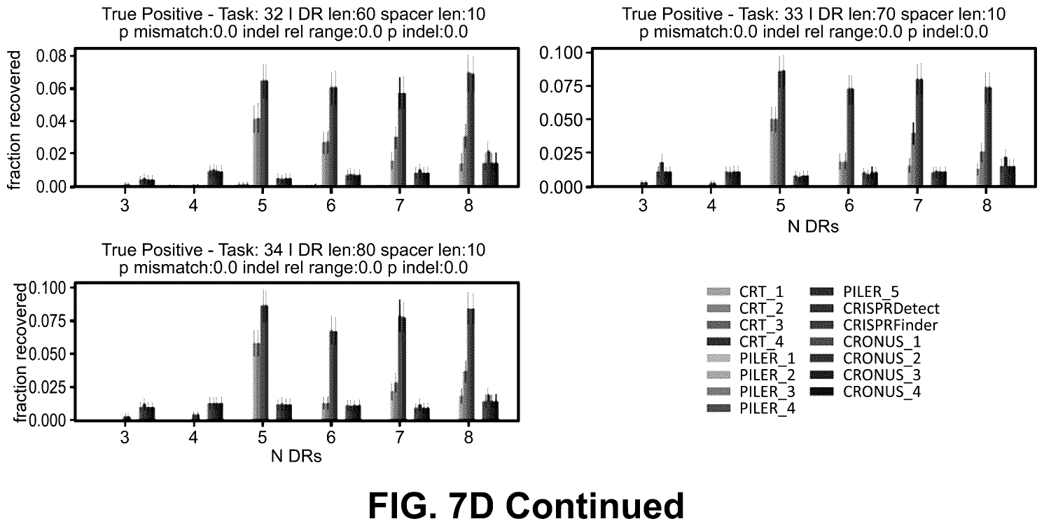

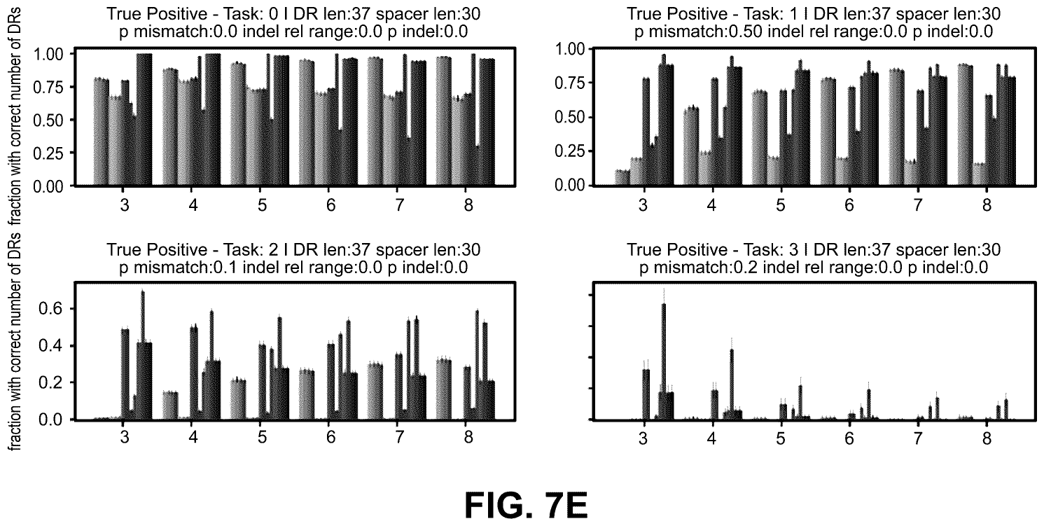

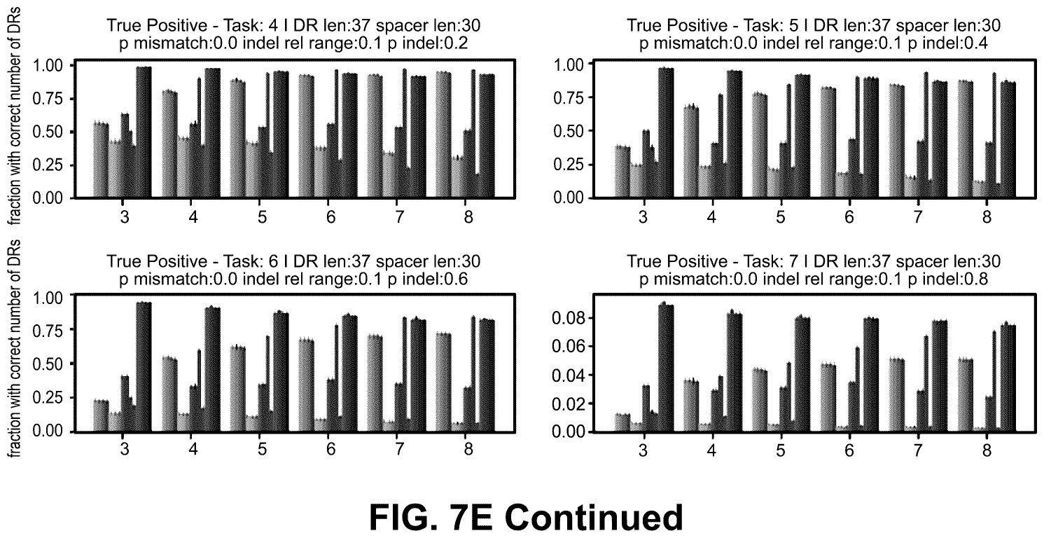

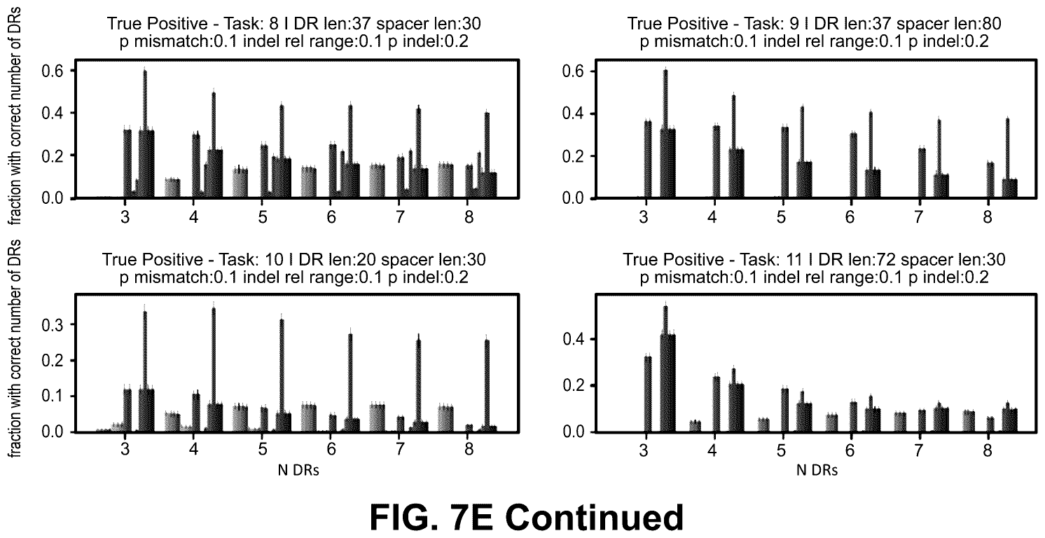

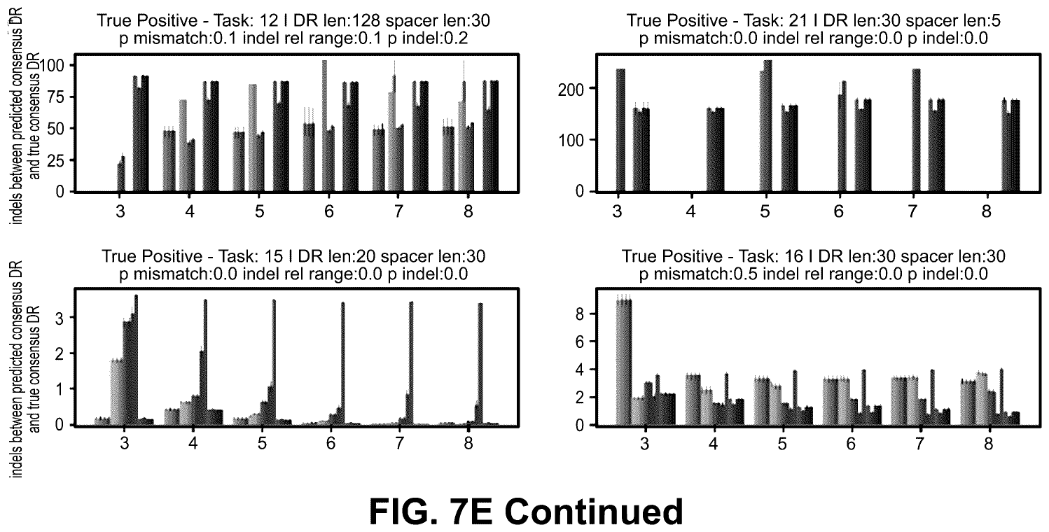

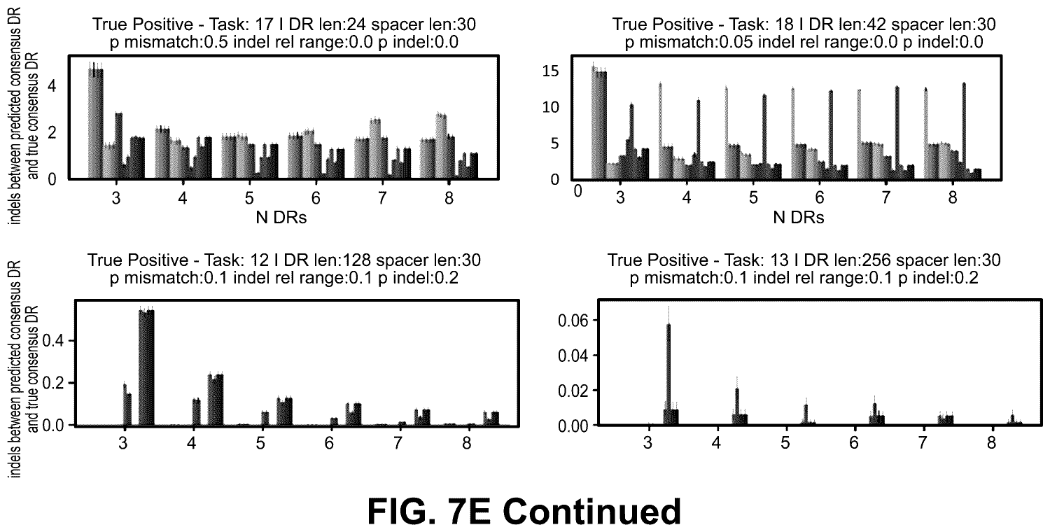

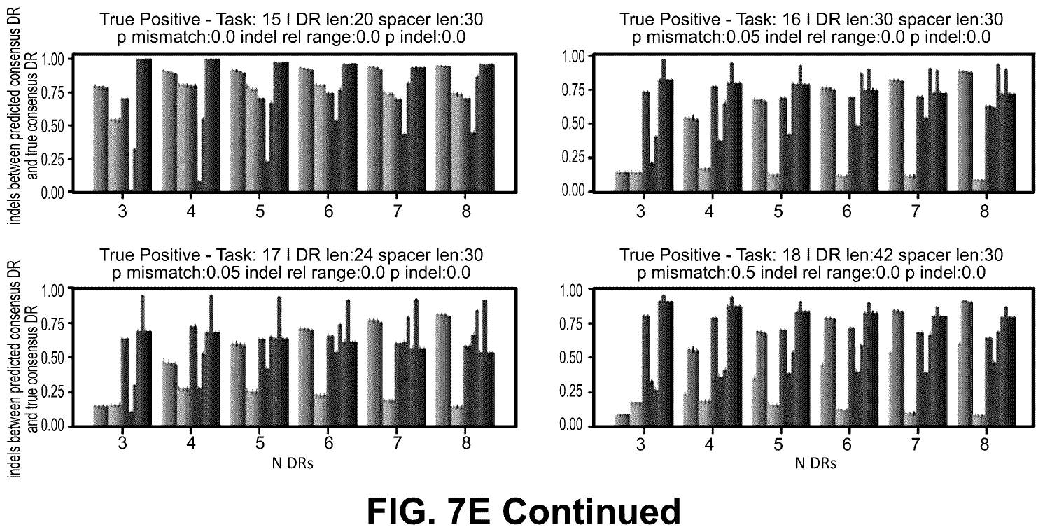

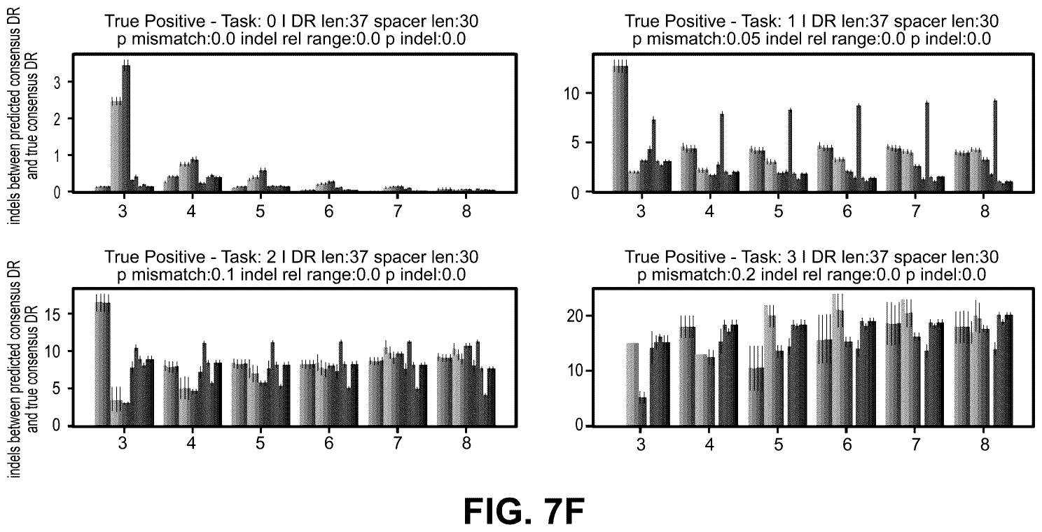

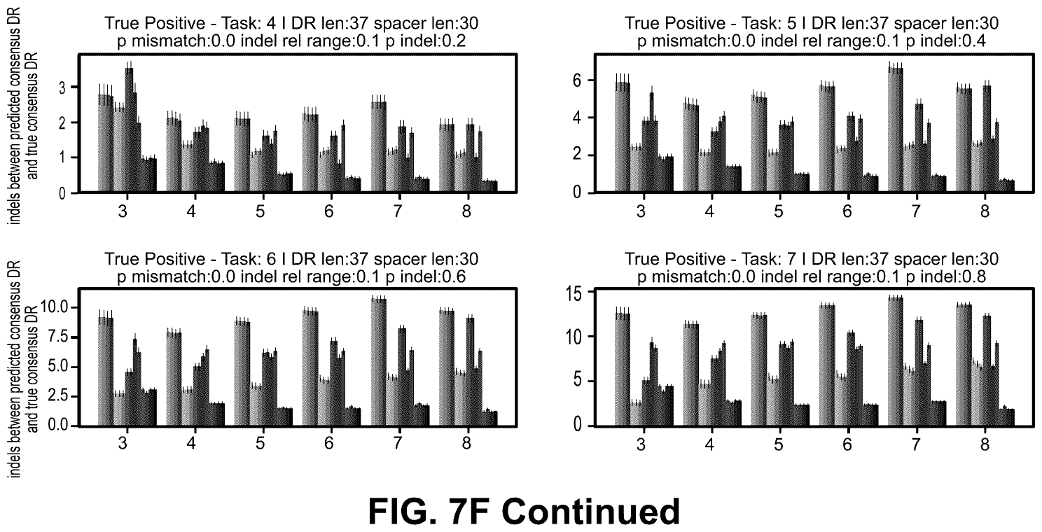

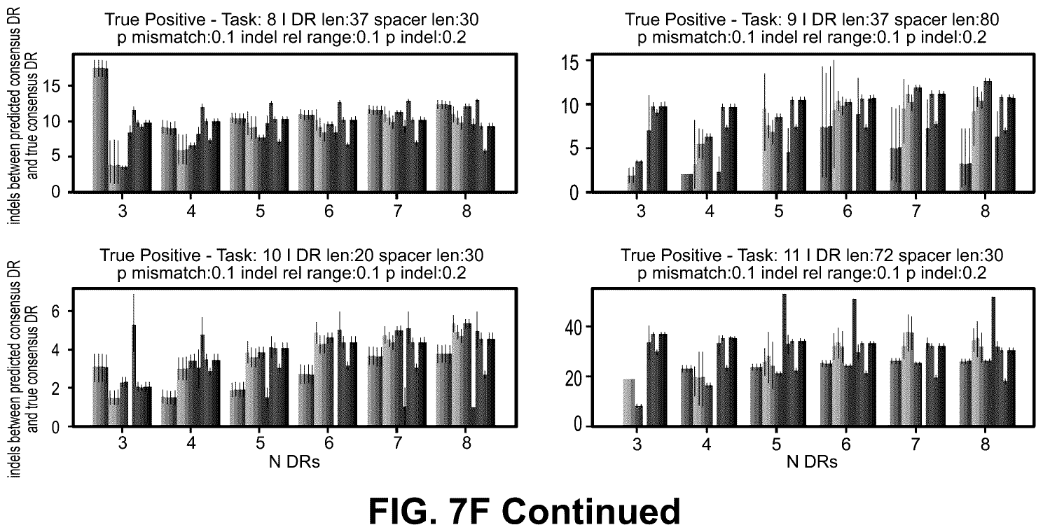

Applicant applied FLSHclust to discover new, rare CRISPR systems. CRISPR systems have diverse architectures and mechanisms and are divided into 33 subtypes (6). To find additional CRISPR systems, which may be extremely rare, Applicant developed a sensitive, linearithmically scaling ( ) N logN) CRISPR discovery pipeline that combines FLSHclust and CRISPR repeat finders to identify deep clusters of proteins that are stably associated with CRISPR arrays (FIG. 2A). Applicant curated a database of 8.8 Tbp of prokaryotic genomic and metagenomic contigs (excluding metagenomic contigs<2 kbp in length) from NCBI, WGS, and JGI (FIG. 2A). All coding sequences were predicted using Genemark (18), and CRISPR arrays were predicted using previously developed CRISPR finders (19-22) and CRONUS, a tool Applicant developed to detect smaller CRISPR arrays including imperfect repeats as well as other repeat arrays with hypervariable spacers (see Materials and Methods, FIG. 7 for benchmarking). The final database contained 8 billion proteins and 10.2 million CRISPR arrays. Using FLSHclust, Applicant iteratively clustered all proteins resulting in 1.3 billion redundancy reduced and 499.9 million deep clusters at 90% and 30% sequence identity, respectively. In contrast to clustering at 50% identity, which produced 646.4 million clusters, clustering at 30% with FLSHclust produced fewer but larger clusters with higher sample sizes (mean cluster size of 2.5 vs 2.0 non-redundant proteins).

To identify genes stably associated with CRISPR arrays, Applicant then computed a CRISPR association score (naive score) for each 30% cluster by calculating the weighted fraction of non-redundant proteins encoded within 3 kbp of a CRISPR array over the effective sample size of the cluster, N_eff, which adjusts for contig truncations that occur in metagenomic data (see Materials and Methods). To capture emerging or degrading CRISPR systems, which often only contain a single direct repeat (DR) or highly diverged DRs (23), for each CRISPR-associated cluster, Applicant selected a representative DR and searched its sequence against all other non-redundant loci in the cluster (24). The identified divergent DR sequences were used to compute an enhanced CRISPR-association score. Finally, to expand the search to find genomically distant components of CRISPR systems, all proteins considered to be CRISPR-associated were used as baits for identifying additional associated proteins (FIG. 2A).

To evaluate the performance of this CRISPR search pipeline, Applicant compared the naive and enhanced CRISPR scores of known CRISPR-associated sequence (cas) genes and found that the mean naive score of cas genes was 0.44, whereas the enhanced score increased to 0.72 (FIG. 2B), highlighting the importance of identifying divergent DRs and mini CRISPR arrays. Using the enhanced score, Applicant compared cas and non-cas genes and empirically determined a cutoff of 0.35, which included the majority of known CRISPR-associated genes while removing most of the non-cas genes (FIG. 2C). Applicant then applied this filter to all protein clusters with an effective sample size N_eff≥3, resulting in ˜130,000 clusters with associations to CRISPR-like repeats (out of 16 million total clusters with N_eff≥3). Many of these CRISPR-associated clusters included proteins or domains not previously linked to CRISPRs.

The abundance and distribution of different CRISPR systems is uneven across sequenced bacterial and archaeal genomes (25). To gauge how the increasing diversity of sequencing data correlates with the detectable CRISPR-Cas diversity with Applicant's pipeline, Applicant back-calculated the time at which the clusters with a minimum of four non-redundant CRISPR-associated loci appeared in the public dataset. These calculations track with the abundance of cas genes, highlighting the importance of diverse environmental sampling for discovering useful biochemical and mechanistic diversity in CRISPR-Cas systems. Notably, the system that Applicant identifies here is rare and appeared in the dataset only recently in the past decade. This system is a candidate Type VII CRISPR system, which Applicant has experimentally characterized. Type VII CRISPR system is a precise RNA-guided RNA endonuclease complex containing a β-CASP nuclease domain protein.

CRISPR systems evolve through modular replacement of Cas components and subdomains. Applicant further identified a distinct system that is represented in diverse archaea and contains a β-CASP nuclease domain protein. This protein is encoded in a predicted operon with Cas7 and Cas5 which, together, likely form a previously unreported minimal effector complex, and in some cases, a Cas6, which is implicated in crRNA processing (FIG. 3A, FIG. 8A The Cas5 and the Cas7 of this system are distantly related to the Type III-D Cas5 and Cas7 proteins, respectively, with an apparent inactivation of the Cas7 catalytic residues that are required for target RNA cleavage in type III systems (FIG. 3B, FIG. 8B-8E).

The β-CASP domain is an ancient nuclease fold found in all domains of life that exhibits RNA endonuclease, 5′ to 3′ RNA exonuclease and/or DNA nuclease activity (37). β-CASP domain proteins are involved in Non-Homologous End Joining DNA repair (NHEJ), V (D) J recombination, RNA surveillance, mRNA/rRNA maturation and RNA decay (38-42). Phylogenetic analysis of the β-CASP family supports the origin of the CRISPR-associated members from a distinct, well-defined clade (FIG. 3C, FIG. 8F). Structural modeling of the β-CASP protein with AlphaFold2 (+3) shows two distinct domains, namely, the N-terminal β-CASP domain (FIG. 9, FIG. 8G), and a C-terminal adaptor domain with structural similarity (but not detectable sequence similarity) to the ˜200 aa C-terminal domain of Cas10 (FIG. 3D), the large subunit of type III systems that is involved in target RNA interaction (44). Given its unique domain composition and association with CRISPR, Applicant propose to designate the β-CASP domain protein Cas15.

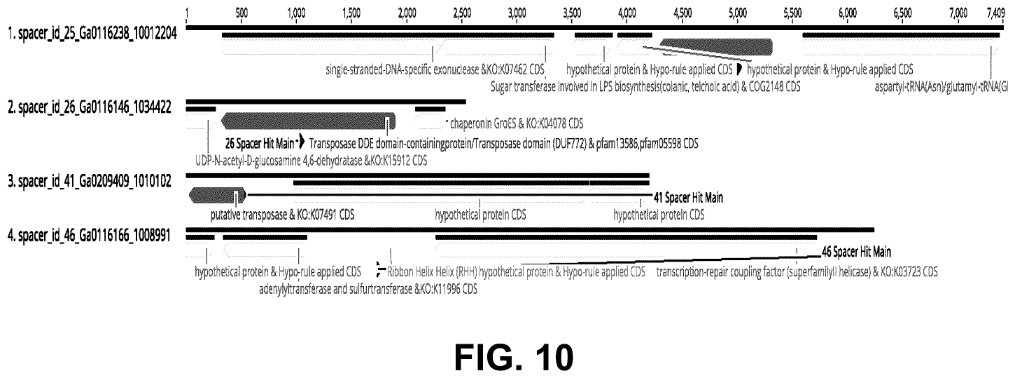

Applicant next searched for protospacer matches to the CRISPR spacers contained in these systems and found that they exhibited a pronounced bias towards the antisense strand of matching target sequences (FIG. 3E), suggesting that these systems target RNA. Applicant further observed that spacers primarily target transposon genes, indicating that the system likely functions as a defense against actively expressed transposons, unlike other known CRISPR types, which primarily target viruses or plasmids (FIG. 3F, FIG. 10).

Applicant hypothesized that the Cas15-containing system carries out interference via the β-CASP nuclease domain, in contrast to CRISPR subtype III-E, which also likely originated from subtype III-D but retains a Cas7-based interference mechanism (6, 45, 46). These findings suggest convergent evolution of minimal effector complexes. Given the distant relationship between the effector complex of the Cas15-containing system and those of other known CRISPR types, and the substitution of the effector nuclease with an unrelated protein domain, β-CASP, Applicant propose that the Cas 15-containing system is classified as type VII CRISPR-Cas system.

Small RNA-seq on purified Cas7/Cas5 RNP complexes showed that Cas7 and Cas5 form a complex that co-purifies with a processed crRNA that contains both a 5′ and 3′ DR tag, similar to Type I and IV systems (FIG. 3G) (27, 35). The complex is stable only in the presence of the corresponding crRNA, suggesting a specific functional association of the Cas7-Cas5 proteins in this system with the crRNA component encoded nearby. (FIG. 3H). To test cleavage activity, Applicant separately purified the Cas15 protein and mixed it with the purified Cas7/Cas5 RNP complex and labeled target RNA. Applicant observed precise target RNA cleavage with two cleavage sites only in the presence of all proteins and the cognate target sequence (FIG. 3I), suggesting that Cas15 is the nuclease effector in these systems. Target RNA cleavage by Cas15 and associated Cas7-Cas5 RNP was further evaluated at various temperatures. Cleavage was apparent in a range from 37° C. to 52° C. (FIG. 3J).

### Putative Novel CRISPR Variants and CRISPR-Associated Genes

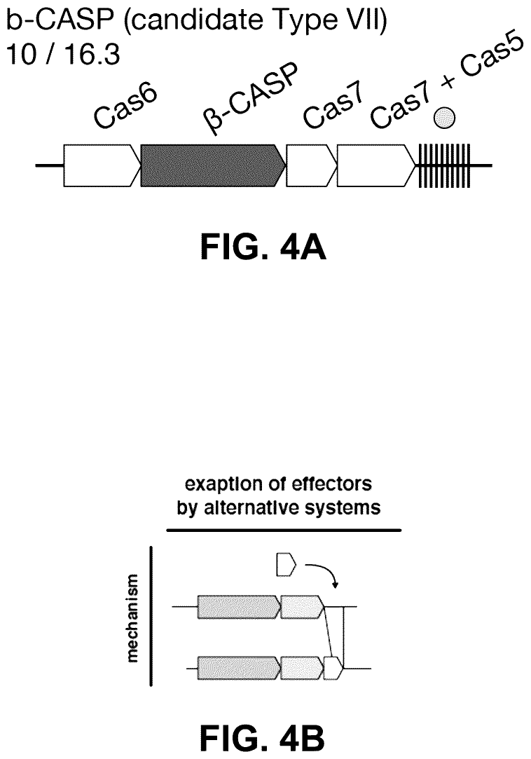

Applicant's biodiscovery pipeline identified many more putative novel systems that collectively highlight expansive diversity in the mechanisms of CRISPR-associated systems, reveal previously unreported enzymatic functions linked to RNA-guided activity, and suggest evolutionary trajectories leading to the creation and loss of CRISPR systems. In total, Applicant identified 193 CRISPR-linked genes that, to the best of Applicant's knowledge, have not been reported previously. From these findings, Applicant identified the candidate Type VII system described herein comprising new gene associations between the β-CASP domain and the Cas5, Cas7, and optionally Cas6 domains (FIG. 4A), potentially evolved via addition of the β-CASP domain into an alternate system (e.g., Class 1 Type III) system (FIG. 4B).

### DISCUSSION

The continuing and accelerating proliferation of public sequence data has the potential to transform biology, but realizing this potential requires computational approaches that can keep pace with the database growth. Central to this effort is moving away from all-to-all comparisons. Here, Applicant used LSH to develop FLSHclust, an algorithm for clustering proteins and applied it to identify CRISPR systems. Applicant found many previously unreported CRISPR systems and associated genes. The systems identified are rare, with many encompassing only a single cluster out of the ˜130,000 CRISPR-linked clusters identified, highlighting the power of this approach. The discovery of new cas genes and CRISPR systems substantially expands the known CRISPR diversity, emphasizing the functional versatility of CRISPR whereby new proteins and domains are often recruited, either replacing pre-existing components or, as in the candidate Type VII system, conferring new functions to the pre-existing scaffold of Cas proteins (FIG. 4B).

Applicant observed many new domains and proteins associated with CRISPR effector modules, several of which appear to replace the function of lost components (FIG. 4A), highlighting the modular evolution of CRISPR effectors. Applicant identified the notable case of the candidate type VII CRISPR system discovered here, in which the enzymatic domains of Cas10 were functionally replaced by the unrelated β-CASP nuclease (FIG. 3D). Similarly, the discovery of a broad variety of proteins and domains associated with CRISPR adaptation modules suggests the existence of many functional and mechanistic variations in this first stage of the CRISPR function. CRISPR systems can also be co-opted for other RNA-guided functions, such as transposition (56).

Due to the ability of CRISPR-Cas systems to programmably sense specific nucleic acids and subsequently enact enzymatic functions, the discovery and characterization of novel CRISPR effectors and downstream auxiliary functions has the potential to enable a wide range of applications and improve existing CRISPR-based technologies, such as genome editing.

### Materials and Methods

### FLSHclust Implementation

The FLSHclust algorithm was implemented in Python 3 using PySpark for distributed computation on commodity clusters without shared memory or disk. Described below are the general implementation details (see FIG. S1 for visual depiction of the algorithm).

The algorithm has the following core parameters: min_seq_id: The minimum sequence identity for including proteins in a cluster, cov: The minimum coverage between sequences for alignment, cov_approx_factor: an approximation parameter for filtering out sequences pairs that will not match the clustering criteria without actually aligning the sequences, k: The main kmer length used for clustering.

The family of hash functions used for locality-sensitive hashing is random position masking, which involves dropping random positions in each kmer before matching them with a hash table. Each hash function drops different combinations of positions. Thus, the set of hash functions can be reduced to the set of position combinations that are dropped, {Hᵢ}, which essentially is a boolean matrix of positions that are to be dropped out. Optimal functions are generated using Markov Chain Monte Carlo (MCMC) as follows. Applicants use the following method to achieve hash functions with no false negatives for up to r mismatches. All combinations of mismatch positions are enumerated up until a value m max and stored asApplicant's input X. For an initial set of hash functions, {Hᵢ}, LSH is performed on the entirety of X. The fraction of recovered sequences via LSH as a function of the number of mismatches, m, is stored as R. An energy function of the form exp (−R 7) is used for MCMC to maximize R as a function of {Hᵢ}. Optimal hash functions can be obtained and then stored for use. Perfect hash functions are obtained when R 1 at the end of optimization. Not all combinations of parameters will result in perfect hash functions.

Proteins are assigned random numeric IDs at the start. Next, all protein amino acid sequences are first converted to a reduced alphabet that groups related amino acids together. The reduced alphabet has 13 tokens, which allows each token to be represented by 4 bits, allowing for more space efficient representations than using a full char (8 bits). The map between tokens and amino acids are shown below:
[(O, [′T′, ‘A’, ‘S’]), (1, [′L′, ‘M’]), (2, [′I′, ‘V’]), (3, [′R′, ‘K’]), (4, [′Q′, ‘E’]), (5, [′D′, ‘N’]), (6, [′Y′, ‘F’]), (7, [′W′]), (8, [′C′]), (9, [′G′]), (10, [′H′]), (11, [′P′]), (12, [′U′, ‘O’, ‘J’, ‘Z’, ‘X’, ‘B’, ‘*’])]

Kmers of length k are then generated for each protein, while excluding any kmers that contain amino acid stop codons (′*′) characters. Kmers are stored in compressed format as integers when performing joins and merges to reduce space usage. Compressed formats are calculated efficiently using a rolling calculation with bitshifting. Each reduced amino acid token is stored as 4 bits in the final representation, and the bits are stored in an 8-byte integer.

Calculations comparing kmers and number of mismatches between kmers are performed directly on the bit representations when possible. To assist with reducing noise, for each kmer, Applicant additionally added a paired kmer of the same size that is equal to the length k sequence immediately upstream from the kmer, which Applicant referred to as the paired kmer (kmer_pre). Using the paired kmer mode is optional.

Next, Applicant iterated over all L hash functions, starting with i=0. All kmers (represented as integers) are mapped to their respective hash values using the hash function Hᵢ. Hash values are matched. Buckets with more than 1 kmer are retained. Two representative protein ids (rep_proteins) are then selected for each bucket: L_rep, which is the protein id corresponding to the longest sequence in the bucket (using protein id as a tiebreaker), and H_rep, which is the protein with the largest protein id (which was randomly selected at the start), using length (in descending order) as a tiebreaker. All proteins in the bucket are then paired to each of the two representative protein ids. For each protein/rep_protein pair, if cov*cov_approx_factor*length (rep_seq)>length (protein), then remove the pair from further consideration. If paired kmer mode is used, then additionally, the paired kmers between the protein and the rep_protein are compared according to their hamming distances as computed from their bit representations. If the hamming distance divided by k is less than the min_seq_id, Applicant discarded the pair from future consideration. All remaining pairs are merged into a persistent bucketed table, and i is incremented from 0 to 1-1. The persistent bucketed table is maintained across all iterations and holds all pairs of sequences to be aligned. The bucketing is performed on the join keys during save to prevent complete repartitioning of both the persistent and update table at each iteration, drastically reducing runtime. The maximum number of pairs in the table will be K*N*L., where K is the maximum sequence length. The iterations can be performed in batches as memory and disk allows.

After all iterations have been completed, the final persistent table is used to generate alignments. Due to the massive size of the table, the task of creating alignments is split further into A iterations, where A is determined by the size of the persistent table as well as the cluster resources so as to prevent consuming the entire storage of the cluster. For each of the A iterations, j 0, . . . ,A, all rep_seq ids with a modulo equal to j are considered for alignment. Then all passing protein/rep_protein pairs are materialized using a join to a database containing the original sequences. The sequence identity between each protein and the rep protein sequences are then determined according to the user's choice of 1) a fast edit distance-based estimate of sequence identity or 2) a slower but more Smith-Waterman accurate local sequence alignment using a Blosum62 scoring matrix with a gap open penalty of 11 and a gap extension penalty of 1 (which can be adjusted if desired). Minimum sequence coverage and minimum sequence identity are then enforced according to the clustering criterion min_seq_id and cov, resulting in a set of protein/rep_protein pairs that satisfy the clustering criteria. After all A iterations have been performed and their results merged, the next step is the optional graph simplification.

The resulting set of remaining protein/rep protein pairs then form the basis for a directed graph. Graph simplification is essential for reducing long chains in the graph that result from deep homology within a family or fold of proteins. These long chains can result in very large communities detected by connected components and is thus important when clustering at low sequence identity and while using connected components for community detection. Graph simplification may not be necessary if other community detection algorithms are used.

The main graph simplification step works by clustering together nodes that are closely connected, in a way that preserves the overall structure of the graph. The first step is to create a new table of nodes and their degrees, where the degree of a node is the number of edges that it is connected to. Next, Applicant grouped the edges by their destination node and aggregate the information for each node in a list of (degree, source) tuples. The winning edge for each node is then determined as the edge with the highest degree and smallest source node.

The next step is to determine the winning edges for each source and destination node.

This is done by ranking the edges for each node based on their winning degree and then selecting the top-ranked edge. Finally, the winning edges for the source nodes are combined with the winning edges for the destination nodes to form the final set of edges for the simplified graph.

Any edges that connect a node to itself are filtered out.

After the graph simplification, a distributed connected components algorithm is performed on the graph to identify communities. Within each community, all sequences are realigned to the representative protein. Extremely large communities are processed separately to avoid skew times due to allocation to a single CPU.

The node with the max original degree in the graph (prior to simplification) within each community is selected as the megarep node. All nodes within the community that have not been aligned to the megarep node are realigned to the megarep node, forming new edges as required. For each cluster, all nodes are then sorted by the number of edges it is connected to within the cluster. The list of sorted nodes is then traversed and for each node visited that has not been assigned to a cluster, a new cluster is created consisting of all nodes that it is connected to that has not been clustered, along with itself. The final list of clusters are then combined across all communities to form the final clustering of the entire dataset. Numeric protein ids were then reassigned back to their original ids using a join.

### Clustering Comparison

UniRef100, UniRef90, and UniRef50 were downloaded from UniProt on 2022 Apr. 18 for comparing clustering methods. Clustering was performed either on UniRef90 or UniRef50 using multiple software packages as possible to compare their clustering results. For timing comparisons, different clustering software were tested on a single 32 CPU node against various random samples of differing size from UniRef50 to test their runtimes as a function of the number of input sequences. Linear models were fitted to linear/linearithmic scaling algorithms on the data transformation using a least squares fit on the log-log transformed data/model predictions (log time vs log sequences). Quadratic models were fitted in the same manner for quadratic algorithms but using a quadratic model in place of a linear model.

A 1 million sample subset of UniRef50 was generated at random. The subset was then searched against UniRef50 on a 160 CPU machine with 3.8 TB shared memory using MMSeqs2 with sensitivity of 7.5, a minimum coverage of 0.8, and maximum 100 sequences per query, with the—realign mode. For each query, the top 100 hits were then realigned to the query using the standard Smith-Waterman algorithm with a Blosum62 matrix, a gap open penalty of 11 and a gap extension penalty of 1. If the number of aligned residues in the query divided by the length of the query was less than the min_cov of 0.8, then the query/target pair was ignored. Similarly, if the number of aligned residues in the target divided by the length of the target was less than the min_cov of 0.8, then the query/target pair was ignored. Of the passing query/target pairs, the target with the maximum sequence identity was selected to be the nearest neighbor.

### CRONUS CRISPR Repeat Tool

CRONUS was implemented with python 3 and operates as follows. For main parameters, CRONUS uses the minimum number of repeats-min_repeats (default 3), the minimum repeat length-min_len (default 15), the minimum sequence identity between repeats-min_id (default 85), the maximal size of the direct repeats-max_repeat_sz (default 200), the maximum spacer length-max_spacer_len (default 200), the seed size of the initial search-seed_size (default 5), the maximum number of repeat types per array-max_repeat_types (default 2). A general description of the program is given below.

First, vmatch2 is run with the query (len N) against itself with a seed length seed_size and a min identity min_id and a max edit distance based on the min_len, min_id, and seed_size. A sparse N×N coverage matrix was then formed containing all of the pairwise matches from vmatch2 with max genomic distance of 400. Connected components were then obtained from treating the coverage matrix as an adjacency matrix. Clusters of related sequences were determined by the connected components algorithm. Match groups were formed based on the connected components, consisting of a sequence along with all of its matches if it belongs to a connected component. For each match group the following was performed: A focus region was defined as the entire region spanning the sequences found in the component plus an additional 200 bp on either side. Next, length 5 kmers from all sequences covered by the connected component are grouped into a list to serve as passable kmers. All kmers of length 5 in the focus region were then identified and counted, provided that they exist in the list of passable kmers.

Kmers with a count of 3 or more were included for further processing. For each kmer, the positions in the focus were taken into a list d′in sorted order. If med len, the median difference in adjacent positions (called diff) in d was below 15, the kmer was skipped. The spacing regularity was defined as mean (abs (diff−med_len) % med len), where % is the modulo operator. The normalized regularity was equal to the spacing regularity divided by the med len. If the normalized regularity was >0.35 or if med len>=300, then the kmers were discarded. For the remaining kmers, if any of them overlap with the positions of the initial matches from the match group, the match was retained. If the final number of matches was less than min_repeats, then the entire match group was skipped. Next, the distances between all remaining matches were calculated. If the 80th percentile of the distance between matches (defined as soft_max_neigh_dist) is greater than 2*max_spacer_len, then the entire match group was skipped. Next, the match group was filtered to remove deviating matches. Specifically, if a match on either end of the match group is more than 1.5*soft_max_neigh_dist from the rest of the list, it is removed. This is repeated until no more matches are removed.

Next, Applicant constructed a list of start and end positions for each putative DR, as determined by the current match group. Applicant then sorted these positions by their starting location. Next, Applicant applied a merging step to combine overlapping DRs into longer contiguous regions. This is done by checking if two adjacent DRs have an overlap of at least overlap_buffer base pairs, and if so, merging them together. After the DRs have been identified and merged, Applicant aligned the putative DR sequences to each other and process the alignment to determine which DRs should be extended to improve the alignment. DRs are extended if they have a critical threshold of mismatches or gaps compared to the other DRs in the alignment. The extended DRs are then re-aligned with the other DRs, and the process is repeated until either no DRs need to be extended or the maximum number of iterations is reached. Using these alignments of the putative DRs, the DRs are further refined. The full alignment is converted into an embedding matrix, E of size N*5*M, where N is the number of sequences and M is the alignment length, by setting E [i, j+5*f (x [i.j])]=1/M for each i.j, and 0 otherwise, where f sends ‘A’ to 0, ‘T’ to 1, ‘G’ to 2, ‘C’ to 3 and ‘-’ to 4. Lastly, clustering is performed using DBSCAN with the dbscan function from scikit-learn. The parameters used are eps=0.55 and p=1. The eps parameter determines the maximum distance between two samples for them to be considered as in the same neighborhood. The p parameter is the power parameter for the Minkowski metric used by DBSCAN. Using the DBSCAN clusters, the repeat boundaries are refined while considering sequence divergence. The spacer regions are then tested for minimum variability.

To limit the extent of all-to-all comparisons within each contig, contigs are split into 20 kb overlapping tiles (with 13334 bp overlaps) and CRONUS is run on each split with resulting overlapping CRISPR arrays deduplicated by prioritizing the longest arrays followed by the arrays with the smallest start site.

### Extended Discussion of FLSHclust Algorithm

Hash-based clustering algorithms divide objects into a set of elements that can be matched individually using hash functions. With proteins, a hash function bucketing scheme allows grouping proteins that share kmers. However, the efficiency of kmer-based grouping for linear time clustering decreases as sequence identity between proteins drops because kmer bucketing requires long strings of identical consecutive matches. Thus, the technique becomes less effective in grouping proteins that share low sequence similarity.

Locality sensitive hashing (LSH) is a well-established technique for approximate nearest neighbor matching (9). By expanding a single hash function into a family of hash functions, LSH allows for inexact matching of elements at the cost of false negatives, false positives, and extra computational time (FIG. 1D). While LSH eliminates the need for long consecutive identical sequence matches, the high number of false negatives can adversely affect clustering performance by causing missed matches. However, a recent theorem for binary LSH functions (functions acting on 01 bit vectors) demonstrates the possibility of implementing these functions in a way that guarantees the absence of binary LSH families that have no false negatives (14).

Applicant applied this theorem to the case of string kmers to generate false negative-free kmer LSH families and subsequently developed the FLSHclust algorithm. Using Markov-Chain Monte Carlo, Applicant generated kmer LSH functions with no false negatives. To do so for a specific kmer length, k, Applicant enumerated all possible combinations of mismatch positions up to r and computed a loss equal to the number of mismatch combinations that go undetected using the entire set of hash functions. The hash functions are then perturbed using MCMC until the loss converges.

The FLSH clustering algorithm is then constructed as follows: first, all amino acids are compressed to a reduced alphabet that groups similar amino acids together. Next, all kmers of size k are extracted from each protein. Then, for each hash function, all kmers are mapped to buckets using the output of the hash function applied to the kmers. 2 representative sequences are deterministically selected per bucket to be temporary cluster centers. All sequences in the bucket are then retrieved and aligned against the cluster centers. A graph edge is formed if the alignment between the two sequences satisfies the clustering sequence identity and minimum coverage criteria. The resulting graph of edges is then simplified using a local density-based transformation that removes weak links that would otherwise create long chains of unrelated sequences in the graph. Next, a community detection algorithm is applied to form groups of sequences. For simplicity, a PREGEL implementation of the breadth first connected components algorithm is used; however notably other algorithms could be used in this place instead, such as the Leiden algorithm (11). The most hub-like sequence from each group is selected as the initial cluster center and all sequences in the group are realigned to the initial cluster center forming new edges. Each group of sequences is then clustered using greedy clustering using the new subgraph. To increase parallelizability, the FLSHclust algorithm replaces the global greedy clustering stage included in LinClust (11) with a parallelized graph simplification and community detection algorithm followed by local greedy clustering within each detected community (FIG. 1E). The algorithm is parallelizable and is bottlenecked by the maximum size of the detected communities. Applicant implemented FLSHclust in Apache Spark, employing Apache Arrow for in-memory access to enhance fault tolerance and scalability on distributed commodity clusters. Other implementations aimed at speed as opposed to fault tolerance may yield performance improvements in other computing environments.

### Performance Comparison of CRISPR Finders on Synthetic CRISPR Array Benchmark

A synthetic benchmark was constructed using parameterized probability distributions over CRISPR arrays. These CRISPR arrays were then embedded in totally random nucleotide sequences and then used as inputs for the various CRISPR finders along with different parameter combinations for the CRISPR finders. Tasks were parallelized using Apache Spark on a compute cluster with 6400 CPUs.

Synthetic CRISPRs were generated according to the following process. Applicant started by setting the seed for the random number generator using the “trial” value. Then, Applicant generated a consensus sequence for the direct repeat (DR) by generating a random sequence of canonical bases (A, C, G, T) of length equal to the prespecified DR length. Applicant also defined a range for the DR size that is equal to [floor (dr_len*(1-rel_indel_range)), ceil (dr_len*(1+rel_indel_range))]. Similarly, Applicant defined a range for the spacer size that takes into account the spacer indel relative range, equal to [floor (dr_len*(1-spacer_rel_indel_range)), ceil (dr_len*(1+spacer_rel_indel_range))].

Next, Applicant created empty lists to hold the DR and spacer sequences generated in the loop. Applicant iterated “n_dr” times to generate the DR sequences. For each DR, Applicant started with the consensus sequence and perform mismatches by randomly selecting a base that is different from the consensus base with a probability of “p_mismatch”. Applicant then checked if an indel should be performed by randomly selecting a value between the defined range for the DR size and comparing it to the original length. If an indel should be performed, Applicant randomly selected the position(s) and type of indel(s) to be made. If an insertion is made, Applicant inserted a randomly chosen base at the selected position(s). If a deletion is made, Applicant removed the base(s) at the selected position(s). The resulting DR sequence is added to the list.

Similarly, Applicant iterated “n_dr-1” times to generate the spacer sequences between the DRs. For each spacer, Applicant started with the consensus spacer sequence and perform mismatches with a probability of “1-p_spacer_similarity”. This allows control over the level of similarity between spacer sequences. For all tests used, p_spacer_similarity is selected to be 0.3, resulting in highly differing spacer sequences. Applicant then checked if an indel should be performed by randomly selecting a value between the defined range for the spacer size and comparing it to the original length. If an indel should be performed, Applicant randomly selected the position(s) and type of indel(s) to be made. If an insertion is made, Applicant inserted a randomly chosen base at the selected position(s). If a deletion is made, Applicant removed the base(s) at the selected position(s). The resulting spacer sequence is added to the list.

The final CRISPR array is formed by interleaving the spacers into the DR sequences and then concatenating the full array into a single sequence. Then, Applicant generated left and right flanking sequences to be added to the CRISPR sequence. Then, Applicant generated a random DNA sequence of length 20000 and insert the synthetic CRISPR array at the randomly selected position in the sequence.

For each CRISPR parameter set of the 35 selected combinations, 2000 random synthetic CRISPRs embedded in random DNA sequences were created using the above procedure, and all CRISPR predictors with selected corresponding parameters were tested against the synthetic sequences, keeping track of time elapsed.

To understand the CRISPR performance of each of the finders, Applicant compiled scores for each CRISPR finder/CRISPR parameter set (condition group) pair as follows. Applicant defined the consensus sequence of a list of DRs to be the DR with the minimum average normalized edit distance to the remaining DRs, where the normalized edit distance is defined as the edit distance between DR_i and DR j divided by the minimum length of the two DRs (DR_i, DR_j). Applicant then calculated 3 simple metrics of performance: “hit” rate, “DR count error” and “boundary position error” (indels between predicted and true DR). For each synthetic CRISPR, a predicted CRISPR was considered a “hit” if it overlaps with the synthetic CRISPR interval (expanded by a 50 bp buffer). If a hit was found, then Applicant aligned the consensus of the synthetic DRs with the consensus of the predicted DRs using Biopython pairwise2 alignment module and calculated the number of gap columns in the alignment as the boundary prediction error (in bp). The absolute value difference between the number of true DRs and predicted DRs was taken to be the DR count error. Applicant then used bootstrapping with 2000 samples to calculate the mean, upper quartile, and lower quartile of the means of all three performance metrics as well as elapsed time in order to compare between all CRISPR predictors.

### Prokaryotic Genome/Metagenome CDS Prediction and Clustering

Applicant used Prodigal (1) to predict all genes in the genomes/metagenomes, using the metagenomic option for metagenomic datasets (WGS metagenomes, JGI, MG-RAST). Next Applicant deduplicated all proteins, then iteratively clustered all proteins at 100%, 98%, 90%, 70%, 50%, and 30% sequence identities with a minimum coverage of 80%. For 100%-50% sequence identity, Applicant clustered using the FLSHclust implementation without the use of LSH functions and used the LSH functions for clustering from 50% to 30% sequence identity. All 6 clusterings along with deduplication were performed with the FLSHclust implementation over the course of ˜1-2 weeks with intermittent breaks on a Hadoop cluster containing 30 compute nodes with 32CPUs, 256 GB memory and 2 TB SSD non-shared storage each.

### Sensitive CRISPR Discovery Pipeline

For CRISPR prediction, 4 CRISPR finders were used with a total of 6 different runs based on parameter combinations selected from a calibration against the synthetic CRISPR array benchmark. Parameters for each CRISPR finder are as follows:
1. PILERCR with minarray=3, mincons-0.9, minid=0.94, maxrepeat=64, maxspacer=64
2. PILERCR with minarray=3, mincons=0.8, minid=0.85, maxrepeat=128, maxspacer=128
3. CRT with minNR=3, minRL=16, maxRL=128, minSL=16, maxSL=128
4. CRISPRFinder with mNS=2
5. CRONUS with seed_size=5, min_len=16, minid=8, max_repeat_size=128
6. CRONUS with seed_size=11, min_len=16, minid=80, max_repeat_size=512

Next, Applicant deduplicated CRISPR array predictions from the various CRISPR finders as follows. For each contig, Applicant created an empty list of intervals. Applicant looped through each CRISPR and checked if it overlapped with any of the existing intervals. If it did, Applicant merged the intervals and added the CRISPR to the corresponding list of CRISPRs in the merged interval. If it did not overlap with any existing intervals, Applicant created a new interval for the CRISPR.

Finally, Applicant selected the best CRISPR from each interval by selecting the CRISPR with the most DRs, highest priority index, longest length, and earliest start position. Here, the priority index of the various CRISPR finders is determined according to the following order: [′CRISPRFinder′, ‘PILER_5’, ‘PILER_1’, ‘CRT_2’, ‘CRONUS_4’, ‘CRONUS_2’]. For consistency across the CRISPR arrays, Applicant then redefined the spacers to be the sequences interleaving the predicted DRs, as not all CRISPR prediction tools may do this.

Applicant defined the distance (interval_distance) between two genomic feature intervals (e.g., a CRISPR array, or CDS) to be equal to zero if the two intervals overlap, and otherwise the minimum distance between all combinations of the endpoints. Applicant further corrected this distance for circular contigs (e.g., plasmids and some phage, entire bacterial genomes) by using the nearest distance along a circle as opposed to a line when necessary.

Applicant then used the following procedure to define operons from the set of predicted proteins within a given contig. Applicant created two lists called “proteins_f” and “proteins_r” to store proteins in forward and reverse directions, respectively. Applicant then sorted the proteins in both lists based on their starting position for forward proteins and ending position for reverse proteins. Applicant created an empty list called “intervals_f” to store forward protein operons. Applicant looped through each forward protein in “proteins_f” and obtained its starting and ending positions. If the “intervals_f” list was empty, Applicant created a new interval with the protein and appended it to the list. Otherwise, Applicant checked if the current protein was within a distance of 200 bp to the last interval in the list. If it was, Applicant added the protein to the last operon and updated its starting and ending positions to encompass all proteins currently in the operon. If it was not, Applicant created a new interval with the protein and appended it to the list. If the DNA strand was circular and there was more than one interval in the “intervals_f” list, Applicant checked if the first and last intervals were within the maximum distance of each other. If they were, Applicant merged them into a single interval that crossed the circular boundary. Applicant repeated the above process for the reverse proteins and stored the resulting intervals in a list called “intervals_r”. The final set of operons is defined by the concatenation of “intervals f” and “intervals_r”.

Applicant then defined the operonic distance between a protein and another feature of interest as the interval_distance between the feature of interest and the operon containing the protein. Applicant then computed the operonic distance between each protein and the nearest CRISPR array. For this study, Applicant set max_association_distance=3000 to be the maximum operonic distance between a protein gene and a CRISPR array at which the protein gene is considered to be associated with a CRISPR array. For each gene, the associated CRISPR array is the CRISPR array closest to the protein within max_association_distance operonic distance from the protein, or an empty array if none exists.

Metagenomic data poses a challenge in inferring associations due to the tendency for short contigs to contain partial systems. However, the information from metagenomic data can still be leveraged for calculating associations by calculating an effective sample size for each protein that incorporates information about distances to the contig edge. For each protein, two quantities are computed, “u_d” and “d_d” which are the upstream and downstream distances to the contig edge from the operon containing the protein. Upstream and downstream are determined relative to the orientation of the protein on the contig. The contig weight for the protein is then computed as the weighted average w=0.5*min (1, u_d/max_association_distance)+0.5*min (1, d_d/max_association_distance). If the protein is not associated with a CRISPR array (i.e., there is no CRISPR array with an operonic distance of less than 3000 to the protein), the effective sample size is the contig weight. If the protein is considered to be associated with a CRISPR array, then the effective sample size is equal to 1.

Proteins are then redundancy reduced as follows. All proteins within the same c90 cluster (90% identity cluster) are grouped. The proteins are then ordered in lexicographic order using the followed ordered criteria: 1) the number of DRs in the associated CRISPR array (descending order), 2) contig weight of the protein (descending order), 3) the protein id as a tiebreaker (descending order). The rank 1 protein from each c90 cluster as determined from the above ordering is then selected to be the representative protein for the c90 cluster with regards to association calculation. The resulting set of proteins is considered to be the redundancy reduced set for the naive score calculation.

The naive association score is then calculated as follows. For each c30 cluster (30% identity cluster), three quantities were computed using all the non-redundant proteins (c90 cluster representatives defined above) contained within the 30% cluster: the number of non-redundant proteins with CRISPR associations (N_cr), and the sum of the effective sample sizes of all non-redundant proteins (N_eff), as well as the total number of non-redundant proteins (N). The naive score (referred to as weighted_icity) was then calculated to be S=N_cr/N_eff.

All 30% clusters with (weighted_icity>=0.02 and N_cr>=2) or (weighted_icity>=0.1 and N_cr>=1) were selected for performing blast searches of the respective DRs in order to calculate the enhanced score. Therefore, extremely low confidence clusters were dropped to avoid performing excessive blast searches. The remaining 30% clusters were considered candidate 30% clusters.

Applicant then searched for divergent DRs for the candidate 30% clusters as follows. All non-redundant 90% clusters were assigned to the consensus DR of the closest CRISPR array of the representative cluster member, which were selected as above. Then, for each of the 30% clusters, the consensus DRs of all 90% clusters were collected into a list. For each protein belonging to the candidate 30% clusters, the 10 kb vicinity was searched against all of the consensus DRs from the 30% cluster using an expect value of le-3 (with the db size equal to the 10kbp window around the protein, accounting for reduced size when necessary due to short contigs), and a word size of 7, a gap open penalty of 6, and a gap extension penalty of 2. Applicant additionally required a minimum coverage of 0.4 to the DR to be considered a divergent DR, that is match length/DR length was required to be >=0.4. Furthermore, Applicant required that all matches be >=16 bp to reduce noise. All remaining matches were considered divergent DRs (alternatively referred to as “searched DRs”). Enhanced CRISPR scores were then calculated in the same manner as the naive score, but using the “searched DRs” in place of the CRISPR arrays when computing all quantities, namely presence of CRISPR within the vicinity (“searched DRs” within 3kbp of the protein) and effective sample size, with the caveat that only “searched DRs” for a given 30% cluster were considered for scoring that cluster (i.e., “searched DRs” for cluster i would not be used to count towards the scores for cluster j).

### Appearance of CRISPR Systems in the Public Dataset

All projects from NCBI, JGI, WGS, and EMBL were mapped to their project add dates whenever possible. Projects that could not be matched were discarded from analysis. All predicted proteins were then assigned a date based on the add date of the corresponding project. The appearance of a cluster in the public data was defined as follows. All proteins with an upstream or downstream distance to the contig edge of <500 bp were discarded due to their low quality and likely presence of protein truncations from having incomplete contigs. Then all proteins with a distance of <=10000 to a predicted CRISPR array were retained. For each 90% cluster, the minimum add date of all proteins within the cluster was taken to be the appearance date of the 90% cluster (referred to as non-redundant protein). This step constitutes the redundancy reduction step for CRISPR-associated non-redundant proteins in the calculation.

Afterward, for each 30% cluster, all of the CRISPR-associated non-redundant proteins were obtained, and their appearance dates sorted. The date of the 4th row in the sorted list was designated the appearance date of the cluster in the public data (corresponding to the minimum date at which 4 or more CRISPR-associated non-redundant proteins existed in the public data. The appearance date of a given CRISPR system was then defined as the minimum appearance date of all clusters from the system, as defined by a given signature gene. The signature gene used for defining the VII system are as follows: β-CASP.

### Spacer Search for Selected CRISPR Systems

CRISPR spacers were obtained from predicted CRISPR arrays, including the left and right cryptic “spacers” outside of the array by extending 30 bp past the first and last DRs. A consensus DR was obtained for each CRISPR array by taking the DR with the minimum average normalized edit distance to all the other DRs in the array, where Applicant define normalized edit distance as the edit distance between DR_i and DR j divided by the maximum length of DR_i and DR j. Each spacer was assigned a representative DR equal to the consensus DR for the CRISPR array it originated from.

Spacers were then searched against the entire genomic/metagenomic database using BLASTN with an e-value cutoff of 1 and word size of 11, and effective database size of 8871099067954. The effective database size was used because the BLASTN search was performed by distributing the query identically over all nodes in the cluster and then using each cluster node to blast the query against different subsets of the full database, such that the full database was searched against across all nodes. Instead of an e-value cutoff, a bit score threshold of 48 was used as a minimum for a significant match. A threshold was selected as opposed to an e-value cutoff to reduce the effect of database size on the search, however, the database size still affected the number of candidate hits below the e-value cutoff of 1. For each query, all target matches were then expanded by 100 bp in each direction. The representative DR for the spacer was then searched against the expanded region using BLASTN with a word size of 7. A target was then considered a self-match if any of the DR representative blast hits occurred within 100 bp of the spacer target match with a minimum bit score of 24. Here, a self-match is a spacer match to a locus that is identical or similar to the original CRISPR array containing the spacer. All target matches with self-matches as defined above were removed. All remaining matches were considered significant matches to the spacer sequence that are considered unrelated to the original locus.

### Expression and Purification of Candidate Type VII Proteins and RNP Complexes

To purify the candidate type VII Cas7/Cas5 RNP complex and Cas15 protein, cognate crRNAs were cloned into a pET45b (+) vector under control of a T7 promoter by Gibson assembly. The E. coli codon optimized operon was synthesized by Twist Biosciences and cloned into the crRNA-containing pET45b (+) backbone by Gibson assembly. A His14 tag was inserted at the N-terminus of the Cas5 gene and a TwinStrep tag was inserted at the N-terminus of the Cas15 gene by Gibson assembly. Proteins were expressed as described above using media supplemented only with 100 μg/ml ampicillin and cell paste was resuspended in His lysis buffer for Cas7/Cas5 RNP complexes (50 mM Tris-HCl pH 8, 250 mM NaCl, 5% glycerol, 40 mM imidazole and 5 mM β-mercaptoethanol) or Strep lysis buffer for Cas15 proteins (50 mM Tris-HCl pH 8, 250 mM NaCl, 5% glycerol, 5 mM B-mercaptoethanol). Cells were lysed and lysate was cleared as described, then the soluble fraction was mixed with Ni Sepharose 6 Fast Flow Affinity Chromatography Media (Cytiva) at 4° C. for Cas7/Cas5 RNP complexes or Strep-Tactin Superflow Plus resin (Qiagen) at 4° C. for Cas15. Each resin was washed on a gravity flow column as described above with the modification for Cas15 that imidazole was removed for all buffers. Bound RNP was then eluted in His elution buffer (50 mM Tris pH 8, 500 mM NaCl, 5% glycerol, 300 mM imidazole and 5 mM β-mercaptoethanol) or Strep elution buffer (50 mM Tris-HCl pH 8, 500 mM NaCl, 5% glycerol, 5 mM desthiobiotin and 5 mM β-mercaptoethanol) as appropriate and dialyzed overnight into dialysis buffer (20 mM Tris-HCl pH 8, 250 mM NaCl, 5% glycerol and 1 mM DTT).

### Small RNA Sequencing

RNPs: Small RNA sequencing of RNPs was performed as previously described (23). Briefly, 300 μL of protein elute was mixed with 900 μL TRI reagent (Zymo) and incubated at room temperature for 5 min. 180 μL of chloroform (Sigma Aldrich) was added and the samples were mixed gently and incubated at room temperature for an additional 3 min, then spun at 12000×g for 15 min at 4° C. The aqueous phase was used as input for RNA extraction using a Direct-zol RNA miniprep plus kit (Zymo), with in-column DNAse treatment. The purified RNA was then subject to treatment as per manufacturer's instructions with 20 U of T4 PNK (NEB) for 1 hour at 37° C., then 20 U of RNA 5′ polyphosphatase (Biosearch Technologies) for 30 min at 37° C., with each enzymatic step followed by cleanup with a Zymo RNA Clean & Concentrator-5 kit as per the manufacturer's instructions. Following enzymatic treatments, purified RNA was subject to library preparation with an NEBNext Multiplex Small RNA Library Prep kit (NEB) as per the manufacturer's instructions, with an extension time of 1 min and 12 cycles in the final PCR. Amplified libraries were gel extracted, quantified by qPCR using a KAPA Library Quantification Kit for Illumina (Roche) on a CFX Opus 384 (BioRad) and sequenced on an Illumina MiSeq with Read 1 45 cycles, Read 2 45 cycles and Index 1 6 cycles. Adapters were trimmed using CutAdapt (6) and mapped to loci of interest using BWA. Filled reads were obtained and filled reads filtered as indicated were visualized using a custom Python script (23).

### In Vitro Candidate Type VII RNA Cleavage Assays

Templates for in vitro transcription of target RNAs were produced by PCR amplification of pUC19 plasmids containing the target site. RNA was in vitro transcribed using the PCR templates with a HiScribe T7 Quick High Yield RNA Synthesis kit (NEB). 50 pmol of target RNA was heated for 3 min at 65° C., then labeled with 100 pmol of pCp-Cy5 (Jena Biosciences) using 68 U T4 RNA ligase 1, ssRNA ligase (High Concentration) (NEB) in a 1X buffer of 50 mM Tris-HCl pH 7.5, 10 mM MgCl2, 2 mM DTT, 2 mM ATP and 10% DMSO at 4° C. overnight and purified using a Zymo RNA Clean & Concentrator-5 column as per the manufacturer's instructions. Target cleavage assays contained 25 nM of labeled RNA substrate, ˜1 μM of Cas7/Cas5 RNP and ˜2 μM of Cas15 protein in a final 1x reaction buffer of 20 mM HEPES pH 8, 100 mM KCl, 40 mM NaCl, 1 mM MgCl₂, and 1 mM ATP. Assays were allowed to proceed at 45° C. or temperature as specified for 1 hour.

Reactions were then treated with 1.6 U of Proteinase K (NEB) at room temperature for 15 min. RNA was resolved by gel electrophoresis on Novex 10% TBE-Urea polyacrylamide gels (Thermo Fisher Scientific) as specified and imaged using a BioRad ChemiDoc imaging system.

### Size Exclusion Chromatography

Candidate Type VII Cas7/Cas5 complexes were purified as described, with the following modifications: buffer A contained 500 mM NaCl, buffer B contained 250 mM NaCl, and elution buffer contained 250 mM NaCl. The elution was directly diluted into to achieve a final concentration of 20 mM Tris-HCl pH 8, 150 mM NaCl, 100 mM imidazole and 3.6% glycerol. The sample was concentrated to 500 μL using a Vivaspin 20 column with a molecular weight cutoff of 50 kDa (Cytiva) and purified through a Superose 6 Increase 10/300 GL column (Cytiva) in 20 mM Tris-HCl pH 8, 150 mM NaCl, 1% glycerol. Fractions were collected and analyzed by SDS-PAGE gel electrophoresis.

### REFERENCES FOR EXAMPLE 1

1. J. Y. Wang, J. A. Doudna, CRISPR technology: A decade of genome editing is only the beginning. Science. 379, eadd8643 (2023).
2. S. A. Shmakov, G. Faure, K. S. Makarova, Y. I. Wolf, K. V. Severinov, E. V. Koonin, Systematic prediction of functionally linked genes in bacterial and archaeal genomes. Nature Protocols. 14 (2019), pp. 3013-3031.
3. W. X. Yan, P. Hunnewell, L. E. Alfonse, J. M. Carte, E. Keston-Smith, S. Sothiselvam, A. J. Garrity, S. Chong, K. S. Makarova, E. V. Koonin, D. R. Cheng, D. A. Scott, Functionally diverse type V CRISPR-Cas systems. Science. 363 (2019), pp. 88-91.
4. S. Shmakov, O. O. Abudayyeh, K. S. Makarova, Y. I. Wolf, J. S. Gootenberg, E. Semenova, L. Minakhin, J. Joung, S. Konermann, K. Severinov, F. Zhang, E. V. Koonin, Discovery and functional characterization of diverse Class 2 CRISPR-Cas systems. Mol. Cell. 60, 385 (2015).
5. F. Hille, H. Richter, S. P. Wong, M. Bratovič, S. Ressel, E. Charpentier, The biology of CRISPR-Cas: Backward and forward. Cell. 172, 1239-1259 (2018).
6 K. S. Makarova, Y. I. Wolf, J. Iranzo, S. A. Shmakov, O. S. Alkhnbashi, S. J. J. Brouns, E. Charpentier, D. Cheng, D. H. Haf\acute{t}, P. Horvath, S. Moineau, F. J. M. Mojica, D. Scott, S. A. Shah, V. Siksnys, M. P. Terns, Č. Venclovas, M. F. White, A. F. Yakunin, W. Yan, F. Zhang, R. A. Garrett, R. Backofen, J. van der Oost, R. Barrangou, E. V. Koonin, Evolutionary classification of CRISPR-Cas systems: a burst of class 2 and derived variants. Nat. Rev. Microbiol. 18, 67-83 (2020).
7. S. Kannan, H. Altae-Tran, X. Jin, V. J. Madigan, R. Oshiro, K. S. Makarova, E. V. Koonin, F. Zhang, Compact RNA editors with small Cas13 proteins. Nat. Biotechnol. 40, 194-197 (2022).
8. S. Shmakov, A. Smargon, D. Scott, D. Cox, N. Pyzocha, W. Yan, O. O. Abudayyeh, J. S. Gootenberg, K. S. Makarova, Y. I. Wolf, K. Severinov, F. Zhang, E. V. Koonin, Diversity and evolution of class 2 CRISPR-Cas systems. Nat. Rev. Microbiol. 15, 169-182 (2017).
9. S. A. Shmakov, K. S. Makarova, Y. I. Wolf, K. V. Severinov, E. V. Koonin, Systematic prediction of genes functionally linked to CRISPR-Cas systems by gene neighborhood analysis. Proc. Natl. Acad. Sci. U.S.A 115, E5307-E5316 (2018).
10. UniProt Consortium, UniProt: a worldwide hub of protein knowledge. Nucleic Acids Res. 47, D506-D515 (2019).
11. M. Steinegger, J. Söding, Clustering huge protein sequence sets in linear time. Nat. Commun. 9, 2542 (2018).
12. S. Doron, S. Melamed, G. Ofir, A. Leavitt, A. Lopatina, M. Keren, G. Amitai, R. Sorek, Systematic discovery of antiphage defense systems in the microbial pangenome. Science. 359 (2018), doi: 10.1126/science.aar4120.
13. L. Gao, H. Altae-Tran, F. Böhning, K. S. Makarova, M. Segel, J. L. Schmid-Burgk, J. Koob, Y. I. Wolf, E. V. Koonin, F. Zhang, Diverse enzymatic activities mediate antiviral immunity in prokaryotes. Science. 369, 1077-1084 (2020).
14. R. Pagh, CoveringLSH. ACM Transactions on Algorithms. 14 (2018), pp. 1-17.
15. R. C. Edgar, Search and clustering orders of magnitude faster than BLAST. Bioinformatics. 26, 2460-2461 (2010).
16. M. Steinegger, J. Söding, MMseqs2 enables sensitive protein sequence searching for the analysis of massive data sets. Nat. Biotechnol. 35, 1026-1028 (2017).
17. B. E. Suzek, Y. Wang, H. Huang, P. B. McGarvey, C. H. Wu, UniProt Consortium, UniRef clusters: a comprehensive and scalable alternative for improving sequence similarity searches. Bioinformatics. 31, 926-932 (2015).
18. A. Lomsadze, K. Gemayel, S. Tang, M. Borodovsky, Modeling leaderless transcription and atypical genes results in more accurate gene prediction in prokaryotes. Genome Res. 28, 1079-1089 (2018).
19. R. C. Edgar, PILER-CR: fast and accurate identification of CRISPR repeats. BMC Bioinformatics. 8, 18 (2007).
20. C. Bland, T. L. Ramsey, F. Sabree, M. Lowe, K. Brown, N. C. Kyrpides, P. Hugenholtz, CRISPR recognition tool (CRT): a tool for automatic detection of clustered regularly interspaced palindromic repeats. BMC Bioinformatics. 8, 209 (2007).
21. I. Grissa, G. Vergnaud, C. Pourcel, CRISPRFinder: a web tool to identify clustered regularly interspaced short palindromic repeats. Nucleic Acids Res. 35, W52-7 (2007).
22. A. Biswas, R. H. J. Staals, S. E. Morales, P. C. Fineran, C. M. Brown, CRISPRDetect: A flexible algorithm to define CRISPR arrays. BMC Genomics. 17, 356 (2016).
23. H. Altae-Tran, S. Kannan, F. E. Demircioglu, R. Oshiro, S. P. Nety, L. J. Mckay, M. Dlakić, W. P. Inskeep, K. S. Makarova, R. K. Macrae, E. V. Koonin, F. Zhang, The widespread IS200/IS605 transposon family encodes diverse programmable RNA-guided endonucleases. Science. 374, 57-65 (2021).
24. G. Faure, S. A. Shmakov, W. X. Yan, D. R. Cheng, D. A. Scott, J. E. Peters, K. S. Makarova, E. V. Koonin, CRISPR-Cas in mobile genetic elements: counter-defence and beyond. Nat. Rev. Microbiol. 17, 513-525 (2019).
25. C. Pourcel, M. Touchon, N. Villeriot, J.-P. Vernadet, D. Couvin, C. Toffano-Nioche, G. Vergnaud, CRISPRCasdb a successor of CRISPRdb containing CRISPR arrays and cas genes from complete genome sequences, and tools to download and query lists of repeats and spacers. Nucleic Acids Res. 48, D535-D544 (2020).
26. H. N. Taylor, E. E. Warner, M. J. Armbrust, V. M. Crowley, K. J. Olsen, R. N. Jackson, Structural basis of Type IV CRISPR RNA biogenesis by a Cas6 endoribonuclease. RNA Biol. 16, 1438-1447 (2019).
27. A. Özcan, P. Pausch, A. Linden, A. Wulf, K. Schühle, J. Heider, H. Urlaub, T. Heimerl, G. Bange, L. Randau, Type IV CRISPR RNA processing and effector complex formation in Aromatoleum aromaticum. Nat Microbiol. 4, 89-96 (2019).
28. R. Pinilla-Redondo, D. Mayo-Muñoz, J. Russel, R. A. Garrett, L. Randau, S. J. Sørensen, S. A. Shah, Type IV CRISPR-Cas systems are highly diverse and involved in competition between plasmids. Nucleic Acids Res. 48, 2000-2012 (2020).
29. X. Guo, M. Sanchez-Londono, J. V. Gomes-Filho, R. Hernandez-Tamayo, S. Rust, L. M. Immelmann, P. Schäfer, J. Wiegel, P. L. Graumann, L. Randau, Characterization of the self-targeting Type IV CRISPR interference system in Pseudomonas oleovorans. Nat Microbiol. 7, 1870-1878 (2022).
30. V. M. Crowley, A. Catching, H. N. Taylor, A. L. Borges, J. Metcalf, J. Bondy-Denomy, R. N. Jackson, A Type IV-A CRISPR-Cas System in Mediates RNA-Guided Plasmid Interference. CRISPR J. 2, 434-440 (2019).
31. A. Moya-Beltrán, K. S. Makarova, L. G. Acuña, Y. I. Wolf, P. C. Covarrubias, S. A. Shmakov, C. Silva, I. Tolstoy, D. B. Johnson, E. V. Koonin, R. Quatrini, Evolution of Type IV CRISPR-Cas Systems: Insights from CRISPR Loci in Integrative Conjugative Elements of. CRISPR J. 4, 656-672 (2021).
32. S. Mulepati, S. Bailey, In vitro reconstitution of an Escherichia coli RNA-guided immune system reveals unidirectional, ATP-dependent degradation of DNA target. J. Biol. Chem. 288, 22184-22192 (2013).
33. M. L. Hochstrasser, D. W. Taylor, P. Bhat, C. K. Guegler, S. H. Sternberg, E. Nogales, J. A. Doudna, CasA mediates Cas3-catalyzed target degradation during CRISPR RNA-guided interference. Proc. Natl. Acad. Sci. U.S.A 111, 6618-6623 (2014).
34 B. Zetsche, J. S. Gootenberg, O. O. Abudayyeh, I. M. Slaymaker, K. S. Makarova, P. Essletzbichler, S. E. Volz, J. Joung, J. van der Oost, A. Regev, E. V. Koonin, F. Zhang, Cpf1 is a single RNA-guided endonuclease of a class 2 CRISPR-Cas system. Cell. 163, 759-771 (2015).
35. R. E. Haurwitz, M. Jinek, B. Wiedenheft, K. Zhou, J. A. Doudna, Sequence0-and structure-specific RNA processing by a CRISPR endonuclease. Science. 329, 1355-1358 (2010).
36. J. L. Schmid-Burgk, L. Gao, D. Li, Z. Gardner, J. Strecker, B. Lash, F. Zhang, Highly Parallel Profiling of Cas9 Variant Specificity. Mol. Cell. 78, 794-800.e8 (2020).
37. Z. Dominski, A. J. Carpousis, B. Clouet-d′Orval, Emergence of the β-CASP ribonucleases: highly conserved and ubiquitous metallo-enzymes involved in messenger RNA maturation and degradation. Biochim. Biophys. Acta. 1829, 532-551 (2013).
38. C. R. Mandel, S. Kaneko, H. Zhang, D. Gebauer, V. Vethantham, J. L. Manley, L. Tong, Polyadenylation factor CPSF-73 is the pre-mRNA 3′-end-processing endonuclease. Nature. 444, 953-956 (2006).
39. D. K. Phung, C. Etienne, M. Batista, P. Langendijk-Genevaux, Y. Moalic, S. Laurent, S. Liuu, V. Morales, M. Jebbar, G. Fichant, M. Bouvier, D. Flament, B. Clouet-d′Orval, RNA processing machineries in Archaea: the 5′-3′ exoribonuclease aRNase J of the β-CASP family is engaged specifically with the helicase ASH-Ski2 and the 3′-5′ exoribonucleolytic RNA exosome machinery. Nucleic Acids Res. 48, 3832-3847 (2020).
40. M. R. Lieber, The mechanism of double-strand DNA break repair by the nonhomologous DNA end-joining pathway. Annu. Rev. Biochem. 79, 181-211 (2010).
41. I. Callebaut, D. Moshous, J.-P. Mornon, J.-P. de Villartay, Metallo-beta-lactamase fold within nucleic acids processing enzymes: the beta-CASP family. Nucleic Acids Res. 30, 3592-3601 (2002).
42. D. Moshous, I. Callebaut, R. de Chasseval, B. Corneo, M. Cavazzana-Calvo, F. Le Deist, I. Tezcan, O. Sanal, Y. Bertrand, N. Philippe, A. Fischer, J. P. de Villartay, Artemis, a novel DNA double-strand break repair/V (D) J recombination protein, is mutated in human severe combined immune deficiency. Cell. 105, 177-186 (2001).
43. J. Jumper, R. Evans, A. Pritzel, T. Green, M. Figurnov, O. Ronneberger, K. Tunyasuvunakool, R. Bates, A. Žídek, A. Potapenko, A. Bridgland, C. Meyer, S. A. A. Kohl, A. J. Ballard, A. Cowie, B. Romera-Paredes, S. Nikolov, R. Jain, J. Adler, T. Back, S. Petersen, D. Reiman, E. Clancy, M. Zielinski, M. Steinegger, M. Pacholska, T. Berghammer, S. Bodenstein, D. Silver, O. Vinyals, A. W. Senior, K. Kavukcuoglu, P. Kohli, D. Hassabis, Highly accurate protein structure prediction with AlphaFold. Nature. 596, 583-589 (2021).
44. L. You, J. Ma, J. Wang, D. Artamonova, M. Wang, L. Liu, H. Xiang, K. Severinov, X. Zhang, Y. Wang, Structure Studies of the CRISPR-Csm Complex Reveal Mechanism of Co-transcriptional Interference. _{Cell}. 176, 239-253.e16 (2019).
45. A. Özcan, R. Krajeski, E. Ioannidi, B. Lee, A. Gardner, K. S. Makarova, E. V. Koonin, O. O. Abudayyeh, J. S. Gootenberg, Programmable RNA targeting with the single-protein CRISPR effector Cas7-11. Nature. 597, 720-725 (2021).
46. S. P. B. van Beljouw, A. C. Haagsma, A. Rodríguez-Molina, D. F. van den Berg, J. N. A. Vink, S. J. J. Brouns, The gRAMP CRISPR-Cas effector is an RNA endonuclease complexed with a caspase-like peptidase. Science. 373, 1349-1353 (2021).
47. J. E. Peters, K. S. Makarova, S. Shmakov, E. V. Koonin, Recruitment of CRISPR-Cas systems by Tn7-like transposons. Proc. Natl. Acad. Sci. U.S.A 114, E7358-E7366 (2017).
48. J. Strecker, F. E. Demircioglu, D. Li, G. Faure, M. E. Wilkinson, J. S. Gootenberg, O. O. Abudayyeh, H. Nishimasu, R. K. Macrae, F. Zhang, RNA-activated protein cleavage with a CRISPR-associated endopeptidase. Science. 378, 874-881 (2022).
49. K. Kato, S. Okazaki, C. Schmitt-Ulms, K. Jiang, W. Zhou, J. Ishikawa, Y. Isayama, S. Adachi, T. Nishizawa, K. S. Makarova, E. V. Koonin, O. O. Abudayyeh, J. S. Gootenberg, H. Nishimasu, RNA-triggered protein cleavage and cell growth arrest by the type III-E CRISPR nuclease-protease. Science. 378, 882-889 (2022).
50. R. M. Harshey, Transposable Phage Mu. Microbiol Spectr. 2 (2014), doi: 10.1128/microbiolspec.MDNA3-0007-2014.
51. M. Saito, A. Ladha, J. Strecker, G. Faure, E. Neumann, H. Altae-Tran, R. K. Macrae, F. Zhang, Dual modes of CRISPR-associated transposon homing. Cell. 184, 2441-2453.e18 (2021).
52. W. Y. Wu, P. Mohanraju, C. Liao, B. Adiego-Pérez, S. C. A. Creutzburg, K. S. Makarova, K. Keessen, T. A. Lindeboom, T. S. Khan, S. Prinsen, R. Joosten, W. X. Yan, A. Migur, C. Laffeber, D. A. Scott, J. H. G. Lebbink, E. V. Koonin, C. L. Beisel, J. van der Oost, The miniature CRISPR-Cas12m effector binds DNA to block transcription. Mol. Cell. 82, 4487-4502.e7 (2022).
53. G. Kaur, A. M. Burroughs, L. M. Iyer, L. Aravind, Highly regulated, diversifying NTP-dependent biological conflict systems with implications for the emergence of multicellularity. Elife. 9 (2020), doi: 10.7554/eLife.52696.
54. A. Maikova, J. Peltier, P. Boudry, E. Hajnsdorf, N. Kint, M. Monot, I. Poquet, I. Martin-Verstraete, B. Dupuy, O. Soutourina, Discovery of new type I toxin-antitoxin systems adjacent to CRISPR arrays in Clostridium difficile. Nucleic Acids Res. 46, 4733-4751 (2018).
55 H. Altae-Tran, L. Gao, J. Strecker, R. K. Macrae, F. Zhang, Computational Identification of Repeat-Containing Proteins and Systems. ORB Discovery. 1 (2020),, doi: 10.1017/qrd.2020.14.
56. P. Mohanraju, C. Saha, P. van Baarlen, R. Louwen, R. H. J. Staals, J. van der Oost, Alternative functions of CRISPR-Cas systems in the evolutionary arms race. Nat. Rev. Microbiol. 20, 351-364 (2022).
57. M. Kazlauskiene, G. Kostiuk, Č. Venclovas, G. Tamulaitis, V. Siksnys, A cyclic oligonucleotide signaling pathway in type III CRISPR-Cas systems. Science. 357, 605-609 (2017).
58. O. Niewoehner, C. Garcia-Doval, J. T. Rostøl, C. Berk, F. Schwede, L. Bigler, J. Hall, L. A. Marraffini, M. Jinek, Type III CRISPR-Cas systems produce cyclic oligoadenylate second messengers. Nature. 548, 543-548 (2017).
59 J. T. Rostøl, W. Xie, V. Kuryavyi, P. Maguin, K. Kao, R. Froom, D. J. Patel, L. A. Marraffini, The Card1 nuclease provides defence during type III CRISPR immunity. Nature. 590, 624-629 (2021).
60. C. Rouillon, N. Schneberger, H. Chi, K. Blumenstock, S. Da Vela, K. Ackermann, J. Moecking, M. F. Peter, W. Boenigk, R. Seifert, B. E. Bode, J. L. Schmid-Burgk, D. Svergun, M. Geyer, M. F. White, G. Hagelueken, Antiviral signalling by a cyclic nucleotide activated CRISPR protease. Nature. 614, 168-174 (2023).
61. S. L. Shipman, J. Nivala, J. D. Macklis, G. M. Church, Molecular recordings by directed CRISPR spacer acquisition. Science. 353, aaf1175 (2016).
62 F. Schmidt, M. Y. Cherepkova, R. J. Platt, Transcriptional recording by CRISPR spacer acquisition from RNA. Nature. 562, 380-385 (2018).
63. D. Hyatt, G.-L. Chen, P. F. Locascio, M. L. Land, F. W. Larimer, L. J. Hauser, Prodigal: prokaryotic gene recognition and translation initiation site identification. BMC Bioinformatics. 11, 119 (2010).
64. A. B. Crawley, J. R. Henriksen, R. Barrangou, CRISPRdisco: An Automated Pipeline for the Discovery and Analysis of CRISPR-Cas Systems. CRISPR J. 1, 171-181 (2018).
65 F. W. Studier, Protein production by auto-induction in high density shaking cultures. Protein Expr. Purif. 41, 207-234 (2005).
66. M. Martin, Cutadapt removes adapter sequences from high-throughput sequencing reads. EMBnet.journal. 17 (2011), p. 10.
67. K. Clement, H. Rees, M. C. Canver, J. M. Gehrke, R. Farouni, J. Y. Hsu, M. A. Cole, D. R. Liu, J. K. Joung, D. E. Bauer, L. Pinello, CRISPResso2 provides accurate and rapid genome editing sequence analysis. Nat. Biotechnol. 37, 224-226 (2019).
68. Piotr Indyk Department of Computer Science, Stanford University, Stanford, CA, Rajeev Motwani Department of Computer Science, Stanford University, Stanford, CA, Approximate nearest neighbors, doi: 10.1145/276698.276876.
69. V. A. Traag, L. Waltman, N. J. van Eck, From Louvain to Leiden: guaranteeing well-connected communities. Sci. Rep. 9, 5233 (2019).

### Example 2—Exemplary Small Type II Polypeptides

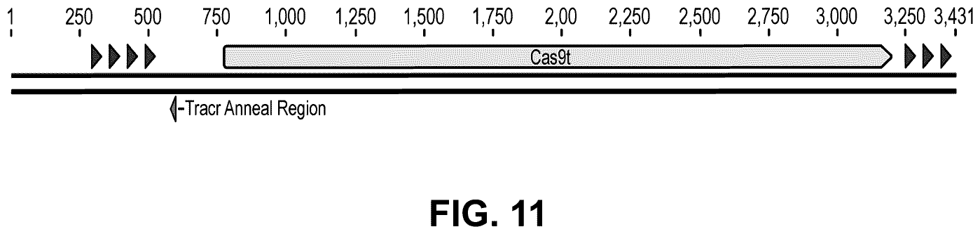

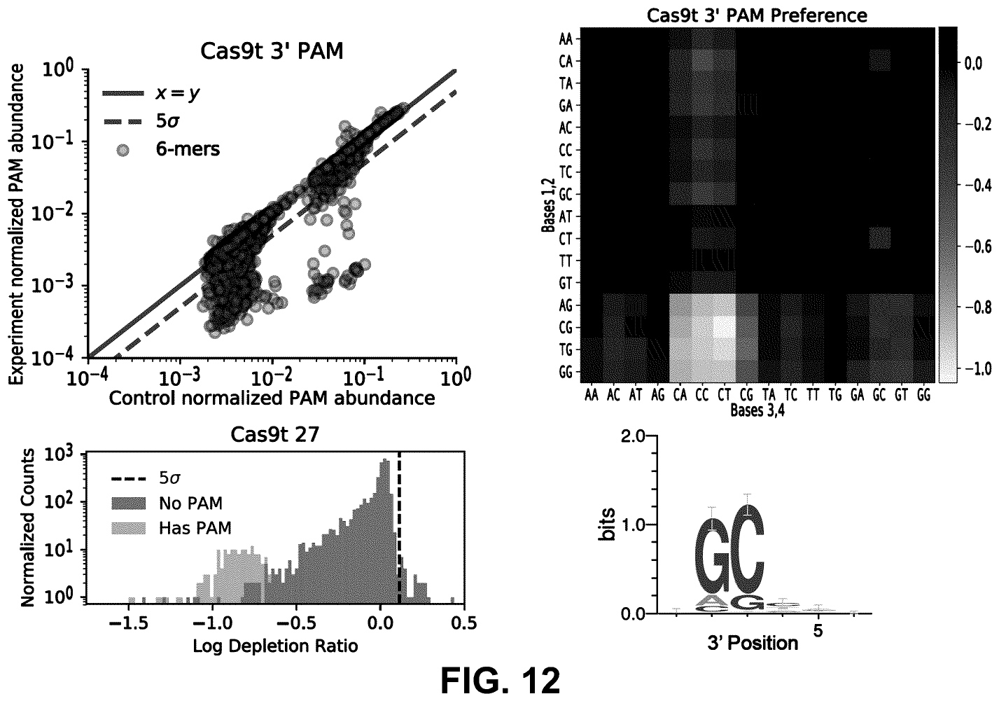

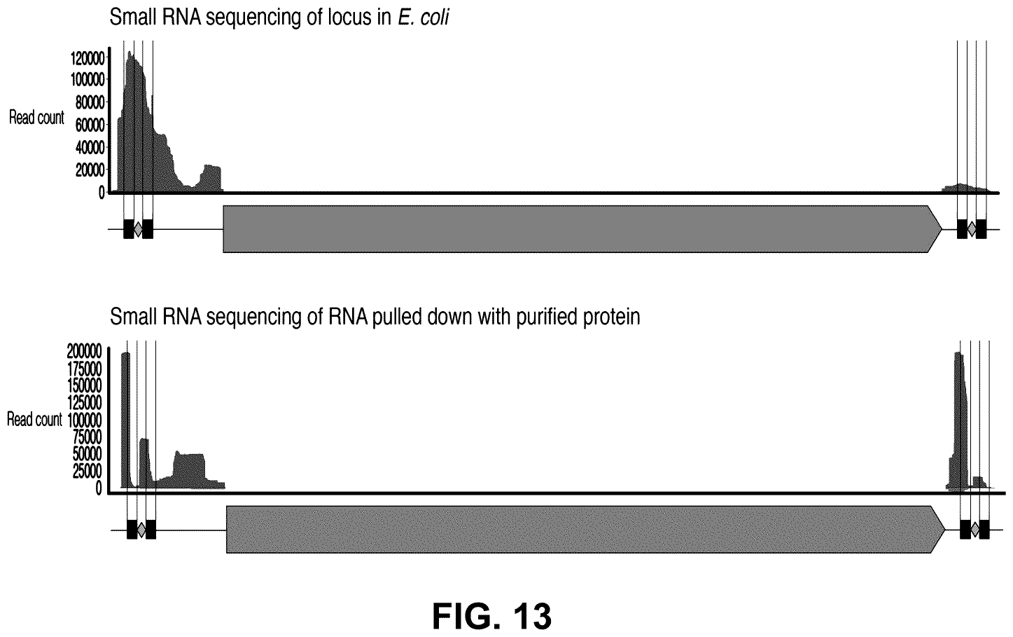

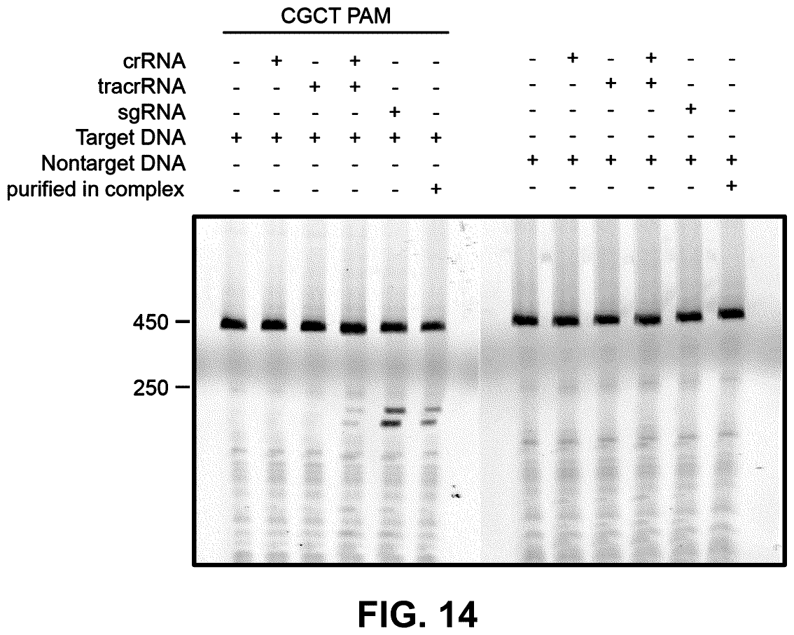

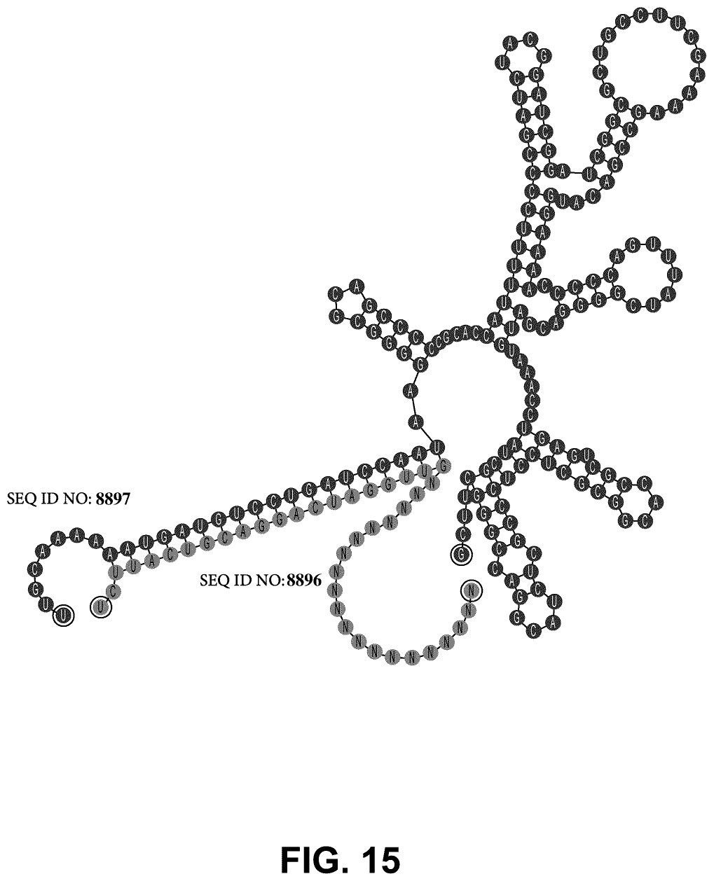

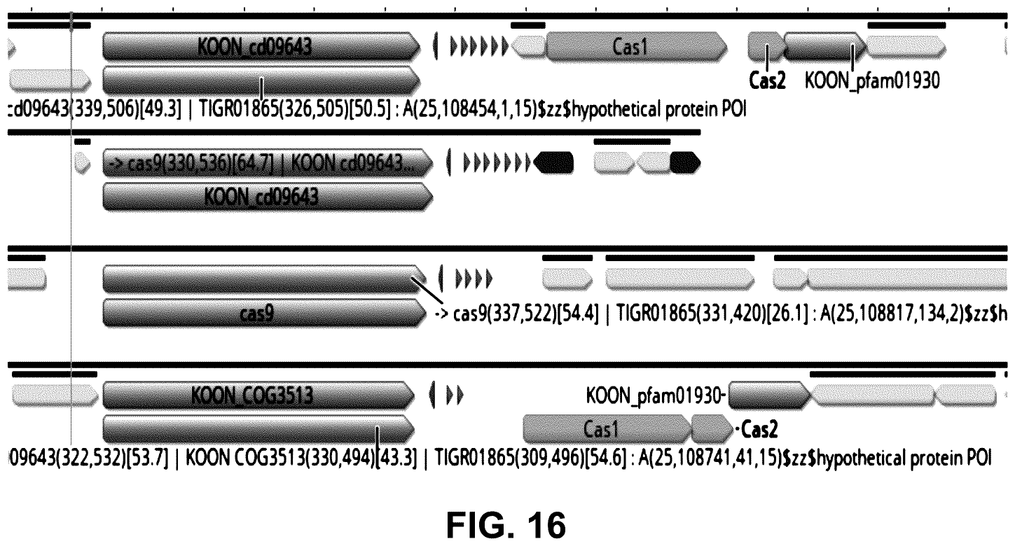

An exemplary small Type II-C Cas9 (Cas9-t) polypeptide is shown in FIG. 11. The exemplary Cas9-t has an NGCH PAM (FIG. 12). The purification pulldown shows more distinct associated small RNAs (FIG. 13). The purified Cas9-t was able to cleave DNA in vitro at 37° C. with a single guide RNA (FIG. 14). FIG. 15 shows the structure of the crRNA and tracrRNA in the form of a complex. Some exemplary small Type II-B Cas9 polypeptides are shown in FIG. 16.

### Example 3—Exemplary Methods of Identifying and Characterizing Small Type II Polypeptides

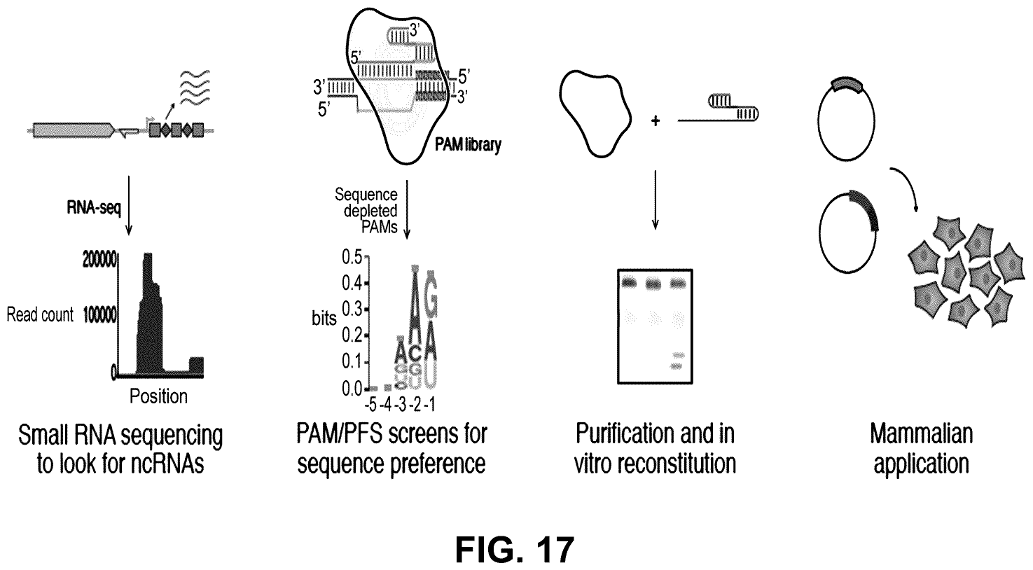

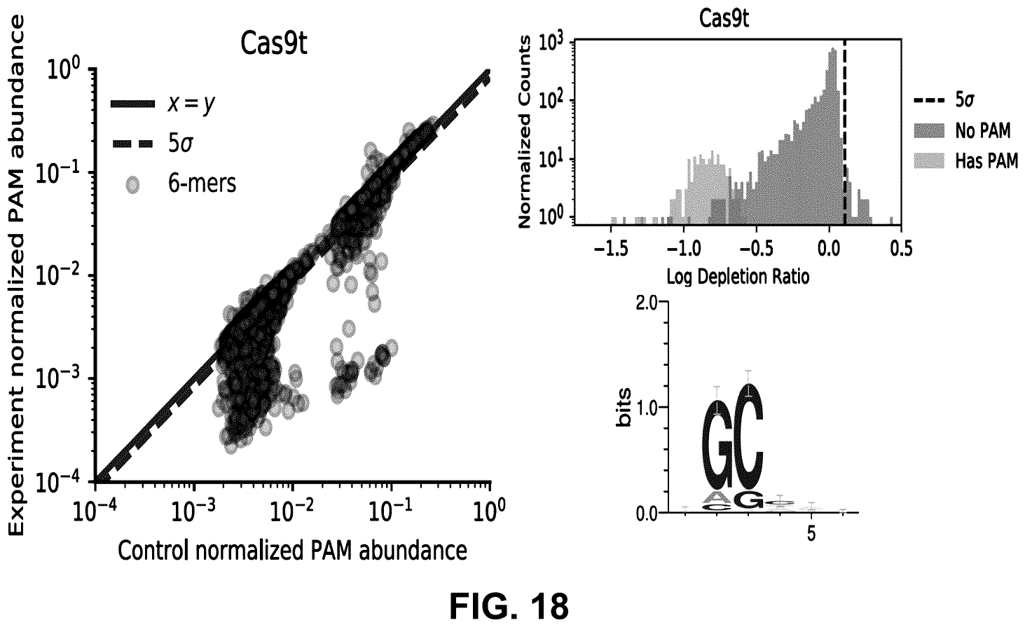

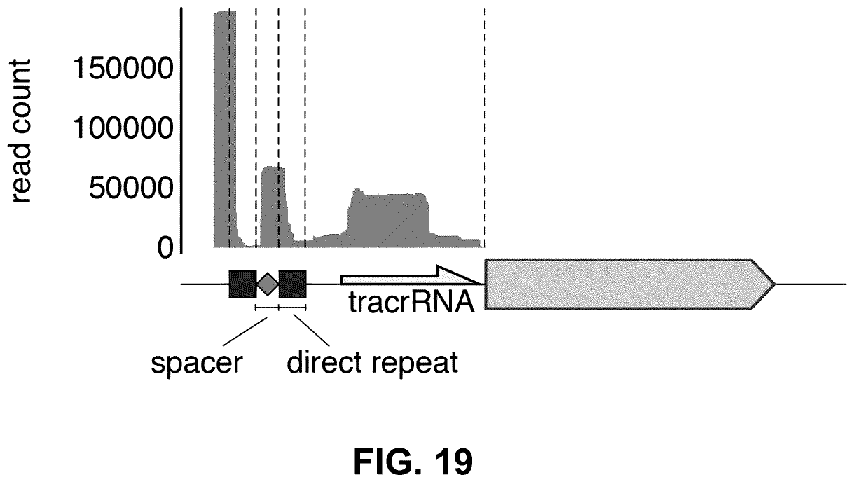

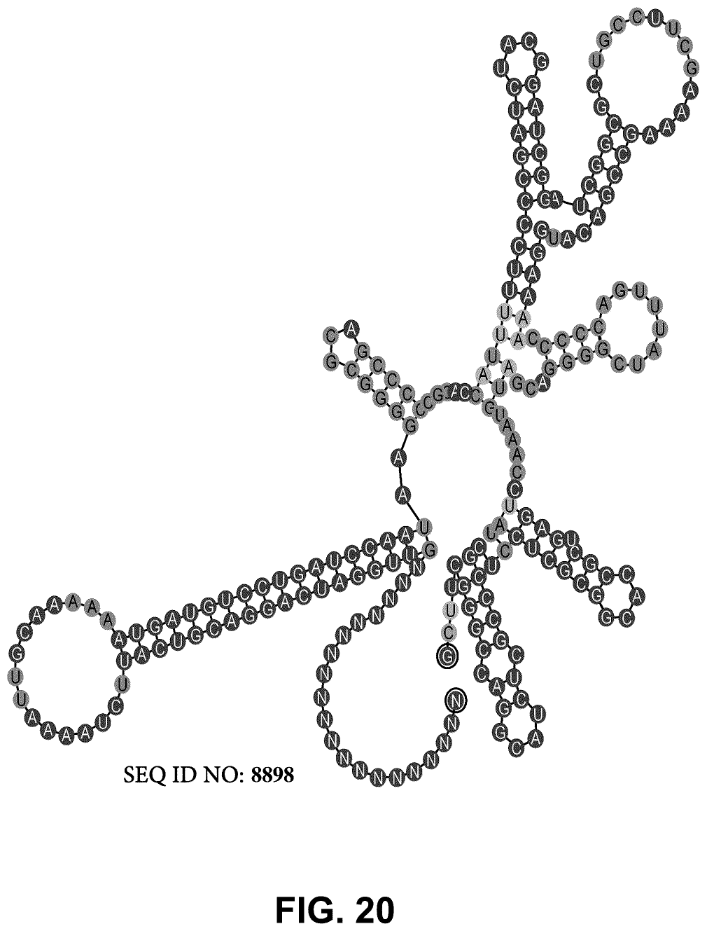

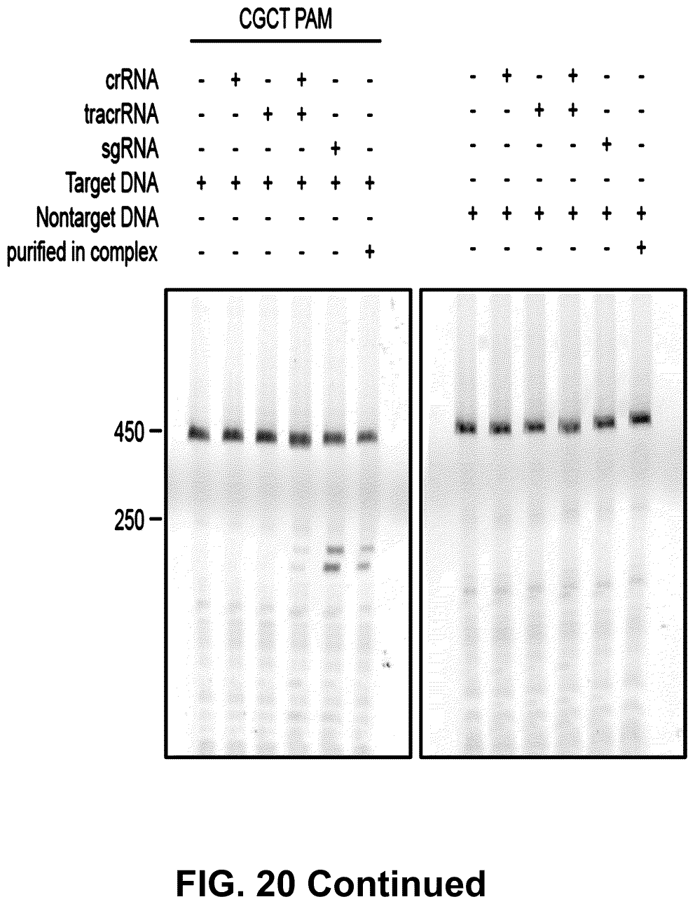

FIG. 17 shows an exemplary method of identifying and characterizing Cas polypeptides. Applicants tested the interference activity showed that an exemplary Cas9-t had interference activity with NGCH PAM (FIG. 18). Pulldown of the Cas9-t polypeptide bound to ncRNAs revealed processed CRISPR and tracrRNA (FIG. 19). The Cas9-t cleaved dsDNA in vitro using an sgRNA (FIG. 20).

### Example 4—Exemplary Type II-D Cas Polypeptides

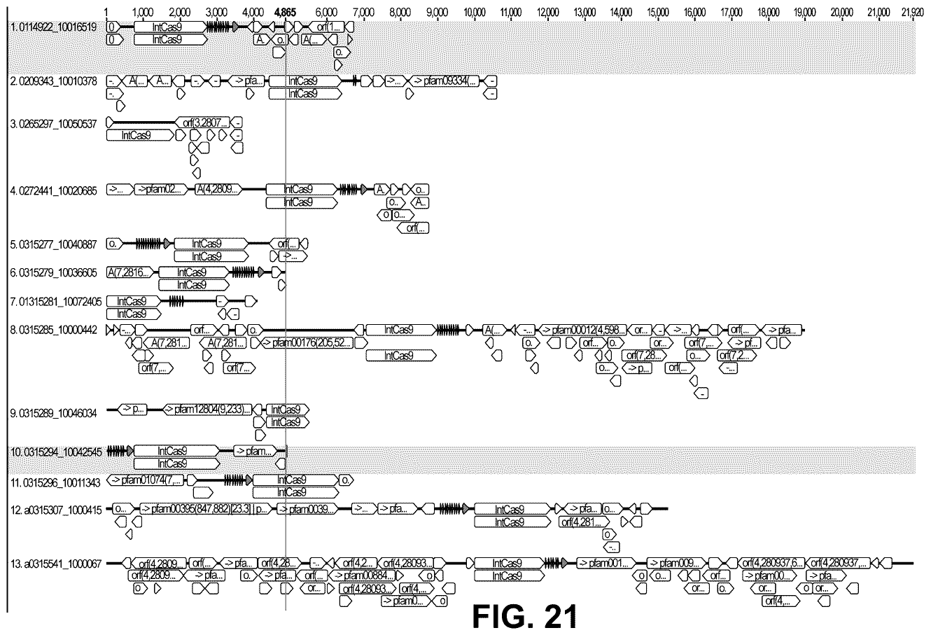

Exemplary loci for Type II-D Cas polypeptides and systems and components thereof are shown in FIG. 21. Relevant polypeptide sequences are shown in SEQ ID NO.: 8899-9520. Exemplary Type II-D Small Cas proteins can be demonstrated in this Example. Such exemplary Cas proteins are referred to in this Example “Small Cas9” and “IntCas9 (s)”. In other words, both the “Small Cas9” and “IntCas9” polypeptides and encoding polynucleotides of this Example can be demonstrative of the Type II-D small Cas polypeptides of the present invention described and provided elsewhere herein. The Small Cas9 and IntCas9 (s) demonstrated in this Example are about 950 amino acids or less in size. The exemplary IntCas9s have sizes from 650-776 amino acids. The exemplary Small Cas9s are about 950 amino acids or less.

The loci for the IntCas9s (about 650-700 amino acids) have a strong CRISPR association and tracrRNA association. The DR length is variable and was between about 29 base pairs and 36 base pairs. IntCas9s were not associated with Cas1, Cas2, Cas4, or Csn2.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A non-naturally occurring, engineered composition comprising:
(a) a β-CASP polypeptide, wherein the β-CASP polypeptide comprises an N-terminal β-CASP domain and a C-terminal adapter domain, wherein the C-terminal adapter domain comprises an α-helical domain having homology to the C-terminus of a Cas10 protein, wherein the β-CASP polypeptide comprises a plurality of residues capable of coordinating with Zⁿ²⁺ ions; and
(b) a plurality of Cas polypeptides, wherein (a) and (b) are capable of forming a non-naturally occurring, engineered multimeric CRISPR-Cas complex in the presence of a guide molecule, and wherein the guide molecule is capable of directing sequence-specific binding of the non-naturally occurring, engineered multimeric CRISPR-Cas complex to a target sequence in a target polynucleotide.

2. (canceled)

3. (canceled)

4. (canceled)

5. The composition of claim 1, wherein the plurality of Cas polypeptides comprise a Cas5 family polypeptide, a Cas7 family polypeptide, and optionally a Cas6 family polypeptide, wherein the Cas5 family polypeptide is a Type III Csx10 polypeptide, a homolog thereof, or an ortholog thereof; wherein the Cas7 family polypeptide is a Type III Csm3 polypeptide, a homolog thereof, or an ortholog thereof; and/or wherein the Cas6 family polypeptide is a Type III Cas6 polypeptide, a homolog thereof, or an ortholog thereof.

6. (canceled)

7. The composition of claim 1, wherein one or more of the β-CASP polypeptide and/or the Cas polypeptides has catalytic activity; wherein one or more of the β-CASP polypeptide and/or the Cas polypeptides lacks catalytic activity; wherein one or more of the β-CASP polypeptide and/or the Cas polypeptides is or is engineered to have nickase activity; wherein the catalytic activity is RNAse activity; wherein one or more of the β-CASP polypeptide and/or the Cas polypeptides further comprise one or more additional modifications that increase nuclease efficiency, target polynucleotide binding efficiency, or reduce off-target nuclease activity.

8. (canceled)

9. (canceled)

10. (canceled)

11. (canceled)

12. The composition of claim 1, wherein the β-CASP polypeptide and/or one or more of the Cas polypeptides is/are further linked to or otherwise capable of associating with a heterologous functional domain, wherein the heterologous functional domain is a nucleotide deaminase, a transposase, a reverse transcriptase, a recombinase, a methylase, a demethylase, an acetylase, or a deacetylase.

13. (canceled)

14. The composition according to claim 1, wherein the β-CASP polypeptide and/or one or more of the Cas polypeptides is/are derived from one or more bacteria and/or one or more archaea, wherein:
(a) the one or more bacteria each independently belong to the phylum selected from the group consisting of: Bacillota; and DTHG01000077 4 candidate division White Oak River group 3 (WOR-3), and/or the one or more bacteria each independently belong to the Staphylococcus genus, and optionally one of the bacteria is 6NBT Staphylococcus epidermis;
(b) the one or more archaea each independently belong to the phylum selected from the group consisting of MBU4492343 1/HEQ78297 1/Euryarchaeota; RLE40065.1 Candidatus Woesearchaeota; NHI92075 1 Candidatus Lokiarchaeota; and PKP54316 1 Candidatus Altiarchaeales archaeon, and/or the order selected from: PXF52022 1/RJS85311 1/Methanophagales; and MCD4797691.1/CAG0966219 1/RLG33181 1 Methanosarcinales, and/or the family MCG2727882 1 Candidatus Methanoperedenaceae, optionally WP 0972978485 1 Candidatus Methanoperedens sp BLZ2, and/or the genus WP 0972978485 1 Candidatus Methanoperedens, and/or the species selected from: 4QTS (Csm3) Methanocaldococcus jannaschii; and WP 012965105 1 Ferroglobus placidus, optionally WP 012965105 1 Ferroglobus placidus DSM 10642; and
(c) each of the β-CASP polypeptide and the one or more Cas polypeptides are optionally derived from a same species or from one or more different species, wherein optionally the β-CASP polypeptide is derived from a first species, and the one or more Cas polypeptides are derived from a second species different from the first species.

15. (canceled)

16. (canceled)

17. (canceled)

18. (canceled)

19. The composition of claim 1, further comprising one or more guide molecules, wherein the guide molecules comprise a guide sequence capable of hybridizing to a target sequence of the target molecule, and wherein the composition is optionally in the form of the non-naturally occurring, engineered multimeric CRISPR-Cas complex, wherein the at least one guide molecule is a crRNA comprising a spacer sequence flanked on the 5′ and 3′ ends by direct repeat sequences.

20. (canceled)

21. A nucleic acid molecule comprising a nucleotide sequence encoding one or more components of the composition of claim 1.

22. A vector comprising a polynucleotide comprising one or more nucleic acid molecules of claim 21, wherein the vector is a viral vector.

23. (canceled)

24. A delivery vehicle comprising one or more components of the composition of claim 1, wherein the delivery vehicle is a lipid nanoparticle, a viral capsid, an engineered retroelement vector, a polynucleotide-based nano-structure, or an extracellular contractile injection system.

25. (canceled)

26. An engineered cell comprising the composition of claim 1, wherein the engineered cell is an engineered eukaryotic cell or an engineered prokaryotic cell.

27. (canceled)

28. An organism comprising the cell according to claim 26, wherein the organism is an animal or a plant.

29. (canceled)

30. A pharmaceutical composition for treatment of a disease or disorder, comprising the composition of claim 1 and a pharmaceutically acceptable carrier.

31. A method of modifying a target polynucleotide, the method comprising contacting a sample comprising a target polynucleotide with the composition of claim 1, wherein contacting results in modification of a gene product or modification of the amount or expression of a gene product, wherein the target polynucleotide is a disease- or disorder-associated target polynucleotide.

32. (canceled)

33. (canceled)

34. A non-naturally occurring or engineered nucleic acid targeting composition comprising:
a Cas polypeptide comprising a RuvC domain and an HNH domain, wherein the Cas polypeptide is less than 850 amino acids in size, wherein the Cas polypeptide comprises one or more nuclear localization signals, two or more nuclear localization signals, and/or comprises one or more nuclear export signals, and wherein the Cas polypeptide is catalytically inactive and a nickase; and
a nucleic acid guide molecule capable of forming a complex with the Cas polypeptide and directing sequence-specific binding of the complex to a target sequence in a target polynucleotide, wherein the nucleic acid guide molecule is optionally capable of hybridizing to one or more target sequences in a prokaryotic cell or in a eukaryotic cell,
wherein the Cas polypeptide is a Type II-B Cas polypeptide selected from the group consisting of SEQ ID NOs: 189-269, or
wherein the Cas polypeptide is a Type II-C Cas polypeptide selected from the group consisting of SEQ ID NOs: 4583-8895.

35. (canceled)

36. (canceled)

37. (canceled)

38. (canceled)

39. (canceled)

40. (canceled)

41. (canceled)

42. (canceled)

43. The composition of claim 34, wherein the Cas polypeptide is associated with one or more functional domains; wherein the one or more functional domains comprises one or more heterologous functional domains; wherein the one or more functional domains cleaves the target sequence; wherein the one or more functional domains modifies transcription or translation of the target sequence the one or more functional domains comprises one or more transcriptional activation domains, optionally VP64; the one or more functional domains comprises one or more transcriptional repression domains, optionally a KRAB domain or a SID domain; the one or more functional domains comprises one or more nuclease domains, optionally Fok1; and the one or more functional domains has one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity.

44. (canceled)

45. (canceled)

46. (canceled)

47. (canceled)

48. (canceled)

49. (canceled)

50. (canceled)

51. (canceled)

52. (canceled)

53. (canceled)

54. The composition of claim 34, further comprising a recombination template, wherein the recombination template is inserted by homology-directed repair (HDR), and wherein the composition further comprises a tracrRNA.

55. (canceled)

56. (canceled)

57. The composition of claim 34, wherein the Cas polypeptide is a chimeric protein comprising a first fragment from a first Cas polypeptide and a second fragment from a second Cas polypeptide.

58. The composition of claim 34, further comprising a nucleotide deaminase or a catalytic domain thereof, wherein the nucleotide deaminase is an adenosine deaminase; wherein the nucleotide deaminase or catalytic domain thereof is covalently or non-covalently linked to the Cas polypeptide or the nucleic acid guide molecule, or is adapted to link thereof after delivered to a cell; wherein the nucleotide deaminase or catalytic domain thereof has been modified to increase its activity against a DNA-RNA heteroduplex to reduce off-target effects; wherein the composition is capable of modifying one or more nucleotides in the target sequence; wherein modification of the one or more nucleotides in the target sequence remedies a disease caused by a G→A or C→T point mutation or a pathogenic SNP, wherein the disease is cancer, haemophilia, beta-thalassemia, Marfan syndrome, or Wiskott-Aldrich syndrome; wherein modification of the one or more nucleotides in the target sequence remedies a disease caused by a T→C or A→G point mutation or a pathogenic SNP; modification of the one or more nucleotides at the target sequence inactivates a gene; and modification of the one or more nucleotides modifies gene product encoded at the target sequence or expression of the gene product.

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. The composition of claim 34, further comprising a reverse transcriptase or functional fragment thereof.

71. A non-naturally occurring or engineered nucleic acid targeting composition comprising one or more polynucleotide sequences encoding
wherein the Cas polypeptide comprises a RuvC domain and an HINH domain, wherein the Cas polypeptide is less than 900 amino acids in size; and
a nucleic acid guide molecule capable of forming a complex with the Cas polypeptide and directing sequence-specific binding of the complex to a target sequence in a target polynucleotide,
wherein the one or more polynucleotide sequences encode a Type II-B Cas polypeptide and are selected from the group consisting of SEQ ID NOs: 108-188, or
wherein the one or more polynucleotide sequences encode a Type II-C Cas polypeptide and are selected from the group consisting of SEQ ID NOs: 270-4582, wherein the one or more polynucleotide sequences are codon optimized to express in a eukaryote, wherein the one or more polynucleotide sequences is mRNA, wherein the one or more polynucleotide sequences further encode a reverse transcriptase or functional fragment thereof.

72. (canceled)

73. (canceled)

74. (canceled)

75. A vector system comprising the one or more polynucleotide sequences of claim 71, wherein the vector system comprises:
a first regulatory element operably linked to the polynucleotide sequence encoding the Cas polypeptide; and
a second regulatory element operably linked to the polynucleotide sequence encoding the nucleic acid guide molecule,
wherein the first and/or second regulatory element is a promoter, a minimal promoter, a Mecp2 promoter, tRNA promoter, or U6 promoter, and
wherein the vector system is comprised in a single vector;
the one or more vectors comprises viral vectors; and
the one or more vectors comprises retroviral, lentiviral, adenoviral, adeno-associated, or herpes simplex viral vectors.

76. (canceled)

77. (canceled)

78. (canceled)

79. (canceled)

80. (canceled)

81. (canceled)

82. (canceled)

83. A delivery system comprising the system of claim 34 and a delivery vehicle, wherein the delivery vehicle comprises lipids, sugars, metals, proteins, liposomes, nanoparticles, exosomes, microvesicles, nucleic acid nanoassemblies, a gene gun, an implantable device, or a vector system; and wherein the delivery vehicle comprises ribonucleoproteins.

84. (canceled)

85. (canceled)

86. A cell comprising the composition of claim 34, wherein the cell is a eukaryotic cell, a human or non-human animal cell, a therapeutic T cell, antibody-producing B-cell, a stem cell, or a plant cell.

87. (canceled)

88. A tissue, organ, or organism comprising the cell of claim 86, or a cell product from the cell of claim 86.

89. (canceled)

90. A method of modifying one or more target sequences, the method comprising contacting the one or more target sequences with a composition of claim 34,
wherein the composition further comprises a recombination template, and wherein modifying the one or more target sequences comprises insertion of the recombination template or a portion thereof;
wherein the one or more target sequences is in a prokaryotic cell; or
the one or more target sequences is in a eukaryotic cell; or
the one or more target sequences is comprised in a nucleic acid molecule in vitro.

91. (canceled)

92. (canceled)

93. (canceled)

94. (canceled)

95. A cell obtained from the method of claim 90, or progeny thereof, wherein the cell is a eukaryotic cell, a human or non-human animal cell, a therapeutic T cell, antibody-producing B-cell, a stem cell, or a plant cell, or a non-human animal or plant comprising the modified cell or progeny thereof.

96. (canceled)

97. (canceled)

98. A modified cell or progeny thereof of claim 95 for use in therapy.

99. A method of treating a disease, disorder, or infection comprising administering an effective amount of the composition of claim 34 in a subject in need thereof.

100. A method of identifying a trait of interest in an organism where the trait of interest is encoded by one or more target polynucleotides, the method comprising contacting the organism or a sample therefrom comprising polynucleotides with non-naturally occurring or engineered nucleic acid targeting composition of claim 34, wherein the composition is directed to the one or more target polynucleotides by the nucleic acid guide molecule, whereby one or more target polynucleotides, and thereby one or more traits, are identified, wherein the one or more target polynucleotides are modified by the non-naturally occurring or engineered nucleic acid targeting composition;
wherein the method is performed in vitro, in situ, ex vivo, or in vivo;
wherein the organism is a plant, non-human animal, or human; and
wherein for a plant organism, the method comprises contacting a plant cell with the composition, thereby either modifying or introducing a gene of interest, and regenerating a plant from the plant cell.

101. (canceled)

102. (canceled)

103. (canceled)

104. (canceled)

105. An engineered nucleic acid targeting composition comprising:
a Cas polypeptide comprising a RuvC domain and an HNH domain, wherein the Cas protein is about 950 amino acids or less in size, less than or equal to 780 amino acids in size, wherein the Cas polypeptide has no association with Cas1, Cas2, Cas4, or Csn2, wherein optionally the Cas protein is operably coupled to one or more nuclear localization signals and/or one or more nuclear export signals, and wherein optionally the Cas protein lacks one or more catalytic activities, lacks nuclease activity, or is a nickase; and
a nucleic acid guide molecule capable of forming a complex with the Cas polypeptide and directing sequence-specific binding of the complex to a target sequence in a target polynucleotide, wherein the Cas polypeptide is capable of forming a complex with two or more nucleic guide molecules, wherein each guide molecule is capable of sequence-specific binding of a target nucleic acid sequence, wherein each target sequence is different and wherein the target sequences are on the same or are on different target polynucleotides, and wherein the guide molecule or the two or more guide molecules are capable of sequence-specific binding a target sequence in vitro, in situ, ex vivo, or in vivo, and/or in a prokaryotic cell, eukaryotic cell, a virus, or a combination thereof,
wherein the Cas polypeptide is selected from the group consisting of SEQ ID NOs: 8899-9520, and wherein the Cas protein is operably coupled to or associated with one or more functional domains, one or more heterologous functional domains, wherein the one or more functional domains has one or more activities selected from deaminase activity, methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, nucleic acid binding activity, transposition activity, reverse transcription activity, or a combination thereof, and wherein the one or more functional domains is capable of cleaving the target polynucleotide and/or modifying transcription or translation of the target polynucleotide.

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. The engineered nucleic acid targeting system of claim 105, further comprising a recombination template,
wherein the recombination template is operably coupled to, complexed with, or is associated with the Cas protein, the nucleic acid guide molecule, or both;
wherein the recombination template is a homology-directed repair (HDR) recombination template;
the nucleic acid targeting system comprises a tracrRNA; and
wherein the Cas protein is a chimeric protein comprising a first polypeptide fragment from a first Cas protein and a second polypeptide fragment from a second Cas protein.

123. (canceled)

124. (canceled)

125. (canceled)

126. (canceled)

127. The engineered nucleic acid targeting system of claim 105, further comprising a deaminase or catalytic domain thereof,
wherein the deaminase is an adenosine deaminase or a cytidine deaminase;
wherein the deaminase or catalytic domain thereof is operably coupled to, complexed with, or otherwise associated with the Cas protein, a guide molecule, or both or is capable of operably coupling to, complexing with, or otherwise associated with the Cas protein, a guide molecule, or both after delivery to a cell;
wherein the nucleotide deaminase or catalytic domain thereof has been modified to increase its activity against a DNA-RNA heteroduplex, to reduce off-target effects, or both; and
wherein the system further comprising a reverse transcriptase or functional domain thereof, wherein the reverse transcriptase or functional domain thereof is optionally operably coupled to, is capable of complexing with, or is otherwise associated with the Cas protein, the guide molecule, or both.

128. (canceled)

129. (canceled)

130. (canceled)

131. (canceled)

132. The engineered nucleic acid targeting system of claim 105, further comprising one or more nucleic acid guide molecules, wherein each of the one or more nucleic acid guide molecules is capable of capable of forming a complex or is complexed with the Cas protein, and wherein each of the one or more nucleic acid guide molecules is capable of sequence specific binding of a target sequence in a target polynucleotide,
wherein the engineered nucleic acid targeting system is capable of modifying a sequence of the target polynucleotide;
wherein the modification is: (a) insertion of one or more polynucleotides; (b) deletion of one or more polynucleotides; (c) conversion of a C•G base pair to a T•A base pair; (d) conversion of an A•T base pair to a G•C base pair; or (e) a combination thereof;
wherein the modification alters a transcription product of the target polynucleotide, a translation product of the target polynucleotide, or both; and
wherein the modification alters transcription, translation, or both of the target polynucleotide.

133. (canceled)

134. (canceled)

135. (canceled)

136. (canceled)

137. A polynucleotide comprising one or more nucleic acid sequences that encode one or more components of the engineered nucleic acid system of claim 105, wherein the polynucleotide is codon optimized for expression in a eukaryotic cell, and wherein the eukaryotic cell is a human cell or a non-human animal cell.

138. (canceled)

139. (canceled)

140. A vector system comprising:
one or more vectors comprising one or more polynucleotides of claim 137, and optionally one or more regulatory elements operably coupled to one or more polynucleotides, wherein the one or more of the one or more vectors are viral vectors; and wherein the viral vector(s) is/are a retroviral vector(s), lentiviral vector(s), adenoviral vector(s), adeno-associated viral vector(s), herpes simplex viral vector(s), or a combination thereof.

141. (canceled)

142. (canceled)

143. (canceled)

144. (canceled)

145. (canceled)

146. (canceled)

147. (canceled)

148. (canceled)

149. A method of modifying one or more target polynucleotides, the method comprising contacting the one or more target polynucleotides with an engineered nucleic acid targeting system of claim 105, wherein the engineered nucleic acid targeting system is directed to the one or more target sequences by the guide nucleic acid guide molecule(s) of the engineered nucleic acid targeting system, whereby one or more target polynucleotides is/are modified,
the modification comprises: (a) insertion of one or more polynucleotides; (b) deletion of one or more polynucleotides; (c) conversion of a C•G base pair to a T•A base pair; (d) conversion of an A•T base pair to a G•C base pair; or (e) a combination thereof;
wherein contacting occurs in vitro, in situ, ex vivo, or in vivo; and
wherein contacting occurs within a cell.

150. (canceled)

151. (canceled)

152. (canceled)

153. A modified polynucleotide or modified cell or progeny thereof produced from a method as in claim 149,
wherein the cell is a eukaryotic cell or progeny thereof;
wherein the cell or progeny thereof is a human cell or progeny thereof or a non-human animal cell or progeny thereof; and
wherein the cell or progeny thereof is a plant cell.

154. (canceled)

155. (canceled)

156. (canceled)

157. (canceled)

158. A method of treating and/or preventing a disease, condition, or a symptom thereof in a subject or cell thereof, the method comprising:
modifying one or more target polynucleotides in or from the subject or cell thereof by contacting the one or more target polynucleotides with an engineered nucleic acid targeting system of claim 105, wherein the engineered nucleic acid targeting system is directed to the one or more target sequences in one or more target polynucleotides by the guide nucleic acid guide molecule(s) of the engineered nucleic acid targeting system, whereby one or more target polynucleotides is/are modified, wherein contacting occurs in vitro, in situ, ex vivo, or in vivo; and wherein contacting occurs ex vivo in a cell obtained from the subject or progeny thereof and wherein the method further comprises administering cell or obtained from the subject or progeny to the subject after contacting the cell or progeny thereof with the engineered targeting system.

159. (canceled)

160. (canceled)

161. A method of generating a modified organism, the method comprising:
modifying one or more target polynucleotides in a cell by a method as in claim 149, wherein the organism is a non-human animal or a plant.

162. (canceled)

163. (canceled)

164. A method of identifying a trait of interest in an organism where the trait of interest is encoded by one or more target polynucleotides, the method comprising:
contacting the organism or a sample therefrom comprising polynucleotides with an engineered nucleic acid targeting system of claim 105, wherein the engineered nucleic acid targeting system is directed to the one or more target sequences by the guide nucleic acid guide molecule(s) of the engineered nucleic acid targeting system, whereby one or more target polynucleotides, and thereby the one or more traits, are identified, wherein one or more target polynucleotides are modified by the engineered nucleic acid targeting system; wherein the method is performed in vitro, in situ, ex vivo, or in vivo; and wherein the organism is a plant, non-human animal, or human.

165. (canceled)

166. (canceled)

167. (canceled)

168. A method of identifying a polynucleotide modifier, the method comprising:
exposing one or more polynucleotides to one or more candidate agents; and
detecting one or more modified polynucleotides by contacting the one or more polynucleotides exposed to one or more candidate agents with an engineered nucleic acid targeting system of claim 105, wherein the engineered nucleic acid targeting system is directed to the one or more target sequences of one or more modified target polynucleotides present in the sample by the guide nucleic acid guide molecule(s) of the engineered nucleic acid targeting system, whereby one or more modified target polynucleotides present in the sample are identified.

169. A method of detecting one or more target polynucleotide present in a sample comprising polynucleotides, the method comprising:
contacting, in vitro, one or more target polynucleotides present in the sample with an engineered nucleic acid targeting system of claim 105, wherein the engineered nucleic acid targeting system is directed to the one or more target sequences of one or more target polynucleotides present in the sample by the guide nucleic acid guide molecule(s) of the engineered nucleic acid targeting system, whereby one or more target polynucleotides present in the sample are identified.
